(19) United States
(12) Patent Application Publication
(10) Pub. No.: US 2025/0059293 A1
(43) Pub. Date: Feb. 20, 2025

(54) CD73 INHIBITOR AND A2A/A2B ADENOSINE RECEPTOR INHIBITOR COMBINATION THERAPY

(71) Applicant: Incyte Corporation, Wilmington, DE (US)

(72) Inventors: Horacio G. Nastri, West Chester, PA (US); Shaun M. Stewart, Chadds Ford, PA (US); Juan Carlos Almagro, Cambridge, MA (US); Jing Zhou, Boxborough, MA (US); Rebecca A. Buonpane, Wilmington, DE (US); Hui Wang, Hockessin, DE (US); Yingnan Chen, Wilmington, DE (US); Xiaozhao Wang, Moorestown, NJ (US); Peter Niels Carlsen, Claymont, DE (US); Yong Li, Newark, DE (US); Chao Qi, Newark, DE (US); Liangxing Wu, Wilmington, DE (US); Wenqing Yao, Chadds Ford, PA (US); Wenyu Zhu, Media, PA (US); Taisheng Huang, Wilmington, DE (US)

(21) Appl. No.: 18/768,783

(22) Filed: Jul. 10, 2024

Related U.S. Application Data
(63) Continuation of application No. 17/138,306, filed on Dec. 30, 2020, now Pat. No. 12,060,433.
(60) Provisional application No. 62/956,840, filed on Jan. 3, 2020.

(51) Int. Cl. C07K 16/28; A61K 31/135; A61K 31/4985; A61K 39/00; A61P 35/00

(52) U.S. Cl. CPC C07K 16/2896; A61K 31/135; A61K 31/4985; A61P 35/00; A61K 2039/505

(57) ABSTRACT

Disclosed are combination therapies comprising administration of a CD73 inhibitor and an adenosine A2A or A2B receptor inhibitor. The disclosed combination therapies are useful in the treatment of diseases related to the activity of adenosine receptors and/or CD73 including, for example, cancer, inflammatory diseases, cardiovascular diseases, and neurodegenerative diseases. Anti-CD73 antibodies and A2A/A2B inhibitors are also disclosed.

### RELATED APPLICATIONS

This application is a continuation application of and claims priority to U.S. application Ser. No. 17/138,306, filed on Dec. 30, 2020, which claims the benefit of U.S. Provisional Application No. 62/956,840, filed Jan. 3, 2020, each of which is incorporated herein by reference in its entirety.

### SEQUENCE LISTING

This application contains a Sequence Listing that has been submitted electronically as an XML file named “20443-0645002_ST26_SL.XML.” The XML file, created on Jul. 10, 2024, is 103,516 bytes in size. The material in the XML file is hereby incorporated by reference in its entirety.

### TECHNICAL FIELD

Disclosed are combination therapies comprising administration of a CD73 inhibitor and an A2A and/or A2B adenosine receptor inhibitor. The disclosed combination therapies are useful in the treatment of diseases related to the activity of A2A and/or A2B adenosine receptors and/or CD73 including, for example, cancer, inflammatory diseases, cardiovascular diseases, and neurodegenerative diseases. Anti-CD73 inhibitors and A2A/A2B adenosine receptor inhibitors are also disclosed.

### BACKGROUND

Cluster of differentiation 73 (CD73) is a glycosyl phosphatidyl inositol- (GPI-) linked membrane protein that catalyzes the conversion of extracellular adenosine monophosphate (AMP) to adenosine. It functions as a homodimer, and can be shed and is active as a soluble protein in circulation. In addition to its enzymatic function, CD73 also is a cellular adhesion molecule and plays a role in regulation of leukocyte trafficking. CD73 levels are known to be upregulated due to tissue injury or hypoxic conditions, and a number of solid tumors have elevated CD73 levels. Upregulation of CD73 within the tumor contributes to the adenosine-rich tumor microenvironment, which has numerous pro-tumor and immuno-suppressive effects.

Adenosine is an extracellular signaling molecule that can modulate immune responses through many immune cell types. Adenosine was first recognized as a physiologic regulator of coronary vascular tone by Drury and Szent-Györgyu (Sachdeva, S. and Gupta, M. Saudi Pharmaceutical Journal, 2013, 21, 245-253), however it was not until 1970 that Sattin and Rall showed that adenosine regulates cell function via occupancy of specific receptors on the cell surface (Sattin, A., and Rall, T. W., 1970. Mol. Pharmacol. 6, 13-23; Haskó, G., at al., 2007, Pharmacol. Ther. 113, 264-275).

Adenosine plays a vital role in various other physiological functions. It is involved in the synthesis of nucleic acids, when linked to three phosphate groups; it forms ATP, the integral component of the cellular energy system. Adenosine can be generated by the enzymatic breakdown of extracellular ATP, or can be also released from injured neurons and glial cells by passing the damaged plasma membrane (Tautenhahn, M. et al. Neuropharmacology, 2012, 62, 1756-1766). Adenosine produces various pharmacological effects, both in periphery and in the central nervous system, through an action on specific receptors localized on cell membranes (Matsumoto, T. et al. Pharmacol. Res., 2012, 65, 81-90). Alternative pathways for extracellular adenosine generation have been described. These pathways include the production of adenosine from nicotinamide dinucleotide (NAD) instead of ATP by the concerted action of CD38, CD203a and CD73. CD73-independent production of adenosine can also occur by other phosphates such as alkaline phosphatase or prostate-specific phosphatase.

There are four known subtypes of adenosine receptor in humans including A1, A2A, A2B and A3 receptors. A1 and A2A are high affinity receptors, whereas A2B and A3 are low affinity receptors. Adenosine and its agonists can act via one or more of these receptors and can modulate the activity of adenylate cyclase, the enzyme responsible for increasing cyclic AMP (cAMP). The different receptors have differential stimulatory and inhibitory effects on this enzyme. Increased intracellular concentrations of cAMP can suppress the activity of immune and inflammatory cells (Livingston, M. et al., Inflamm. Res., 2004, 53, 171-178).

The A2A adenosine receptor can signal in the periphery and the CNS, with agonists explored as anti-inflammatory drugs and antagonists explored for neurodegenerative diseases (Carlsson, J. et al., J. Med. Chem., 2010, 53, 3748-3755). In most cell types the A2A subtype inhibits intracellular calcium levels whereas the A2B potentiates them. The A2A receptor generally appears to inhibit inflammatory response from immune cells (Borrmann, T. et al., J. Med. Chem., 2009, 52(13), 3994-4006).

A2B receptors are highly expressed in the gastrointestinal tract, bladder, lung and on mast cells (Antonioli, L. et al., Nature Reviews Cancer, 2013, 13, 842-857). The A2B receptor, although structurally closely related to the A2A receptor and able to activate adenylate cyclase, is functionally different. It has been postulated that this subtype may utilize signal transduction systems other than adenylate cyclase (Livingston, M. et al., Inflamm. Res., 2004, 53, 171-178). Among all the adenosine receptors, the A2B adenosine receptor is a low affinity receptor that is thought to remain silent under physiological conditions and to be activated in consequence of increased extracellular adenosine levels (Ryzhov, S. et al. Neoplasia, 2008, 10, 987-995). Activation of A2B adenosine receptor can stimulate adenylate cyclase and phospholipase C through activation of Gs and Gq proteins, respectively. Coupling to mitogen activated protein kinases has also been described (Borrmann, T. et al., J. Med. Chem., 2009, 52(13), 3994-4006).

In the immune system, engagement of adenosine signaling can be a critical regulatory mechanism that protects tissues against excessive immune reactions. Adenosine can negatively modulate immune responses through many immune cell types, including T-cells, natural-killer cells, macrophages, dendritic cells, mast cells and myeloid-derived suppressor cells (Allard, B. et al. Current Opinion in Pharmacology, 2016, 29, 7-16).

In tumors, this pathway is hijacked by the tumor micro-environment and sabotages the antitumor capacity of the immune system, promoting cancer progression. In the tumor micro-environment, adenosine is mainly generated from extracellular ATP by two ectonucleotidases CD39 and CD73. Multiple cell types can generate adenosine by expressing CD39 and CD73. This is the case for tumor cells, T-effector cells, T-regulatory cells, tumor associated macrophages, myeloid derived suppressive cells (MDSCs), endothelial cells, cancer-associated fibroblast (CAFs) and mesenchymal stromal/stem cells (MSCs). Additionally, hypoxia and inflammation, conditions common to the tumor micro-environment induces expression of CD39 and CD73, leading to increased adenosine production. As a result, the adenosine level in solid tumors is higher compared to normal physiological conditions.

A2A are mostly expressed on lymphoid-derived cells, including T-effector cells, T regulatory cells and natural killer (NK) cells. Blocking A2A receptor can prevent downstream immunosuppressive signals that temporarily inactivate T cells. A2B receptors are mainly expressed on monocyte-derived cells including dendritic cells, tumor-associated macrophages, myeloid derived suppressive cells (MDSCs), and mesenchymal stromal/stem cells (MSCs). Blocking A2B receptor in preclinical models can suppress tumor growth, block metastasis, and increase the presentation of tumor antigens.

In terms of safety profile of ADORA2A/ADORA2B (A2A/A2B) blockage, the A2A and A2B receptor knockout (KO) mice are all viable, showing no growth abnormalities and are fertile (Allard, B. et al. Current Opinion in Pharmacology, 2016, 29, 7-16). A2A KO mice displayed increased levels of pro-inflammatory cytokines only upon challenge with lipopolysaccharides (LPS) and no evidence of inflammation at baseline (Antonioli, L. et al., Nature Reviews Cancer, 2013, 13, 842-857). A2B KO mice exhibited normal platelet, red blood, and white blood cell counts but increased inflammation at baseline such as TNF-alpha and IL-6)(Antonioli, L. et al., Nature Reviews Cancer, 2013, 13, 842-857). Further increase in production of TNF-alpha and IL-6 was detected following LPS treatment. A2B KO mice also exhibited increased vascular adhesion molecules that mediate inflammation as well leukocyte adhesion/rolling; enhanced mast-cell activation; increased sensitivity to IgE-mediated anaphylaxis and increased vascular leakage and neutrophil influx under hypoxia (Antonioli, L. et al., Nature Reviews Cancer, 2013, 13, 842-857).

### SUMMARY

In a first aspect, the disclosure provides a method for treating a cancer in a human subject in need thereof, comprising administering to the human subject an effective amount of an inhibitor of human CD73 and an inhibitor of A2A adenosine receptor and/or A2B adenosine receptor.

In some embodiments, the (1) the inhibitor of human CD73 comprises:
(a) an antibody comprising a variable heavy (VH) domain comprising VH complementarity determining region (CDR)1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
comprising a variable light (VL) domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6);
(b) an antibody that binds to human CD73 at an epitope within amino acids 40-53 of SEQ ID NO:70;
(c) an antibody that binds to human CD73 and competes for binding to human CD73 with an antibody that has a heavy chain comprising the amino acid sequence of SEQ ID NO:24 and a light chain comprising the amino acid sequence of SEQ ID NO:25; or
(d) an antibody comprising a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
an antibody comprising a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) an antibody that binds to human CD73 at an epitope within amino acids 386-399 and 470-489 of SEQ ID NO:70;
(f) an antibody that binds to human CD73 and competes for binding to human CD73 with an antibody that has a heavy chain comprising the amino acid sequence of SEQ ID NO:30 and a light chain comprising the amino acid sequence of SEQ ID NO:31;
(g) an antibody that binds to human CD73 and competes for binding to human CD73 with an antibody that has a heavy chain comprising the amino acid sequence of SEQ ID NO:33 and a light chain comprising the amino acid sequence of SEQ ID NO:31;
(h) an antibody selected from the group consisting of 11E1, Medi9447, CPI-006, and BMS-986179; or
(i) an inhibitor selected from the group consisting of CB-708 and AB680.

In some embodiments, the inhibitor of A2A adenosine receptor and/or A2B adenosine receptor (A2A/A2B) comprises a compound of:
(a) Formula (I):

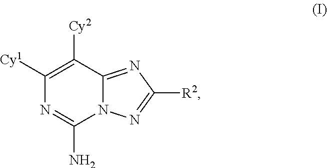

or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl;
(b) Formula (II):

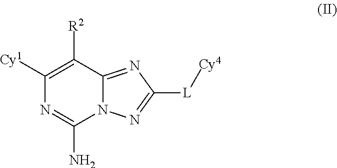

or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{a81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl;
(c) Formula (III):

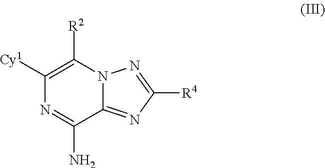

or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl; or (d) Formula (IV):

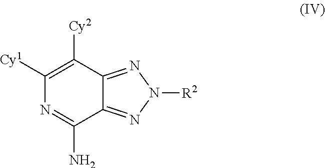

or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl,
or a pharmaceutically acceptable salt thereof.

In some embodiments, the inhibitor of human CD73 comprises:
(a) an antibody comprising a variable heavy (VH) domain comprising VH complementarity determining region (CDR)1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
comprising a variable light (VL) domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and the VL CDR3 comprises the amino acid sequence QQHYNTPYT (SEQ ID NO:6);
(b) an antibody that binds to human CD73 at an epitope within amino acids 40-53 of SEQ ID NO:70;
(c) an antibody that binds to human CD73 and competes for binding to human CD73 with an antibody that has a heavy chain comprising the amino acid sequence of SEQ ID NO:24 and a light chain comprising the amino acid sequence of SEQ ID NO:25; or
(d) an antibody comprising a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
an antibody comprising a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) an antibody that binds to human CD73 at an epitope within amino acids 386-399 and 470-489 of SEQ ID NO:70;
(f) an antibody that binds to human CD73 and competes for binding to human CD73 with an antibody that has a heavy chain comprising the amino acid sequence of SEQ ID NO:30 and a light chain comprising the amino acid sequence of SEQ ID NO:31;
(g) an antibody that binds to human CD73 and competes for binding to human CD73 with an antibody that has a heavy chain comprising the amino acid sequence of SEQ ID NO:33 and a light chain comprising the amino acid sequence of SEQ ID NO:31;
(h) an antibody selected from the group consisting of 11E1, Medi9447, CPI-006, and BMS-986179; or
(i) an inhibitor selected from the group consisting of CB-708 and AB680; and the inhibitor of A2A adenosine receptor and/or A2B adenosine receptor (A2A/A2B) comprises a compound of:
(a) Formula (I):

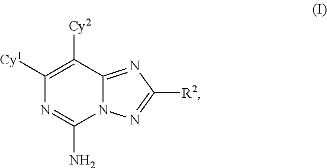

or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl;
(b) Formula (II):

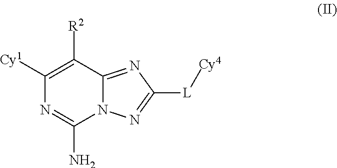

or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl;
(c) Formula (III):

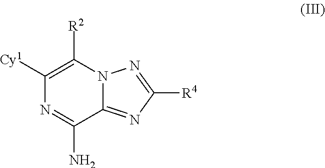

or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl; or
(d) Formula (IV):

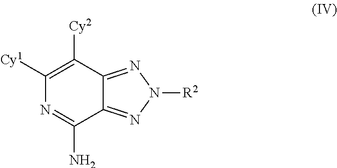

or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl.
or a pharmaceutically acceptable salt thereof.

In some embodiments, the inhibitor of A2A/A2B is a compound of Formula (I):

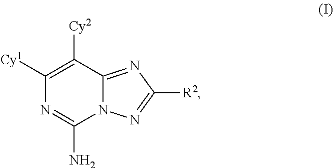

or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl. In some instances, the inhibitor of A2A/A2B is selected from: 3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof; 3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof, 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof, 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof, 3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof, and 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile, or a pharmaceutically acceptable salt thereof.

In some embodiments, the inhibitor of A2A/A2B is a compound of Formula (II):

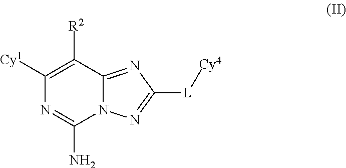

or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents; each R^{8B} is independently selected from halo and C₁₋₃ alkyl; and
each R^{8D} is independently selected from OH, CN, halo, C₁₋₆ alkyl, and C₁₋₆ haloalkyl. In some instances, the inhibitor of A2A/A2B is selected from: 3-(5-Amino-2-(hydroxy(phenyl)methyl)-[1,2,4]triazolo[1,5-c]pyrimidin-7-yl)benzonitrile, or a pharmaceutically acceptable salt thereof; 3-(5-Amino-2-((2,6-difluorophenyl)(hydroxy)methyl)-[1,2,4]triazolo[1,5-c]pyrimidin-7-yl)-2-fluorobenzonitrile, or a pharmaceutically acceptable salt thereof; 5-Amino-7-(3-cyano-2-fluorophenyl)-2-((2,6-difluorophenyl)(hydroxy)methyl)-[1,2,4]triazolo[1,5-c]pyrimidine-8-carbonitrile, or a pharmaceutically acceptable salt thereof; and 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile, or a pharmaceutically acceptable salt thereof.

In some embodiments, the inhibitor of A2A/A2B is a compound of Formula (III):

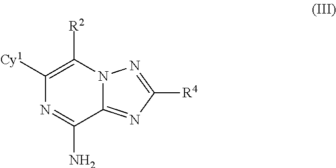

or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl. In some instances, the inhibitor of A2A/A2B is selected from: 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile; 3-(8-Amino-2-((2,6-difluorophenyl)(hydroxy)methyl)-5-(pyrimidin-4-yl)-[1,2,4]triazolo[1,5-a]pyrazin-6-yl)benzonitrile, or a pharmaceutically acceptable salt thereof, 3-(8-amino-2-(amino(2,6-difluorophenyl)methyl)-5-(4-methyloxazol-5-yl)-[1,2,4]triazolo[1,5-a]pyrazin-6-yl)benzonitrile, or a pharmaceutically acceptable salt thereof, and 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile, or a pharmaceutically acceptable salt thereof.

In some embodiments, the inhibitor of A2A/A2B is a compound of Formula (IV):

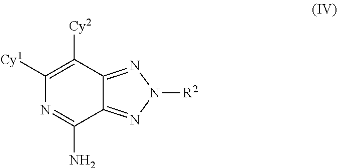

or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl,
or a pharmaceutically acceptable salt thereof. In some instances, the inhibitor of A2A/A2B is selected from: 3-(4-amino-2-(pyridin-2-ylmethyl)-7-(pyrimidin-4-yl)-2H-[1,2,3]triazolo[4,5-c]pyridin-6-yl)benzonitrile, or a pharmaceutically acceptable salt thereof; 3-(4-amino-2-((3-fluoropyridin-2-yl)methyl)-7-(pyrimidin-4-yl)-2H-[1,2,3]triazolo[4,5-c]pyridin-6-yl)benzonitrile, or a pharmaceutically acceptable salt thereof, 3-(4-amino-2-((3-fluoropyridin-2-yl)methyl)-7-(pyridin-4-yl)-2H-[1,2,3]triazolo[4,5-c]pyridin-6-yl)benzonitrile, or a pharmaceutically acceptable salt thereof; and 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile, or a pharmaceutically acceptable salt thereof.

In some embodiments, the inhibitor of A2A/A2B is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof.

In some embodiments, the inhibitor of A2A/A2B is 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof.

In some embodiments, the inhibitor of A2A/A2B is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof.

In some embodiments, the inhibitor of human CD73 comprises an antibody comprising:
a VH domain comprising VH CDR1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
a VL domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and the VL CDR3 comprises the amino acid sequence QQHYNTPYT (SEQ ID NO:6).

In some embodiments, the inhibitor of human CD73 comprises an antibody that binds to human CD73 at an epitope within amino acids 40-53 of SEQ ID NO:70.

In some embodiments, the inhibitor of human CD73 comprises an antibody that binds to human CD73 and competes for binding to human CD73 with an antibody that has a heavy chain comprising the amino acid sequence of SEQ ID NO:24 and a light chain comprising the amino acid sequence of SEQ ID NO:25.

In some embodiments, the inhibitor of human CD73 comprises an antibody comprising:
a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39).

In some embodiments, the inhibitor of human CD73 comprises an antibody that binds to human CD73 at an epitope within amino acids 386-399 and 470-489 of SEQ ID NO:70.

In some embodiments, the inhibitor of human CD73 comprises an antibody that binds to human CD73 and competes for binding to human CD73 with an antibody that has a heavy chain comprising the amino acid sequence of SEQ ID NO:30 and a light chain comprising the amino acid sequence of SEQ ID NO:31.

In some embodiments, the inhibitor of human CD73 comprises an antibody that binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the inhibitor of human CD73 comprises an antibody selected from the group consisting of 11E1, Medi9447, CPI-006, and BMS-986179.

In some embodiments, the inhibitor of human CD73 is selected from the group consisting of CB-708 and AB680.

In some embodiments, the inhibitor of human CD73 comprises an antibody comprising a VH domain comprising the amino acid sequence set forth in SEQ ID NO:22 and a VL domain comprising the amino acid sequence set forth in SEQ ID NO:23, and the inhibitor of A2A/A2B comprises 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the inhibitor of human CD73 comprises an antibody comprising a heavy chain and a light chain, and wherein the heavy chain comprises the amino acid sequence set forth in SEQ ID NO:24 and the light chain comprises the amino acid sequence set forth in SEQ ID NO:25, and the inhibitor of A2A/A2B comprises 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the inhibitor of human CD73 comprises an antibody comprising a VH domain comprising the amino acid sequence set forth in SEQ ID NO:62 and a VL domain comprising the amino acid sequence set forth in SEQ ID NO:61, and the inhibitor of A2A/A2B comprises 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the inhibitor of human CD73 comprises an antibody comprising a heavy chain and a light chain, and wherein the heavy chain comprises the amino acid sequence set forth in SEQ ID NO:30 and the light chain comprises the amino acid sequence set forth in SEQ ID NO:31, and the inhibitor of A2A/A2B comprises 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the inhibitor of human CD73 comprises an antibody comprising a VH domain comprising the amino acid sequence set forth in SEQ ID NO:63 and a VL domain comprising the amino acid sequence set forth in SEQ ID NO:61, and the inhibitor of A2A/A2B comprises 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the inhibitor of human CD73 comprises an antibody comprising a heavy chain and a light chain, and wherein the heavy chain comprises the amino acid sequence set forth in SEQ ID NO:33 and the light chain comprises the amino acid sequence set forth in SEQ ID NO:31, and the inhibitor of A2A/A2B comprises 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In a second aspect, the disclosure provides a method for treating a cancer in a human subject in need thereof, comprising administering to the human subject an effective amount of an antibody that binds to human CD73 and an inhibitor of A2A adenosine receptor and/or A2B adenosine receptor, wherein the antibody:
(a) comprises a VH domain comprising VH CDR1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
comprises a VL domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6);
(b) binds to human CD73 at an epitope within amino acids 40-53 of SEQ ID NO:70;
(c) binds to human CD73 and competes for binding to human CD73 with an antibody that has a heavy chain comprising the amino acid sequence of SEQ ID NO:24 and a light chain comprising the amino acid sequence of SEQ ID NO:25;
(d) comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) binds to human CD73 at an epitope within amino acids 386-399 and 470-489 of SEQ ID NO:70;
(f) binds to human CD73 and competes for binding to human CD73 with an antibody that has a heavy chain comprising the amino acid sequence of SEQ ID NO:30 and a light chain comprising the amino acid sequence of SEQ ID NO:31; or
(g) binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the antibody comprises a VH domain comprising VH CDR1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
wherein the antibody comprises a VL domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6). In some instances, the VH domain comprises the amino acid sequence set forth in SEQ ID NO:22. In some instances, the antibody comprises a heavy chain and wherein the heavy chain comprises the amino acid sequence set forth in SEQ ID NO:24. In some instances, the VL domain comprises the amino acid sequence set forth in SEQ ID NO:23. In some instances, the antibody comprises a light chain and wherein the light chain comprises the amino acid sequence set forth in SEQ ID NO:25. In some instances, the VH domain is at least 80% identical to the amino acid sequence set forth in SEQ ID NO:22 and the VL domain is at least 80% identical to the amino acid sequence set forth in SEQ ID NO:23. In some instances, the VH domain comprises the amino acid sequence set forth in SEQ ID NO:22 and the VL domain comprises the amino acid sequence set forth in SEQ ID NO:23. In some instances, the antibody comprises a heavy chain and a light chain, and wherein the heavy chain comprises the amino acid sequence set forth in SEQ ID NO:24 and the light chain comprises the amino acid sequence set forth in SEQ ID NO:25.

In some embodiments, the antibody binds to human CD73 at an epitope within amino acids 40-53 of SEQ ID NO:70.

In some embodiments, the antibody binds to human CD73 and competes for binding to human CD73 with an antibody that has a heavy chain comprising the amino acid sequence of SEQ ID NO:24 and a light chain comprising the amino acid sequence of SEQ ID NO:25.

In some embodiments, the antibody comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39).

In some embodiments, the antibody comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35);
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39). In some instances, the VH domain comprises the amino acid sequence set forth in SEQ ID NO: 62. In some instances, the antibody comprises a heavy chain and wherein the heavy chain comprises the amino acid sequence set forth in SEQ ID NO:30. In some instances, the VL domain comprises the amino acid sequence set forth in SEQ ID NO: 61. In some instances, the antibody comprises a light chain and wherein the light chain comprises the amino acid sequence set forth in SEQ ID NO: 31. In some instances, the VH domain is at least 80% identical to the amino acid sequence set forth in SEQ ID NO: 62 and the VL domain is at least 80% identical to the amino acid sequence set forth in SEQ ID NO: 61. In some instances, the VH domain comprises the amino acid sequence set forth in SEQ ID NO: 62 and the VL domain comprises the amino acid sequence set forth in SEQ ID NO: 61. In some instances, the antibody comprises a heavy chain and a light chain, and wherein the heavy chain comprises the amino acid sequence set forth in SEQ ID NO: 30 and the light chain comprises the amino acid sequence set forth in SEQ ID NO: 31.

In some embodiments, the antibody comprises a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYEGSNK (SEQ ID NO:40);
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39). In some instances, the VH domain comprises the amino acid sequence set forth in SEQ ID NO: 63. In some instances, the antibody comprises a heavy chain and wherein the heavy chain comprises the amino acid sequence set forth in SEQ ID NO:33. In some instances, the VL domain comprises the amino acid sequence set forth in SEQ ID NO: 61. In some instances, the antibody comprises a light chain and wherein the light chain comprises the amino acid sequence set forth in SEQ ID NO: 31. In some instances, the VH domain is at least 80% identical to the amino acid sequence set forth in SEQ ID NO: 63 and the VL domain is at least 80% identical to the amino acid sequence set forth in SEQ ID NO: 61. In some instances, the VH domain comprises the amino acid sequence set forth in SEQ ID NO: 63 and the VL domain comprises the amino acid sequence set forth in SEQ ID NO: 61. In some instances, the antibody comprises a heavy chain and a light chain, and wherein the heavy chain comprises the amino acid sequence set forth in SEQ ID NO: 33 and the light chain comprises the amino acid sequence set forth in SEQ ID NO: 31.

In some embodiments, the antibody binds to human CD73 at an epitope within amino acids 386-399 and 470-489 of SEQ ID NO:70.

In some embodiments, the antibody binds to human CD73 and competes for binding to human CD73 with an antibody that has a heavy chain comprising the amino acid sequence of SEQ ID NO:30 and a light chain comprising the amino acid sequence of SEQ ID NO:31.

In some embodiments, the antibody binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments, the inhibitor is selected from the group consisting of: 7-(5-methylfuran-2-yl)-3-[[6-[[(3S)-oxolan-3-yl]oxymethyl]pyridin-2-yl]methyl]triazolo[4,5-d]pyrimidin-5-amine; 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile; 3-[2-Amino-6-[1-[[6-(2-hydroxypropan-2-yl)pyridin-2-yl]methyl]triazol-4-yl]pyrimidin-4-yl]-2-methylbenzonitrile; 6-(2-Chloro-6-methylpyridin-4-yl)-5-(4-fluorophenyl)-1,2,4-triazin-3-amine, 5-Bromo-2,6-di(1H-pyrazol-1-yl)pyrimidin-4-amine; and EOS100850.

In some embodiments, the inhibitor is selected from the group consisting of: 7-(5-methylfuran-2-yl)-3-[[6-[[(3S)-oxolan-3-yl]oxymethyl]pyridin-2-yl]methyl]triazolo[4,5-d]pyrimidin-5-amine; 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile; 3-[2-Amino-6-[1-[[6-(2-hydroxypropan-2-yl)pyridin-2-yl]methyl]triazol-4-yl]pyrimidin-4-yl]-2-methylbenzonitrile; 6-(2-Chloro-6-methylpyridin-4-yl)-5-(4-fluorophenyl)-1,2,4-triazin-3-amine, 5-Bromo-2,6-di(1H-pyrazol-1-yl)pyrimidin-4-amine; and EOS100850.

In some embodiments of the foregoing methods, the cancer has a high adenosine signature. In some embodiments of the foregoing methods, the cancer is head and neck cancer, colorectal cancer, lung cancer, melanoma, ovarian, bladder, liver cancer, or renal cell carcinoma.

### BRIEF DESCRIPTION OF THE DRAWINGS

FIG. 1A shows the heavy chain variable domain (VH) and light chain variable domain (VL) amino acid sequences for humanized CL25 antibodies CL_hu10-4, HzCL25, CL25_hu_10-6, and CL25_hu_11-4.

FIG. 1B shows the VH and VL amino acid sequences for humanized CL25 antibodies CL25_hu_11-5, CL25_hu_11-6, CL25_hu_8-4, and CL25_hu_8-5.

FIG. 1C shows the VH and VL amino acid sequences for humanized CL25 antibodies CL25_hu_8-6, CL25_hu_9-4, CL25_hu_9-5, and CL25_hu_9-6.

FIG. 1D shows an alignment of the VH for CL25 and humanized CL25 antibodies. CDRs according to the IMGT definition are underlined.

FIG. 1E shows an alignment of the VL for CL25 and humanized CL25 antibodies. CDRs according to the IMGT definition are underlined.

FIG. 2A is a graph depicting the cell binding (measured by geometric mean fluorescence intensity [GMFI]) for the indicated antibodies at the indicated concentrations on MDA-MB-231 cells.

FIG. 2B is a graph depicting the cell binding (measured by GMFI) for CL25 or isotype control (iso ctrl) at the indicated concentrations on A375 cells.

FIG. 3A is a graph depicting the cellular CD73 inhibition on A375 cells treated with the indicated antibodies or isotype control (iso ctrl) at the indicated concentrations.

FIG. 3B is a graph depicting the cellular CD73 inhibition on MDA-MB-231 cells treated with the indicated antibodies or isotype control (iso ctrl) at the indicated concentrations.

FIG. 3C is a graph depicting the cellular CD73 inhibition on MDA-MB-231 cells treated with the indicated antibodies or isotype control (iso ctrl) at the indicated concentrations.

FIG. 4 is a graph depicting inhibition of recombinant CD73 treated with the indicated antibodies or isotype control (iso ctrl) at the indicated concentrations.

FIG. 5 is a map of the crystal structure of human CD73 (4H2F.pdb) with the CL25 antibody epitope indicated in dark grey (with arrows).

FIG. 6 is a graph depicting surface CD73 levels after 24 hour incubation with the indicated antibody or isotype control (iso ctrl), or without treatment (NT), as measured with a directly conjugated non-competing antibody.

FIG. 7A is a graph depicting the cell binding (GMFI) for antibody 3-F03 at the indicated concentrations on MDA-MB-231 cells.

FIG. 7B is a graph depicting the cell binding (measured by GMFI) for 3-F03 or isotype control (Iso ctrl) at the indicated concentrations on A375 cells.

FIG. 8A is a graph depicting the cellular CD73 inhibition on A375 cells treated with 3-F03 or isotype control (Iso ctrl) at the indicated concentrations.

FIG. 8B is a graph depicting the cellular CD73 inhibition on MDA-MB-231 cells treated with 3-F03 or isotype control (Iso ctrl) at the indicated concentrations.

FIG. 9 is a graph depicting inhibition of recombinant CD73 treated with 3-F03 or isotype control (Iso control) at the indicated concentrations.

FIG. 10 is a map of the crystal structure of human CD73 (4H2F.pdb) with the 3-F03 antibody epitope indicated in dark grey (with arrows).

FIG. 11 is a graph depicting CD73 surface levels after 24 hour incubation with 3-F03, isotype control (iso ctrl), or not treated (NT) as measured with a directly conjugated non-competing antibody, CL43-Dy650.

FIGS. 12A-12J show the VH and VL amino acid sequences of 3-F03 and exemplary 3-F03 variants.

FIG. 13 is a graph depicting cell binding (GMFI) for the indicated antibodies at the indicated concentrations on MDA-MB-231 cells.

FIG. 14 is a graph depicting the cellular CD73 inhibition on MDA-MB-231 cells treated with the indicated antibodies or isotype control at the indicated concentrations.

FIG. 15 shows exemplary amino acid sequences of a human IgG1 heavy chain CH1-hinge-CH2-CH3 with an N297A mutation (SEQ ID NO:73), a human IgG1 heavy chain CH1-hinge-CH2-CH3 with an N297A mutation with C-terminal lysine (SEQ ID NO:75), and a human kappa light chain constant region (SEQ ID NO:74).

FIG. 16A is a graph showing the IFNγ homogeneous time-resolved fluorescence (HTRF) signal ratio in CD4⁺ T cells treated with (i) titrated HzCL25, (ii) titrated Compound 9, or (iii) titrated HzCL25 and 0.5 μM of Compound 9.

FIG. 16B is a graph showing the IFNγ HTRF signal ratio in CD4⁺ T cells treated with (i) titrated Antibody A, (ii) titrated Compound 9, or (iii) titrated Antibody A and 0.5 μM of Compound 9.

FIG. 16C is a graph showing the IFNγ HTRF signal ratio in CD4⁺ T cells treated with (i) titrated Antibody B, (ii) titrated Compound 9, or (iii) titrated Antibody B and 0.5 μM of Compound 9.

FIG. 17A is a graph showing the IFNγ HTRF signal ratio in CD4+ T cells treated with (i) titrated HzCL25, (ii) titrated Compound 9, or (iii) titrated Compound 9 and 1 μg/mL of HzCL25.

FIG. 17B is a graph showing the IFNγ HTRF signal ratio in CD4+ T cells treated with (i) titrated HzCL25, (ii) titrated Compound A, or (iii) titrated Compound A and 1 μg/mL of HzCL25.

FIG. 17C is a graph showing the IFNγ HTRF signal ratio in CD4+ T cells treated with (i) titrated HzCL25, (ii) titrated Compound B, or (iii) titrated Compound B and 1 μg/mL of HzCL25.

FIG. 18A is a graph showing the percent CD4⁺ T cell proliferation in donor cells treated with: (i) titrated Compound 9, (ii) titrated HzCL25, or (iii) titrated HzCL25 and 0.5 μM of Compound 9.

FIG. 18B is a graph showing the percent CD4+ T cell proliferation in donor cells treated with: (i) titrated Compound 9, (ii) titrated Antibody A, or (iii) titrated Antibody A and 0.5 μM of Compound 9.

FIG. 18C is a graph showing the percent CD4+ T cell proliferation in donor cells treated with: (i) titrated Compound 9, (ii) titrated Antibody B, or (iii) titrated Antibody B and 0.5 μM of Compound 9.

FIG. 19A is a graph depicting the tumor volume in mice administered (i) human IgG1 and vehicle (circles), or (ii) HzCL25 and Compound 9 (squares).

FIG. 19B is a graph depicting the tumor volume in mice administered (i) human IgG1 and vehicle (circles), or (ii) 3-F03_413 and Compound 9 (squares).

FIG. 20A shows the DNA sequences encoding the HzCL25 heavy chain and light chain.

FIG. 20B shows the DNA sequence encoding the 3-F03_411 heavy chain.

FIG. 20C shows the DNA sequences encoding the 3-F03_411 and 3-F03_413 light chain and 3-F03_413 heavy chain.

### DETAILED DESCRIPTION

Provided herein are combination therapies comprising administration of a CD73 inhibitor and an A2A and/or A2B adenosine receptor inhibitor. The disclosed combination therapies are useful in the treatment of diseases related to the activity of A2A and/or A2B adenosine receptors and/or CD73 including, for example, cancer, inflammatory diseases, cardiovascular diseases, and neurodegenerative diseases. CD73 inhibitors and A2A/A2B adenosine receptor inhibitors are also disclosed.

### CD73

CD73 (also known as “5′-nucleotidase” and “ecto-5′-nucleotidase”) is a dimeric enzyme (EC:3.1.3.5) that functions as a homodimer bound by a GPI linkage to the external face of the plasma membrane. CD73 can be shed and is active as a soluble protein in circulation. CD73 catalyzes the conversion of extracellular AMP to adenosine. CD73 enzymatic activity requires substrate binding in the open CD73 conformation. After the substrate binding, CD73 goes through a large conformational change from open to closed conformation to convert AMP to adenosine (see, e.g., Knapp et al., 2012, Structure, 20(12):2161-73). CD73 also functions as a cellular adhesion molecule and plays a role in regulation of leukocyte trafficking.

CD73 enzymatic activity plays a role in the promotion and metastasis of cancer (see, e.g., Stagg and Smyth, 2010, Oncogene, 29:5346-5358; Salmi and Jalkanen, 2012, OncoImmunology, 1:247-248, 2012; Stagg, 2012, OncoImmunology, 1:217-218; Zhang, 2012, OncoImmunology, 167-70). Overexpression of CD73 in cancer cells impairs adaptive antitumor immune responses, enhancing tumor growth and metastasis (see, e.g., Niemela et al., 2004, J. Immunol., 172:1646-1653; Sadej et al., 2006, Nucleosides Nucleotides Nucleic Acids, 25:1119-1123; Braganhol et al., 2007, Biochim. Biophys. Acta., 1770:1352-1359; Zhang, 2010, Cancer Res., 70:6407-6411; Zhang, 2012, OncoImmunology, 1:67-70).

An exemplary amino acid sequence of the mature human CD73 protein (amino acids 27-549 of GenBank Accession No. NP_002517) is:

(SEQ ID NO: 70)
WELTILHTNDVHSRLEQTSEDSSKCVNASRCMGGVARLFTKVQQIRRAEP
NVLLLDAGDQYQGTIWFTVYKGAEVAHFMNALRYDAMALGNHEFDNGVEG
LIEPLLKEAKFPILSANIKAKGPLASQISGLYLPYKVLPVGDEVVGIVGY
TSKETPFLSNPGTNLVFEDEITALQPEVDKLKTLNVNKIIALGHSGFEMD
KLIAQKVRGVDVVVGGHSNTFLYTGNPPSKEVPAGKYPFIVTSDDGRKVP
VVQAYAFGKYLGYLKIEFDERGNVISSHGNPILLNSSIPEDPSIKADINK
WRIKLDNYSTQELGKTIVYLDGSSQSCRFRECNMGNLICDAMINNNLRHT
DEMFWNHVSMCILNGGGIRSPIDERNNGTITWENLAAVLPFGGTFDLVQL
KGSTLKKAFEHSVHRYGQSTGEFLQVGGIHVVYDLSRKPGDRVVKLDVLC
TKCRVPSYDPLKMDEVYKVILPNFLANGGDGFQMIKDELLRHDSGDQDIN
VVSTYISKMKVIYPAVEGRIKFS.

An exemplary amino acid sequence of the mature murine CD73 protein (amino acids 29-551 of GenBank Accession No. NP_035981) is:

(SEQ ID NO: 71)
WELTILHTNDVHSRLEQTSDDSTKCLNASLCVGGVARLFTKVQQIRKEEP
NVLFLDAGDQYQGTIWFTVYKGLEVAHFMNILGYDAMALGNHEFDNGVEG
LIDPLLRNVKFPILSANIKARGPLAHQISGLFLPSKVLSVGGEVVGIVGY
TSKETPFLSNPGTNLVFEDEISALQPEVDKLKTLNVNKIIALGHSGFEMD
KLIAQKVRGVDIVVGGHSNTFLYTGNPPSKEVPAGKYPFIVTADDGRQVP
VVQAYAFGKYLGYLKVEFDDKGNVITSYGNPILLNSSIPEDATIKADINQ
WRIKLDNYSTQELGRTIVYLDGSTQTCRFRECNMGNLICDAMINNNLRHP
DEMFWNHVSMCIVNGGGIRSPIDEKNNGTITWENLAAVLPFGGTFDLVQL
KGSTLKKAFEHSVHRYGQSTGEFLQVGGIHVVYDINRKPWNRVVQLEVLC
TKCRVPIYEPLEMDKVYKVTLPSYLANGGDGFQMIKDELLKHDSGDQDIS
VVSEYISKMKVVYPAVEGRIKFS.

An exemplary amino acid sequence of the mature cynomolgus CD73 protein is:

(SEQ ID NO: 72)
WELTILHTNDVHSRLEQTSEDSSKCVNASRCMGGVARLFTKVQQIRRAEP
NVLLLDAGDQYQGTIWFTVYKGAEVAHEMNALRYDAMALGNHEFDNGVEG
LIEPLLKEAKFPILSANIKAKGPLASQISGLYLPYKVLPVGDEVVGIVGY
TSKETPFLSNPGTNLVFEDEITALQPEVDKLKTLNVNKIIALGHSGFETD
KLIAQKVRGVDVVVGGHSNTFLYTGNPPSKEVPAGKYPFIVTSDDGRKVP
VVQAYAFGKYLGYLKIEFDERGNVISSHGNPILLNSSIPEDPSIKADINK
WRIKLDNYSTQELGKTIVYLDGSSQSCRFRECNMGNLICDAMINNNLRHA
DEMFWNHVSMCILNGGGIRSPIDERNNGTITWENLAAVLPFGGTFDLVQL
KGSTLKKAFEHSVHRYGQSTGEFLQVGGIHVVYDLSRKPGDRVVKLDVLC
TKCRVPSYDPLKMDEIYKVILPNFLANGGDGFQMIKDELLRHDSGDQDIN
VVSTYISKMKVIYPAVEGRIKFS.

### Anti-CD73 Antibodies

This disclosure provides anti-CD73 antibodies that are useful in combination with an A2A and/or A2B adenosine receptor inhibitor in treating diseases, e.g., cancer. These anti-CD73 antibodies can bind human CD73.

In some instances, these antibodies bind human CD73 and cynomolgus CD73. In some instances, these antibodies bind human CD73 and cynomolgus CD73 and do not bind murine CD73. Such anti-CD73 antibodies include the sequences of an anti-CD73 monoclonal antibody, CL25, and a humanized version thereof, HzCL25, which humanized version thereof binds with high affinity to both human and cynomolgus CD73, and has undetectable binding to mouse CD73.

In some instances, these antibodies bind human CD73, cynomolgus CD73, and murine CD73. Such anti-CD73 antibodies includes the sequences of a human anti-CD73 monoclonal antibody, 3-F03, which binds with high affinity to the open conformation of each of human, cynomolgus, and murine CD73.

### Antibody HzCL25

Antibody HzCL25 is a humanized IgG1/kappa monoclonal antibody with alanine at position Asparagine-297 (N297, according to EU numbering) of the heavy chain constant region to reduce effector function. It specifically binds human and cynomolgus CD73 with high affinity (K_{D}≤0.5 nM) and has low effector functionality.

HzCL25 was constructed from a chimeric version of the CL25 antibody. The CL25 murine heavy chain variable domain (VH) and light chain variable domain (VL) were obtained from a mouse immunized with recombinant human CD73 (SEQ ID NO:70) comprising a HIS-tag. Antibody sequences of the B cells were determined and the murine heavy chain variable domain (VH) (SEQ ID NO:26) and light chain variable domain (VL) (SEQ ID NO:27) were expressed as chimeras with human IgG1 Fc (heavy chain constant region comprising the amino acid sequence of SEQ ID NO:73 and kappa light chain constant region comprising the amino acid sequence of SEQ ID NO:74). Table 1, below, shows the amino acid sequences of the CL25 complementarity determining regions (CDRs) according to IMGT, Chothia, AbM, Kabat, and Contact numbering. Table 1, below, also shows the amino acid sequences of the CL25 mature VH and VL.

TABLE 1
CL25 CDRs, VH, and VL

| | IMGT | Chothia | AbM | Kabat | Contact |
|---|---|---|---|---|---|
| VH | GYTFTSYG | GYTFTSY | GYTFTSYGLS | SYGLS | TSYGLS |
| CDR1 | (SEQ ID NO: 1) | (SEQ ID NO: 7) | (SEQ ID NO: 12) | (SEQ ID NO: 14) | (SEQ ID NO: 16) |
| VH | IYPGSGNT | YPGSGN | EIYPGSGNTY | EIYPGSGNTYY | WIGEIYPGSGN |
| CDR2 | (SEQ ID NO: 2) | (SEQ ID NO: 8) | (SEQ ID NO: 13) | NEKFKG | TY |
| | | | | (SEQ ID NO: 15) | (SEQ ID NO: 28) |
| VH | ARYDYLGSSY | YDYLGSSYGFD | YDYLGSSYGFD | YDYLGSSYGFD | ARYDYLGSSY |
| CDR3 | GFDY | Y | Y | Y | GFD |
| | (SEQ ID NO: 3) | (SEQ ID NO: 9) | (SEQ ID NO: 9) | (SEQ ID NO: 9) | (SEQ ID NO: 18) |
| VL | QDVSTA | KASQDVSTAV | KASQDVSTAV | KASQDVSTAV | STAVAWY |
| CDR1 | (SEQ ID NO: 4) | A | A | A | (SEQ ID NO: 19) |
| | | (SEQ ID NO: 10) | (SEQ ID NO: 10) | (SEQ ID NO: 10) | |
| VL | SAS | SASYRYN | SASYRYN | SASYRYN | LLIYSASYRY |
| CDR2 | (SEQ ID NO: 5) | (SEQ ID NO: 29) | (SEQ ID NO: 29) | (SEQ ID NO: 29) | (SEQ ID NO: 20) |
| VL | QQHYNTPYT | QQHYNTPYT | QQHYNTPYT | QQHYNTPYT | QQHYNTPY |
| CDR3 | (SEQ ID NO: 6) | (SEQ ID NO: 6) | (SEQ ID NO: 6) | (SEQ ID NO: 6) | (SEQ ID NO: 21) |

| | |
|---|---|
| VH | QVQLQQSGAELARPGASVKLSCRASGYTFTSYGLSWVKQRTGQGLEWIGEIYPGSGNTYYNE |
| | KFKGKATLTADKSSSTAYMELRSLTSEDSAVYFCARYDYLGSSYGFDYWGQGTTLTVSS |
| | (SEQ ID NO: 26) |
| VL | DIVMTQSHKFMSTSVGDRVSITCKASQDVSTAVAWYQQKPGQSPKLLIYSASYRYNGVPDRF |
| | TGSGSGTDFTFTISSVQAEDLAVYYCQQHYNTPYTFGGGTKLEIK (SEQ ID NO: 27) |

To construct HzCL25, the CL25 VH and VL sequences were aligned to a database of human VH and VK genes. The CDRs (Table 1) from the murine CL25 antibody were grafted into human VH and VK genes.

Table 2, below, shows the amino acid sequences of the HzCL25 CDRs according to IMGT, Chothia, AbM, Kabat, and Contact numbering. Table 2, below, also shows the amino acid sequences of the HzCL25 mature VH, VL, heavy chain, and light chain.

TABLE 2
Amino acid sequences of HzCL25 CDRs, VH, VL, heavy chain, and light chain

| | IMGT | Chothia | AbM | Kabat | Contact |
|---|---|---|---|---|---|
| VH | GYTFTSYG | GYTFTSY | GYTFTSYGLS | SYGLS | TSYGLS |
| CDR1 | (SEQ ID NO: 1) | (SEQ ID NO: 7) | (SEQ ID NO: 12) | (SEQ ID NO: 14) | (SEQ ID NO: 16) |
| VH | IYPGSGNT | YPGSGN | EIYPGSGNTY | EIYPGSGNTYY | WMGEIYPGSG |
| CDR2 | (SEQ ID NO: 2) | (SEQ ID NO: 8) | (SEQ ID NO: 13) | NEKFKG | NTY |
| | | | | (SEQ ID NO: 15) | (SEQ ID NO: 17) |
| VH | ARYDYLGSSY | YDYLGSSYGFD | YDYLGSSYGFD | YDYLGSSYGFD | ARYDYLGSSY |
| CDR3 | GFDY | Y | Y | Y | GFD |
| | (SEQ ID NO: 3) | (SEQ ID NO: 9) | (SEQ ID NO: 9) | (SEQ ID NO: 9) | (SEQ ID NO: 18) |
| VL | QDVSTA | KASQDVSTAV | KASQDVSTAV | KASQDVSTAV | STAVAWY |
| CDR1 | (SEQ ID NO: 4) | A | A | A | (SEQ ID NO: 19) |
| | | (SEQ ID NO: 10) | (SEQ ID NO: 10) | (SEQ ID NO: 10) | |
| VL | SAS | SASYRYS | SASYRYS | SASYRYS | LLIYSASYRY |
| CDR2 | (SEQ ID NO: 5) | (SEQ ID NO: 11) | (SEQ ID NO: 11) | (SEQ ID NO: 11) | (SEQ ID NO: 20) |
| VL | QQHYNTPYT | QQHYNTPYT | QQHYNTPYT | QQHYNTPYT | QQHYNTPY |
| CDR3 | (SEQ ID NO: 6) | (SEQ ID NO: 6) | (SEQ ID NO: 6) | (SEQ ID NO: 6) | (SEQ ID NO: 21) |

| | |
|---|---|
| VH | EVQLVQSGAEVKKPGESLKISCKGSGYTFTSYGLSWVRQMPGKGLEWMGEIYPGSGNTYYN |
| | EKFKGQVTISADKSISTAYLQWSSLKASDTAMYYCARYDYLGSSYGFDYWGAGTTVTVSS |
| | (SEQ ID NO: 22) |
| VL | DIVMTQSPDSLAVSLGERATINCKASQDVSTAVAWYQQKPGQPPKLLIYSASYRYSGVPDRFS |
| | GSGSGTDFTLTISSLQAEDVAVYYCQQHYNTPYTFGGGTKLEIK (SEQ ID NO: 23) |
| Heavy | EVQLVQSGAEVKKPGESLKISCKGSGYTFTSYGLSWVRQMPGKGLEWMGEIYPGSGNTYYN |
| Chain | EKFKGQVTISADKSISTAYLQWSSLKASDTAMYYCARYDYLGSSYGFDYWGAGTTVTVSSA |
| | STKGPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCPAPELLGGPSVFLFPP |
| | KPKDTLMISRTPEVTCVVVDVSHEDPEVKFNWYVDGVEVHNAKTKPREEQYASTYRVVSVL |
| | TVLHQDWLNGKEYKCKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKSRWQQGNVFSCSVMHE |
| | ALHNHYTQKSLSLSPG (SEQ ID NO: 24) |
| Light | DIVMTQSPDSLAVSLGERATINCKASQDVSTAVAWYQQKPGQPPKLLIYSASYRYSGVPDRFS |
| Chain | GSGSGTDFTLTISSLQAEDVAVYYCQQHYNTPYTFGGGTKLEIKRTVAAPSVFIFPPSDEQLKS |
| | GTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 25) |

The anti-CD73 antibodies can encompass the VH CDR1, VH CDR2, and VH CDR3 and the VL CDR 1, VL CDR2, and VL CDR3 of HzCL25 or CL25. In some instances, the anti-CD73 antibody comprises a VH comprising VH CDR1, VH CDR2, and VH CDR3 of HzCL25 (see Table 2). In some instances, the anti-CD73 antibody comprises a VL comprising VL CDR1, VL CDR2, and VL CDR3 of HzCL25 (see Table 2). In some instances, the anti-CD73 antibody comprises a VH comprising VH CDR1, VH CDR2, and VH CDR3 of HzCL25 (see Table 2) and a VL comprising VL CDR1, VL CDR2, and VL CDR3 of HzCL25 (see Table 2). In some instances, the anti-CD73 antibody comprises a VH comprising VH CDR1, VH CDR2, and VH CDR3 of CL25 (see Table 1). In some instances, the anti-CD73 antibody comprises a VL comprising VL CDR1, VL CDR2, and VL CDR3 of CL25 (see Table 1). In some instances, the anti-CD73 antibody comprises a VH comprising VH CDR1, VH CDR2, and VH CDR3 of CL25 (see Table 1) and a VL comprising VL CDR1, VL CDR2, and VL CDR3 of CL25 (see Table 1). In some instances, the anti-CD73 antibodies can have, e.g., 1, 2, or 3 substitutions within one or more (i.e., 1, 2, 3, 4, 5, or 6) of the six CDRs of HzCL25 or CL25. In some instances, the antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 3); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 4); and/or (iii) bind human or cynomolgus monkey CD73 in the open conformation with high affinity (e.g., K_{D}≤0.5 nM) but do not significantly bind CD73 in the open conformation from mice (e.g., as determined by the binding assay described in Example 5); and/or (iv) bind human or cynomolgus monkey CD73 in the closed conformation with high affinity (e.g., K_{D}≤0.5 nM) but do not significantly bind CD73 in the closed conformation from mice; and/or (v) bind to an epitope within amino acids 40-53 of SEQ ID NO:70 (i.e., within TKVQQIRRAEPNVL (SEQ ID NO:76)) (e.g., as determined by the binding assay described in Example 5); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 16); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 17); and/or (ix) reduce free surface CD73 on cells (e.g., cancer cells, e.g., melanoma cancer cancers, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control).

The anti-CD73 antibodies can comprise the VH CDR1, VH CDR2, and VH CDR3 of HzCL25 or CL25 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VH CDR1 and/or VH CDR2 and/or VH CDR3 of HzCL25 or CL25. In some embodiments, the anti-CD73 antibodies further comprise the VL CDR1, VL CDR2, and VL CDR3 of HzCL25 or CL25 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VL CDR1 and/or VL CDR2 and/or VL CDR3 of HzCL25 or CL25. In some instances, the anti-CD73 antibody comprises a VH comprising the VH CDR1, VH CDR2, and VH CDR3 set forth in SEQ ID NOs: 1, 2, and 3, respectively, and a VL comprising the VL CDR1, VL CDR2, and VL CDR3 set forth in SEQ ID NOs: 4, 5, and 6, respectively. In some instances, the anti-CD73 antibody comprises a VH comprising the VH CDR1, VH CDR2, and VH CDR3 set forth in SEQ ID NOs: 7, 8, and 9, respectively, and a VL comprising the VL CDR1, VL CDR2, and VL CDR3 set forth in SEQ ID NOs: 10, 11, and 6, respectively. In some instances, the anti-CD73 antibody comprises a VH comprising the VH CDR1, VH CDR2, and VH CDR3 set forth in SEQ ID NOs: 12, 13, and 9, respectively, and a VL comprising the VL CDR1, VL CDR2, and VL CDR3 set forth in SEQ ID NOs: 10, 11, and 6, respectively. In some instances, the anti-CD73 antibody comprises a VH comprising the VH CDR1, VH CDR2, and VH CDR3 set forth in SEQ ID NOs: 14, 15, and 9, respectively, and a VL comprising the VL CDR1, VL CDR2, and VL CDR3 set forth in SEQ ID NOs: 10, 11, and 6, respectively. In some instances, the anti-CD73 antibody comprises a VH comprising the VH CDR1, VH CDR2, and VH CDR3 set forth in SEQ ID NOs: 16, 17, and 18, respectively, and a VL comprising the VL CDR1, VL CDR2, and VL CDR3 set forth in SEQ ID NOs: 19, 20, and 21, respectively. In some instances, the anti-CD73 antibody comprises a VH comprising the VH CDR1, VH CDR2, and VH CDR3 set forth in SEQ ID NOs: 7, 8, and 9, respectively, and a VL comprising the VL CDR1, VL CDR2, and VL CDR3 set forth in SEQ ID NOs: 10, 29, and 6, respectively. In some instances, the anti-CD73 antibody comprises a VH comprising the VH CDR1, VH CDR2, and VH CDR3 set forth in SEQ ID NOs: 12, 13, and 9, respectively, and a VL comprising the VL CDR1, VL CDR2, and VL CDR3 set forth in SEQ ID NOs: 10, 29, and 6, respectively. In some instances, the anti-CD73 antibody comprises a VH comprising the VH CDR1, VH CDR2, and VH CDR3 set forth in SEQ ID NOs: 14, 15, and 9, respectively, and a VL comprising the VL CDR1, VL CDR2, and VL CDR3 set forth in SEQ ID NOs: 10, 29, and 6, respectively. In some instances, the anti-CD73 antibody comprises a VH comprising the VH CDR1, VH CDR2, and VH CDR3 set forth in SEQ ID NOs: 16, 28, and 18, respectively, and a VL comprising the VL CDR1, VL CDR2, and VL CDR3 set forth in SEQ ID NOs: 19, 20, and 21, respectively. In some instances these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 3); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 4); and/or (iii) bind human or cynomolgus monkey CD73 in the open conformation with high affinity (e.g., K_{D}≤0.5 nM) but do not significantly bind CD73 in the open conformation from mice (e.g., as determined by the binding assay described in Example 5); and/or (iv) bind human or cynomolgus monkey CD73 in the closed conformation with high affinity (e.g., K_{D}≤0.5 nM) but do not significantly bind CD73 in the closed conformation from mice; and/or (v) bind to an epitope within amino acids 40-53 of SEQ ID NO:70 (i.e., within TKVQQIRRAEPNVL (SEQ ID NO:76)) (e.g., as determined by the binding assay described in Example 5); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 16); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 17); and/or (ix) reduce free surface CD73 on cells (e.g., cancer cells, e.g., melanoma cancer cancers, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control).

In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In certain embodiments, the anti-CD73 antibodies comprise a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24. In some embodiments, the anti-CD73 antibodies comprise a heavy chain comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24. In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In certain embodiments, the anti-CD73 antibodies comprise a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In some embodiments, the anti-CD73 antibodies comprise a light chain comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In certain embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84; and (ii) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In certain embodiments, the anti-CD73 antibodies comprise: (i) a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84; and (ii) a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In some embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in SEQ ID NOs:24; and (ii) an amino acid sequence having one or more (e.g., 1, 2, 3, 4, 5, 6, 7, 8, 9, 10) amino acid substitutions, additions, and/or deletions relative to the amino acid sequence set forth in SEQ ID NOs:25. In some embodiments, the anti-CD73 antibodies comprise: (i) a heavy chain comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24; and (ii) a light chain comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84. In certain embodiments, the anti-CD73 antibodies comprise a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 23, 27, 80, and 81. In certain embodiments, the anti-CD73 antibodies comprise a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 23, 27, 80, and 81. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84 and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in SEQ ID NO: 23, 27, 80, and 81. In certain embodiments, the anti-CD73 antibodies comprise: (i) a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84, and (ii) a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 23, 27, 80, and 81.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22). In certain embodiments, the anti-CD73 antibodies comprise a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22). In certain embodiments, the anti-CD73 antibodies comprise a VH comprising the amino acid sequence set forth in SEQ ID NO:22. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24). In some embodiments, the anti-CD73 antibodies comprise a heavy chain comprising a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24). In certain embodiments, the anti-CD73 antibodies comprise a heavy chain comprising the amino acid sequence set forth in SEQ ID NO:24. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise a VL comprising the amino acid sequence set forth in SEQ ID NO:23. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise a light chain comprising a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In certain embodiments, the anti-CD73 antibodies comprise a light chain comprising the amino acid sequence set forth in SEQ ID NO:25. In certain embodiments, the anti-CD73 antibodies comprise: (i) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22); and (ii) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise: (i) a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22), and (ii) a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise: a VH comprising the amino acid sequence set forth in SEQ ID NO:22, and (ii) a VL comprising the amino acid sequence set forth in SEQ ID NO:23. In some embodiments, the anti-CD73 antibodies comprise: (i) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24); and (ii) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise: (i) a heavy chain comprising a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24), and (ii) a light chain comprising a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise: (i) a heavy chain comprising the amino acid sequence set forth in SEQ ID NO:24, and (ii) a light chain comprising the amino acid sequence set forth in SEQ ID NO:25.

The CD73-binding epitope of HzCL25 is within the amino acid sequence TKVQQIRRAEPNVL (SEQ ID NO:76) (i.e., amino acids 40-53 of the amino acid sequence set forth in SEQ ID NO:70). This disclosure features antibodies that bind to CD73 within the sequence TKVQQIRRAEPNVL (SEQ ID NO:76). This disclosure features antibodies that bind to the same epitope as HzCL25. This disclosure also features antibodies that competitively inhibit binding of HzCL25 to human CD73.

In some embodiments, the VH of HzCL25 is linked to a heavy chain constant region comprising a CH1 domain and a hinge region. In some embodiments, the VH of HzCL25 is linked to a heavy chain constant region comprising a CH3 domain. In some embodiments, the CH3 domain lacks the C-terminal lysine (K) amino acid residue. In some embodiments, the CH3 domain contains the C-terminal lysine (K) amino acid residue. In certain embodiments, the VH of HzCL25 is linked to a heavy chain constant region comprising a CH1 domain, hinge region, CH2 domain, and CH3 domain from human IgG1. In some embodiments, the CH3 domain from human IgG1 lacks the C-terminal lysine (K) amino acid residue. In some embodiments, the CH3 domain from human IgG1 contains the C-terminal lysine (K) amino acid residue. In certain embodiments such an antibody contains one or more additional mutations in the heavy chain constant region that increase the stability of the antibody. In certain embodiments, the heavy chain constant region includes substitutions that modify the properties of the antibody (e.g., decrease Fc receptor binding, increase or decrease antibody glycosylation, decrease binding to C1q). In certain embodiments, the heavy chain constant region includes an alanine at position Asparagine-297 (N297, according to EU numbering) of the heavy chain constant region to reduce effector function.

In certain embodiments, the anti-CD73 antibody is an IgG antibody. In one embodiment, the antibody is an IgG1 antibody. In one embodiment, the antibody is an IgG4 antibody. In another embodiment, the antibody is an IgG2 antibody. In certain embodiments, the anti-CD73 antibody comprises a heavy chain constant region lacking one or more lysine (K) amino acid residues relative to a wild type heavy chain constant region. For example, in certain embodiments, the antibody comprises heavy chain constant region lacking the C-terminal lysine (K) amino acid residue of the CH3 domain of the heavy chain constant region. In certain embodiments, the anti-CD73 antibody comprises a heavy chain constant region having the amino acid sequence set forth in SEQ ID NO:73. In certain embodiments, the anti-CD73 antibody comprises a heavy chain constant region having the amino acid sequence set forth in SEQ ID NO:75. In certain embodiments, the anti-CD73 antibody comprises a light chain constant region having the amino acid sequence set forth in SEQ ID NO:74. In certain embodiments, the anti-CD73 antibody comprises a heavy chain constant region having the amino acid sequence set forth in SEQ ID NO:73 and a light chain constant region having the amino acid sequence set forth in SEQ ID NO:74. In certain embodiments, the anti-CD73 antibody comprises a heavy chain constant region having the amino acid sequence set forth in SEQ ID NO:75 and a light chain constant region having the amino acid sequence set forth in SEQ ID NO:74.

### Antibody 3-F03

Antibody 3-F03 is a human IgG1/kappa monoclonal antibody with alanine at position Asparagine-297 (N297, according to EU numbering) of the heavy chain constant region to reduce effector function. 3-F03 specifically binds human, cynomolgus, and murine CD73 with high affinity (K_{D}≤2 nM) and has low effector functionality.

3-F03 was engineered from sequences obtained by multiple selection rounds of single donor library. scFv cassettes from this pool were then recombined into a yeast display vector library, which was subjected to FACs selection with murine CD73 (SEQ ID NO:71). The amino acid sequences of the yeast 3-F03 scFv cassette are set forth in SEQ ID NOs:77 and 65, respectively:

(SEQ ID NO: 77)
EVQLVESGGGLVQPGGSLRLSCAASGFTFSSYDMHWVRQAPGKGLEWVAV
MSYDGSNKYYADSVKGRFTISRDNSKNTLYLQMNSLRAEDTAVYYCATEI
AAKGDYWGQGTLVTVSS;
and
(SEQ ID NO: 65)
AIQMTQSPSSLSASVGDRVTITCRASQGISNYLAWYQQKPGKAPKLLIYA
ASTLQSGVPSRFSGSGSGTDFTLTISSLQPEDFATYYCQQSYSTPHFGQG
TRLEIK.

To construct the 3-F03 antibody, the yeast 3-F03 VH and VL were modified as follows and cloned into a human IgG1/kappa scaffold. For the VH, the N-terminal glutamate (E) of yeast 3-F03 VH (SEQ ID NO:77) was removed and the threonine (T) at Kabat position H77 of SEQ ID NO:77 (i.e., position 78 of SEQ ID NO:77) was substituted with an alanine (A). For the VL, the N-terminal alanine (A) of SEQ ID NO:65 was removed. The resulting full-length human 3-F03 antibody contains the VH and VL set forth in the amino acid sequences of SEQ ID NOs:60 and 61, respectively. The resulting full-length human 3-F03 antibody is referred to herein as “3-F03”. Table 3, below, shows the amino acid sequences of the 3-F03 CDRs according to IMGT, Chothia, AbM, Kabat, and Contact numbering. Table 3, below, also shows the amino acid sequences of the 3-F03 mature VH, VL, heavy chain, and light chain.

TABLE 3
Amino acid sequences of 3-F03 CDRs, VH, and VL

| | IMGT | Chothia | AbM | Kabat | Contact |
|---|---|---|---|---|---|
| VH | GFTFSSYD (SEQ | GFTFSSY (SEQ | GFTFSSYDMH | SYDMH (SEQ | SSYDMH (SEQ |
| CDR1 | ID NO: 34) | ID NO: 41) | (SEQ ID NO: 46) | ID NO: 49) | ID NO: 53) |
| VH | MSYDGSNK | SYDGSN (SEQ | VMSYDGSNKY | VMSYDGSNKY | WVAVMSYDGS |
| CDR2 | (SEQ ID | ID NO: 42) | (SEQ ID NO: 47) | YADSVKG (SEQ | NKY (SEQ ID |
| | NO: 35) | | | ID NO: 50) | NO: 54) |
| VH | ATEIAAKGDY | EIAAKGDY | EIAAKGDY | EIAAKGDY | ATEIAAKGD |
| CDR3 | (SEQ ID | (SEQ ID NO: 52) | (SEQ ID NO: 52) | (SEQ ID NO: 52) | (SEQ ID NO: 56) |
| | NO: 36) | | | | |
| VL | QGISNY (SEQ | RASQGISNYLA | RASQGISNYLA | RASQGISNYLA | SNYLAWY |
| CDR1 | ID NO: 37) | (SEQ ID NO: 44) | (SEQ ID NO: 44) | (SEQ ID NO: 44) | (SEQ ID NO: 57) |
| VL | AAS (SEQ ID | AASTLQS (SEQ | AASTLQS (SEQ | AASTLQS (SEQ | LLIYAASTLQ |
| CDR2 | NO: 38) | ID NO: 45) | ID NO: 45) | ID NO: 45) | (SEQ ID NO: 58) |
| VL | QQSYSTPH | QQSYSTPH | QQSYSTPH | QQSYSTPH | QQSYSTP (SEQ |
| CDR3 | (SEQ ID | (SEQ ID NO: 39) | (SEQ ID NO: 39) | (SEQ ID NO: 39) | ID NO: 59) |
| | NO: 39) | | | | |

| | |
|---|---|
| VH | VQLVESGGGLVQPGGSLRLSCAASGFTFSSYDMHWVRQAPGKGLEWVAVMSYDGSNKYYA |
| | DSVKGRFTISRDNSKNALYLQMNSLRAEDTAVYYCATEIAAKGDYWGQGTLVTVSS (SEQ |
| | ID NO: 60) |
| VL | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIK (SEQ ID NO: 61) |
| HC | VQLVESGGGLVQPGGSLRLSCAASGFTFSSYDMHWVRQAPGKGLEWVAVMSYDGSNKYYA |
| | DSVKGRFTISRDNSKNALYLQMNSLRAEDTAVYYCATEIAAKGDYWGQGTLVTVSSASTKG |
| | PSVFPLAPSSKSTSGGTAALGCLVKDYFPEPVTVSWNSGALTSGVHTFPAVLQSSGLYSLSSV |
| | VTVPSSSLGTQTYICNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVFSCSVMHEALHN |
| | HYTQKSLSLSPGK (SEQ ID NO: 66) |
| LC | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIKRTVAAPSVFIFPPSDEQLKSGTAS |
| | VVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 31) |

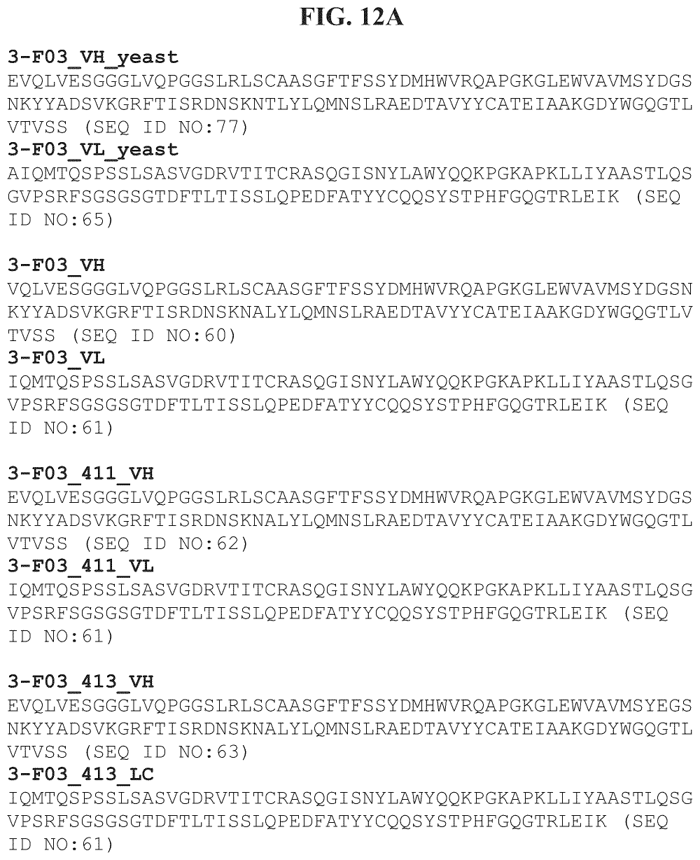

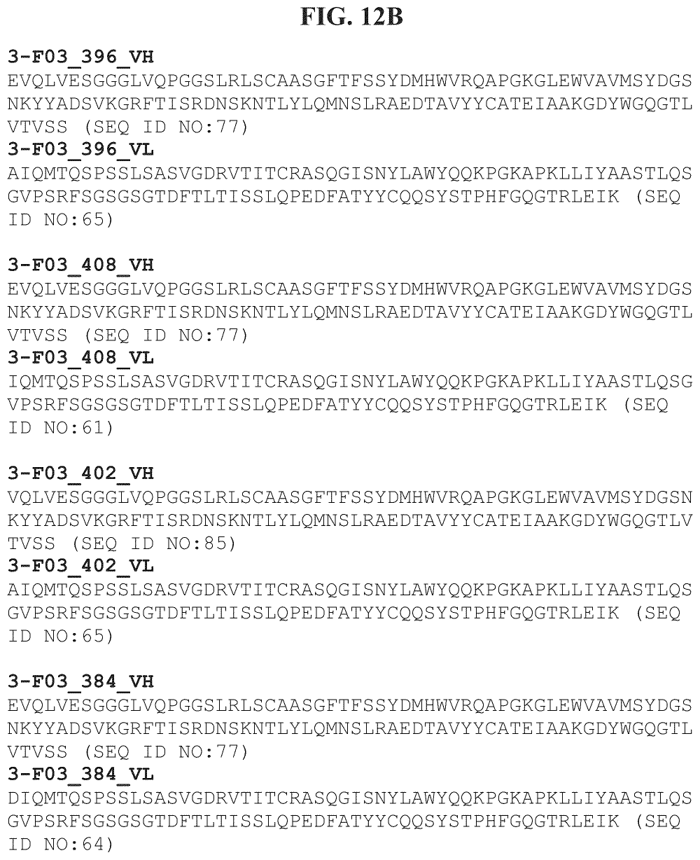

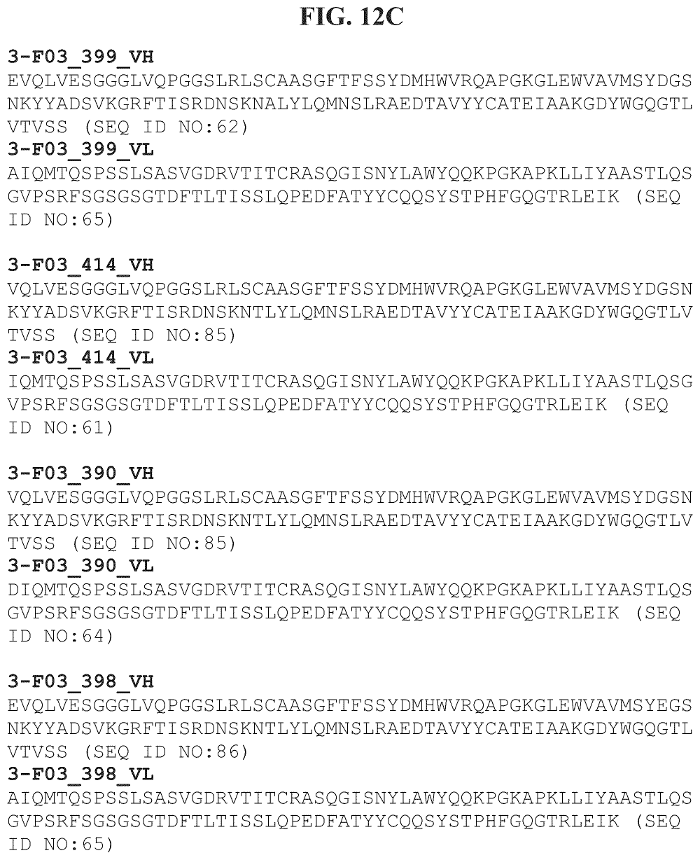

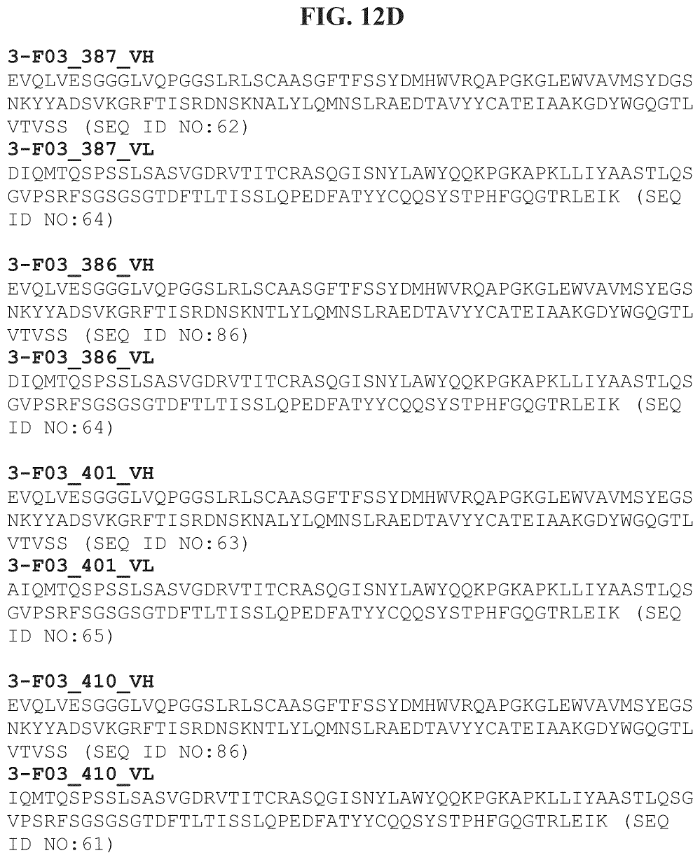

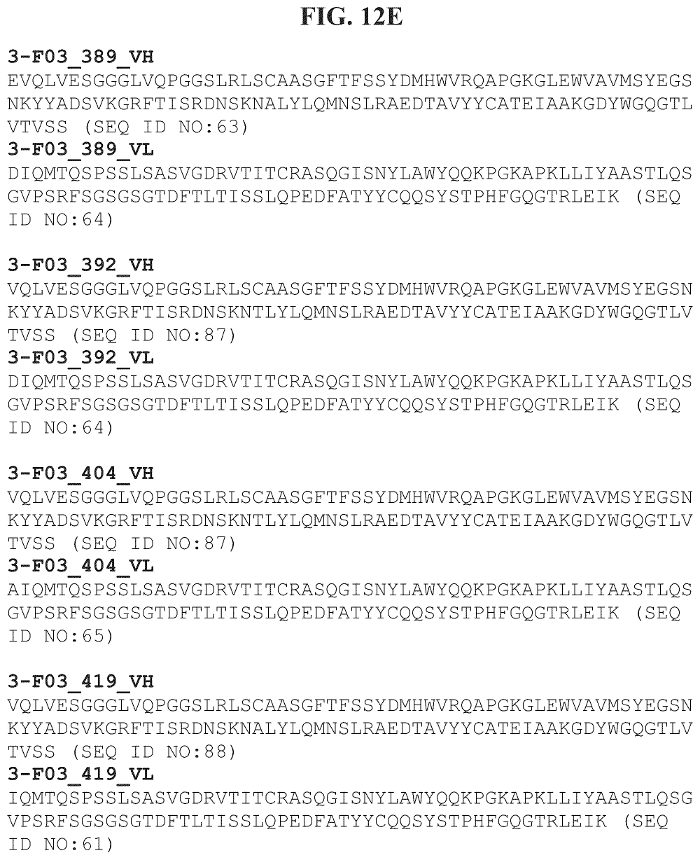

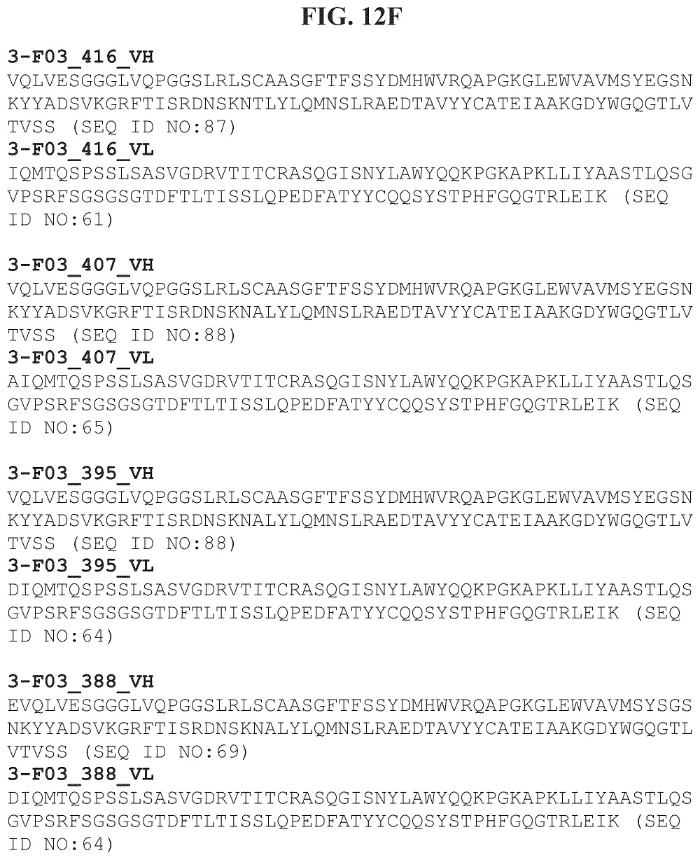

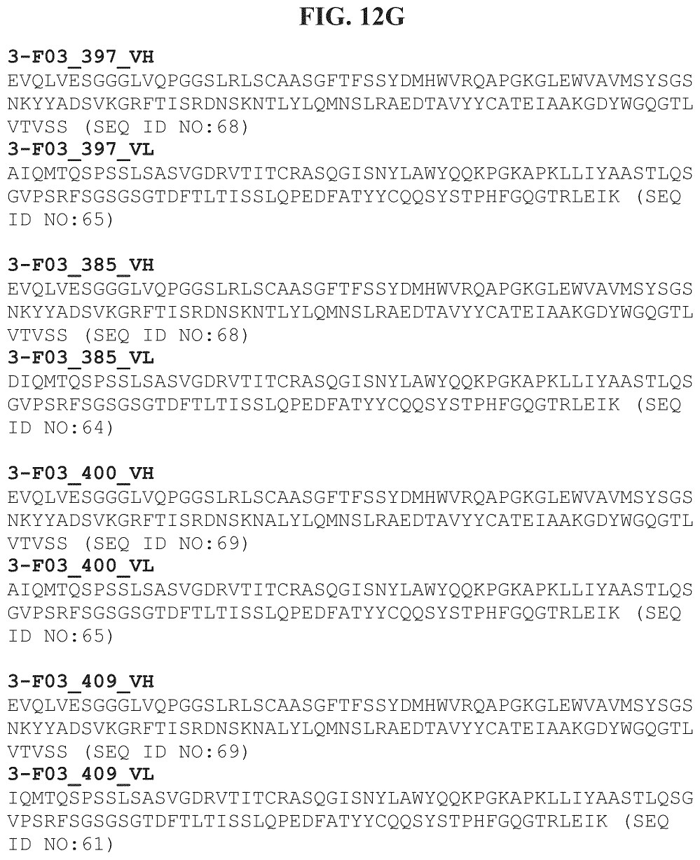

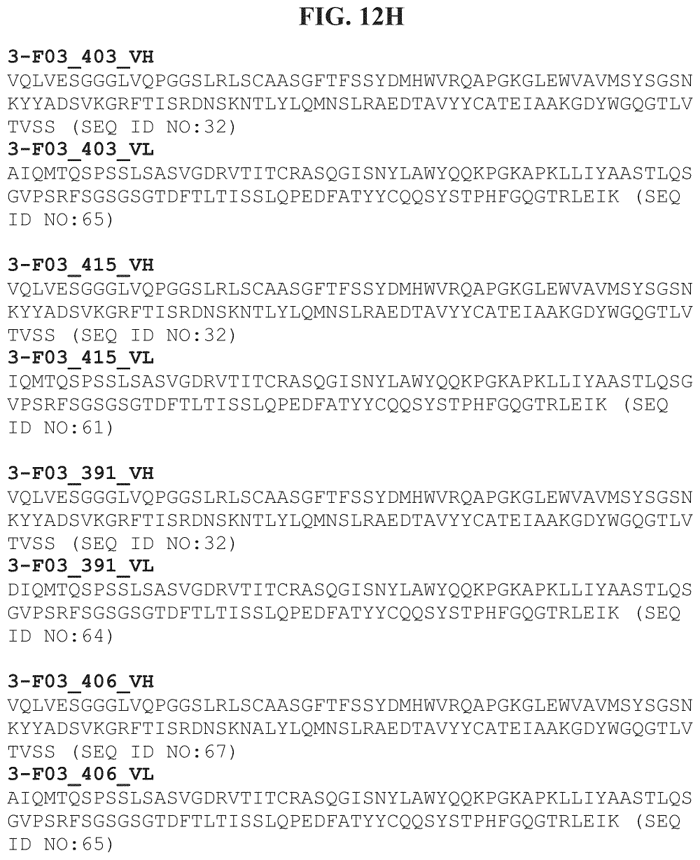

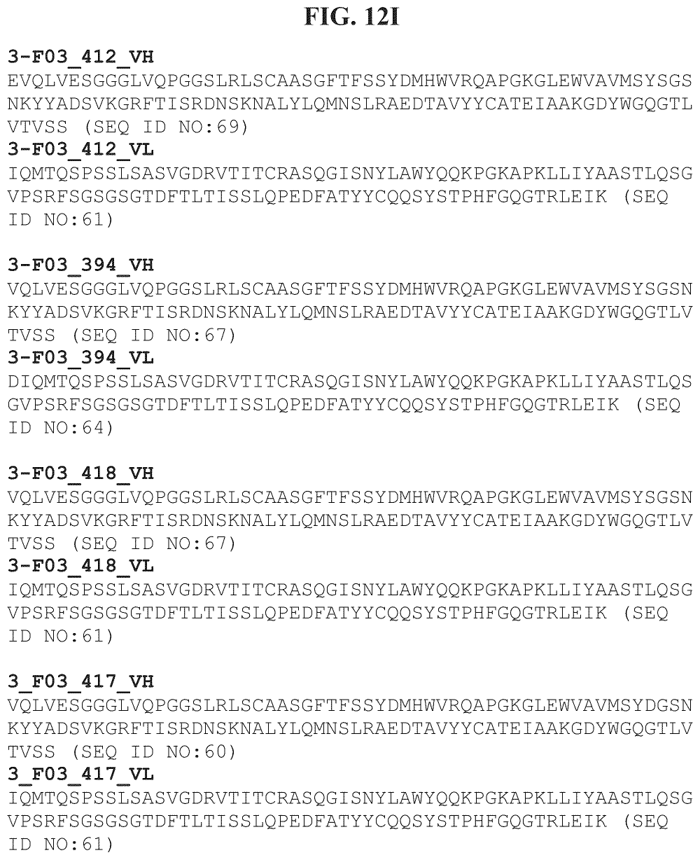

Variants of 3-F03 are also described herein. 3-F03_411 is identical to 3-F03, except that the 3-F03_411 heavy chain (i) contains an N-terminal glutamate (E) that is lacking in 3-F03 and (ii) does not include the C-terminal lysine present in 3-F03. Table 4, below, shows the amino acid sequences of the 3-F03_411 mature VH, VL, heavy chain and light chain. 3-F03_413 is identical to 3-F03_411, except that it contains a glutamate (E) at VH Kabat position H53 (position 54 of SEQ ID NO:60) instead of an aspartic acid (D). Table 5, below, shows the amino acid sequences of the 3-F03_413 CDRs according to IMGT, Chothia, AbM, Kabat, and Contact numbering. Table 5, below, also shows the amino acid sequences of the 3-F03_413 mature VH, VL, heavy chain, and light chain. Additional variants are described in the Examples below (see FIG. 12A-FIG. 12J).

TABLE 4
Amino acid sequences of 3-F03_411 HC and LC

| | SEQUENCE |
|---|---|
| VH | EVQLVESGGGLVQPGGSLRLSCAASGFTFSSYDMHWVRQAPGKGLEWVAVMSYDGSNKYY |
| | ADSVKGRFTISRDNSKNALYLQMNSLRAEDTAVYYCATEIAAKGDYWGQGTLVTVSS (SEQ |
| | ID NO: 62) |
| VL | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIK (SEQ ID NO: 61) |
| Heavy | EVQLVESGGGLVQPGGSLRLSCAASGFTFSSYDMHWVRQAPGKGLEWVAVMSYDGSNKYY |
| Chain | ADSVKGRFTISRDNSKNALYLQMNSLRAEDTAVYYCATEIAAKGDYWGQGTLVTVSSASTK |
| | GPSVFPLAPSSKSTSGGTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAPELLGGPSVFLFPPKPK |
| | DTLMISRTPEVTCVVVDVSHEDPEVKFNWYVDGVEVHNAKTKPREEQYASTYRVVSVLTVL |
| | HQDWLNGKEYKCKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGNVFSCSVMHEALH |
| | NHYTQKSLSLSPG (SEQ ID NO: 30) |
| Light | IQMTQSPSSLSASVGDRVTITCRASQGISNYLAWYQQKPGKAPKLLIYAASTLQSGVPSRFSGS |
| Chain | GSGTDFTLTISSLQPEDFATYYCQQSYSTPHFGQGTRLEIKRTVAAPSVFIFPPSDEQLKSGTAS |
| | VVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 31) |

TABLE 5
Amino acid sequences of 3-F03_413 CDRs, VH, VL, HC, LC

| | IMGT | Chothia | AbM | Kabat | Contact |
|---|---|---|---|---|---|
| VH | GFTFSSYD (SEQ | GFTFSSY (SEQ | GFTFSSYDMH | SYDMH (SEQ | SSYDMH (SEQ |
| CDR1 | ID NO: 34) | ID NO: 41) | (SEQ ID NO: 46) | ID NO: 49) | ID NO: 53) |
| VH | MSYEGSNK | SYEGSN (SEQ | VMSYEGSNKY | VMSYEGSNKY | WVAVMSYEGS |
| CDR2 | (SEQ ID | ID NO: 43) | (SEQ ID NO: 48) | YADSVKG (SEQ | NKY (SEQ ID |
| | NO: 40) | | | ID NO: 51) | NO: 55) |
| VH | ATEIAAKGDY | EIAAKGDY | EIAAKGDY | EIAAKGDY | ATEIAAKGD |
| CDR3 | (SEQ ID | (SEQ ID NO: 52) | (SEQ ID NO: 52) | (SEQ ID NO: 52) | (SEQ ID NO: 56) |
| | NO: 36) | | | | |
| VL | QGISNY (SEQ | RASQGISNYLA | RASQGISNYLA | RASQGISNYLA | SNYLAWY |
| CDR1 | ID NO: 37) | (SEQ ID NO: 44) | (SEQ ID NO: 44) | (SEQ ID NO: 44) | (SEQ ID NO: 57) |
| VL | AAS (SEQ ID | AASTLQS (SEQ | AASTLQS (SEQ | AASTLQS (SEQ | LLIYAASTLQ |
| CDR2 | NO: 38) | ID NO: 45) | ID NO: 45) | ID NO: 45) | (SEQ ID NO: 58) |
| VL | QQSYSTPH | QQSYSTPH | QQSYSTPH | QQSYSTPH | QQSYSTP (SEQ |
| CDR3 | (SEQ ID | (SEQ ID NO: 39) | (SEQ ID NO: 39) | (SEQ ID NO: 39) | ID NO: 59) |
| | NO: 39) | | | | |

| | |
|---|---|
| VH | EVQLVESGGGLVQPGGSLRLSCAASGFTFSSYDMHWVRQAPGKGLEWVAVMSYEGSNKYY |
| | ADSVKGRFTISRDNSKNALYLQMNSLRAEDTAVYYCATEIAAKGDYWGQGTLVTVSS (SEQ |
| | ID NO: 63) |
| VL | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIK (SEQ ID NO: 61) |
| HC | EVQLVESGGGLVQPGGSLRLSCAASGFTFSSYDMHWVRQAPGKGLEWVAVMSYEGSNKYY |
| | ADSVKGRFTISRDNSKNALYLQMNSLRAEDTAVYYCATEIAAKGDYWGQGTLVTVSSASTK |
| | GPSVFPLAPSSKSTSGGTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAPELLGGPSVFLFPPKPK |
| | DTLMISRTPEVTCVVVDVSHEDPEVKFNWYVDGVEVHNAKTKPREEQYASTYRVVSVLTVL |
| | HQDWLNGKEYKCKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGNVFSCSVMHEALH |
| | NHYTQKSLSLSPG (SEQ ID NO: 33) |
| LC | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIKRTVAAPSVFIFPPSDEQLKSGTAS |
| | VVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 31) |

The anti-CD73 antibodies can encompass the VH CDR1, VH CDR2, and VH CDR3 and the VL CDR1, VL CDR2, and VL CDR3 of 3-F03 or 3-F03_413. In some instances, the anti-CD73 antibody comprises a VH comprising VH CDR1, VH CDR2, and VH CDR3 of 3-F03 (see Table 3). In some instances, the anti-CD73 antibody comprises a VL comprising VL CDR1, VL CDR2, and VL CDR3 of 3-F03 (see Table 3). In some instances, the anti-CD73 antibody comprises a VH comprising VH CDR1, VH CDR2, and VH CDR3 of 3-F03 (see Table 3) and a VL comprising VL CDR1, VL CDR2, and VL CDR3 of 3-F03 (see Table 3). In some instances, the anti-CD73 antibody comprises a VH comprising VH CDR1, VH CDR2, and VH CDR3 of 3-F03_413 (see Table 5). In some instances, the anti-CD73 antibody comprises a VL comprising VL CDR1, VL CDR2, and VL CDR3 of 3-F03_413 (see Table 5). In some instances, the anti-CD73 antibody comprises a VH comprising VH CDR1, VH CDR2, and VH CDR3 of 3-F03_413 (see Table 5) and a VL comprising VL CDR1, VL CDR2, and VL CDR3 of 3-F03_413 (see Table 5). In some instances, the anti-CD73 antibodies can have, e.g., 1, 2, or 3 substitutions within one or more (i.e., 1, 2, 3, 4, 5, or 6) of the six CDRs of 3-F03 or 3-F03_413. In some instances, these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 10); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 11); and/or (iii) bind human, cynomolgus monkey, or murine CD73 in the open conformation with high affinity (e.g., K_{D}≤2 nM) (e.g., as determined by the binding assay described in Example 12); and/or (iv) do not bind human, cynomolgus monkey, or murine CD73 in the closed conformation; and/or (v) bind to an epitope within amino acids 386-399 of SEQ ID NO:70 (i.e., within AAVLPFGGTFDLVQ (SEQ ID NO:78) amino acids 470-489 of SEQ ID NO:70 (i.e., within ILPNFLANGGDGFQMIKDEL (SEQ ID NO:79)) (e.g., as determined by the binding assay described in Example 12); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 16); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 17).

The anti-CD73 antibodies can comprise the VH CDR1, VH CDR2, and VH CDR3 of 3-F03 or 3-F03_413 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VH CDR1 and/or VH CDR2 and/or VH CDR3 of 3-F03 or 3-F03_413. In some embodiments, the anti-CD73 antibodies further comprise the VL CDR1, VL CDR2, and VL CDR3 of 3-F03 or 3-F03_413 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VL CDR1 and/or VL CDR2 and/or VL CDR3 of 3-F03 or 3-F03_413. In some instances, the anti-CD73 antibody comprises a VH comprising the VH CDR1, VH CDR2, and VH CDR3 set forth in SEQ ID NOs: 34, 35, and 36, respectively, and a VL comprising the VL CDR1, VL CDR2, and VL CDR3 set forth in SEQ ID NOs: 37, 38, and 39, respectively. In some instances, the anti-CD73 antibody comprises a VH comprising the VH CDR1, VH CDR2, and VH CDR3 set forth in SEQ ID NOs: 41, 42, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 46, 47, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 49, 50, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 53, 54, and 56, respectively, and a VL comprising the VL CDR1, VL CDR2, and VL CDR3 set forth in SEQ ID NOs: 57, 58, and 59, respectively. In some instances, the anti-CD73 antibody comprises a VH comprising the VH CDR1, VH CDR2, and VH CDR3 set forth in SEQ ID NOs: 34, 40, and 36, respectively, and a VL comprising the VL CDR1, VL CDR2, and VL CDR3 set forth in SEQ ID NOs: 37, 38, and 39, respectively. In some instances, the anti-CD73 antibody comprises a VH comprising the VH CDR1, VH CDR2, and VH CDR3 set forth in SEQ ID NOs: 41, 43, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 46, 48, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 49, 51, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 53, 55, and 56, respectively, and a VL comprising the VL CDR1, VL CDR2, and VL CDR3 set forth in SEQ ID NOs: 57, 58, and 59, respectively. In some instances, these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 10); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 11); and/or (iii) bind human, cynomolgus monkey, or murine CD73 in the open conformation with high affinity (e.g., K_{D}≤2 nM) (e.g., as determined by the binding assay described in Example 12); and/or (iv) do not bind human, cynomolgus monkey, or murine CD73 in the closed conformation; and/or (v) bind to an epitope within amino acids 386-399 of SEQ ID NO:70 (i.e., within AAVLPFGGTFDLVQ (SEQ ID NO:78) amino acids 470-489 of SEQ ID NO:70 (i.e., within ILPNFLANGGDGFQMIKDEL (SEQ ID NO:79)) (e.g., as determined by the binding assay described in Example 12); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 16); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 17).

In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88. In certain embodiments, the anti-CD73 antibodies comprise a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 34-36, respectively, or SEQ ID NOs:34, 40 and 36, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 30, 33, and 66. In some embodiments, the anti-CD73 antibodies comprise a heavy chain comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs:34, 40, and 36, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 30, 33, and 66. In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In certain embodiments, the anti-CD73 antibodies comprise a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 37-39, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some embodiments, the anti-CD73 antibodies comprise a light chain comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In certain embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88; and (ii) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In certain embodiments, the anti-CD73 antibodies comprise: (i) a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs: 34, 40, and 36, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88; and (ii) a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 37-39, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In some embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in SEQ ID NOs: 30, 33, and 66; and (ii) an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some embodiments, the anti-CD73 antibodies comprise: (i) a heavy chain comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 34-36, respectively, or SEQ ID NOs:34, 40, and 36, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 30, 33, and 66; and (ii) a light chain comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88. In certain embodiments, the anti-CD73 antibodies comprise a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs:34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 61, 64, and 65. In certain embodiments, the anti-CD73 antibodies comprise a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 61, 64, and 65. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88 and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in SEQ ID NO: 61, 64, and 65. In certain embodiments, the anti-CD73 antibodies comprise: (i) a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs:34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88, and (ii) a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 61, 64, and 65.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 or 3-F03_413 (i.e., the amino acid sequence set forth in SEQ ID NO:62 or 63, respectively). In certain embodiments, the anti-CD73 antibodies comprise a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 (i.e., the amino acid sequence set forth in SEQ ID NO:62). In certain embodiments, the anti-CD73 antibodies comprise a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 (i.e., the amino acid sequence set forth in SEQ ID NO:63). In some embodiments, the anti-CD73 antibodies comprise a VH comprising the amino acid sequence set forth in SEQ ID NO:62. In some embodiments, the anti-CD73 antibodies comprise a VH comprising the amino acid sequence set forth in SEQ ID NO:63. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 or 3-F03_F13 (i.e., the amino acid sequence set forth in SEQ ID NO:30 or 33, respectively). In some embodiments, the anti-CD73 antibodies comprise a heavy chain comprising a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 (i.e., the amino acid sequence set forth in SEQ ID NO:30). In some embodiments, the anti-CD73 antibodies comprise a heavy chain comprising a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_413 (i.e., the amino acid sequence set forth in SEQ ID NO:33). In some embodiments, the anti-CD73 antibodies comprise a heavy chain comprising the amino acid sequence set forth in SEQ ID NO:30. In some embodiments, the anti-CD73 antibodies comprise a heavy chain comprising the amino acid sequence set forth in SEQ ID NO:33. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In certain embodiments, the anti-CD73 antibodies comprise a VL comprising the VL CDR1, VL CDR2, and VL CDR3 of 3-F03_411 or 3-F03_413 (see Table 3, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In some embodiments, the anti-CD73 antibodies comprise a VL comprising the amino acid sequence set forth in SEQ ID NO:61. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise a light chain comprising a VL comprising the VL CDR1, VL CDR2, and VL CDR3 of 3-F03_411 or 3-F03_413 (see Table 5, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise a light chain comprising the amino acid sequence set forth in SEQ ID NO:31. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 or 3-F03_413 (i.e., the amino acid sequence set forth in SEQ ID NO:62 or 63, respectively) and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In certain embodiments, the anti-CD73 antibodies comprise: (i) a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03 (i.e., the amino acid sequence set forth in SEQ ID NO:62), and (ii) a VL comprising the VL CDR1, VL CDR2, and VL CDR3 of 3-F03_411 (see Table 3, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03 (i.e., the amino acid sequence set forth in SEQ ID NO:61). In certain embodiments, the anti-CD73 antibodies comprise: (i) a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_413 (i.e., the amino acid sequence set forth in SEQ ID NO:63), and (ii) a VL comprising the VL CDR1, VL CDR2, and VL CDR3 of 3-F03_413 (see Table 5, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_413 (i.e., the amino acid sequence set forth in SEQ ID NO:61). In some embodiments, the anti-CD73 antibody comprises: (i) a VH comprising the amino acid sequence set forth in SEQ ID NO:62; and (ii) a VL comprising the amino acid sequence set forth in SEQ ID NO:61. In some embodiments, the anti-CD73 antibody comprises: (i) a VH comprising the amino acid sequence set forth in SEQ ID NO:63; and (ii) a VL comprising the amino acid sequence set forth in SEQ ID NO:61. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 or 3-F03_413 (i.e., the amino acid sequence set forth in SEQ ID NO:30 or 33) and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise: (i) a heavy chain comprising the a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 (i.e., the amino acid sequence set forth in SEQ ID NO:30), and (ii) a light chain comprising a VL comprising the VL CDR1, VL CDR2, and VL CDR3 of 3-F03_411 (see Table 3, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03 (i.e., the amino acid sequence set forth in SEQ ID NO:31). In some embodiments, the anti-CD73 antibodies comprise: (i) a heavy chain comprising the a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03 (i.e., the amino acid sequence set forth in SEQ ID NO:33), and (ii) a light chain comprising a VL comprising the VL CDR1, VL CDR2, and VL CDR3 of 3-F03_413 (see Table 5, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_413 (i.e., the amino acid sequence set forth in SEQ ID NO:31). In some embodiments, the anti-CD73 antibody comprises: (i) a heavy chain comprising the amino acid sequence set forth in SEQ ID NO:30; and (ii) a light chain comprising the amino acid sequence set forth in SEQ ID NO:31. In some embodiments, the anti-CD73 antibody comprises: (i) a heavy chain comprising the amino acid sequence set forth in SEQ ID NO:33; and (ii) a light chain comprising the amino acid sequence set forth in SEQ ID NO:31.

The CD73-binding epitope of 3-F03 (and variants thereof, e.g., 3-F03_411 and 3-F03_413) contains AAVLPFGGTFDLVQ (SEQ ID NO:78) (i.e., amino acids 386-399 of the amino acid sequence set forth in SEQ ID NO:70) and ILPNFLANGGDGFQMIKDEL (SEQ ID NO:79) (i.e., amino acids 470-489 of the amino acid sequence set forth in SEQ ID NO:70). This disclosure features antibodies that bind to CD73 an epitope within AAVLPFGGTFDLVQ (SEQ ID NO:78) and ILPNFLANGGDGFQMIKDEL (SEQ ID NO:79). This disclosure features antibodies that bind to the same epitope as 3-F03 (or a variant thereof, e.g., 3-F03_411 or 3-F03_413). This disclosure also features antibodies that competitively inhibit binding of 3-F03 (or a variant thereof, e.g., 3-F03_411 or 3-F03_413) to human CD73.

In some embodiments, the VH of 3-F03 (or a variant thereof, e.g., 3-F03_411 or 3-F03_413) is linked to a heavy chain constant region comprising a CH1 domain and a hinge region. In some embodiments, the VH of 3-F03 (or a variant thereof, e.g., 3-F03_411 or 3-F03_413) is linked to a heavy chain constant region comprising a CH3 domain. In some embodiments, the CH3 domain lacks the C-terminal lysine (K) amino acid residue. In some embodiments, the CH3 domain contains the C-terminal lysine (K) amino acid residue. In certain embodiments, the VH of 3-F03 (or a variant thereof, e.g., 3-F03_411 or 3-F03_413) is linked to a heavy chain constant region comprising a CH1 domain, hinge region, CH2 domain, and CH3 domain from human IgG1. In some embodiments, the CH3 domain from human IgG1 lacks the C-terminal lysine (K) amino acid residue. In some embodiments, the CH3 domain from human IgG1 contains the C-terminal lysine (K) amino acid residue. In certain embodiments such an antibody contains one or more additional mutations in the heavy chain constant region that increase the stability of the antibody. In certain embodiments, the heavy chain constant region includes substitutions that modify the properties of the antibody (e.g., decrease Fc receptor binding, increase or decrease antibody glycosylation, decrease binding to C1q). In certain embodiments, the heavy chain constant region includes an alanine (A) at position Asparagine-297 (N297, according to EU numbering) of the heavy chain constant region to reduce effector function.

In certain embodiments, the anti-CD73 antibody is an IgG antibody. In one embodiment, the antibody is an IgG1 antibody. In one embodiment, the antibody is an IgG4 antibody. In another embodiment, the antibody is an IgG2 antibody. In certain embodiments, the anti-CD73 antibody comprises a heavy chain constant region lacking one or more lysine (K) amino acid residues relative to a wild type heavy chain constant region. For example, in certain embodiments, the antibody comprises heavy chain constant region lacking the C-terminal lysine (K) amino acid residue of the CH3 domain of the heavy chain constant region. In certain embodiments, the anti-CD73 antibody comprises a heavy chain constant region having the amino acid sequence set forth in SEQ ID NO:73. In certain embodiments, the anti-CD73 antibody comprises a heavy chain constant region having the amino acid sequence set forth in SEQ ID NO:75. In certain embodiments, the anti-CD73 antibody comprises a light chain constant region having the amino acid sequence set forth in SEQ ID NO:74. In certain embodiments, the anti-CD73 antibody comprises a heavy chain constant region having the amino acid sequence set forth in SEQ ID NO:73 and a light chain constant region having the amino acid sequence set forth in SEQ ID NO:74. In certain embodiments, the anti-CD73 antibody comprises a heavy chain constant region having the amino acid sequence set forth in SEQ ID NO:75 and a light chain constant region having the amino acid sequence set forth in SEQ ID NO:74.

### Additional Anti-CD73 Antibodies and Inhibitors

This disclosure provides additional anti-CD73 antibodies and CD73 inhibitors that are useful in combination with an A2A and/or A2B adenosine receptor inhibitor in treating diseases, e.g., cancer.

Other anti-CD73 antibodies useful in combination with an inhibitor of A2A and/or A2B adenosine receptor in the methods described herein are known in the art. See, e.g., U.S. Pat. Nos. 9,090,697, 9,388,249, 9,605,080, 9,938,356, 10,100,129, and 10,287,362, US Patent Application Publication Nos. 2004/0142342, 2007/0009518, 2011/0300136, 2018/0009899, 2018/0030144, 2018/0237536, 2018/0264107, 2019/0031766, 2019/0225703, 2019/0077873, and 2019/0256598, and international patent application publication nos. WO 2004/079013, WO 2011/089004, WO 2014/153424, WO 2017/100670, WO 2001/080884, WO 2018/110555, WO 2018/137598, WO 2018/187512, WO 2018/215535, WO 2018/237173, WO 2019/170131, WO 2019/173692, and WO 2019/173291, each of which is incorporated by reference herein in its entirety.

In some instances, the anti-CD73 antibody comprises a VH comprising a VH CDR1, a VH CDR2, and a VH CDR3 of a VH comprising the amino acid sequence EIQLQQSGPELVKPGASVKVSCKASGYAFTSYNMYWVKQSHGKSLEWIGYIDPYNG GTSYNQKFKGKATLTVDKSSSTAYMHLNSLTSEDSAVYYCARGYGNYKAWFAYW GQGTLVTVSA (SEQ ID NO:100), and a VL comprising a VL CDR1, a VL CDR2, and a VL CDR3 of a VL comprising the amino acid sequence DAVMTQTPKFLLVSAGDRVTITCKASQSVTNDVAWYQQKPGQSPKLLIYYASNRYT GVPDRFTGSGYGTDFTFTISTVQAEDLAVYFCQQDYSSLTFGAGTKLELK (SEQ ID NO:101). In some instances, the anti-CD73 antibody comprises a VH comprising the amino acid sequence set forth in SEQ ID NO: 100 and a VL comprising the amino acid sequence set forth in SEQ ID NO:101. In some instances, the anti-CD73 antibody is 11E1 (see US patent application publication no. 2018/0237536, which is incorporated by reference herein in its entirety). In some instances, the anti-CD73 antibody comprises a heavy chain comprising the amino acid sequence set forth in SEQ ID NO:102. In some instances, the anti-CD73 antibody comprises a light chain comprising the amino acid sequence set forth in SEQ ID NO:103. In some instances, the anti-CD73 antibody comprises a heavy chain comprising the amino acid sequence set forth in SEQ ID NO:102 and a light chain comprising the amino acid sequence set forth in SEQ ID NO: 103.

In some instances, the anti-CD73 antibody comprises a VH comprising a VH CDR1, a VH CDR2, and a VH CDR3 of a VH comprising the amino acid sequence EVQLLESGGGLVQPGGSLRLSCAASGFTFSSYAYSWVRQAPGKGLEWVSAISGSGGR TYYADSVKGRFTISRDNSKNTLYLQMNSLRAEDTAVYYCARLGYGRVDEWGRGTL VTVSS (SEQ ID NO:96), and a VL comprising a VL CDR1, a VL CDR2, and a VL CDR3 of a VL comprising the amino acid sequence QSVLTQPPSASGTPGQRVTISCSGSLSNIGRNPVNWYQQLPGTAPKLLIYLDNLRLSG VPDRFSGSKSGTSASLAISGLQSEDEADYYCATWDDSHPGWTFGGGTKLTVL (SEQ ID NO:97). In some instances, the anti-CD73 antibody comprises a VH comprising the amino acid sequence set forth in SEQ ID NO:96 and a VL comprising the amino acid sequence set forth in SEQ ID NO:97. In some instances, the anti-CD73 antibody is Medi9447 (see U.S. Pat. No. 10,287,362, which is incorporated by reference herein in its entirety). In some instances, the anti-CD73 antibody comprises a heavy chain comprising the amino acid sequence set forth in SEQ ID NO:98. In some instances, the anti-CD73 antibody comprises a light chain comprising the amino acid sequence set forth in SEQ ID NO:99. In some instances, the anti-CD73 antibody comprises a heavy chain comprising the amino acid sequence set forth in SEQ ID NO:98 and a light chain comprising the amino acid sequence set forth in SEQ ID NO:99.

In some instances, the CD73 inhibitor is CPI-006 (Corvus; see US Patent Application Publication No. US 2018/0009899 A1 and international patent application publication no. WO 2017/100670 A1, each of which is incorporated by reference herein in its entirety).

In some instances, the CD73 inhibitor is CB-708 SM (Calithera).

In some instances, the CD73 inhibitor is AB680 (Arcus).

In some instances, the CD73 inhibitor is BMS-986179 (BMS).

### Antibody Fragments

In some instances, the anti-CD73 antibody is an antibody fragment. Fragments of the antibodies described herein (e.g., Fab, Fab′, F(ab′)₂, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

### Minibodies

In some instances, the anti-CD73 antibody is a minibody. Minibodies of anti-CD73 antibodies include diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see, e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-CD73 antibody described herein.

An sc(Fv)₂ is a minibody in which two VHs and two VLs are linked by a linker to form a single chain (Hudson, et al., J. Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

### Bispecific Antibodies

In some instances, the anti-CD73 antibody is a bispecific antibody. Bispecific antibodies are antibodies that have binding specificities for at least two different epitopes. Exemplary bispecific antibodies may bind to two different epitopes of the CD73 protein. Other such antibodies may combine a CD73 binding site with a binding site for another protein. Bispecific antibodies can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In some instances, the anti-CD73 antibody is a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

In some instances, the anti-CD73 antibody is a conjugated antibody. The antibodies disclosed herein may be conjugated antibodies, which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y ¹³¹I), fluorescent substances, luminescent substances, haptens, enzymes, metal chelates, drugs, and toxins (e.g., calcheamicin, Pseudomonas exotoxin A, ricin (e.g. deglycosylated ricin A chain)).

In one embodiment, to improve the cytotoxic actions of anti-CD73 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CD73 antibody is modified with a moiety that improves its stabilization and/or retention in circulation, e.g., in blood, serum, or other tissues, e.g., by at least 1.5, 2, 5, 10, or 50 fold. For example, the anti-CD73 antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CD73 antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies, respectively, or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CD73 antibody (e.g., CL25, HzCL25, 3-F03, 3-F03_411, or 3-F03_413) is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Polynucleotides, Expression Vectors, and Cells

The disclosure also provides polynucleotides and vectors encoding an anti-CD73 antibody or portion thereof (e.g., VH, VL, HC, or LC) described herein. The polynucleotides of the disclosure can be in the form of RNA or in the form of DNA. In some instances, the polynucleotide is DNA. In some instances, the polynucleotide is complementary DNA (cDNA). In some instances, the polynucleotide is RNA.

In some instances, the polynucleotide encodes a VH comprising the VH CDR1, VH CDR2, and VH CDR3 of any antibody described herein (see, e.g., Tables 1, 2, 3, and 5). In some instances, the polynucleotide encodes a VL comprising the VL CDR1, VL CDR2, and VL CDR3 of any antibody described herein (see, e.g., Tables 1, 2, 3, and 5). In some instances, the polynucleotide encodes a heavy chain comprising a VH comprising the VH CDR1, VH CDR2, and VH CDR3 of any antibody described herein (see, e.g., Tables 1, 2, 3, and 5). In some instances, the polynucleotide encodes a light chain comprising a VL comprising the VL CDR1, VL CDR2, and VL CDR3 of any antibody described herein (see, e.g., Tables 1, 2, 3, and 5). In some instances, the polynucleotide is operably linked to a promoter.

In some instances, the polynucleotide comprises: (i) a first nucleic acid sequence encoding a first polypeptide, wherein the first polypeptide comprises a VH comprising the VH CDR1, VH CDR2, and VH CDR3 of any antibody described herein (see, e.g., Tables 1, 2, 3, and 5); and (ii) a second nucleic acid sequence encoding a second polypeptide, wherein the second polypeptide comprises a VL comprising the VL CDR1, VL CDR2, and VL CDR3 of any antibody described herein (see, e.g., Tables 1, 2, 3, and 5). In some instances, the polynucleotide comprises: (i) a first nucleic acid sequence encoding a first polypeptide, wherein the first polypeptide comprises a heavy chain comprising a VH comprising the VH CDR1, VH CDR2, and VH CDR3 of any antibody described herein (see, e.g., Tables 1, 2, 3, and 5); and (ii) a second nucleic acid sequence encoding a second polypeptide, wherein the second polypeptide comprises a light chain comprising a VL comprising the VL CDR1, VL CDR2, and VL CDR3 of any antibody described herein (see, e.g., Tables 1, 2, 3, and 5). In some instances, the first nucleic acid is operably linked to a first promoter and the second nucleic acid is operably linked to a second promoter.

In some instances, the polynucleotide encodes the VH of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:22, 26, and 82-84. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In some instances, the polynucleotide encodes a polypeptide comprising the amino acid sequence set forth in any one of SEQ ID NOs:22, 26, and 82-84. In some instances, the polynucleotide encodes a polypeptide comprising the amino acid sequence set forth in SEQ ID NO:22. In some instances, the polynucleotide is operably linked to a promoter.

In some instances, the polynucleotide encodes the VL of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:23, 27, 80, and 81. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO:23, 27, 80, and 81. In some instances, the polynucleotide encodes a polypeptide comprising the amino acid sequence set forth in any one of SEQ ID NOs:23, 27, 80, and 81. In some instances, the polynucleotide encodes a polypeptide comprising the amino acid sequence set forth in SEQ ID NO:23. In some instances, the polynucleotide is operably linked to a promoter.

In some instances, the polynucleotide comprises: (i) a first nucleic acid encoding a first polypeptide, wherein the first polypeptide comprises the VH of CL25 or a variant thereof (e.g., a humanized version thereof, e.g., HzCL25); and (ii) a second nucleic acid encoding a second polypeptide, wherein the second polypeptide comprises the VL of CL25 or a variant thereof (e.g., a humanized version thereof, e.g., HzCL25). In some instances, the polynucleotide comprises: (i) a first nucleic acid sequence encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NOs:22, 26, and 82-84, and (ii) a second nucleic acid sequence encoding a second polypeptide, wherein the second polypeptide comprises an amino acid sequence having at least 80%, at least 85%, at least 90%, at least 95%, at least 97%, or 100% identity to the amino acid sequence set forth in any one of SEQ ID NOs:23, 27, 80, and 81. In some instances, the polynucleotide comprises: (i) a first nucleic acid sequence encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84; and (ii) a second nucleic acid sequence encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In some instances, the first nucleic acid encodes the amino acid sequence set forth in any one of SEQ ID NOs:22, 26, and 82-84 and the second nucleic acid encodes the amino acid sequence set forth in any one of SEQ ID NOs:23, 27, 80, and 81. In some instances, the first nucleic acid encodes the amino acid sequence set forth in SEQ ID NO:22 and the second nucleic acid encodes the amino acid sequence set forth in SEQ ID NO:23. In some instances, the first nucleic acid is operably linked to a first promoter and the second nucleic acid is operably linked to a second promoter.

In some instances, the polynucleotide encodes the heavy chain of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:24. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24. In some instances, the polynucleotide encodes a polypeptide comprising the amino acid sequence set forth in SEQ ID NO:24. In some instances, the polynucleotide comprises the sequence set forth in SEQ ID NO:89. In some instances, the polynucleotide is operably linked to a promoter.

In some instances, the polynucleotide encodes the light chain of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:25. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In some instances, the polynucleotide encodes a polypeptide comprising the amino acid sequence set forth in SEQ ID NO:25. In some instances, the polynucleotide comprises the sequence set forth in SEQ ID NO:90. In some instances, the polynucleotide is operably linked to a promoter.

In some instances, the polynucleotide encodes the heavy chain of CL25 or a variant thereof (e.g., a humanized version thereof, e.g., HzCL25) and the light chain of CL25 or a variant thereof (e.g., a humanized version thereof, e.g., HzCL25). In some instances, the polynucleotide comprises: (i) a first nucleic acid encoding a first polypeptide, wherein the first polypeptide comprises an amino acid sequence having at least 80%, at least 85%, at least 90%, at least 95%, at least 97%, or 100% identity to the amino acid sequence set forth in SEQ ID NO:24, and (ii) a second nucleic acid encoding a second polypeptide, wherein the second polypeptide comprises an amino acid sequence having at least 80%, at least 85%, at least 90%, at least 95%, at least 97%, or 100% identity to the amino acid sequence set forth in SEQ ID NO:25. In some instances, the polynucleotide comprises: (i) a first nucleic acid encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24, and (ii) a second nucleic acid encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In some instances, the polynucleotide comprises: (i) a first nucleic acid encoding a first polypeptide, wherein the first polypeptide comprises the amino acid sequence set forth in SEQ ID NO:24, and (ii) a second nucleic acid encoding a second polypeptide, wherein the second polypeptide comprises the amino acid sequence set forth in SEQ ID NO:25. In some instances, the polynucleotide comprises: (i) a first nucleic acid comprising the sequence set forth in SEQ ID NO:89, and (ii) a second nucleic acid comprising the sequence set forth in SEQ ID NO:90. In some instances, the first nucleic acid is operably linked to a first promoter and the second nucleic acid is operably linked to a second promoter.

In some instances, the polynucleotide encodes the VH of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs: 32, 60, 62, 63, 67-69, 77, and 85-88. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88. In some instances, the polynucleotide encodes a polypeptide comprising the amino acid sequence set forth in any one of SEQ ID NOs: 32, 60, 62, 63, 67-69, 77, and 85-88. In some instances, the polynucleotide encodes a polypeptide comprising the amino acid sequence set forth in SEQ ID NO:62. In some instances, the polynucleotide encodes a polypeptide comprising the amino acid sequence set forth in SEQ ID NO:63. In some instances, the polynucleotide is operably linked to a promoter.

In some instances, the polynucleotide encodes the VL of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:61, 64, and 65. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO: 61, 64, and 65. In some instances, the polynucleotide encodes a polypeptide comprising the amino acid sequence set forth in any one of SEQ ID NOs: 61, 64, and 65. In some instances, the polynucleotide encodes a polypeptide comprising the amino acid sequence set forth in SEQ ID NO:61. In some instances, the polynucleotide is operably linked to a promoter.

In some instances, the polynucleotide comprises: (i) a first nucleic acid encoding a first polypeptide, wherein the first polypeptide comprises the VH of 3-F03 or a variant thereof (e.g., 3-F03_411 or 3-F03_413); and (ii) a second nucleic acid encoding a second polypeptide, wherein the second polypeptide comprises the VL of 3-F03 or a variant thereof (e.g., 3-F03_411 or 3-F03_413). In some instances, the polynucleotide comprises: (i) a first nucleic acid sequence encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NOs: 32, 60, 62, 63, 67-69, 77, and 85-88, and (ii) a second nucleic acid sequence encoding a second polypeptide, wherein the second polypeptide comprises an amino acid sequence having at least 80%, at least 85%, at least 90%, at least 95%, at least 97%, or 100% identity to the amino acid sequence set forth in any one of SEQ ID NOs:61, 64, and 65. In some instances, the polynucleotide comprises: (i) a first nucleic acid sequence encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88; and (ii) a second nucleic acid sequence encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:61, 64, and 65. In some instances, the first nucleic acid encodes the amino acid sequence set forth in any one of SEQ ID NOs: 32, 60, 62, 63, 67-69, 77, and 85-88 and the second nucleic acid encodes the amino acid sequence set forth in any one of SEQ ID NOs:61, 64, and 65. In some instances, the first nucleic acid encodes the amino acid sequence set forth in SEQ ID NO:62 and the second nucleic acid encodes the amino acid sequence set forth in SEQ ID NO:61. In some instances, the first nucleic acid encodes the amino acid sequence set forth in SEQ ID NO:63 and the second nucleic acid encodes the amino acid sequence set forth in SEQ ID NO:61. In some instances, the first nucleic acid is operably linked to a first promoter and the second nucleic acid is operably linked to a second promoter.

In some instances, the polynucleotide encodes the heavy chain of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NO:30, 33, and 66. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO: 30, 33, and 66. In some instances, the polynucleotide encodes a polypeptide comprising the amino acid sequence set forth in any one of SEQ ID NO: 30, 33, and 66. In some instances, the polynucleotide encodes a polypeptide comprising the amino acid sequence set forth in SEQ ID NO:30. In some instances, the polynucleotide encodes a polypeptide comprising the amino acid sequence set forth in SEQ ID NO:33. In some instances, the polynucleotide comprises the sequence set forth in SEQ ID NO:91. In some instances, the polynucleotide comprises the sequence set forth in SEQ ID NO:93. In some instances, the polynucleotide is operably linked to a promoter.

In some instances, the polynucleotide encodes the light chain of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:31. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some instances, the polynucleotide encodes a polypeptide comprising the amino acid sequence set forth in SEQ ID NO: 31. In some instances, the polynucleotide encodes a polypeptide comprising the amino acid sequence set forth in SEQ ID NO:31. In some instances, the polynucleotide comprises the sequence set forth in SEQ ID NO:92. In some instances, the polynucleotide is operably linked to a promoter.

In some instances, the polynucleotide encodes the heavy chain of 3-F03 or a variant thereof (e.g., 3-F03_411 or 3-F03_413) and the light chain of 3-F03 or a variant thereof (e.g., 3-F03_411 or 3-F03_413). In some instances, the polynucleotide comprises: (i) a first nucleic acid encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NO: 30, 33, and 66, and (ii) a second nucleic acid encoding a second polypeptide, wherein the second polypeptide comprises an amino acid sequence having at least 80%, at least 85%, at least 90%, at least 95%, at least 97%, or 100% identity to the amino acid sequence set forth in SEQ ID NO: 31. In some instances, the polynucleotide comprises: (i) a first nucleic acid encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO: 30, 33, and 66, and (ii) a second nucleic acid encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some instances, the polynucleotide comprises: (i) a first nucleic acid encoding a first polypeptide, wherein the first polypeptide comprises the amino acid sequence set forth in any one of SEQ ID NO: 30, 33, and 66, and (ii) a second nucleic acid encoding a second polypeptide, wherein the second polypeptide comprises the amino acid sequence set forth in SEQ ID NO: 31. In some instances, the polynucleotide comprises: (i) a first nucleic acid encoding a first polypeptide, wherein the first polypeptide comprises the amino acid sequence set forth in SEQ ID NO:30, and (ii) a second nucleic acid encoding a second polypeptide, wherein the second polypeptide comprises the amino acid sequence set forth in SEQ ID NO:31. In some instances, the polynucleotide comprises: (i) a first nucleic acid encoding a first polypeptide, wherein the first polypeptide comprises the amino acid sequence set forth in SEQ ID NO:33, and (ii) a second nucleic acid encoding a second polypeptide, wherein the second polypeptide comprises the amino acid sequence set forth in SEQ ID NO:31. In some instances, the polynucleotide comprises: (i) a first nucleic acid comprising the sequence set forth in SEQ ID NO:91, and (ii) a second nucleic acid comprising the sequence set forth in SEQ ID NO:92. In some instances, the polynucleotide comprises: (i) a first nucleic acid comprising the sequence set forth in SEQ ID NO:93, and (ii) a second nucleic acid comprising the sequence set forth in SEQ ID NO:92. In some instances, the first nucleic acid is operably linked to a first promoter and the second nucleic acid is operably linked to a second promoter.

In some embodiments, a polynucleotide described herein is isolated.

Also provided herein are expression vectors encoding the anti-CD73 antibodies or portions thereof (e.g., VH, VL, HC, and/or LC) described herein. Also provided herein are expression vectors comprising one or more polynucleotides described herein. Various types of expression vectors are known in the art and described herein (e.g., see the section “Methods of Producing Antibodies” above).

Also provided herein are cells comprising the anti-CD73 antibodies described herein. Also provided herein are cells comprising one or more polynucleotides described herein. Also provided herein are cells comprising one or more expression vectors described herein. Various types of cells are known in the art and described herein (e.g., see the section “Methods of Producing Antibodies” above).

Anti-CD73 Antibodies with Altered Glycosylation

Different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to C1q protein (Wright A. & Morrison SL. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995). Thus, in some instances, the anti-CD73 antibody contains an Asn297Ala substitution relative to a wild type constant region.

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632). Aglycosylated forms of the anti-CD73 antibody also have reduced effector function.

Importantly, while removal of the glycans in the CH2 domain appears to have a significant effect on effector function, other functional and physical properties of the antibody remain unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

The anti-CD73 antibodies of the present invention may be modified or altered to elicit increased or decreased effector function(s) (compared to a second CD73-specific antibody). Methods for altering glycosylation sites of antibodies are described, e.g., in U.S. Pat. Nos. 6,350,861 and 5,714,350, WO 05/18572 and WO 05/03175; these methods can be used to produce anti-CD73 antibodies of the present invention with altered, reduced, or no glycosylation.

### A2A/A2B Adenosine Receptor

A2A adenosine receptor is a high affinity receptor, whereas A2B is a low affinity receptor. Adenosine and its agonists can act via one or both of these receptors and can modulate the activity of adenylate cyclase, the enzyme responsible for increasing cyclic AMP (cAMP). The different receptors have differential stimulatory and inhibitory effects on this enzyme. Increased intracellular concentrations of cAMP can suppress the activity of immune and inflammatory cells.

The A2A adenosine receptor can signal in the periphery and the CNS, with agonists explored as anti-inflammatory drugs and antagonists explored for neurodegenerative diseases. In most cell types the A2A subtype inhibits intracellular calcium levels whereas the A2B potentiates them. The A2A receptor generally appears to inhibit inflammatory response from immune cells. A2A are mostly expressed on lymphoid-derived cells, including T-effector cells, T regulatory cells and nature killing cells.

A2B adenosine receptor is a low affinity receptor. A2B receptors are highly expressed in the gastrointestinal tract, bladder, lung and on mast cells. The A2B receptor, although structurally closely related to the A2A receptor and able to activate adenylate cyclase, is functionally different. It has been postulated that this subtype may utilize signal transduction systems other than adenylate cyclase. Among all the adenosine receptors, the A2B adenosine receptor is a low affinity receptor that is thought to remain silent under physiological conditions and to be activated in consequence of increased extracellular adenosine levels. Activation of A2B adenosine receptor can stimulate adenylate cyclase and phospholipase C through activation of Gs and Gq proteins, respectively. Coupling to mitogen activated protein kinases has also been described. A2B receptors are mainly expressed on monocyte-derived cells including dendritic cells, tumor-associated macrophages, myeloid derived suppressive cells (MDSCs), and mesenchymal stromal/stem cells (MSCs).

An exemplary amino acid sequence of human A2A adenosine receptor protein (GenBank Accession No. NP_001265428) is:

(SEQ ID NO: 94)
MPIMGSSVYITVELAIAVLAILGNVLVCWAVWLNSNLQNVTNYFVVSLAA
ADIAVGVLAIPFAITISTGFCAACHGCLFIACFVLVLTQSSIFSLLAIAI
DRYIAIRIPLRYNGLVTGTRAKGIIAICWVLSFAIGLTPMLGWNNCGQPK
EGKNHSQGCGEGQVACLFEDVVPMNYMVYFNFFACVLVPLLLMLGVYLRI
FLAARRQLKQMESQPLPGERARSTLQKEVHAAKSLAIIVGLFALCWLPLH
IINCFTFFCPDCSHAPLWLMYLAIVLSHTNSVVNPFIYAYRIREFRQTFR
KIIRSHVLRQQEPFKAAGTSARVLAAHGSDGEQVSLRLNGHPPGVWANGS
APHPERRPNGYALGLVSGGSAQESQGNTGLPDVELLSHELKGVCPEPPGL
DDPLAQDGAGVS.

An exemplary amino acid sequence of human A2B adenosine receptor protein (GenBank Accession No. NP_000667) is:

(SEQ ID NO: 95)
MLLETQDALYVALELVIAALSVAGNVLVCAAVGTANTLQTPTNYELVSLA
AADVAVGLFAIPFAITISLGFCTDFYGCLFLACFVLVLTQSSIFSLLAVA
VDRYLAICVPLRYKSLVTGTRARGVIAVLWVLAFGIGLTPFLGWNSKDSA
TNNCTEPWDGTTNESCCLVKCLFENVVPMSYMVYENFFGCVLPPLLIMLV
IYIKIFLVACRQLQRTELMDHSRTTLQREIHAAKSLAMIVGIFALCWLPV
HAVNCVTLFQPAQGKNKPKWAMNMAILLSHANSVVNPIVYAYRNRDFRYT
FHKIISRYLLCQADVKSGNGQAGVQPALGVGL.

### A2A/A2B Adenosine Receptor Inhibitors

In some embodiments, the inhibitor of A2A/A2B is a compound selected from Table 6, or a pharmaceutically acceptable salt thereof.

TABLE 6

| Comp. | | |
|---|---|---|
| No. | Name | Structure |
| 1 | 3-(5-Amino-2-(pyridin-2-ylmethyl)-8- (pyrimidin-4-yl)-[1,2,4]triazolo[1,5- c]pyrimidin-7-yl)benzonitrile | 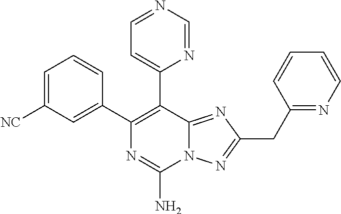 [SMILES: N#Cc1cccc(-c2nc(N)n3nc(Cc4ccccn4)nc3c2-c2ccncn2)c1] |
| 2 | 3-(5-Amino-2-((2,6- difluorophenyl)(hydroxy)methyl)-8- (pyrimidin-4-yl)-[1,2,4]triazolo[1,5- c]pyrimidin-7-yl)benzonitrile | 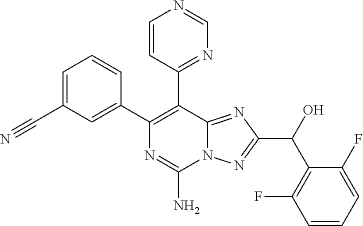 [SMILES: N#Cc1cccc(-c2nc(N)n3nc(C(O)c4c(F)cccc4F)nc3c2-c2ccncn2)c1] |
| 3A | 3-(5-amino-2-((5-(pyridin-2-yl)-2H- tetrazol-2-yl)methyl)-8-(pyrimidin-4-yl)- [1,2,4]triazolo[1,5-c]pyrimidin-7- yl)benzonitrile | 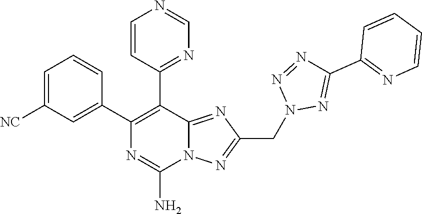 [SMILES: N#Cc1cccc(-c2nc(N)n3nc(Cn4nnc(-c5ccccn5)n4)nc3c2-c2ccncn2)c1] |
| 3B | 3-(5-Amino-2-((5-(pyridin-2-yl)-1H- tetrazol-1-yl)methyl)-8-(pyrimidin-4-yl)- [1,2,4]triazolo[1,5-c]pyrimidin-7- yl)benzonitrile | 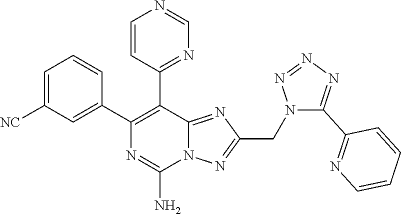 [SMILES: N#Cc1cccc(-c2nc(N)n3nc(Cn4nnnc4-c4ccccn4)nc3c2-c2ccncn2)c1] |
| 4 | 3-(5-Amino-2-((3-methylpyridin-2- yl)methoxy)-8-(pyrimidin-4-yl)- [1,2,4]triazolo[1,5-c]pyrimidin-7- yl)benzonitrile | 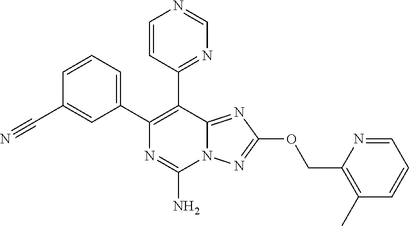 [SMILES: Cc1cccnc1COc1nc2c(-c3ccncn3)c(-c3cccc(C#N)c3)nc(N)n2n1] |
| 5 | 3-(5-Amino-2-(hydroxy(phenyl)methyl)- [1,2,4]triazolo[1,5-c]pyrimidin-7- yl)benzonitrile | 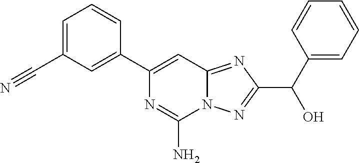 [SMILES: N#Cc1cccc(-c2cc3nc(C(O)c4ccccc4)nn3c(N)n2)c1] |
| 6 | 3-(5-Amino-2-((2,6- difluorophenyl)(hydroxy)methyl)- [1,2,4]triazolo[1,5-c]pyrimidin-7-yl)-2- fluorobenzonitrile | 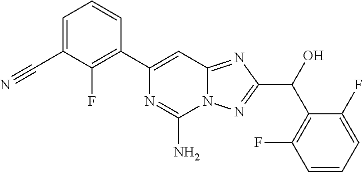 [SMILES: N#Cc1cccc(-c2cc3nc(C(O)c4c(F)cccc4F)nn3c(N)n2)c1F] |
| 7 | 5-Amino-7-(3-cyano-2-fluorophenyl)-2- ((2,6-difluorophenyl)(hydroxy)methyl)- [1,2,4]triazolo[1,5-c]pyrimidine-8- carbonitrile | 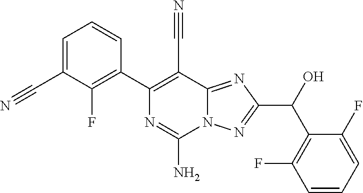 [SMILES: N#Cc1cccc(-c2nc(N)n3nc(C(O)c4c(F)cccc4F)nc3c2C#N)c1F] |
| 8 | 3-(5-Amino-2-((2-fluoro-6-(((1-methyl-2- oxopyrrolidin-3- yl)amino)methyl)phenyl)(hydroxy)methyl)- [1,2,4]triazolo[1,5-c]pyrimidin-7-yl)-2- fluorobenzonitrile | 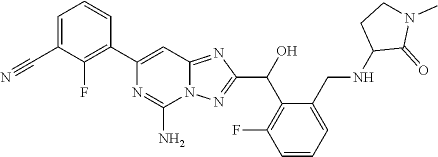 [SMILES: CN1CCC(NCc2cccc(F)c2C(O)c2nc3cc(-c4cccc(C#N)c4F)nc(N)n3n2)C1=O] |
| 9 | 3-(8-Amino-5-(1-methyl-6-oxo-1,6- dihydropyridazin-3-yl)-2-(pyridin-2- ylmethyl)-[1,2,4]triazolo[1,5-a]pyrazin-6- yl)benzonitrile | 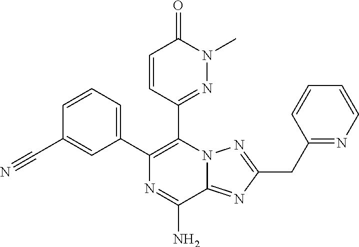 [SMILES: Cn1nc(-c2c(-c3cccc(C#N)c3)nc(N)c3nc(Cc4ccccn4)nn23)ccc1=O] |
| 10 | 3-(8-Amino-2-((2,6- difluorophenyl)(hydroxy)methyl)-5- (pyrimidin-4-yl)-[1,2,4]triazolo[1,5- a]pyrazin-6-yl)benzonitrile | 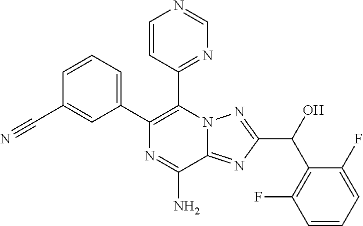 [SMILES: N#Cc1cccc(-c2nc(N)c3nc(C(O)c4c(F)cccc4F)nn3c2-c2ccncn2)c1] |
| 11 | 3-(8-amino-2-(amino(2,6- difluorophenyl)methyl)-5-(4- methyloxazol-5-yl)-[1,2,4]triazolo[1,5- a]pyrazin-6-yl)benzonitrile | 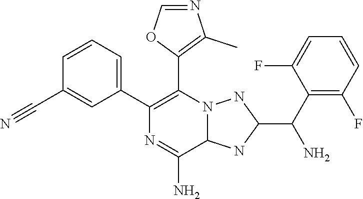 [SMILES: Cc1ncoc1C1=C(c2cccc(C#N)c2)N=C(N)C2NC(C(N)c3c(F)cccc3F)NN12] |
| 12 | 3-(8-amino-2-((2,6- difluorophenyl)(hydroxy)methyl)-5-(2,6- dimethylpyridin-4-yl)-[1,2,4]triazolo[1,5- a]pyrazin-6-yl)benzonitrile | 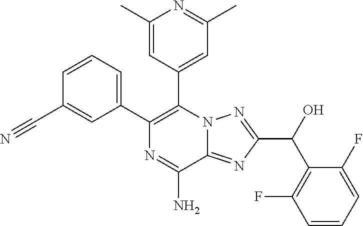 [SMILES: Cc1cc(-c2c(-c3cccc(C#N)c3)nc(N)c3nc(C(O)c4c(F)cccc4F)nn23)cc(C)n1] |
| 13 | 3-(4-amino-2-(pyridin-2-ylmethyl)-7- (pyrimidin-4-yl)-2H-[1,2,3]triazolo[4,5- c]pyridin-6-yl)benzonitrile | 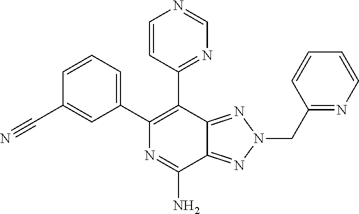 [SMILES: N#Cc1cccc(-c2nc(N)c3nn(Cc4ccccn4)nc3c2-c2ccncn2)c1] |
| 14 | 3-(4-amino-2-((3-fluoropyridin-2- yl)methyl)-7-(pyrimidin-4-yl)-2H- [1,2,3]triazolo[4,5-c]pyridin-6- yl)benzonitrile | 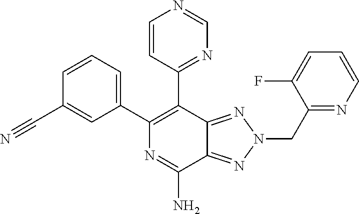 [SMILES: N#Cc1cccc(-c2nc(N)c3nn(Cc4ncccc4F)nc3c2-c2ccncn2)c1] |
| 15 | 3-(4-amino-2-((3-fluoropyridin-2- yl)methyl)-7-(pyridin-4-yl)-2H- [1,2,3]triazolo[4,5-c]pyridin-6- yl)benzonitrile | 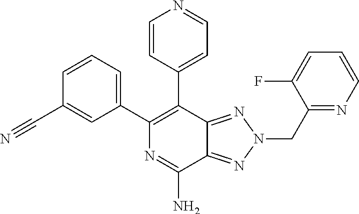 [SMILES: N#Cc1cccc(-c2nc(N)c3nn(Cc4ncccc4F)nc3c2-c2ccncc2)c1] |
| 16 | 3-(4-amino-7-(1-methyl-1H-pyrazol-5- yl)-2-(pyridin-2-ylmethyl)-2H- [1,2,3]triazolo[4,5-c]pyridin-6-yl)-2- fluorobenzonitrile | 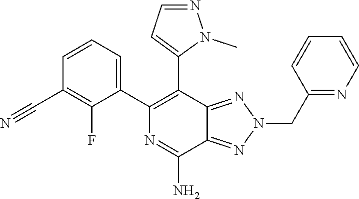 [SMILES: Cn1nccc1-c1c(-c2cccc(C#N)c2F)nc(N)c2nn(Cc3ccccn3)nc12] |
| 17 | 7-(1-((5-Chloropyridin-3-yl)methyl)-1H- pyrazol-4-yl)-3-methyl-9-pentyl-6,9- dihydro-5H-pyrrolo[3,2- d][1,2,4]triazolo[4,3-a]pyrimidin-5-one | 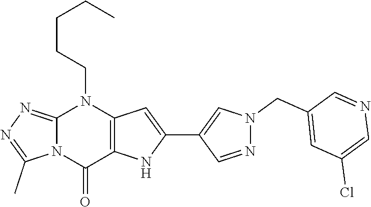 [SMILES: CCCCCn1c2cc(-c3cnn(Cc4cncc(Cl)c4)c3)[nH]c2c(=O)n2c(C)nnc12] |
| 18 | 3-Methyl-7-(1-((5-methylpyridin-3- yl)methyl)-1H-pyrazol-4-yl)-9-pentyl-6,9- dihydro-5H-pyrrolo[3,2- d][1,2,4]triazolo[4,3-a]pyrimidin-5-one | 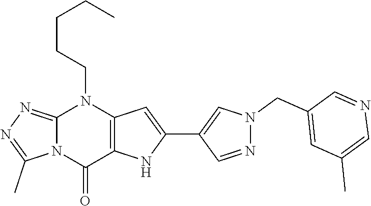 [SMILES: CCCCCn1c2cc(-c3cnn(Cc4cncc(C)c4)c3)[nH]c2c(=O)n2c(C)nnc12] |
| 19 | 3-Methyl-9-pentyl-7-(1-(thieno[3,2- b]pyridin-6-ylmethyl)-1H-pyrazol-4-yl)- 6,9-dihydro-5H-pyrrolo[3,2- d][1,2,4]triazolo[4,3-a]pyrimidin-5-one | 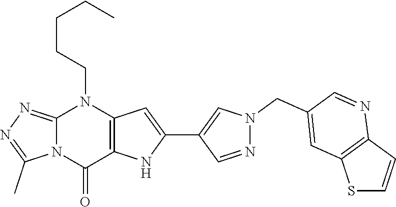 [SMILES: CCCCCn1c2cc(-c3cnn(Cc4cnc5ccsc5c4)c3)[nH]c2c(=O)n2c(C)nnc12] |
| 20 | 7-(1-((2-(2-(Dimethylamino)acetyl)- 1,2,3,4-tetrahydroisoquinolin-6- yl)methyl)-1H-pyrazol-4-yl)-3-methyl-9- pentyl-6,9-dihydro-5H-pyrrolo[3,2- d][1,2,4]triazolo [4,3-a]pyrimidin-5-one | 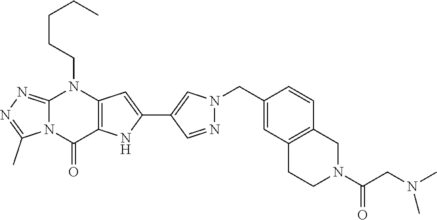 [SMILES: CCCCCn1c2cc(-c3cnn(Cc4ccc5c(c4)CCN(C(=O)CN(C)C)C5)c3)[nH]c2c(=O)n2c(C)nnc12] |
| 21A | 3-(2-((5-(1H-Pyrazol-1-yl)-2H-tetrazol-2- yl)methyl)-5-amino-8-(pyrimidin-4-yl)- [1,2,4]triazolo[1,5-c]pyrimidin-7- yl)benzonitrile | 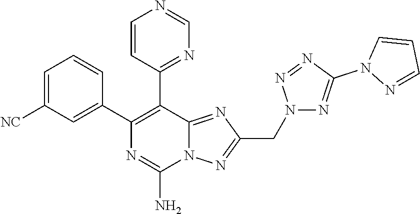 [SMILES: N#Cc1cccc(-c2nc(N)n3nc(Cn4nnc(-n5cccn5)n4)nc3c2-c2ccncn2)c1] |
| 21B | 3-(2-((5-(1H-Pyrazol-1-yl)-1H-tetrazol-1- yl)methyl)-5-amino-8-(pyrimidin-4-yl)- [1,2,4]triazolo[1,5-c]pyrimidin-7- yl)benzonitrile | 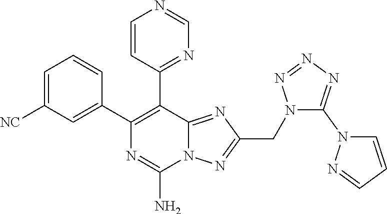 [SMILES: N#Cc1cccc(-c2nc(N)n3nc(Cn4nnnc4-n4cccn4)nc3c2-c2ccncn2)c1] |

In some embodiments, the inhibitor of A2A/A2B is a compound of Formula (I):

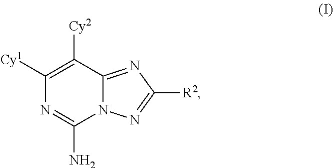

or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl.

In some embodiments of the compound of Formula (I), Cy² is pyrimidinyl.

In some embodiments of the compound of Formula (I), R² is selected from pyridin-2-ylmethyl, (2,6-difluorophenyl)(hydroxy)methyl, (5-(pyridin-2-yl)-1H-tetrazol-1-yl)methyl, (3-methylpyridin-2-yl)methoxy, and (5-(1H-Pyrazol-1-yl)-1H-tetrazol-1-yl)methyl.

In some embodiments, the compound of Formula (I), or a pharmaceutically acceptable salt thereof, is 3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof (see Compound 1, Table 6).

In some embodiments, the compound of Formula (I), or a pharmaceutically acceptable salt thereof, is 3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof (see Compound 2, Table 6).

In some embodiments, the compound of Formula (I), or a pharmaceutically acceptable salt thereof, is 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof (see Compound 3A, Table 6).

In some embodiments, the compound of Formula (I), or a pharmaceutically acceptable salt thereof, is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof (see Compound 3B, Table 6).

In some embodiments, the compound of Formula (I), or a pharmaceutically acceptable salt thereof, is 3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof (see Compound 4, Table 6).

In some embodiments, the compound of Formula (I), or a pharmaceutically acceptable salt thereof, is 3-(2-((5-(1H-Pyrazol-1-yl)-2H-tetrazol-2-yl)methyl)-5-amino-8-(pyrimidin-4-yl)-[1,2,4]triazolo[1,5-c]pyrimidin-7-yl)benzonitrile, or a pharmaceutically acceptable salt thereof (see Compound 21A, Table 6).

In some embodiments, the compound of Formula (I), or a pharmaceutically acceptable salt thereof, is 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile, or a pharmaceutically acceptable salt thereof (see Compound 21B, Table 6).

The synthesis and characterization of compounds of Formula (I) can be found in WO2019/168847 and U.S. 62/891,685, both of which are hereby incorporated by reference in their entireties.

In some embodiments, the inhibitor of A2A/A2B is a compound of Formula (II):

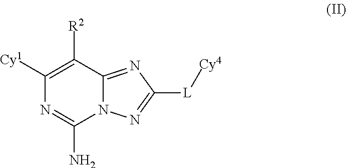

or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
R^{8D} is independently selected from OH, CN, halo, C₁₋₆ alkyl, and C₁₋₆ haloalkyl.

In some embodiments, the compound of Formula (II), or a pharmaceutically acceptable salt thereof, is 3-(5-Amino-2-(hydroxy(phenyl)methyl)-[1,2,4]triazolo[1,5-c]pyrimidin-7-yl)benzonitrile, or a pharmaceutically acceptable salt thereof (see Compound 5, Table 6).

In some embodiments, the compound of Formula (II), or a pharmaceutically acceptable salt thereof, is 3-(5-Amino-2-((2,6-difluorophenyl)(hydroxy)methyl)-[1,2,4]triazolo[1,5-c]pyrimidin-7-yl)-2-fluorobenzonitrile, or a pharmaceutically acceptable salt thereof (see Compound 6, Table 6).

In some embodiments, the compound of Formula (II), or a pharmaceutically acceptable salt thereof, is 5-Amino-7-(3-cyano-2-fluorophenyl)-2-((2,6-difluorophenyl)(hydroxy)methyl)-[1,2,4]triazolo[1,5-c]pyrimidine-8-carbonitrile, or a pharmaceutically acceptable salt thereof (see Compound 7, Table 6).

In some embodiments, the compound of Formula (II), or a pharmaceutically acceptable salt thereof, is 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile, or a pharmaceutically acceptable salt thereof (see Compound 8, Table 6).

The synthesis and characterization of compounds of Formula (II) can be found in WO2019/222677, which is hereby incorporated by reference in its entirety.

In some embodiments, the inhibitor of A2A/A2B is a compound of Formula (III):

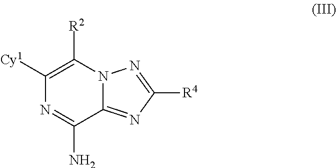

or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl.

In some embodiments, the compound of Formula (III), or a pharmaceutically acceptable salt thereof, is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof (see Compound 9, Table 6).

In some embodiments, the compound of Formula (III), or a pharmaceutically acceptable salt thereof, is 3-(8-Amino-2-((2,6-difluorophenyl)(hydroxy)methyl)-5-(pyrimidin-4-yl)-[1,2,4]triazolo[1,5-a]pyrazin-6-yl)benzonitrile, or a pharmaceutically acceptable salt thereof (See Compound 10, Table 6).

In some embodiments, the compound of Formula (III), or a pharmaceutically acceptable salt thereof, is 3-(8-amino-2-(amino(2,6-difluorophenyl)methyl)-5-(4-methyloxazol-5-yl)-[1,2,4]triazolo[1,5-a]pyrazin-6-yl)benzonitrile, or a pharmaceutically acceptable salt thereof (see Compound 11, Table 6).

In some embodiments, the compound of Formula (III), or a pharmaceutically acceptable salt thereof, is 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile, or a pharmaceutically acceptable salt thereof (see Compound 12, Table 6).

The synthesis and characterization of compounds of Formula (III) can be found in PCT/US2019/040496, which is hereby incorporated by reference in its entirety.

In some embodiments, the inhibitor of A2A/A2B is a compound of Formula (IV):

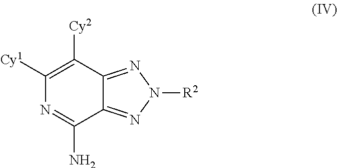

or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl.

In some embodiments, the compound of Formula (IV), or a pharmaceutically acceptable salt thereof, is 3-(4-amino-2-(pyridin-2-ylmethyl)-7-(pyrimidin-4-yl)-2H-[1,2,3]triazolo[4,5-c]pyridin-6-yl)benzonitrile, or a pharmaceutically acceptable salt thereof (see Compound 13, Table 6).

In some embodiments, the compound of Formula (IV), or a pharmaceutically acceptable salt thereof, is 3-(4-amino-2-((3-fluoropyridin-2-yl)methyl)-7-(pyrimidin-4-yl)-2H-[1,2,3]triazolo[4,5-c]pyridin-6-yl)benzonitrile, or a pharmaceutically acceptable salt thereof (see Compound 14, Table 6).

In some embodiments, the compound of Formula (IV), or a pharmaceutically acceptable salt thereof, is 3-(4-amino-2-((3-fluoropyridin-2-yl)methyl)-7-(pyridin-4-yl)-2H-[1,2,3]triazolo[4,5-c]pyridin-6-yl)benzonitrile, or a pharmaceutically acceptable salt thereof (see Compound 15, Table 6).

In come embodiments, the compound of Formula (IV), or a pharmaceutically acceptable salt thereof, is 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile, or a pharmaceutically acceptable salt thereof (see Compound 16, Table 6).

The synthesis and characterization of compounds of Formula (IV) can be found in U.S. 62/798,180, which is hereby incorporated by reference in its entirety.

In some embodiments the inhibitor of A2A/A2B is a compound of Formula (V):

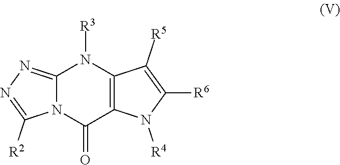

or a pharmaceutically acceptable salt thereof, wherein
R² is selected from H, D, halo, C₁₋₆ alkyl and C₁₋₆ haloalkyl;
R³ is selected from H and C₁₋₆ alkyl;
R⁴ is selected from H and C₁₋₆ alkyl;
R⁵ is selected from H, halo, CN, C₁₋₆ alkyl;
R⁶ is selected from phenyl, C₃₋₇ cycloalkyl, 5-7 membered heteroaryl, and 4-7 membered heterocycloalkyl wherein said phenyl, C₃₋₇ cycloalkyl, 5-7 membered heteroaryl, and 4-7 membered heterocycloalkyl of R⁶ are optionally substituted by 1, 2, or 3 independently selected R^{A} substituents;
each R^{A} is independently selected from (5-10 membered heteroaryl)-C₁₋₃ alkyl- and (4-10 membered heterocycloalkyl)-C₁₋₃ alkyl-, wherein the (5-10 membered heteroaryl)-C₁₋₃ alkyl- and (4-10 membered heterocycloalkyl)-C₁₋₃ alkyl- of R^{A} are each optionally substituted with 1 or 2 independently selected R^{B} substituents;
each R^{B} is independently selected from halo, C₁₋₆ alkyl, and C(O)R^{b26};
R^{b26} is independently selected from H and C₁₋₃ alkyl, wherein the C₁₋₃ alkyl of R^{b26} is optionally substituted with 1 or 2 independently selected R^{C} substituents
each R^{C} is independently selected from halo, C₁₋₆ alkyl, CN, OR^{a36}, and NR^{c36}R^{d36}; and
each R^{a36}, R^{c36}, and R^{d36} is independently selected from H and C₁₋₆ alkyl.

In some embodiments, the compound of Formula (V), or a pharmaceutically acceptable salt thereof, is 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one, or a pharmaceutically acceptable salt thereof (see Compound 17, Table 6).

In some embodiments, the compound of Formula (V), or a pharmaceutically acceptable salt thereof, is 3-Methyl-7-(1-((5-methylpyridin-3-yl)methyl)-1H-pyrazol-4-yl)-9-pentyl-6,9-dihydro-5H-pyrrolo[3,2-d][1,2,4]triazolo[4,3-a]pyrimidin-5-one, or a pharmaceutically acceptable salt thereof (see Compound 18, Table 6).

In some embodiments, the compound of Formula (V), or a pharmaceutically acceptable salt thereof, is 3-Methyl-9-pentyl-7-(1-(thieno[3,2-b]pyridin-6-ylmethyl)-1H-pyrazol-4-yl)-6,9-dihydro-5H-pyrrolo[3,2-d][1,2,4]triazolo[4,3-a]pyrimidin-5-one, or a pharmaceutically acceptable salt thereof (see Compound 19, Table 6).

In some embodiments, the compound of Formula (V), or a pharmaceutically acceptable salt thereof, is 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one, or a pharmaceutically acceptable salt thereof (see Compound 20, Table 6).

The synthesis and characterization of compounds of Formula (V) can be found in US-2019-0337957, which is hereby incorporated by reference in its entirety.

As used herein, “about” when referring to a measurable value such as an amount, a dosage, a temporal duration, and the like, is meant to encompass variations of ±10%. In certain embodiments, “about” can include variations of ±5%, ±1%, or ±0.1% from the specified value and any variations there between, as such variations are appropriate to perform the disclosed methods.

In some embodiments, the compound disclosed herein is the (S)-enantiomer of the compound, or a pharmaceutically acceptable salt thereof. In some embodiments, the compound is the (R)-enantiomer of the compound, or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl (i.e., C₆ aryl).

As used herein, “halo” or “halogen” refers to F, Cl, Br, or I. In some embodiments, a halo is F, Cl, or Br. In some embodiments, a halo is F or Cl. In some embodiments, a halo is F. In some embodiments, a halo is Cl.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl and the like. For example, heterocycloalkyl groups include the following groups (with and without N-methyl substitution):

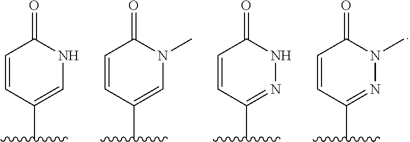

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's PharmaceuticalSciences, 17th ed., Mack Publishing Company, Easton, Pa., 1985, p. 1418 and Journal of Pharmaceutical Science, 66, 2 (1977), each of which is incorporated herein by reference in its entirety.

Compounds described herein, including salts thereof, can be prepared using known organic synthesis techniques and can be synthesized according to any of numerous possible synthetic routes.

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (THPLC) (“Preparative LC-MS Purification: Improved Compound Specific Method Optimization” Karl F. Blom, et al. J. Combi. Chem. 2004, 6(6), 874-883, which is incorporated herein by reference in its entirety) and normal phase silica chromatography.

The compounds described herein can modulate activity of one or more of various G-protein coupled receptors (GPCRs) including, for example, A2A/A2B. The term “modulate” is meant to refer to an ability to increase or decrease the activity of one or more members of the A2A/A2B family. Accordingly, the compounds described herein can be used in methods of modulating A2A/A2B by contacting the A2A/A2B with any one or more of the compounds or compositions described herein. In some embodiments, compounds of the present invention can act as inhibitors of one or both of A2A and A2B. In further embodiments, the compounds described herein can be used to modulate activity of A2A/A2B in an individual in need of modulation of the receptor by administering a modulating amount of a compound described herein, or a pharmaceutically acceptable salt thereof. In some embodiments, modulating is inhibiting.

Given that cancer cell growth and survival is impacted by multiple signaling pathways, the present invention is useful for treating disease states characterized by drug resistant mutants. In addition, different GPCR inhibitors, exhibiting different preferences in the GPCRs which they modulate the activities of, may be used in combination. This approach could prove highly efficient in treating disease states by targeting multiple signaling pathways, reduce the likelihood of drug-resistance arising in a cell, and reduce the toxicity of treatments for disease.

GPCRs to which the present compounds bind and/or modulate (e.g., inhibit) include any member of the A2A/A2B family.

In some embodiments, more than one compound described herein is used to inhibit the activity of one GPCR (e.g., A2A).

In some embodiments, more than one compound described herein is used to inhibit more than one GPCR, such as at least two GPCRs (e.g., A2A and A2B).

In some embodiments, one or more of the compounds is used in combination with another GPCR antagonist to inhibit the activity of one GPCR (e.g., A2A or A2B).

The inhibitors of A2A/A2B described herein can be selective. By “selective” is meant that the compound binds to or inhibits a GPCR with greater affinity or potency, respectively, compared to at least one other GPCR. In some embodiments, the compounds described herein are selective inhibitors of A2A or A2B. In some embodiments, the compounds described herein are selective inhibitors of A2A (e.g., over A2B). In some embodiments, the compounds described herein are selective inhibitors of A2B (e.g., over A2A). In some embodiments, selectivity can be at least about 2-fold, 5-fold, 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the biochemical affinity against each GPCR. In some embodiments, the selectivity of compounds described herein can be determined by cellular assays associated with particular A2A/A2B activity.

As used herein, the term “contacting” refers to the bringing together of indicated moieties in an in vitro system or an in vivo system. For example, “contacting” A2A/A2B with a compound described herein includes the administration of a compound of the present invention to an individual or patient, such as a human, having a A2A/A2B, as well as, for example, introducing a compound described herein into a sample containing a cellular or purified preparation containing the A2A/A2B.

Additional Inhibitors of A2A and/or A2B Adenosine Receptor

Other inhibitors of A2A and/or A2B adenosine receptor useful in combination with an inhibitor of CD73 in the methods described herein are known in the art.

In some instances, the inhibitor of A2A and/or A2B adenosine receptor is CPI-444 (also referred to herein as “Compound B”; 7-(5-methylfuran-2-yl)-3-[[6-[[(3S)-oxolan-3-yl]oxymethyl]pyridin-2-yl]methyl]triazolo[4,5-d]pyrimidin-5-amine).

In some instances, the inhibitor of A2A and/or A2B adenosine receptor is AB928 (3-[2-Amino-6-[1-[[6-(2-hydroxypropan-2-yl)pyridin-2-yl]methyl]triazol-4-yl]pyrimidin-4-yl]-2-methylbenzonitrile).

In some instances, the inhibitor of A2A and/or A2B adenosine receptor is AZD4635 (6-(2-Chloro-6-methylpyridin-4-yl)-5-(4-fluorophenyl)-1,2,4-triazin-3-amine).

In some instances, the inhibitor of A2A and/or A2B adenosine receptor is NIR-178 (5-Bromo-2,6-di(1H-pyrazol-1-yl)pyrimidin-4-amine).

In some instances, the inhibitor of A2A and/or A2B adenosine receptor is EOS100850.

In some instances, the inhibitor of A2A and/or A2B adenosine receptor is a compound, pharmaceutically acceptable salt thereof, or stereoisomer thereof described in US patent application publication no. 2019/0292188, which is incorporated by reference herein in its entirety. In some instances, the inhibitor of A2A and/or A2B adenosine receptor comprises or consists of

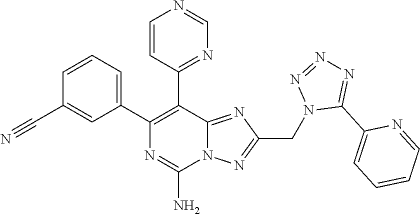 [SMILES: N#Cc1cccc(-c2nc(N)n3nc(Cn4nnnc4-c4ccccn4)nc3c2-c2ccncn2)c1]

(3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile), or a pharmaceutically acceptable salt thereof or a stereoisomer thereof.

In some instances, the inhibitor of A2A and/or A2B adenosine receptor comprises or consists of

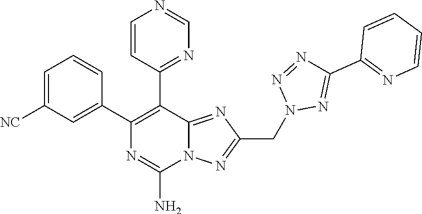 [SMILES: N#Cc1cccc(-c2nc(N)n3nc(Cn4nnc(-c5ccccn5)n4)nc3c2-c2ccncn2)c1]

(3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile), or a pharmaceutically acceptable salt thereof or a stereoisomer thereof.

### Labeled Agents and Assay Methods

Another aspect of the present disclosure relates to labeled agents (i.e., labeled inhibitors of CD73 and inhibitors of A2A and/or A2B adenosine receptor) of the disclosure (radio-labeled, fluorescent-labeled, etc.) that would be useful not only in imaging techniques but also in assays, both in vitro and in vivo, for localizing and quantitating CD73 or A2A and/or A2B receptors in tissue samples, including human, and for identifying CD73 or A2A and/or A2B antagonists by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes adenosine receptor (e.g., A2A and/or A2B) assays that contain such labeled or substituted compounds.

The present disclosure further includes isotopically-labeled agents of the disclosure. An “isotopically” or “radio-labeled” agent is an agent of the disclosure where one or more atoms are replaced or substituted by an atom having an atomic mass or mass number different from the atomic mass or mass number typically found in nature (i.e., naturally occurring). Suitable radionuclides that may be incorporated in agents of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as -CD₃ being substituted for —CH₃). In some embodiments, alkyl groups in any of the disclosed Formulas, e.g., Formula (I), can be perdeuterated.

One or more constituent atoms of the agents presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as -CD₃ being substituted for —CH₃). In some embodiments, the agent includes two or more deuterium atoms. In some embodiments, the agent includes 1-2, 1-3, 1-4, 1-5, or 1-6 deuterium atoms. In some embodiments, all of the hydrogen atoms in an agent can be replaced or substituted by deuterium atoms.

In some embodiments, 1, 2, 3, 4, 5, 6, 7, or 8 hydrogen atoms, attached to carbon atoms of any “alkyl”, “alkenyl”, “alkynyl”, “aryl”, “phenyl”, “cycloalkyl”, “heterocycloalkyl”, or “heteroaryl” substituents or “—C₁₋₆ alkyl-”, “alkylene”, “alkenylene” and “alkynylene” linking groups, as described herein, are each optionally replaced by a deuterium atom.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled agents can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled agents will depend on the specific application of that radio-labeled agent. For example, for in vitro adenosine receptor labeling and competition assays, agents that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled agent” is an agent that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into agents of the disclosure. Synthetic methods for incorporating radio-isotopes into organic compounds and antibodies are well known in the art, and an ordinary skill in the art will readily recognize the methods applicable for the compounds and antibodies of disclosure.

A labeled agent of the disclosure can be used in a screening assay to identify/evaluate agents. For example, a newly synthesized or identified agent (i.e., test agent) which is labeled can be evaluated for its ability to bind an adenosine receptor or CD73 by monitoring its concentration variation when contacting with the adenosine receptor or CD73, respectively, through tracking of the labeling. For example, a test agent (labeled) can be evaluated for its ability to reduce binding of another agent which is known to bind to an adenosine receptor or CD73 (i.e., standard agent). Accordingly, the ability of a test agent to compete with the standard agent for binding to the adenosine receptor or CD73 directly correlates to its binding affinity. Conversely, in some other screening assays, the standard agent is labeled and test agents are unlabeled. Accordingly, the concentration of the labeled standard agent is monitored in order to evaluate the competition between the standard agent and the test agent, and the relative binding affinity of the test agent is thus ascertained.

### Combination Therapy and Indications

The CD73 inhibitors of the present disclosure can modulate the activity of CD73. Accordingly, the CD73 inhibitors described herein can be used in methods of inhibiting CD73 by contacting CD73 with any one or more of the antibodies or compositions thereof described herein. Likewise, the A2A and/or A2B inhibitors of the present disclosure can modulate the activity of A2A and/or A2B adenosine receptor. Accordingly, the A2A and/or A2B adenosine receptor inhibitors, salts or stereoisomers described herein can be used in methods of inhibiting A2A and/or A2B adenosine receptor by contacting A2A and/or A2B adenosine receptor, respectively with any one or more of the A2A and/or A2B adenosine receptor inhibitors or compositions thereof described herein. CD73 inhibitors of the present disclosure and inhibitors of A2A and/or A2B adenosine receptor of the present disclosure can function synergistically, e.g., to treat a disease or disorder, e.g., cancer. Accordingly, the CD73 inhibitors described herein can be used in combination with the inhibitors of A2A and/or A2B adenosine receptor described herein in methods of inhibiting CD73 and A2A and/or A2B adenosine receptor by contacting CD73 with any one or more of the CD73 inhibitors or compositions thereof described herein and contacting A2A and/or A2B adenosine receptor with any one or more of the inhibitors of A2A and/or A2B adenosine receptor or compositions thereof described herein.

In some embodiments, the CD73 inhibitors and A2A and/or A2B adenosine receptor inhibitors can be used in combination in methods of inhibiting: (i) activity of CD73 in an individual/patient in need of the inhibition, and (ii) activity of A2A and/or A2B adenosine receptor in an individual/patient in need of the inhibition, by administering an effective amount of a CD73 inhibitor described herein and an inhibitor of A2A and/or A2B adenosine receptor described herein. In some embodiments, modulating is inhibiting. In some embodiments, the contacting is in vivo. In some embodiments, the contacting is ex vivo or in vitro.

Another aspect of the present disclosure pertains to methods of treating a CD73- and/or A2A and/or A2B adenosine receptor-associated disease or disorder in an individual (e.g., patient) by administering to the individual in need of such treatment a therapeutically effective amount or dose of one or more CD73 inhibitors of the present disclosure or a pharmaceutical composition thereof and a therapeutically effective amount or dose of one or more inhibitors of A2A and/or A2B adenosine receptor of the present disclosure or a pharmaceutical composition thereof. A CD73-associated disease or disorder can include any disease, disorder or condition that is directly or indirectly linked to expression or activity of CD73, including overexpression and/or abnormal activity levels. An A2A and/or A2B adenosine receptor-associated disease or disorder can include any disease, disorder or condition that is directly or indirectly linked to expression or activity of A2A and/or A2B adenosine receptor, including overexpression and/or abnormal activity levels. A CD73- and/or A2A and/or A2B adenosine receptor-associated disease or disorder can include any disease, disorder or condition that is directly or indirectly linked to expression or activity of CD73 and/or A2A and/or A2B adenosine receptor, including overexpression and/or abnormal activity levels of CD73 and/or A2A and/or A2B adenosine receptor.

Another aspect of the present disclosure pertains to methods of treating a disease or disorder (e.g., cancer) in an individual (e.g., patient) by administering to the individual in need of such treatment a therapeutically effective amount or dose of one or more inhibitors of CD73 of the present disclosure or a pharmaceutical composition thereof and a therapeutically effective amount or dose of one or more inhibitors of A2A and/or A2B adenosine receptor of the present disclosure or a pharmaceutical composition thereof, wherein the disease or disorder has a high adenosine signature. Methods of determining that a disease or disorder has a high adenosine signature are known in the art. For instance, gene expression analysis of tumor tissue may be performed using a defined panel of adenosine-responsive genes.

The CD73 inhibitors and inhibitors of A2A and/or A2B adenosine receptor of the present disclosure are useful in combination in the treatment of diseases related to the activity of CD73 and/or A2A and/or A2B adenosine receptor including, for example, cancer, inflammatory diseases, cardiovascular diseases, neurodegenerative diseases, immunomodulatory disorders, central nerve system diseases, and diabetes.

In some embodiments, the inhibitor of human CD73 comprises:
(a) an antibody comprising a variable heavy (VH) domain comprising VH complementarity determining region (CDR)₁, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
comprising a variable light (VL) domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6);
(b) an antibody that binds to human CD73 at an epitope within amino acids 40-53 of SEQ ID NO:70;
(c) an antibody that binds to human CD73 and competes for binding to human CD73 with an antibody that has a heavy chain comprising the amino acid sequence of SEQ ID NO:24 and a light chain comprising the amino acid sequence of SEQ ID NO:25; or
(d) an antibody comprising a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
an antibody comprising a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) an antibody that binds to human CD73 at an epitope within amino acids 386-399 and 470-489 of SEQ ID NO:70;
(f) an antibody that binds to human CD73 and competes for binding to human CD73 with an antibody that has a heavy chain comprising the amino acid sequence of SEQ ID NO:30 and a light chain comprising the amino acid sequence of SEQ ID NO:31;
(g) an antibody that binds to human CD73 and competes for binding to human CD73 with an antibody that has a heavy chain comprising the amino acid sequence of SEQ ID NO:33 and a light chain comprising the amino acid sequence of SEQ ID NO:31;
(h) an antibody selected from the group consisting of 11E1, Medi9447, CPI-006, and BMS-986179; or
(i) an inhibitor selected from the group consisting of CB-708 and AB680.

In some embodiments, the inhibitor of A2A adenosine receptor and/or A2B adenosine receptor (A2A/A2B) comprises a compound of:
(a) Formula (I):

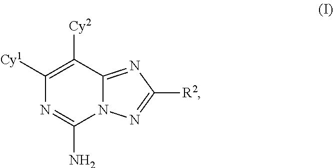

or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and each R^{a4}, R^{c4}, and R^{d4} are independently selected from H and C₁₋₆ alkyl;
(b) Formula (II):

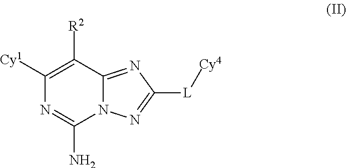

or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl;
(c) Formula (III):

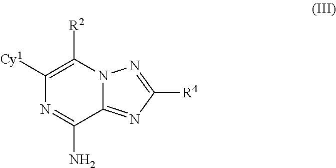

or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl; or
(d) Formula (IV):

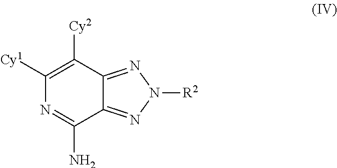

or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl.
or a pharmaceutically acceptable salt thereof.

In some embodiments, the inhibitor of human CD73 comprises:
(a) an antibody comprising a variable heavy (VH) domain comprising VH complementarity determining region (CDR)₁, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
comprising a variable light (VL) domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6);
(b) an antibody that binds to human CD73 at an epitope within amino acids 40-53 of SEQ ID NO:70;
(c) an antibody that binds to human CD73 and competes for binding to human CD73 with an antibody that has a heavy chain comprising the amino acid sequence of SEQ ID NO:24 and a light chain comprising the amino acid sequence of SEQ ID NO:25; or
(d) an antibody comprising a VH domain comprising VH CDR1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
an antibody comprising a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39);
(e) an antibody that binds to human CD73 at an epitope within amino acids 386-399 and 470-489 of SEQ ID NO:70;
(f) an antibody that binds to human CD73 and competes for binding to human CD73 with an antibody that has a heavy chain comprising the amino acid sequence of SEQ ID NO:30 and a light chain comprising the amino acid sequence of SEQ ID NO:31;
(g) an antibody that binds to human CD73 and competes for binding to human CD73 with an antibody that has a heavy chain comprising the amino acid sequence of SEQ ID NO:33 and a light chain comprising the amino acid sequence of SEQ ID NO:31;
(h) an antibody selected from the group consisting of 11E1, Medi9447, CPI-006, and BMS-986179; or
(i) an inhibitor selected from the group consisting of CB-708 and AB680; and the inhibitor of A2A adenosine receptor and/or A2B adenosine receptor (A2A/A2B) comprises a compound of:
(a) Formula (I):

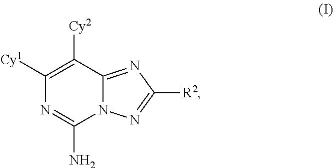

or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl;
(b) Formula (II):

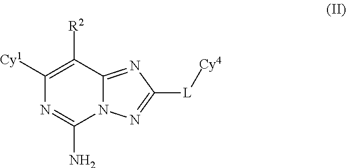

or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl;
(c) Formula (III):

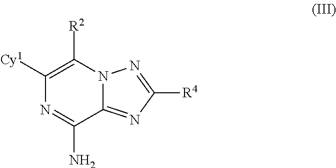

or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl; or
(d) Formula (IV):

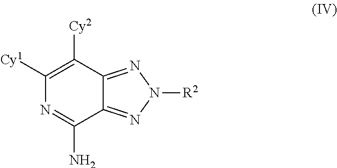

or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl.
or a pharmaceutically acceptable salt thereof.

In some embodiments, the CD73 inhibitor comprises an antibody comprising a VH domain comprising the amino acid sequence set forth in SEQ ID NO:22 and a VL domain comprising the amino acid sequence set forth in SEQ ID NO:23, and the inhibitor of A2a and/or A2B adenosine receptor comprises the compound 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the CD73 inhibitor comprises an antibody comprising a heavy chain comprising the amino acid sequence set forth in SEQ ID NO:24 and a light chain comprising the amino acid sequence set forth in SEQ ID NO:25, and the inhibitor of A2a and/or A2B adenosine receptor comprises the compound 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the CD73 inhibitor comprises an antibody comprising a VH domain comprising the amino acid sequence set forth in SEQ ID NO:62 and a VL domain comprising the amino acid sequence set forth in SEQ ID NO:61, and the inhibitor of A2a and/or A2B adenosine receptor comprises the compound 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the CD73 inhibitor comprises an antibody comprising a heavy chain comprising the amino acid sequence set forth in SEQ ID NO:30 and a light chain comprising the amino acid sequence set forth in SEQ ID NO:31, and the inhibitor of A2a and/or A2B adenosine receptor comprises the compound 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the CD73 inhibitor comprises an antibody comprising a VH domain comprising the amino acid sequence set forth in SEQ ID NO:63 and a VL domain comprising the amino acid sequence set forth in SEQ ID NO:61, and the inhibitor of A2a and/or A2B adenosine receptor comprises the compound 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

In some embodiments, the CD73 inhibitor comprises an antibody comprising a heavy chain comprising the amino acid sequence set forth in SEQ ID NO:33 and a light chain comprising the amino acid sequence set forth in SEQ ID NO:31, and the inhibitor of A2a and/or A2B adenosine receptor comprises the compound 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile.

Based on the compelling roles of CD73 and A2A and/or A2B adenosine receptor in multiple immunosuppressive mechanisms, combination therapy can boost the immune system to suppress tumor progression. CD73 inhibitors and inhibitors of A2A and/or A2B adenosine receptor can be used in combination to treat, optionally in further combination with other therapies, bladder cancer, lung cancer (e.g., non-small cell lung cancer (NSCLC), lung metastasis), melanoma (e.g., metastatic melanoma), breast cancer, cervical cancer, ovarian cancer, colorectal cancer, pancreatic cancer, esophageal cancer, prostate cancer, kidney cancer, skin cancer, thyroid cancer, liver cancer (e.g., hepatocellular carcinoma), uterine cancer, head and neck cancer (e.g., head and neck squamous cell carcinoma), and renal cell carcinoma. In some embodiments, the prostate cancer is metastatic castrate-resistant prostate carcinoma (mCRPC). In some embodiments, the colorectal cancer is colorectal carcinoma (CRC).

In some embodiments, the disease or disorder is lung cancer (e.g., non-small cell lung cancer), melanoma, pancreatic cancer, breast cancer, head and neck squamous cell carcinoma, prostate cancer, liver cancer, color cancer, endometrial cancer, bladder cancer, skin cancer, cancer of the uterus, renal cancer, gastric cancer, or sarcoma. In some embodiments, the sarcoma is Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, or undifferentiated pleomorphic sarcoma.

In some embodiments, the disease or disorder is head and neck cancer (e.g., head and neck squamous cell carcinoma), colorectal cancer, lung cancer (e.g., non-small cell lung cancer (NSCLC)), melanoma, ovarian, bladder, liver cancer (e.g., hepatocellular carcinoma), or renal cell carcinoma.

In some embodiments, the disease or disorder is mesothelioma or adrenocarcinoma. In some embodiments, the disease or disorder is mesothelioma. In some embodiments, the disease or disorder is adrenocarcinoma.

MDSC (myeloid-derived suppressor cells) are a heterogenous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. In some embodiments, the compounds, etc. described herein can be used in methods related to cancer tissue (e.g., tumors) with high infiltration of MDSCs, including Solid tumors with high basal level of macrophage and/or MDSC infiltration.

In some embodiments, the CD73 inhibitors and inhibitors of A2A and/or A2B adenosine receptor of the disclosure can be used in combination in treating pulmonary inflammation, including bleomycin-induced pulmonary fibrosis and injury related to adenosine deaminase deficiency.

In some embodiments, the CD73 inhibitors and inhibitors of A2A and/or A2B adenosine receptor of the disclosure can be used in combination as a treatment for inflammatory disease such as allergic reactions (e.g., CD73- and/or A2A and/or A2B adenosine receptor-dependent allergic reactions) and other CD73- and/or A2A and/or A2B adenosine receptor-immune reactions. Further inflammatory diseases that can be treated by combination of the CD73 inhibitors and A2A and/or A2B adenosine receptor inhibitors of the disclosure include respiratory disorders, sepsis, reperfusion injury, and thrombosis.

In some embodiments, the CD73 inhibitors and A2A and/or A2B adenosine receptor inhibitors of the disclosure can be used in combination as a treatment for cardiovascular disease such as coronary artery disease (myocardial infarction, angina pectoris, heart failure), cerebrovascular disease (stroke, transient ischemic attack), peripheral artery disease, and aortic atherosclerosis and aneurysm. Atherosclerosis is an underlying etiologic factor in many types of cardiovascular disease. Atherosclerosis begins in adolescence with fatty streaks, which progress to plaques in adulthood and finally results in thrombotic events that cause occlusion of vessels leading to clinically significant morbidity and mortality.

In some embodiments, the CD73 inhibitors and A2A and/or A2B adenosine receptor inhibitors of the disclosure can be used in combination as a treatment for disorders in motor activity; deficiency caused by degeneration of the striatonigral dopamine system; and Parkinson's disease; some of the motivational symptoms of depression.

In some embodiments, the CD73 inhibitors and A2A and/or A2B adenosine receptor inhibitors of the disclosure can be used in combination as a treatment for diabetes and related disorders, such as insulin resistance. Diabetes affects the production of adenosine and the expression of A2B adenosine receptors (A2BRs) that stimulate IL-6 and CRP production, insulin resistance, and the association between A2BR gene single-nucleotide polymorphisms (ADORA2B SNPs) and inflammatory markers. The increased A2BR signaling in diabetes may increase insulin resistance in part by elevating pro-inflammatory mediators. Selective CD73 inhibitors may be useful to treat insulin resistance.

The terms “individual” or “patient” or “subject”, used interchangeably, refer to any animal, including mammals, preferably mice, rats, other rodents, rabbits, dogs, cats, swine, cattle, sheep, horses, or primates, and most preferably humans (i.e., a human subject).

The phrase “therapeutically effective amount” refers to the amount of active antibody or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the CD73 inhibitors and A2A and/or A2B adenosine receptor inhibitors of the disclosure are useful in combination in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Pharmaceutical Compositions

The CD73 inhibitors and inhibitors of A2A and/or A2B adenosine receptor described herein can be formulated as pharmaceutical compositions for administration to a subject, e.g., to treat a disorder described herein. In some instances, the pharmaceutical composition comprises a CD73 inhibitor as a single agent. In some instances, the pharmaceutical composition comprises an inhibitor of A2A and/or A2B adenosine receptor as a single agent. In some instances, the pharmaceutical compsotion comprises a CD73 inhibitor and an inhibitor of A2A and/or A2B adenosine receptor.

Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the CD73 inhibitor and/or inhibitor of A2A and/or A2B adenosine receptor may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

### Administration

The CD73 inhibitor and inhibitor of A2A and/or A2B adenosine receptor can be administered to a subject, e.g., a subject in need thereof, for example, a human subject, by a variety of methods. In some instances, the CD73 inhibitor and the inhibitor of A2A and/or A2B adenosine receptor are administered to the subject by the same route. In some instances, the CD73 inhibitor and the inhibitor of A2A and/or A2B adenosine receptor are administered to the subject by different routes. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the CD73 inhibitor or inhibitor of A2A and/or A2B adenosine receptor can also be tailored for the individual case, e.g., by monitoring the subject, e.g., using tomographic imaging, e.g., to visualize a tumor.

Each of the CD73 inhibitor and the inhibitor of A2A and/or A2B adenosine receptor can be administered as a fixed dose, or in a mg/kg patient weight dose. The dose can also be chosen to reduce or avoid production of antibodies against the CD73 inhibitor or inhibitor of A2A and/or A2B adenosine receptor. Dosage regimens are adjusted to provide the desired response, e.g., a therapeutic response or a combinatorial therapeutic effect. Generally, doses of the CD73 inhibitor and of the inhibitor of A2A and/or A2B adenosine receptor can be used in order to provide a subject with the agent in bioavailable quantities.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody and/or inhibitor may be administered via continuous infusion.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example A: Activity of A2A/A2B Inhibitors

### I. A2A Tag-lite® HTRF Assay

Assays were conducted in black low volume 384-well polystyrene plates (Greiner 784076-25) in a final volume of 10 μL. Test compounds were first serially diluted in DMSO and 100 nl added to the plate wells before the addition of other reaction components. The final concentration of DMSO was 1%. Tag-lite® Adenosine A2A labeled cells (CisBio C1TT1A2A) were diluted 1:5 into Tag-lite buffer (CisBio LABMED) and spun 1200 g for 5 mins. The pellet was resuspended at a volume 10.4× the initial cell suspension volume in Tag-lite buffer, and Adenosine A2A Receptor Red antagonist fluorescent ligand (CisBio L0058RED) added at 12.5 nM final concentration. 10 ul of the cell and ligand mix was added to the assay wells and incubated at room temperature for 45 minutes before reading on a PHERAstar FS plate reader (BMG Labtech) with HTRF 337/620/665 optical module. Percent binding of the fluorescent ligand was calculated; where 100 nM of A2A antagonist control ZM 241385 (Tocris 1036) displaces the ligand 100% and 1% DMSO has 0% displacement. The % binding data versus the log of the inhibitor concentration was fitted to a one-site competitive binding model (GraphPad Prism version 7.02) where the ligand constant=12.5 nM and the ligand Kd=1.85 nM. The Kᵢ data obtained via this method are shown in Table 7.

### II. Adenosine A2B Receptor Cyclic AMP GS Assay

Stably transfected HEK-293 cells expressing the human adenosine A2B receptor (Perkin Elmer) were maintained in MEM culture medium with 10% FBS and 100 μg/ml Geneticin (Life Technologies). 18 to 24 hours prior to assay, geneticin was removed from culture. The cisbio cAMP-GS Dynamic kit utilizing the FRET (Fluorescence Resonance Energy Transfer) technology was used to measure cAMP accumulation in the cells. Compounds of the present disclosure at an appropriate concentration were mixed with 10000 cells/well in white 96 well half area plates (Perkin Elmer) for 30 min at RT gently shaking. Agonist, NECA (R&D Technologies) at 12 nM was added to each well for 60 min at RT gently shaking. Detection reagents, d2-labeled cAMP (acceptor) and anti-cAMP cryptate (donor) were added to each well for 60 min at RT gently shaking. Plates were read on Pherastar (BMG Labtech), fluorescence ratio 665/620 was calculated and EC₅₀ determination was performed by fitting the curve of percent of control versus the log of the compound concentration using GraphPad Prism. The EC₅₀ data obtained via this method are shown in Table 7.

TABLE 7
The A_{2A}_Ki data (Example A(I)) and A_{2B}_cAMP_EC₅₀ data
(Example A(II)) are provided below.

| Comp. | A_{2A}_Ki | A_{2B}_cAMP_EC₅₀ |
|---|---|---|
| No. | (nM) | (nM) |
| 1 | † | † |
| 2 | † | † |
| 3 | † | † |
| 4 | † | † |
| 5 | † | † |
| 6 | † | † |
| 7 | † | † |
| 8 | † | †† |
| 9 | † | † |
| 10 | † | † |
| 11 | † | † |
| 12 | † | †† |
| 13 | † | † |
| 14 | † | † |
| 15 | † | † |
| 16 | † | † |
| 17 | † | †† |
| 18 | † | † |
| 19 | † | † |
| 20 | † | † |
| 21 | † | † |

† indicates A_{2A}_Kᵢ or A_{2B}_cAMP_EC₅₀ ≤ 10 nM,
†† indicates A_{2A}_Kᵢ or A_{2B}_cAMP_EC₅₀ > 10 nM but ≤ 100 nM,
††† indicates A_{2A}_Kᵢ or A_{2B}_cAMP_EC₅₀ > 100 nM but ≤ 1 μM,
†††† indicates A_{2A}_Kᵢ or A_{2B}_cAMP_EC₅₀ is greater than 1 μM.

### Example A1: Synthesis of 3-(5-Amino-2-(pyridin-2-ylmethyl)-8-(pyrimidin-4-yl)-[1,2,4]triazolo[1,5-c]pyrimidin-7-yl)benzonitrile (Compound 1)

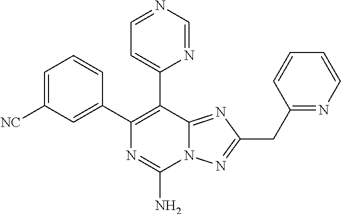 [SMILES: N#Cc1cccc(-c2nc(N)n3nc(Cc4ccccn4)nc3c2-c2ccncn2)c1]

### Step 1: 3-(2-Amino-6-chloropyrimidin-4-yl)benzonitrile

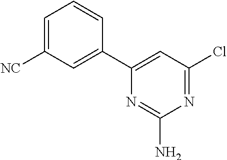 [SMILES: N#Cc1cccc(-c2cc(Cl)nc(N)n2)c1]

A mixture of 4,6-dichloropyrimidin-2-amine (2.5 g, 15.2 mmol), (3-cyanophenyl)boronic acid (2.02 g, 13.7 mmol), tetrakis(triphenylphosphine)palladium(0) (1.06 g, 0.92 mmol) and sodium carbonate (3.23 g, 30.5 mmol) in 1,4-dioxane (60 mL), and water (5 mL) was degassed with nitrogen, then the resulting mixture was heated and stirred at 60° C. for two days. After cooled to room temperature (r.t.), the mixture was concentrated, diluted with water, and extracted with DCM (30 mL×3). The combined organic layers were dried over MgSO₄, filtered, and concentrated. The resulting residue was purified by flash chromatography on a silica gel column eluting with 8% EtOAc in dichloromethane to afford the desired product. LCMS calculated for C₁₁H₈ClN₄ (M+H)⁺: 231.0. Found: 231.0.

### Step 2: 2-(Pyridin-2-yl)acetohydrazide

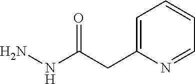 [SMILES: NNC(=O)Cc1ccccn1]

Hydrazine (4.15 mL, 132 mmol) was added to a ethanol (66 mL) solution of methyl 2-(pyridin-2-yl)acetate (10 g, 66.2 mmol) at r.t. The mixture was heated and stirred at 85° C. for 4 h, and then cooled to r.t. White solid was formed upon standing, which was collected via filtration and used in next step without further purification. LCMS calculated for C₇H₁₀N₃O (M+H)⁺: 152.1. Found: 152.0.

### Step 3: 3-(5-Amino-2-(pyridin-2-ylmethyl)-[1,2,4]triazolo[1,5-c]pyrimidin-7-yl)benzonitrile

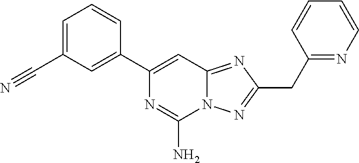 [SMILES: N#Cc1cccc(-c2cc3nc(Cc4ccccn4)nn3c(N)n2)c1]

2-(pyridin-2-yl)acetohydrazide (2.62 g, 17.34 mmol) was added to a ethanol (35 mL) solution of 3-(2-amino-6-chloropyrimidin-4-yl)benzonitrile (4.00 g, 17.34 mmol) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₈H₁₄N₇(M+H)⁺: 328.1; found 328.1.

### Step 4: 3-(5-Amino-8-bromo-2-(pyridin-2-ylmethyl)-[1,2,4]triazolo[1,5-c]pyrimidin-7-yl)benzonitrile

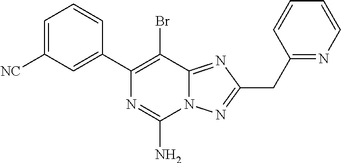 [SMILES: N#Cc1cccc(-c2nc(N)n3nc(Cc4ccccn4)nc3c2Br)c1]

To a mixture of 3-(5-amino-2-(pyridin-2-ylmethyl)-[1,2,4]triazolo[1,5-c]pyrimidin-7-yl)benzonitrile (2 g, 6.11 mmol) in DMF (12 mL) at −30° C. was added NBS (1.09 g, 6.11 mmol) portion-wise. The reaction mixture was allowed to slowly warm to 0° C., resulting a homogenous solution. After stirring at 0° C. for 1 h, the reaction mixture was diluted with saturated NaHCO₃ solution and the resulting solid was collected by filtration. The solid was then purified by flash chromatography on a silica gel column eluting with 0 to 10% MeOH in DCM to afford the desired product. LCMS calculated for C₁₈H₁₃BrN₇ (M+H)⁺: 406.0; found 406.0.

### Step 5: 3-(5-Amino-2-(pyridin-2-ylmethyl)-8-(pyrimidin-4-yl)-[1,2,4]triazolo[1,5-c]pyrimidin-7-yl)benzonitrile

Pd(Ph₃P)₄ (284 mg, 0.246 mmol) was added to a mixture of 4-(tributylstannyl)pyrimidine (1090 mg, 2.95 mmol), 3-(5-amino-8-bromo-2-(pyridin-2-ylmethyl)-[1,2,4]triazolo[1,5-c]pyrimidin-7-yl)benzonitrile (1000 mg, 2.46 mmol), and copper(I) chloride (244 mg, 2.46 mmol) in 1,4-dioxane (12 mL). The reaction mixture was purged with N₂ and stirred at 80° C. for 7 h. The resulting mixture was cooled to r.t., concentrated, diluted with DCM (50 mL) and washed with saturated NH₄OH solution. The organic layer was dried over Na₂SO₄, concentrated, and purified by preparative LC-MS (pH 2, acetonitrile/water with TFA) to afford the product as a TFA salt. LCMS calculated for C₂₂H₁₆N₉(M+H)⁺: 406.2; found 406.2. ¹H NMR (500 MHz, DMSO) δ 8.95 (s, 1H), 8.83 (d, J=5.3 Hz, 1H), 8.59 (d, J=5.1 Hz, 1H), 7.96 (m, 1H), 7.88 (d, J=5.1 Hz, 1H), 7.82 (d, J=7.6 Hz, 1H), 7.76 (s, 1H), 7.60-7.53 (m, 2H), 7.53-7.48 (m, 1H), 7.48-7.42 (m, 1H), 4.49 (s, 2H).

### Example A2: Synthesis of 3-(5-Amino-2-((2,6-difluorophenyl)(hydroxy)methyl)-8-(pyrimidin-4-yl)-[1,2,4]triazolo[1,5-c]pyrimidin-7-yl)benzonitrile (Compound 2)

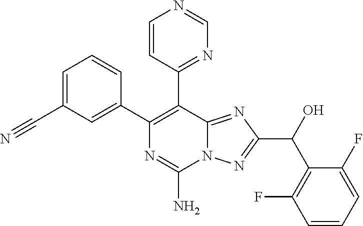 [SMILES: N#Cc1cccc(-c2nc(N)n3nc(C(O)c4c(F)cccc4F)nc3c2-c2ccncn2)c1]

### Step 1: Methyl 2-(2,6-difluorophenyl)-2-hydroxyacetate

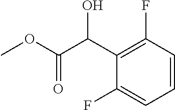

Concentrated sulfuric acid (1.42 mL, 27 mmol) was added to a methanol (45 mL) solution of 2,6-difluoromandelic acid (5 g, 27 mmol) at 0° C. The mixture was stirred at r.t. for 4 h before being concentrated. To the resulting slurry was added saturated NaHCO₃ solution (30 mL). The resulting mixture was extracted with DCM (3×20 mL). The combined organic layers were washed with water, dried over Mg₂SO₄, filtered, and concentrated to afford the crude product, which was used in the next step without further purification. LC-MS calculated for C₁₁H₁₂F₂NO₃ (M+H+MeCN)⁺: m/z=244.1; found 244.2.

### Step 2: 3-(5-Amino-2-((2,6-difluorophenyl)(hydroxy)methyl)-8-(pyrimidin-4-yl)-[1,2,4]triazolo[1,5-c]pyrimidin-7-yl)benzonitrile

This compound was prepared using similar procedures as described for Example A1, with methyl 2-(2,6-difluorophenyl)-2-hydroxyacetate replacing methyl 2-(pyridin-2-yl)acetate in Step 2. The two enantiomers were separated by chiral SFC using a Phenomenex Lux Cellulose-1 column (21.2×250 mm, 5 m particle size) eluting with an isocratic mobile phase 25% MeOH in CO₂ with a flow rate of 80 mL/minute. Peak 1 was isolated, and further purified by prep-LCMS (pH=2, MeCN/water with TFA) to give the desired product as a TFA salt. LC-MS calculated for C₂₃H₁₅F₂N₈O (M+H)⁺: m/z=457.1; found 457.1. ¹H NMR (500 MHz, DMSO) δ 8.94 (d, J=1.3 Hz, 1H), 8.81 (d, J=5.2 Hz, 1H), 7.85 (dd, J=5.3, 1.4 Hz, 1H), 7.81 (dt, J=7.4, 1.5 Hz, 1H), 7.76 (t, J=1.7 Hz, 1H), 7.55 (dt, J=7.8, 1.5 Hz, 1H), 7.49 (t, J=7.8 Hz, 1H), 7.44 (tt, J=8.4, 6.4 Hz, 1H), 7.09 (t, J=8.3 Hz, 2H), 6.27 (s, 1H).

### Example A3: Synthesis of 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Compound 3A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Compound 3B)

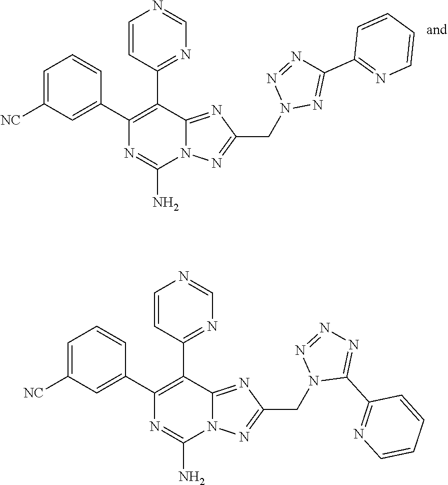 [SMILES: N#Cc1cccc(-c2nc(N)n3nc(Cn4nnc(-c5ccccn5)n4)nc3c2-c2ccncn2)c1 ; N#Cc1cccc(-c2nc(N)n3nc(Cn4nnnc4-c4ccccn4)nc3c2-c2ccncn2)c1]

### Step 1: 3-(5-Amino-2-(hydroxymethyl)-[1,2,4]triazolo[1,5-c]pyrimidin-7-yl)benzonitrile

2-Hydroxyacetohydrazide (2.34 g, 26.01 mmol) was added to a ethanol (35 mL) solution of 3-(2-amino-6-chloropyrimidin-4-yl)benzonitrile (4.00 g, 17.34 mmol) (Example A1, Step 1) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO3 solution. The resulting precipitate was collected by filtration, and dried to obtain the desired product as a brown solid. LCMS calculated for C13H11N6O (M+H)+: 267.1; found 267.1.

### Step 2: 3-(5-Amino-8-bromo-2-(hydroxymethyl)-[1,2,4]triazolo[1,5-c]pyrimidin-7-yl)benzonitrile

To a mixture of 3-(5-amino-2-(hydroxymethyl)-[1,2,4]triazolo[1,5-c]pyrimidin-7-yl)benzonitrile (1.0 g, 3.76 mmol) in DMF (12 mL) at −30° C. was added NBS (0.67 g, 3.76 mmol) portion-wise. The reaction mixture was allowed to slowly warm to 0° C., resulting a homogenous solution. After stirring at 0° C. for 1 h, the reaction mixture was diluted with saturated NaHCO3 solution and the desired product was collected by filtration and dried. LCMS calculated for C₁₃H₁₀BrN₆O (M+H)⁺: 345.0; found 345.0.

### Step 3: 3-(5-Amino-2-(hydroxymethyl)-8-(pyrimidin-4-yl)-[1,2,4]triazolo[1,5-c]pyrimidin-7-yl)benzonitrile

Tetrakis(triphenylphosphine)palladium(0) (0.067 g, 0.058 mmol) was added to a mixture of 4-(tributylstannyl)pyrimidine (0.321 g, 0.869 mmol), 3-(5-amino-8-bromo-2-(hydroxymethyl)-[1,2,4]triazolo[1,5-c]pyrimidin-7-yl)benzonitrile (0.20 g, 0.579 mmol), CsF (0.176 g, 1.159 mmol), and copper(I)iodide (0.022 g, 0.116 mmol) in 1,4-dioxane (5.0 mL). The reaction mixture was purged with N2 and stirred at 80° C. for 7 h. The resulting mixture was cooled to r.t., concentrated and purified by flash column chromatography eluting with 0% to 10% methanol in DCM to afford the product. LC-MS calculated for C17H13N8O (M+H)⁺: 345.1; found 345.1.

### Step 4: 3-(5-Amino-2-(chloromethyl)-8-(pyrimidin-4-yl)-[1,2,4]triazolo[1,5-c]pyrimidin-7-yl)benzonitrile

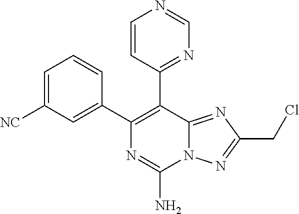 [SMILES: N#Cc1cccc(-c2nc(N)n3nc(CCl)nc3c2-c2ccncn2)c1]

To a mixture of 3-(5-amino-2-(hydroxymethyl)-8-(pyrimidin-4-yl)-[1,2,4]triazolo[1,5-c]pyrimidin-7-yl)benzonitrile (0.1 g, 0.290 mmol) in Acetonitrile (10 ml) was added thionyl chloride (0.212 ml, 2.90 mmol) at r.t. The reaction mixture was stirred at r.t. for 5 h, concentrated, and purified by flash chromatography eluting with 0% to 5% methanol in DCM to afford the product. LC-MS calculated for C₁₇H₁₂ClN₈ (M+H)⁺: 363.1; found 363.1.

### Step 5: Mixture of 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Compound 3A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Compound 3B)

A mixture of 3-(5-amino-2-(chloromethyl)-8-(pyrimidin-4-yl)-[1,2,4]triazolo[1,5-c]pyrimidin-7-yl)benzonitrile (10 mg, 0.028 mmol), 2-(1H-tetrazol-5-yl)pyridine (8.1 mg, 0.055 mmol) and Cs₂CO₃ (20.7 mg, 0.064 mmol) in DMF (1 mL) was stirred at 100° C. for 10 min. The reaction mixture was then cooled to r.t., diluted with methanol (4 mL), and purified by preparative LC-MS (pH 2, acetonitrile/water with TFA) to afford the product as a TFA salt. LCMS calculated for C₂₃H₁₆N₁₃ (M+H)⁺: 474.2; found 474.2.

Compound 3A: ¹H NMR (500 MHz, DMSO) δ 8.99 (d, J=1.4 Hz, 1H), 8.85 (d, J=5.3 Hz, 1H), 8.80-8.71 (m, 1H), 8.71-8.39 (b, 2H), 8.18 (d, J=7.7, 1.1 Hz, 1H), 8.04 (t, J=7.8, 1.8 Hz, 1H), 7.85 (m, 2H), 7.80-7.76 (m, 1H), 7.62-7.55 (m, 2H), 7.53 (t, J=7.8 Hz, 1H), 6.39 (s, 2H).

### Example A4: Synthesis of 3-(5-Amino-2-((3-methylpyridin-2-yl)methoxy)-8-(pyrimidin-4-yl)-[1,2,4]triazolo[1,5-c]pyrimidin-7-yl)benzonitrile (Compound 4)

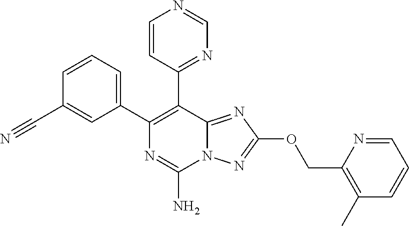 [SMILES: Cc1cccnc1COc1nc2c(-c3ccncn3)c(-c3cccc(C#N)c3)nc(N)n2n1]

### Step 1: 6-Chloro-N²,N²-bis(4-methoxybenzyl)pyrimidine-2,4-diamine

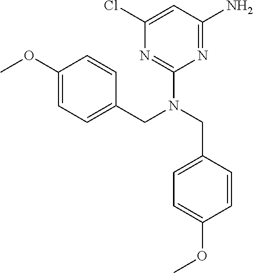 [SMILES: COc1ccc(CN(Cc2ccc(OC)cc2)c2nc(N)cc(Cl)n2)cc1]

To a solution of 2,6-dichloropyrimidin-4-amine (5.0 g, 31 mmol) in 2-propanol (31 mL) was added N,N-diisopropylethylamine (6.4 ml, 37 mmol) and bis(4-methoxybenzyl)amine (7.9 g, 31 mmol). The resulting solution was stirred at 100° C. for 16 h, cooled to r.t., diluted with water (100 mL), and extracted with EtOAc (100 mL). The organic layer was washed with water and brine, dried over anhydrous sodium sulfate, and concentrated to yield the crude product, which was used in the next step without further purification. LC-MS calculated for C₂₀H₂₂ClN₄O₂(M+H)⁺. 385.1; found 385.1.

### Step 2: 7-Chloro-N⁵,N⁵-bis(4-methoxybenzyl)-[1,2,4]triazolo[1,5-c]pyrimidine-2,5-diamine

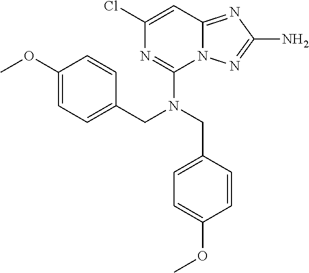 [SMILES: COc1ccc(CN(Cc2ccc(OC)cc2)c2nc(Cl)cc3nc(N)nn23)cc1]

O-ethyl carbonisothiocyanatidate (3.1 mL, 26 mmol) was added to a 1,4-dioxane (5.0 mL) solution of 6-chloro-N²,N²-bis(4-methoxybenzyl)pyrimidine-2,4-diamine (1.0 g, 2.6 mmol) at r.t. The reaction mixture was then stirred at 90° C. overnight, cooled to r.t., and concentrated. The resulting material was dissolved in methanol (12 mL) and ethanol (12 mL), and N,N-diisopropylethylamine (0.91 mL, 5.2 mmol) was added, followed by hydroxylamine hydrochoride (0.54 g, 7.8 mmol). The reaction mixture was stirred at 45° C. for 2 h, cooled to r.t., and concentrated. The resulting material was taken into EtOAc, washed with water, dried over anhydrous sodium sulfate, and concentrated. The crude material was then purified by silica gel chromatography eluting with 0% to 50% EtOAc in hexanes to afford the product. LC-MS calculated for C₂₁H₂₂ClN₆O₂(M+H)⁺: 425.1; found 425.2.

### Step 3: 3-(2-Amino-5-(bis(4-methoxybenzyl)amino)-[1,2,4]triazolo[1,5-c]pyrimidin-7-yl)benzonitrile

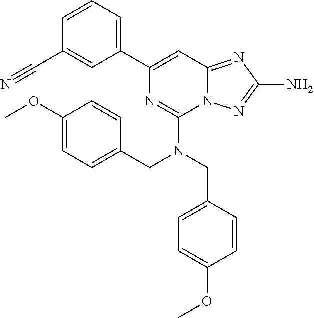 [SMILES: COc1ccc(CN(Cc2ccc(OC)cc2)c2nc(-c3cccc(C#N)c3)cc3nc(N)nn23)cc1]

Chloro(2-dicyclohexylphosphino-2′,4′,6′-tri-i-propyl-1,1′-biphenyl)(2′-amino-1,1′-biphenyl-2-yl) palladium(II) (330 mg, 0.42 mmol) was added to a mixture of (3-cyanophenyl)boronic acid (460 mg, 3.2 mmol), 7-chloro-N⁵,N⁵-bis(4-methoxybenzyl)-[1,2,4]triazolo[1,5-c]pyrimidine-2,5-diamine (890 mg, 2.1 mmol), and sodium carbonate (890 mg, 8.4 mmol) in 1,4-dioxane (8.8 mL) and water (1.8 mL). The mixture was purged with N₂ and stirred at 95° C. overnight. The reaction mixture was then cooled to r.t., concentrated, and purified by silica gel chromatography eluting with 0% to 50% EtOAc in DCM to afford the desired product. LC-MS calculated for C₂₈H₂₆N₇O₂ (M+H)⁺: 492.2; found 492.2.

### Step 4: 3-(2-Amino-5-(bis(4-methoxybenzyl)amino)-8-bromo-[1,2,4]triazolo[1,5-c]pyrimidin-7-yl)benzonitrile

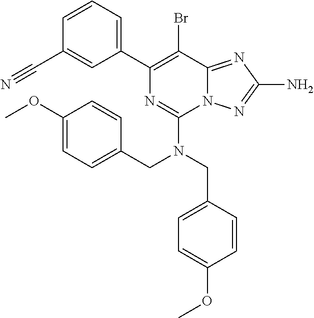 [SMILES: COc1ccc(CN(Cc2ccc(OC)cc2)c2nc(-c3cccc(C#N)c3)c(Br)c3nc(N)nn23)cc1]

To a solution of 3-(2-amino-5-(bis(4-methoxybenzyl)amino)-[1,2,4]triazolo[1,5-c]pyrimidin-7-yl)benzonitrile (330 mg, 0.66 mmol) in DMF (1.4 mL) was slowly added NBS (120 mg, 0.66 mmol) at 0° C. The reaction mixture was then stirred at r.t. for 30 min before water (10 mL) was added. The resulting solid was collected by filtration, and dried to obtain the desired product. LC-MS calculated for C₂₈H₂₅BrN₇O₂(M+H)⁺: m/z=570.1; found 570.2.

### Step 5: 3-(2-Amino-5-(bis(4-methoxybenzyl)amino)-8-(pyrimidin-4-yl)-[1,2,4]triazolo[1,5-c]pyrimidin-7-yl)benzonitrile

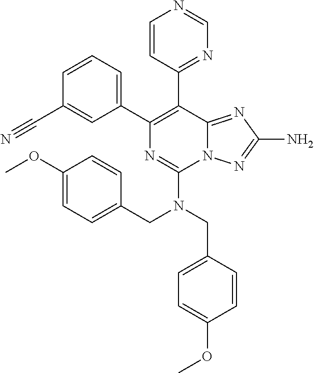 [SMILES: COc1ccc(CN(Cc2ccc(OC)cc2)c2nc(-c3cccc(C#N)c3)c(-c3ccncn3)c3nc(N)nn23)cc1]

A mixture of 3-(2-amino-5-(bis(4-methoxybenzyl)amino)-8-bromo-[1,2,4]triazolo[1,5-c]pyrimidin-7-yl)benzonitrile (350 mg, 0.61 mmol), 4-(tributylstannyl)pyrimidine (210 μL, 0.67 mmol), tetrakis(triphenylphosphine)palladium(0) (70 mg, 0.060 mmol), copper(I) iodide (23 mg, 0.12 mmol) and cesium fluoride (180 mg, 1.2 mmol) in dioxane (4.7 mL) was heated and stirred at 140° C. for 30 min in a microwave reactor. The reaction mixture was then cooled to r.t., filtered through a Celite plug (washed with DCM), and concentrated. The resulting material was purified by silica gel column chromatography eluting with 0-20% MeOH/DCM to give the desired product. LC-MS calculated for C₃₂H₂₈N₉O₂ (M+H)⁺: m/z=570.2; found 570.3.

### Step 6: 3-(5-(Bis(4-methoxybenzyl)amino)-2-bromo-8-(pyrimidin-4-yl)-[1,2,4]triazolo[1,5-c]pyrimidin-7-yl)benzonitrile

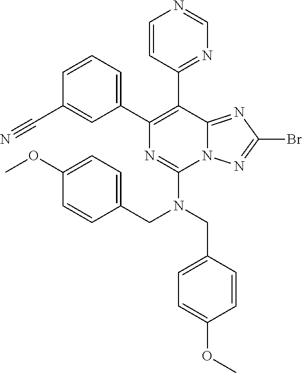 [SMILES: COc1ccc(CN(Cc2ccc(OC)cc2)c2nc(-c3cccc(C#N)c3)c(-c3ccncn3)c3nc(Br)nn23)cc1]

To a mixture of copper(II) bromide (91 mg, 0.407 mmol) and tert-butyl nitrite (0.054 ml, 0.407 mmol) in acetonitrile (3 mL) under nitrogen at 50° C. was added dropwise 3-(2-amino-5-(bis(4-methoxybenzyl)amino)-8-(pyrimidin-4-yl)-[1,2,4]triazolo[1,5-c]pyrimidin-7-yl)benzonitrile (100 mg, 0.203 mmol) in acetonitrile (3 mL). The mixture was stirred at 50° C. for 2 hours. After cooling to room temperature, 1 N aqueous NH₄OH solution (20 mL) was added and the mixture was extracted three times with CH₂Cl₂ (20 mL). The combined organic layers were dried over sodium sulfate, filtered and concentrated. The crude material was purified by silica gel column chromatography eluting with 50-100% ethyl acetate/hexane to give the desired product. LC-MS calculated for C₃₂H₂₆BrN₈O₂(M+H)⁺: m/z=633.1; found 633.2.

### Step 7: 3-(5-Amino-2-((3-methylpyridin-2-yl)methoxy)-8-(pyrimidin-4-yl)-[1,2,4]triazolo[1,5-c]pyrimidin-7-yl)benzonitrile

A suspension of sodium hydride (60% in mineral oil, 3.8 mg, 0.095 mmol), 3-(5-(bis(4-methoxybenzyl)amino)-2-bromo-8-(pyrimidin-4-yl)-[1,2,4]triazolo[1,5-c]pyrimidin-7-yl)benzonitrile (20 mg, 0.032 mmol) and (3-methylpyridin-2-yl)methanol (9.1 μL, 0.095 mmol) in 1,4-dioxane (1 mL) was heated and stirred at 110° C. under nitrogen overnight. The reaction mixture was then cooled to rt, concentrated, and added TFA (1.0 mL). The resulting mixture was then stirred at 110° C. for 30 min, cooled to rt, diluted with acetonitrile, filtered and purified by preparative LC-MS (pH 2, acetonitrile/water with TFA) to give desired product as a TFA salt. LC-MS calculated for C₂₃H₁₈N90 (M+H)⁺: m/z=436.2; found 436.2. ¹H NMR (600 MHz, DMSO) δ 8.97 (d, J=1.4 Hz, 1H), 8.88 (d, J=5.2 Hz, 1H), 8.58-8.52 (m, 1H), 7.97 (d, J=7.8 Hz, 1H), 7.88 (dd, J=5.4, 1.4 Hz, 1H), 7.85 (dt, J=7.5, 1.5 Hz, 1H), 7.78 (t, J=1.8 Hz, 1H), 7.60-7.54 (m, 2H), 7.53 (t, J=7.8 Hz, 1H), 5.69 (s, 2H), 2.48 (s, 3H).

### Example A5: Synthesis of 3-(5-Amino-2-(hydroxy(phenyl)methyl)-[1,2,4]triazolo[1,5-c]pyrimidin-7-yl)benzonitrile (Compound 5)

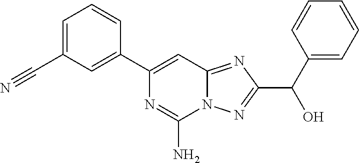 [SMILES: N#Cc1cccc(-c2cc3nc(C(O)c4ccccc4)nn3c(N)n2)c1]

### Step 1: 3-(2-Amino-6-chloropyrimidin-4-yl)benzonitrile

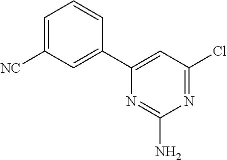 [SMILES: N#Cc1cccc(-c2cc(Cl)nc(N)n2)c1]

A mixture of 4,6-dichloropyrimidin-2-amine (2.5 g, 15.24 mmol), (3-cyanophenyl)boronic acid (2.016 g, 13.72 mmol), tetrakis(triphenylphosphine)palladium(0) (1.057 g, 0.915 mmol) and sodium carbonate (3.23 g, 30.5 mmol) in 1,4-dioxane (60 mL), and water (5 mL) was degassed with nitrogen, then the resulting mixture was heated at 60° C. for two days. After cooled to room temperature (RT), the mixture was concentrated, then diluted with water, and extracted with dichloromethane (DCM, 3×30 mL). The combined organic layers were dried over MgSO₄, filtered, and concentrated. The residue was purified by flash chromatography on a silica gel column with 8% ethyl acetate (EtOAc) in dichloromethane to afford the desired product. LCMS calculated for C₁₁H₈ClN₄ (M+H)⁺: 231.0. Found: 231.0.

### Step 2: 3-(5-Amino-2-(hydroxy(phenyl)methyl)-[1,2,4]triazolo[1,5-c]pyrimidin-7-yl)benzonitrile

A solution of 3-(2-amino-6-chloropyrimidin-4-yl)benzonitrile (100 mg, 0.434 mmol) and 2-hydroxy-2-phenylacetohydrazide (108 mg, 0.650 mmol) in ethanol (2 ml) was heated and stirred at 95° C. for 3 h. After cooling to RT, the reaction mixture was concentrated to dryness, taken into N,O-bis(trimethylsilyl)acetamide (1 mL) and stirred at 120° C. for 7 h. The resulting mixture was cooled to RT, poured onto ice, and stirred for 1 h. The resulting suspension was extracted with DCM three times. The combined organic layers were dried over MgSO₄, filtered, and concentrated. The residue was dissolved in methanol (MeOH) and purified by preparative LC-MS (pH 2, acetonitrile/water with TFA) to afford the product as TFA salt. LCMS calculated for C₁₉H₁₅N60 (M+H)⁺: 343.1; found 343.1.

### Example A6: Synthesis of 3-(5-Amino-2-((2,6-difluorophenyl)(hydroxy)methyl)-[1,2,4]triazolo[1,5-c]pyrimidin-7-yl)-2-fluorobenzonitrile (Compound 6)

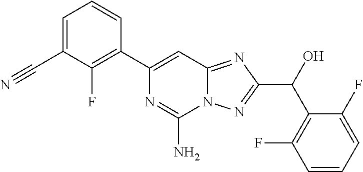 [SMILES: N#Cc1cccc(-c2cc3nc(C(O)c4c(F)cccc4F)nn3c(N)n2)c1F]

### Step 1: 3-(2-Amino-6-chloropyrimidin-4-yl)-2-fluorobenzonitrile

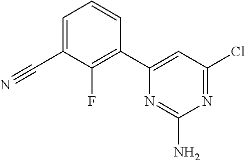 [SMILES: N#Cc1cccc(-c2cc(Cl)nc(N)n2)c1F]

To a solution of 3-bromo-2-fluorobenzonitrile (18.3 g, 91 mmol) in THF (60 mL) cooled to 0° C. was added i-PrMgCl LiCl complex (70.4 mL, 91 mmol) in THE (1.3 M) over 20 min. The mixture was stirred at 0° C. for 50 min, then zinc chloride (48.1 mL, 91 mmol) in 2-MeTHF (1.9 M) was added at 0° C. The reaction was stirred at r.t. for 25 min, at which point 4,6-dichloropyrimidin-2-amine (10 g, 61.0 mmol) was added in one portion. The solution was stirred for 10 min. Tetrakis(triphenylphosphine)palladium (1.41 g, 1.22 mmol) was added to the mixture and the reaction was stirred at r.t. for 16 h. Upon completion, 2,4,6-trimercaptotriazine silica gel (2 g) was added to the reaction solution. The mixture was stirred for 1 h and filtered. The solid was washed with ethyl acetate until the desired product had eluted completely (as detected by LCMS). The filtrate was washed with saturated ammonium chloride solution and water. The organics were concentrated to afford the crude product. Water was added to the crude material and the resulting precipitate was collected by filtration and dried under a stream of nitrogen. The crude material was taken forward without additional purification. LC-MS calculated for C₁₁H₇ClFN₄ (M+H)⁺: m/z=249.0; found 249.0.

### Step 2: Methyl 2-(2,6-difluorophenyl)-2-hydroxyacetate

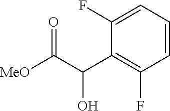 [SMILES: COC(=O)C(O)c1c(F)cccc1F]

Concentrated sulfuric acid (1.4 mL, 27 mmol) was added to a methanol (45 mL) solution of 2,6-difluoromandelic acid (5.0 g, 27 mmol) at 0° C. The mixture was stirred at r.t. for 4 h before being concentrated. To the resulting slurry was added saturated NaHCO₃ solution. The resulting mixture was extracted with DCM. The combined organic layers were washed with water, dried over MgSO₄, filtered, and concentrated to afford the crude product, which was used in the next step without further purification. LC-MS calculated for C₁₁H₁₂F₂NO₃ (M+H+MeCN)⁺: m/z=244.1; found 244.2.

### Step 3: 2-(2,6-Difluorophenyl)-2-hydroxyacetohydrazide

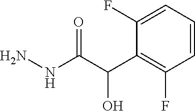 [SMILES: NNC(=O)C(O)c1c(F)cccc1F]

Hydrazine (3.0 mL, 96 mmol) was added to an ethanol (90 mL) solution of methyl 2-(2,6-difluorophenyl)-2-hydroxyacetate (10.8 g, 53 mmol) at RT. The reaction mixture was stirred at 100° C. for 2 h, cooled to RT, concentrated, and used in next step without further purification. LC-MS calculated for C₈H₉F₂N₂O₂ (M+H)⁺: 203.1; found 203.2.

### Step 4: 3-(5-Amino-2-((2,6-difluorophenyl)(hydroxy)methyl)-[1,2,4]triazolo[1,5-c]pyrimidin-7-yl)-2-fluorobenzonitrile

The title compound was prepared using similar procedures as described for Example A5 Step 2, with 3-(2-amino-6-chloropyrimidin-4-yl)-2-fluorobenzonitrile replacing 3-(2-amino-6-chloropyrimidin-4-yl)benzonitrile, and with 2-(2,6-Difluorophenyl)-2-hydroxyacetohydrazide replacing 2-hydroxy-2-phenylacetohydrazide. The two enantiomers were separated by chiral SFC using a Phenomenex (R,R)-Whelk-01 column (21.2×250 mm, 5 m particle size) eluting with an isocratic mobile phase 15% MeOH in CO₂ with a flow rate of 85 mL/minute. The retention times of peak one and peak two were 3.8 min and 5.3 min, respectively. Following concentration, peak two was purified by prep-LCMS (pH=2, MeCN/water with TFA) to give the desired product as a TFA salt. LC-MS calculated for C₁₉H₁₂F₃N₆O (M+H)⁺: 397.1; found 397.1.

### Example A7: Synthesis of 5-Amino-7-(3-cyano-2-fluorophenyl)-2-((2,6-difluorophenyl)(hydroxy)methyl)-[1,2,4]triazolo[1,5-c]pyrimidine-8-carbonitrile (Compound 7)

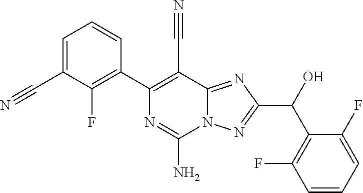 [SMILES: N#Cc1cccc(-c2nc(N)n3nc(C(O)c4c(F)cccc4F)nc3c2C#N)c1F]

### Step 1: 3-(5-Amino-8-bromo-2-((2,6-difluorophenyl)(hydroxy)methyl)-[1,2,4]triazolo[1,5-c]pyrimidin-7-yl)-2-fluorobenzonitrile

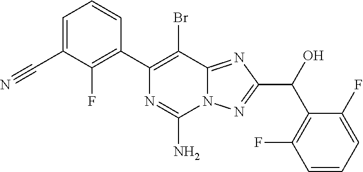 [SMILES: N#Cc1cccc(-c2nc(N)n3nc(C(O)c4c(F)cccc4F)nc3c2Br)c1F]

This compound was prepared using similar procedures as described for Example A1, Step 4, with 3-(5-amino-2-((2,6-difluorophenyl)(hydroxy)methyl)-[1,2,4]triazolo[1,5-c]pyrimidin-7-yl)-2-fluorobenzonitrile (from Example A6) replacing 3-(5-amino-2-(pyridin-2-ylmethyl)-[1,2,4]triazolo[1,5-c]pyrimidin-7-yl)benzonitrile. LCMS calculated for C₁₉H₁₁BrF₃N6O (M+H)⁺: 475.0; found 475.0.

### Step 2: 5-Amino-7-(3-cyano-2-fluorophenyl)-2-((2,6-difluorophenyl)(hydroxy)methyl)-[1,2,4]triazolo[1,5-c]pyrimidine-8-carbonitrile

A mixture of 3-(5-amino-8-bromo-2-((2,6-difluorophenyl)(hydroxy)methyl)-[1,2,4]triazolo[1,5-c]pyrimidin-7-yl)-2-fluorobenzonitrile (0.12 g, 0.25 mmol), ZnCN₂ (0.060 g, 0.51 mmol) and tBuXPhos Pd G3 (0.020 g, 0.025 mmol) in 1,4-dioxane (0.63 mL) and water (0.63 mL) was purged with N₂ and was stirred at 100° C. for 1 h. After cooling to r.t., the reaction was diluted with saturated NaHCO₃ and the organics were extracted with EtOAc (3×). The combined organics were dried over MgSO₄ and concentrated. The two enantiomers were separated by chiral HPLC using a Phenomenex Lux Celluose-4 column (21.2×250 mm, 5 m particle size) eluting with an isocratic mobile phase 60% EtOH in hexanes with a flow rate of 20 mL/minute. The retention times of peak one and peak two were 4.9 min and 7.2 min, respectively. Following concentration, peak one was purified by preparative LC-MS (pH 2, acetonitrile/water with TFA) to give the desired product as a TFA salt. LC-MS calculated for C₂₀H₁₁F₃N₇O (M+H)⁺: 422.1; found 422.1.

### Example A8: Synthesis of 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile (Compound 8)

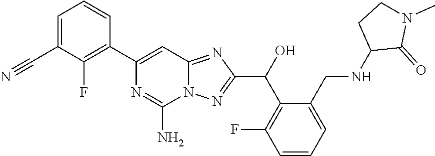 [SMILES: CN1CCC(NCc2cccc(F)c2C(O)c2nc3cc(-c4cccc(C#N)c4F)nc(N)n3n2)C1=O]

### Step 1: Methyl 2-(2-fluoro-6-vinylphenyl)acetate

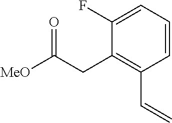

A mixture of methyl 2-(2-bromo-6-fluorophenyl)acetate (6.0 g, 24 mmol), potassium phosphate, tribasic (15.5 g, 73 mmol), palladium(II) acetate (0.55 g, 2.4 mmol), and SPhos (1.0 g, 2.4 mmol) were added to a 500 mL pressure vessel. Next, 4,4,5,5-tetramethyl-2-vinyl-1,3,2-dioxaborolane (6.4 ml, 36 mmol) in dioxane (150 mL) and water (15 mL) was added, the reaction mixture was purged with N2, and stirred at 80° C. for 16 h. The reaction mixture was then cooled to RT, concentrated, and extracted with EtOAc (x3). The combined organic layers were dried over MgSO4, concentrated, and purified by column chromatography (0 to 50% EtOAc in DCM). LC-MS calculated for C11H12FO2 (M+H)⁺: 195.1; found 195.1.

### Step 2: Methyl 2-(2-fluoro-6-vinylphenyl)-2-hydroxyacetate

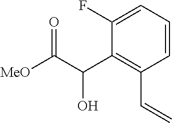

Methyl 2-(2-fluoro-6-vinylphenyl)acetate (2.5 g, 12.9 mmol) was dissolved in THE (130 mL) and cooled to −78° C. LDA (16.7 mL, 16.7 mmol) in THE (1.0 M) was added dropwise, and the resulting solution was stirred at −78° C. for 30 min. Then, 9,9-dimethyltetrahydro-4H-4a,7-methanobenzo[c][1,2]oxazireno[2,3-b]isothiazole 3,3-dioxide (4.7 g, 20.6 mmol) was added dropwise in THF (0.5 M). After 30 min at −78° C., the reaction mixture was warmed to 0° C. and stirred for 1 h. The reaction was quenched with saturated NH₄Cl. The aqueous layer was extracted with DCM (3×). The combined organics were dried over anhydrous Na₂SO₄, filtered, and concentrated under reduced pressure. The crude product was purified by column chromatography eluting with 0 to 50% ethyl acetate in hexanes to afford the desired product. LCMS calculated for C11H11FO3Na (M+Na)+: 233.1; found 233.1.

### Step 3: 2-(2-Fluoro-6-vinylphenyl)-2-hydroxyacetohydrazide

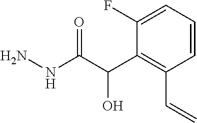

This compound was prepared using similar procedures as described for Example A6, Step 3, with methyl 2-(2-fluoro-6-vinylphenyl)-2-hydroxyacetate replacing methyl 2-(2,6-difluorophenyl)-2-hydroxyacetate. LCMS calculated for C₁₀H₁₂FN₂O₂(M+H)⁺: 211.1; found 211.1.

### Step 4: 3-(5-Amino-2-((2-fluoro-6-vinylphenyl)(hydroxy)methyl)-[1,2,4]triazolo[1,5-c]pyrimidin-7-yl)-2-fluorobenzonitrile

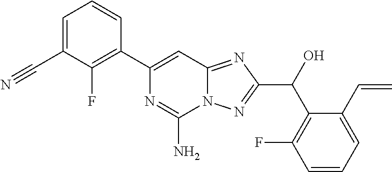 [SMILES: C=Cc1cccc(F)c1C(O)c1nc2cc(-c3cccc(C#N)c3F)nc(N)n2n1]

This compound was prepared using similar procedures as described for Example A6 Step 4, with 2-(2-fluoro-6-vinylphenyl)-2-hydroxyacetohydrazide replacing 2-(2,6-difluorophenyl)-2-hydroxyacetohydrazide. LCMS calculated for C21H15F₂N6O (M+H)⁺: 405.1; found 405.1.

### Step 5: 3-(5-Amino-2-((2-fluoro-6-formylphenyl)(hydroxy)methyl)-[1,2,4]triazolo[1,5-c]pyrimidin-7-yl)-2-fluorobenzonitrile

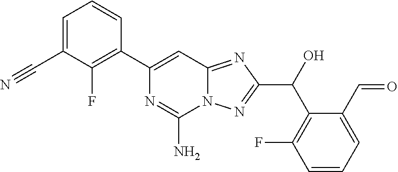 [SMILES: N#Cc1cccc(-c2cc3nc(C(O)c4c(F)cccc4C=O)nn3c(N)n2)c1F]

Osmium tetroxide in water (4% w/w, 0.36 mL, 0.12 mmol) was added to a THE (18 mL) and water (4.6 mL) solution of 3-(5-amino-2-((2-fluoro-6-vinylphenyl)(hydroxy)methyl)-[1,2,4]triazolo[1,5-c]pyrimidin-7-yl)-2-fluorobenzonitrile (930 mg, 2.30 mmol). The reaction mixture was stirred for 5 min at RT and then sodium periodate (2.5 g, 11.5 mmol) was added. After stirring for 1 h, the mixture was diluted with sodium metabisulfite in saturated aq. NaHCO3 (5% w/w, 20 mL) and extracted with EtOAc (×3). The combined organic layers were dried over MgSO4 and concentrated under reduced pressure. The crude material was purified by column chromatography eluting with 0 to 100% ethyl acetate in hexanes to afford the desired product. LCMS calculated for C20H13F2N6O2 (M+H)⁺: 407.1; found 407.1.

### Step 6: 3-(5-Amino-2-((2-fluoro-6-(((I-methyl-2-oxopyrrolidin-3-yl)amino)methyl)phenyl)(hydroxy)methyl)-[1,2,4]triazolo[1,5-c]pyrimidin-7-yl)-2-fluorobenzonitrile

A solution of 3-amino-1-methylpyrrolidin-2-one (63 mg, 0.55 mmol) and 3-(5-amino-2-((2-fluoro-6-formylphenyl)(hydroxy)methyl)-[1,2,4]triazolo[1,5-c]pyrimidin-7-yl)-2-fluorobenzonitrile (150 mg, 0.37 mmol) was stirred at 40° C. for 2 h in 1,2-dichloroethane (1.9 mL). Then sodium triacetoxyborohydride (160 mg, 0.74 mmol) was added and the reaction mixture was stirred at room temperature for 16 h. The reaction was diluted with saturated NaHCO₃ and the organics were extracted with EtOAc (3×). The combined organics were dried over MgSO₄ and concentrated. The diastereomers were separated by chiral HPLC using a Phenomenex Lux Celluose-4 column (21.2×250 mm, 5 m particle size) eluting with an isocratic mobile phase 45% EtOH in hexanes with a flow rate of 20 mL/minute. The retention times of peak one and peak two were 14.9 min and 17.5 min, respectively. Following concentration, peak two was further separated by chiral HPLC using a Phenomenex Lux Celluose-1 column (21.2×250 mm, 5 m particle size) eluting with an isocratic mobile phase 30% EtOH in hexanes with a flow rate of 20 mL/minute. The retention times of peak one and peak two were 11.0 min and 15.5 min, respectively. Following concentration, peak one was purified by preparative LC-MS (pH=2, MeCN/water with TFA) to give the desired product as a TFA salt. LC-MS calculated for C₂₅H₂₃F₂N₈O₂ (M+H)⁺: 505.2; found 505.2.

### Example A9: Synthesis of 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile (Compound 9)

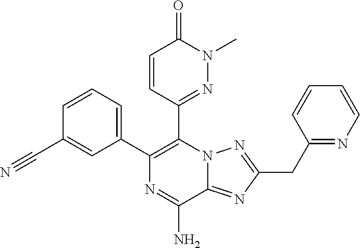 [SMILES: Cn1nc(-c2c(-c3cccc(C#N)c3)nc(N)c3nc(Cc4ccccn4)nn23)ccc1=O]

### Step 1: Methyl 3-bromo-1-(2-(3-cyanophenyl)-2-oxoethyl)-1H-1,2,4-triazole-5-carboxylate

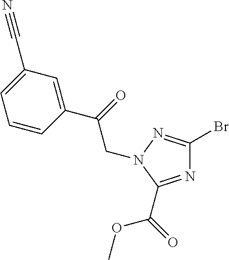 [SMILES: COC(=O)c1nc(Br)nn1CC(=O)c1cccc(C#N)c1]

To a solution of methyl 3-bromo-1H-1,2,4-triazole-5-carboxylate (5.0 g, 24.3 mmol), 3-(2-bromoacetyl)benzonitrile (5.44 g, 24.3 mmol) in DMF (100 mL) was added potassium carbonate (3.35 g, 24.3 mmol). The reaction mixture was stirred at ambient temperature for 2 h. The reaction mixture was then diluted with water and DCM. The organic layer was separated, washed with brine, dried over Na₂SO₄, filtered and concentrated. The resulting residue was purified via flash chromatography to give the desired product as a white solid (5.2 g, 61%). LC-MS calculated for C₁₃H₁₀BrN₄O₃(M+H)⁺: m/z=349.0; found 349.0.

### Step 2: 3-(2-Bromo-8-oxo-7,8-dihydro-[1,2,4]triazolo[1,5-a]pyrazin-6-yl)benzonitrile

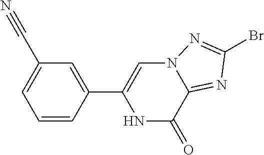 [SMILES: N#Cc1cccc(-c2cn3nc(Br)nc3c(=O)[nH]2)c1]

Methyl 3-bromo-1-(2-(3-cyanophenyl)-2-oxoethyl)-1H-1,2,4-triazole-5-carboxylate (10.5 g, 30.1 mmol) was dissolved in acetic acid (100 mL), and ammonium acetate (23.18 g, 301 mmol) was added. The mixture was stirred at 110° C. for 12 h. After cooling to room temperature, the reaction mixture was diluted with water. The resulting precipitate was collected via filtration, washed with water, and dried under vacuum to afford the product (8.4 g, 88%). LC-MS calculated for C₁₂H₇BrN₅O (M+H)⁺: m/z=316.0; found 316.0.

### Step 3: 3-(2-Bromo-8-chloro-[1,2,4]triazolo[1,5-a]pyrazin-6-yl)benzonitrile

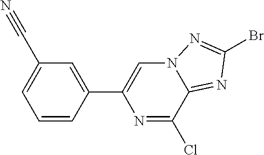 [SMILES: N#Cc1cccc(-c2cn3nc(Br)nc3c(Cl)n2)c1]

A mixture of 3-(2-bromo-8-oxo-7,8-dihydro-[1,2,4]triazolo[1,5-a]pyrazin-6-yl)benzonitrile (8.4 g, 26.6 mmol) and POCl₃ (49.5 mL, 531 mmol) was stirred at 110° C. overnight. After cooling to room temperature, the reaction mixture was slowly added to a flask containing ice and sodium bicarbonate. The resulting precipitate was collected, washed with water, and dried to afford the product (8.8 g, 99%). LC-MS calculated for C₁₂H₆BrClN₅ (M+H)⁺: m/z=333.9; found 334.0.

### Step 4. 3-(8-(Bis(4-methoxybenzyl)amino)-2-bromo-[1,2,4]triazolo[1,5-a]pyrazin-6-yl)benzonitrile

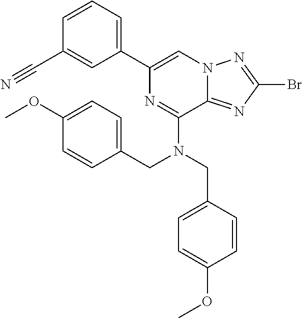 [SMILES: COc1ccc(CN(Cc2ccc(OC)cc2)c2nc(-c3cccc(C#N)c3)cn3nc(Br)nc23)cc1]

A mixture of 3-(2-bromo-8-chloro-[1,2,4]triazolo[1,5-a]pyrazin-6-yl)benzonitrile (8.99 g, 26.9 mmol), bis(4-methoxybenzyl)amine (10.37 g, 40.3 mmol), and DIPEA (9.4 mL, 53.7 mmol) in DMF (134 mL) was stirred at 85° C. overnight. The reaction mixture was cooled to room temperature, and diluted with water. The resulting precipitate was collected via filtration, and dried to afford the product (14.1 g, 94%). LC-MS calculated for C₂₈H₂₄BrN₆O₂(M+H)⁺: m/z=555.1; found 555.1.

### Step 5: 3-(8-(Bis(4-methoxybenzyl)amino)-2-(pyridin-2-ylmethyl)-[1,2,4]triazolo[1,5-a]pyrazin-6-yl)benzonitrile

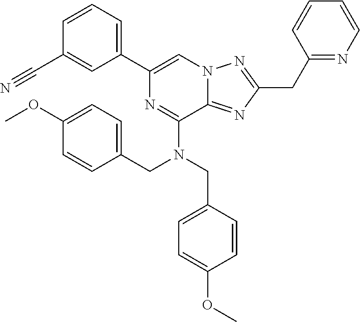 [SMILES: COc1ccc(CN(Cc2ccc(OC)cc2)c2nc(-c3cccc(C#N)c3)cn3nc(Cc4ccccn4)nc23)cc1]

To a solution of 2-methylpyridine (0.050 g, 0.540 mmol) in THF (0.5 mL) vas added 2.5 M n-butyllithium (0.216 mL, 0.540 mmol) at −78° C. The resulting solution was stirred at the same temperature for 1 h, before 1.9 M zinc chloride in 2-methyltetrahydrofuran (0.284 mL, 0.540 mmol) was added, and the resulting mixture was stirred at room temperature for 10 min.

A microwave vial charge with 3-(8-(bis(4-methoxybenzyl)amino)-2-bromo-[1,2,4]triazolo[1,5-a]pyrazin-6-yl)benzonitrile (0.15 g, 0.270 mmol), palladium acetate (1.1 mg, 4.7 μmol), and 2′-(dicyclohexylphosphino)-N,N,N′,N′-tetramethylbiphenyl-2,6-diamine (4.1 mg, 9.5 μmol) was evacuated under high vacuum and backfilled with nitrogen. THE (2.0 mL) and toluene (0.5 mL) was then added to the reaction vial. The mixture was cooled to 0° C. and the zinc reagent prepared from previous step was added slowly via a syringe. The reaction mixture was then stirred at 60° C. overnight, cooled to room temperature, and partitioned between ethylacetate and saturated NH₄Cl solution. The layers were separated and the aqueous layer was extracted with ethylacetate. The combined organic layers were washed with water and brine, dried over MgSO₄, and concentrated. The resulting residue was purified via flash chromatography to afford the product (0.11 g, 71%). LC-MS calculated for C₃₄H₃₀N₇O₂ (M+H)⁺: m/z=568.2; found 568.3.

### Step 6. 3-(8-Amino-2-(pyridin-2-ylmethyl)-[1,2,4]triazolo[1,5-a]pyrazin-6-yl)benzonitrile

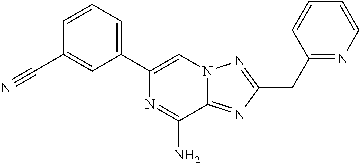 [SMILES: N#Cc1cccc(-c2cn3nc(Cc4ccccn4)nc3c(N)n2)c1]

A mixture of 3-(8-(bis(4-methoxybenzyl)amino)-2-(pyridin-2-ylmethyl)-[1,2,4]triazolo[1,5-a]pyrazin-6-yl)benzonitrile (110 mg, 0.194 mmol) and TFA (746 μL, 9.69 mmol) was stirred at 80° C. for 30 min, cooled to room temperature, and concentrated. The resulting residue was purified via prep-LCMS (pH 2) to give the product as a white solid (TFA salt) (57 mg, 90%). LC-MS calculated for C₁₈H₁₄N₇(M+H)⁺: m/z=328.1; found 328.1.

### Step 7. 3-(8-Amino-5-bromo-2-(pyridin-2-ylmethyl)-[1,2,4]triazolo[1,5-a]pyrazin-6-yl)benzonitrile

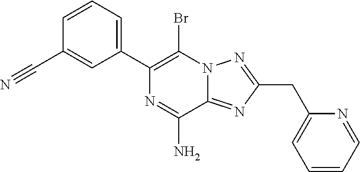 [SMILES: N#Cc1cccc(-c2nc(N)c3nc(Cc4ccccn4)nn3c2Br)c1]

To a solution of 3-(8-amino-2-(pyridin-2-ylmethyl)-[1,2,4]triazolo[1,5-a]pyrazin-6-yl)benzonitrile (TFA salt) (35 mg, 0.079 mmol) in DMF (0.5 mL)/DCM (0.5 mL) was added NBS (14.1 mg, 0.079 mmol). The reaction mixture was then stirred at room temperature for 1 h, and concentrated to afford the crude product, which was used in the next step without further purification. LC-MS calculated for C₁₈H₁₃BrN₇ (M+H)⁺: m/z=406.0; found 406.0.

### Step 8. 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile

A mixture of 6-chloro-2-methylpyridazin-3(2H)-one (30 mg, 0.21 mmol), bis(pinacolato)diboron (53 mg, 0.21 mmol), chloro(2-dicyclohexylphosphino-2′,4′,6′-triisopropyl-1,1′-biphenyl)[2-(2′-amino-1,1′-biphenyl)]palladium(II) (15.7 mg, 0.02 mmol) (XPhos Pd G2) and potassium acetate (61.7 mg, 0.63 mmol) in 1,4-dioxane (1 mL) was stirred at 100° C. for 1 h. 3-(8-Amino-5-bromo-2-(pyridin-2-ylmethyl)-[1,2,4]triazolo[1,5-a]pyrazin-6-yl)benzonitrile (10 mg, 0.025 mmol), cesium carbonate (37.6 mg, 0.116 mmol) and water (0.2 mL) were then added to the reaction mixture. The resulting mixture was heated at 90° C. for 1 h. The mixture was concentrated and purified by preparative LCMS (pH 2, acetonitrile/water with TFA) to afford the desired product as TFA salt. LCMS calculated for C₂₃H₁₈N90 (M+H)⁺: 436.2; found 436.2.

¹H NMR (500 MHz, DMSO) δ 8.66-8.62 (d, J=5.1 Hz, 1H), 8.09-8.02 (d, J=1.8 Hz, 1H), 7.88-7.85 (t, J=1.8 Hz, 1H), 7.85-7.81 (m, 3H), 7.78-7.72 (d, J=9.6 Hz, 1H), 7.66-7.51 (m, 4H), 7.10-7.06 (d, J=9.6 Hz, 1H), 4.59-4.48 (s, 2H), 3.53-3.43 (s, 3H).

### Example A10: Synthesis of 3-(8-Amino-2-((2,6-difluorophenyl)(hydroxy)methyl)-5-(pyrimidin-4-yl)-[1,2,4]triazolo[1,5-a]pyrazin-6-yl)benzonitrile (Compound 10)

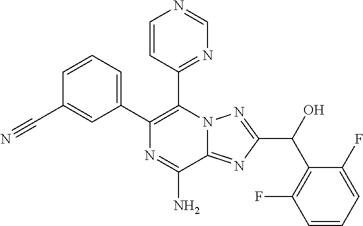 [SMILES: N#Cc1cccc(-c2nc(N)c3nc(C(O)c4c(F)cccc4F)nn3c2-c2ccncn2)c1]

### Step 1: Methyl 3-bromo-1-(2-(3-cyanophenyl)-2-oxoethyl)-1H-1,2,4-triazole-5-carboxylate

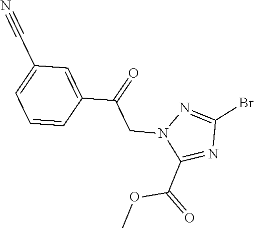 [SMILES: COC(=O)c1nc(Br)nn1CC(=O)c1cccc(C#N)c1]

To a solution of methyl 3-bromo-1H-1,2,4-triazole-5-carboxylate (5.0 g, 24.3 mmol), 3-(2-bromoacetyl)benzonitrile (5.44 g, 24.3 mmol) in DMF (100 mL) was added potassium carbonate (3.35 g, 24.3 mmol). The reaction mixture was stirred at ambient temperature for 2 h. The reaction mixture was then diluted with water and DCM. The organic layer was separated, washed with brine, dried over Na₂SO₄, filtered and concentrated. The resulting residue was purified via flash chromatography to give the desired product as a white solid (5.2 g, 61%). LC-MS calculated for C₁₃H₁₀BrN₄O₃(M+H)⁺: m/z=349.0; found 349.0.

### Step 2: 3-(2-Bromo-8-oxo-7,8-dihydro-[1,2,4]triazolo[1,5-a]pyrazin-6-yl)benzonitrile

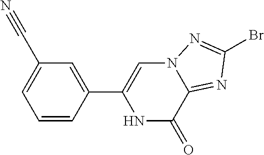 [SMILES: N#Cc1cccc(-c2cn3nc(Br)nc3c(=O)[nH]2)c1]

Methyl 3-bromo-1-(2-(3-cyanophenyl)-2-oxoethyl)-1H-1,2,4-triazole-5-carboxylate (10.5 g, 30.1 mmol) was dissolved in acetic acid (100 mL), and ammonium acetate (23.18 g, 301 mmol) was added. The mixture was stirred at 110° C. for 12 h. After cooling to room temperature, the reaction mixture was diluted with water. The resulting precipitate was collected via filtration, washed with water, and dried under vacuum to afford the product (8.4 g, 88%). LC-MS calculated for C₁₂H₇BrN₅O (M+H)⁺: m/z=316.0; found 316.0.

### Step 3: 3-(2-Bromo-8-chloro-[1,2,4]triazolo[1,5-a]pyrazin-6-yl)benzonitrile

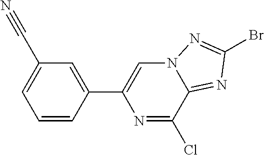 [SMILES: N#Cc1cccc(-c2cn3nc(Br)nc3c(Cl)n2)c1]

A mixture of 3-(2-bromo-8-oxo-7,8-dihydro-[1,2,4]triazolo[1,5-a]pyrazin-6-yl)benzonitrile (8.4 g, 26.6 mmol) and POCl₃ (49.5 mL, 531 mmol) was stirred at 110° C. overnight. After cooling to room temperature, the reaction mixture was slowly added to a flask containing ice and sodium bicarbonate. The resulting precipitate was collected via filtration, washed with water, and dried to afford the product (8.8 g, 99%). LC-MS calculated for C₁₂H₆BrClN₅ (M+H)⁺: m/z=336.0; found 336.0.

### Step 4: 3-(8-(Bis(4-methoxybenzyl)amino)-2-bromo-[1,2,4]triazolo[1,5-a]pyrazin-6-yl)benzonitrile

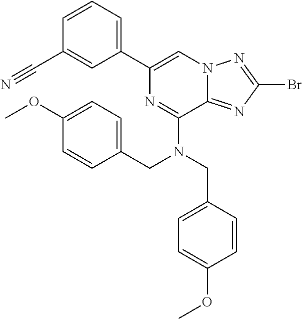 [SMILES: COc1ccc(CN(Cc2ccc(OC)cc2)c2nc(-c3cccc(C#N)c3)cn3nc(Br)nc23)cc1]

A mixture of 3-(2-bromo-8-chloro-[1,2,4]triazolo[1,5-a]pyrazin-6-yl)benzonitrile (8.99 g, 26.9 mmol), bis(4-methoxybenzyl)amine (10.37 g, 40.3 mmol), and DIPEA (9.4 mL, 53.7 mmol) in DMF (134 mL) was stirred at 65° C. overnight. The reaction mixture was cooled to room temperature, and diluted with water. The resulting precipitate was collected via filtration, and dried to afford the product (14.1 g, 94%). LC-MS calculated for C₂₈H₂₄BrN₆O₂(M+H)⁺: m/z=555.1; found 555.1.

### Step 5: 3-(8-(Bis(4-methoxybenzyl)amino)-2-vinyl-[1,2,4]triazolo[1,5-a]pyrazin-6-yl)benzonitrile

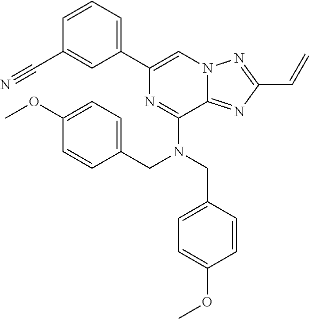 [SMILES: C=Cc1nc2c(N(Cc3ccc(OC)cc3)Cc3ccc(OC)cc3)nc(-c3cccc(C#N)c3)cn2n1]

A mixture of 3-(8-(bis(4-methoxybenzyl)amino)-2-bromo-[1,2,4]triazolo[1,5-a]pyrazin-6-yl)benzonitrile (10.0 g, 18.0 mmol), 4,4,5,5-tetramethyl-2-vinyl-1,3,2-dioxaborolane (3.88 g, 25.2 mmol), potassium phosphate tribasic (9.55 g, 45.0 mmol) and chloro(2-dicyclohexylphosphino-2′,4′,6′-triisopropyl-1,1′-biphenyl)[2-(2′-amino-1,1′-biphenyl)]palladium(11) (567 mg, 0.72 mmol) in 1,4-dioxane (200 mL) and water (50 mL) was stirred at 85° C. for 2 hrs. The reaction mixture was cooled to room temperature, and most of 1,4-dioxane was removed. The resulting precipitate was collected via filtration, washed with water and dried to afford the crude product (9.1 g), which was used in the next step directly. LC-MS calculated for C₃₀H₂₇N₆O₂ (M+H)⁺: m/z=503.2; found 503.1.

### Step 6. 3-(8-(Bis(4-methoxybenzyl)amino)-5-bromo-2-vinyl-[1,2,4]triazolo[1,5-a]pyrazin-6-yl)benzonitrile

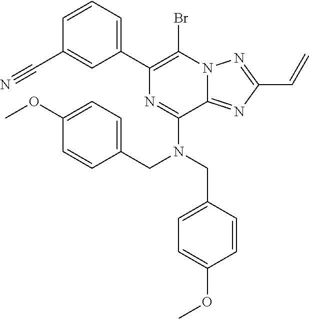 [SMILES: C=Cc1nc2c(N(Cc3ccc(OC)cc3)Cc3ccc(OC)cc3)nc(-c3cccc(C#N)c3)c(Br)n2n1]

To a solution of 3-(8-(bis(4-methoxybenzyl)amino)-2-vinyl-[1,2,4]triazolo[1,5-a]pyrazin-6-yl)benzonitrile (717 mg, 1.43 mmol) in 10 mL of dichloromethane, 1-bromopyrrolidine-2,5-dione (254 mg, 1.43 mmol) was added at 0° C. The resulting mixture was stirred for 4 hrs, and directly purified by a silica gel column to afford the desired product (780 mg, 94%). LC-MS calculated for C₃₀H₂₆BrN₆O₂(M+H)⁺: m/z=581.1; found 581.2.

### Step 7: 3-(8-(Bis(4-methoxybenzyl)amino)-5-(pyrimidin-4-yl)-2-vinyl-[1,2,4]triazolo[1,5-a]pyrazin-6-yl)benzonitrile

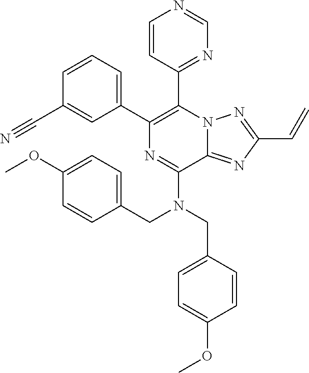 [SMILES: C=Cc1nc2c(N(Cc3ccc(OC)cc3)Cc3ccc(OC)cc3)nc(-c3cccc(C#N)c3)c(-c3ccncn3)n2n1]

A mixture of 3-(8-(bis(4-methoxybenzyl)amino)-5-bromo-2-vinyl-[1,2,4]triazolo[1,5-a]pyrazin-6-yl)benzonitrile (260 mg, 0.45 mmol), 4-(tributylstannyl)pyrimidine (215 mg, 0.58 mmol), lithium chloride (28.4 mg, 0.67 mmol), copper(I) chloride (67 mg, 0.67 mmol), and Tetrakis(triphenylphosphine)palladium(0) (52 mg, 0.045 mmol) in THE (5 mL) was stirred at 90° C. for 45 mins. The reaction mixture was quenched with water and extracted with dichloromethane. The combined organic layers were concentrated, and purified by a silica gel column to afford the desired product (176 mg, 67%). LC-MS calculated for C₃₄H₂₉N₈O₂ (M+H)⁺: m/z=581.2; found 581.1.

### Step 8: 3-(8-(Bis(4-methoxybenzyl)amino)-2-formyl-5-(pyrimidin-4-yl)-[1,2,4]triazolo[1,5-a]pyrazin-6-yl)benzonitrile

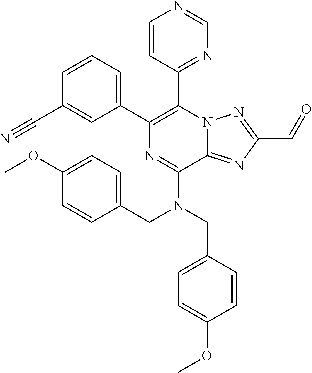 [SMILES: COc1ccc(CN(Cc2ccc(OC)cc2)c2nc(-c3cccc(C#N)c3)c(-c3ccncn3)n3nc(C=O)nc23)cc1]

A mixture of 3-(8-(bis(4-methoxybenzyl)amino)-5-(pyrimidin-4-yl)-2-vinyl-[1,2,4]triazolo[1,5-a]pyrazin-6-yl)benzonitrile (176 mg, 0.3 mmol), osmium(VIII) oxide (3 mg in 0.3 mL water, 0.015 mmol), and sodium periodate (292 mg, 1.36 mmol) in THF/water (1:1, 6 mL) was stirred at 65° C. for 1 h. The reaction mixture was cooled to room temperature, and extracted with dichloromethane. The combined organic layers were concentrated, and purified by silica gel column to afford the desired product (130 mg, 74%).

LC-MS calculated for C₃₃H₂₇N₈O₃ (M+H)⁺: m/z=583.2; found 583.2.

### Step 9: 3-(8-Amino-2-((2,6-difluorophenyl)(hydroxy)methyl)-5-(pyrimidin-4-yl)-[1,2,4]triazolo[1,5-a]pyrazin-6-yl)benzonitrile

Preparation of the Grignard reagent: To a solution of 1,3-difluoro-2-iodobenzene (142 mg, 0.6 mmol) in tetrahydrofuran (1 mL), isopropylmagnesium chloride solution (296 μl, 2 M) was added at −10° C. The resulting mixture was stirred for 1 h, and used directly in the following step.

To a solution of 3-(8-(bis(4-methoxybenzyl)amino)-2-formyl-5-(pyrimidin-4-yl)-[1,2,4]triazolo[1,5-a]pyrazin-6-yl)benzonitrile (120 mg, 0.2 mmol) in THE (2 mL), the freshly prepared Grignard reagent from previous step was added at −10° C. The reaction mixture was stirred for 30 min, quenched with ammonium chloride solution (4 mL), and extracted with dichloromethane. The combined organic layers were concentrated under vacuum. The resulting material was dissolved in TFA (5 mL), and stirred at 80° C. for 20 min. The reaction mixture was then cooled to room temperature, concentrated, and basified by adding aqeuous NaHCO₃ solution.

The crude material was directly purified by a silica gel column to afford the desired product (60 mg, 64%) as a racemic mixture. The product was then separated with chiral HPLC using a chiral column (Phenomenex Lux Sum Cellulose-4, 21.2×250 mm) and 75% EtOH in hexanes (20 mL/min) solvent system.

Peak 2 was isolated, and further purified via preparative LC/MS (pH=2, acetonitrile/water with TFA) to give the desired product as a TFA salt. LC-MS calculated for C₂₃H₁₅F₂N₈O (M+H)⁺: m/z=457.1; found 457.0.

¹H NMR (600 MHz, DMSO-d₆) δ 9.14 (d, J=1.3 Hz, 1H), 8.95 (d, J=5.2 Hz, 1H), 7.90 (dd, J=5.2, 1.4 Hz, 1H), 7.88 (s, 1H), 7.78 (dt, J=7.6, 1.4 Hz, 1H), 7.74 (t, J=1.4 Hz, 1H), 7.54 (dt, J=7.9, 1.3 Hz, 1H), 7.51-7.40 (m, 2H), 7.09 (t, J=8.4 Hz, 2H), 6.27 (s, 1H).

### Example A11: Synthesis of 3-(8-amino-2-(amino(2,6-difluorophenyl)methyl)-5-(4-methyloxazol-5-yl)-[1,2,4]triazolo[1,5-a]pyrazin-6-yl)benzonitrile (Compound 11)

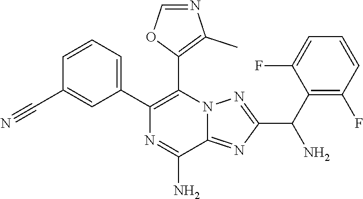 [SMILES: Cc1ncoc1-c1c(-c2cccc(C#N)c2)nc(N)c2nc(C(N)c3c(F)cccc3F)nn12]

### Step 1: 3-(8-(Bis(4-methoxybenzyl)amino)-5-bromo-2-vinyl-[1,2,4]triazolo[1,5-a]pyrazin-6-yl)benzonitrile

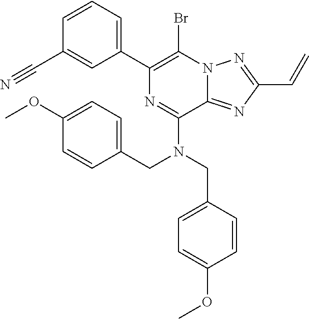 [SMILES: C=Cc1nc2c(N(Cc3ccc(OC)cc3)Cc3ccc(OC)cc3)nc(-c3cccc(C#N)c3)c(Br)n2n1]

To a solution of 3-(8-(bis(4-methoxybenzyl)amino)-2-vinyl-[1,2,4]triazolo[1,5-a]pyrazin-6-yl)benzonitrile (Example A10, Step 5; 241 mg, 0.48 mmol) in DCM (5 mL) was added NBS (84.6 mg, 0.48 mmol). The reaction mixture was then stirred at room temperature for 1 h, and concentrated to afford the crude product, which was used in the next step without further purification. LC-MS calculated for C₃₀H₂₆BrN₆O₂(M+H)⁺: m/z=581.1; found 581.1.

### Step 2: 3-(8-(bis(4-methoxybenzyl)amino)-5-bromo-2-formyl-[1,2,4]triazolo[1,5-a]pyrazin-6-yl)benzonitrile

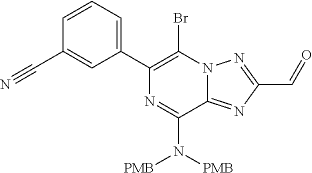

A mixture of 3-(8-(bis(4-methoxybenzyl)amino)-5-bromo-2-vinyl-[1,2,4]triazolo[1,5-a]pyrazin-6-yl)benzonitrile (174 mg, 0.3 mmol), osmium(VIII) oxide (3 mg in 0.3 mL water, 0.015 mmol), and sodium periodate (292 mg, 1.36 mmol) in THF/water (1:1, 6 mL) was stirred at 65° C. for 1 h. The reaction mixture was cooled to room temperature, and extracted with dichloromethane. The combined organic layers were concentrated, and purified by silica gel column to afford the desired product. LC-MS calculated for C₂₉H₂₄N₆O₃Br (M+H)⁺: m/z=583.1; found 583.1.

### Step 3: 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile

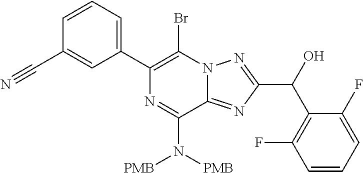

Preparation of the Grignard reagent: To a solution of 1,3-difluoro-2-iodobenzene (142 mg, 0.6 mmol) in tetrahydrofuran (1 mL), isopropylmagnesium chloride solution (296 μl, 2 M) was added at −10° C. The resulting mixture was stirred for 1 h, and used directly in the following step.

To a solution of 3-(8-(bis(4-methoxybenzyl)amino)-5-bromo-2-formyl-[1,2,4]triazolo[1,5-a]pyrazin-6-yl)benzonitrile (120 mg, 0.2 mmol) in THE (2 mL), the freshly prepared Grignard reagent from previous step was added at −10° C. The reaction mixture was stirred for 30 min, quenched with ammonium chloride solution (4 mL), and extracted with dichloromethane. The combined organic layers were concentrated under vacuum and purified by a silica gel column to afford the desired product as a racemic mixture. LC-MS calculated for C₃₅H₂₈N₆O₃BrF₂ (M+H)⁺: m/z=697.1; found 697.1.

### Step 4: 3-(8-(bis(4-methoxybenzyl)amino)-2-((2,6-difluorophenyl)(hydroxy)methyl)-5-(4-methyloxazol-5-yl)-[1,2,4]triazolo[1,5-a]pyrazin-6-yl)benzonitrile

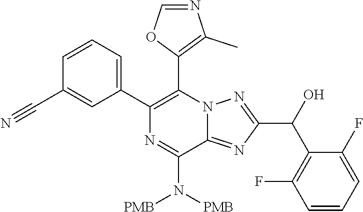

A mixture of 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile (382 mg, 0.55 mmol), 4-methyl-5-(4,4,5,5-tetramethyl-1,3,2-dioxaborolan-2-yl)oxazole (137 mg, 0.65 mmol), dicyclohexyl(2′,4′,6′-triisopropylbiphenyl-2-yl)phosphine-(2′-aminobiphenyl-2-yl)(chloro)palladium (1:1) (17 mg, 21.6 μmol) and Cs₂CO₃ (356 mg, 1.09 mmol) in 1,4-dioxane (2 mL) and water (200 μl) was purged with N₂ and heated at 95° C. for 7 h. The mixture was concentrated and purified via flash chromatography to afford the desired product as a colorless oil. LCMS calculated for C₃₉H₃₂N₇O₄F₂(M+H)⁺: 700.2; found 700.2.

### Step 5: 3-(8-(bis(4-methoxybenzyl)amino)-2-(chloro(2,6-difluorophenyl)methyl)-5-(4-methyloxazol-5-yl)-[1,2,4]triazolo[1,5-a]pyrazin-6-yl)benzonitrile

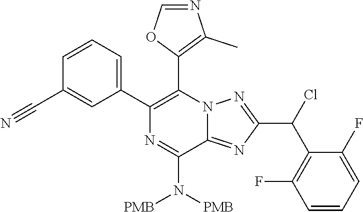

To a solution of 3-(8-(bis(4-methoxybenzyl)amino)-2-((2,6-difluorophenyl)(hydroxy)methyl)-5-(4-methyloxazol-5-yl)-[1,2,4]triazolo[1,5-a]pyrazin-6-yl)benzonitrile (201 mg, 0.29 mmol) in 2 mL of dichloromethane, thionyl chloride (105 μl, 1.435 mmol) was added at rt. The resulting mixture was stirred for 4 h, concentrated and used in next step without any further purification. LC-MS calculated for C₃₉H₃₁N₇O₃ClF₂ (M+H)⁺: m/z=718.2; found 718.2.

### Step 6: 3-(8-amino-2-(amino(2,6-difluorophenyl)methyl)-5-(4-methyloxazol-5-yl)-[1,2,4]triazolo[1,5-a]pyrazin-6-yl)benzonitrile

To a solution of 3-(8-(bis(4-methoxybenzyl)amino)-2-(chloro(2,6-difluorophenyl)methyl)-5-(4-methyloxazol-5-yl)-[1,2,4]triazolo[1,5-a]pyrazin-6-yl)benzonitrile (40 mg, 0.084 mmol) in 1 mL of DMSO was added ammonia solution (1 mL). The mixture was heated with microwave condition at 100° C. for 10 h before diluted with water and extracted with EtOAc. The combined organic layers were washed with water and brine, dried over MgSO₄, and concentrated. The resulting residue was dissolved in TFA (1 mL), and stirred at 80° C. for 20 min. The reaction mixture was then cooled to room temperature, concentrated, and basified by adding aq. NaHCO₃ solution. The crude material was directly purified by a silica gel column to afford the desired product as a racemic mixture. The product was then separated with chiral HPLC using a chiral column (AM-1) and 45% EtOH in hexanes (20 mL/min) solvent system. Peak 1 was isolated, and further purified via preparative LC/MS (pH=2, acetonitrile/water with TFA) to give the desired product as a TFA salt. LC-MS calculated for C₂₃H₁₇F₂N₈O (M+H)⁺: m/z=459.1; found 459.0.

### Example A12: Synthesis of 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile (Compound 12)

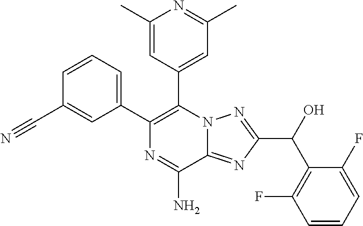 [SMILES: Cc1cc(-c2c(-c3cccc(C#N)c3)nc(N)c3nc(C(O)c4c(F)cccc4F)nn23)cc(C)n1]

To a solution of 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile (Example A11, Step 3; 0.518 g, 0.638 mmol), 2,6-dimethyl-4-(4,4,5,5-tetramethyl-1,3,2-dioxaborolan-2-yl)pyridine (0.346 g, 1.48 mmol), and dicyclohexyl(2′,4′,6′-triisopropylbiphenyl-2-yl)phosphine-(2′-aminobiphenyl-2-yl)(chloro)palladium (1:1) (0.058 g, 0.074 mmol) in dioxane (3.0 mL) and water (0.60 mL) was added potassium phosphate tribasic (0.472 g, 2.23 mmol). The reaction mixture was stirred at 90° C. for 1 h. The reaction mixture was then diluted with water and DCM. The layers were separated, the aqueous layer was extracted with DCM, and the combined organic fractions were dried over MgSO₄, filtered and concentrated. The crude material was dissolved in TFA (5 mL) and heated to 80° C. for 20 minutes. The reaction mixture was then cooled to room temperature, concentrated, and basified by adding aqueous NaHCO₃ solution. The crude material was directly purified by a silica gel column to afford the desired product (257 mg, 72%) as a racemic mixture.

The product was then separated with chiral HPLC using a chiral column (Phenomenex Lux Sum Cellulose-2, 21.1×250 mm) and 35% EtOH in Hexanes (20 mL/min) solvent system. Peak 2 was isolated, and further purified using preparative LC/MS (pH=2, acetonitrile/water with TFA) to give the desired product as a TFA salt. LC-MS calculated for C₂₆H₂₀F₂N₇O (M+H)⁺: m/z=484.2; found 484.2. ¹H NMR (500 MHz, DMSO-d₆) δ 7.92 (s, 2H), 7.85 (s, 1H), 7.83 (d, J=7.6 Hz, 1H), 7.56 (d, J=8.0 Hz, 1H), 7.53-7.40 (m, 4H), 7.10 (t, J=8.4 Hz, 2H), 6.27 (s, 1H), 2.51 (s, 6H).

### Example A13: Synthesis of 3-(4-amino-2-(pyridin-2-ylmethyl)-7-(pyrimidin-4-yl)-2H-[1,2,3]triazolo[4,5-c]pyridin-6-yl)benzonitrile (Compound 13)

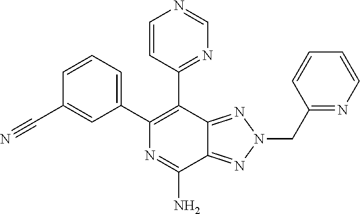 [SMILES: N#Cc1cccc(-c2nc(N)c3nn(Cc4ccccn4)nc3c2-c2ccncn2)c1]

### Step 1. 4,6-dichloro-3H-[1,2,3]triazolo[4,5-c]pyridine

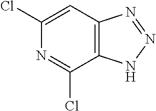 [SMILES: Clc1cc2nn[nH]c2c(Cl)n1]

A solution of NaNO₂ (3.88 g, 56.2 mmol) in water (3 mL) was added to a solution of 2,6-dichloropyridine-3,4-diamine (10 g, 56 mmol) in hydrochloric Acid, 37% (5 mL) at 0° C. The solution was stirred for 30 min. Water (20 mL) was added and the white precipitate was filtered, washed with water, and dried to give the desired product. LC-MS calculated for C₅H₃Cl₂N₄: 189.0 (M+H)⁺; found: 189.0 (M+H)⁺.

### Step 2. 6-chloro-N-(2,4-dimethoxybenzyl)-3H-[1,2,3]triazolo[4,5-c]pyridin-4-amine

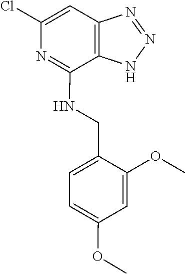 [SMILES: COc1ccc(CNc2nc(Cl)cc3nn[nH]c23)c(OC)c1]

The mixture of 4,6-dichloro-3H-[1,2,3]triazolo[4,5-c]pyridine (600 mg, 3.17 mmol), (2,4-dimethoxyphenyl)methanamine (0.53 mL, 3.49 mmol) and triethylamine (0.53 mL, 3.81 mmol) in 1,4-dioxane (10 mL) was stirred at 110° C. for 3 days. Direct purification on silica gel column afforded the desired product (875 mg, 86%). LC-MS calculated for C₁₄H₁₅ClN₅O₂: 320.1 (M+H)⁺; found: 320.3 (M+H)⁺.

### Step 3. 6-chloro-N-(2,4-dimethoxybenzyl)-2-(pyridin-2-ylmethyl)-2H-[1,2,3]triazolo[4,5-c]pyridin-4-amine

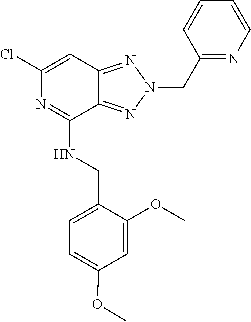 [SMILES: COc1ccc(CNc2nc(Cl)cc3nn(Cc4ccccn4)nc23)c(OC)c1]

The mixture of 6-chloro-N-(2,4-dimethoxybenzyl)-3H-[1,2,3]triazolo[4,5-c]pyridin-4-amine (875 mg, 2.74 mmol), pyridin-2-ylmethanol (0.317 mL, 3.28 mmol) and triphenylphosphine (1436 mg, 5.47 mmol) in DCM (20 mL) was added diisopropyl azodicarboxylate (0.647 mL, 3.28 mmol) at 0° C. The resulting mixture was stirred at 0° C. for 1 h. Direct purification on silica gel column afforded the desired product (375 mg, 33.4% yield). LC-MS calculated for C₂₀H₂₀ClN₆O₂: 411.1 (M+H)⁺; found: 411.2 (M+H)⁺.

### Step 4. 3-(4-((2,4-dimethoxybenzyl)amino)-2-(pyridin-2-ylmethyl)-2H-[1,2,3]triazolo[4,5-c]pyridin-6-yl)benzonitrile

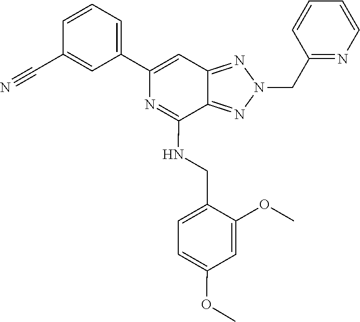 [SMILES: COc1ccc(CNc2nc(-c3cccc(C#N)c3)cc3nn(Cc4ccccn4)nc23)c(OC)c1]

To the mixture of 6-chloro-N-(2,4-dimethoxybenzyl)-2-(pyridin-2-ylmethyl)-2H-[1,2,3]triazolo[4,5-c]pyridin-4-amine (375 mg, 0.913 mmol) and (3-cyanophenyl)boronic acid (268 mg, 1.825 mmol) in 1,4-dioxane (10 mL) and water (1.00 mL) was added cesium carbonate (595 mg, 1.825 mmol). The resulting mixture was purged with N₂ and then chloro(2-dicyclohexylphosphino-2′,4′,6′-triisopropyl-1,1′-biphenyl)[2-(2′-amino-1,1′-biphenyl)]palladium(II) (71.8 mg, 0.091 mmol) was added. The reaction mixture was stirred at 120° C. under microwave irradiation for 90 min. The reaction was quenched with 20 mL of ethyl acetate and 20 mL of water. The organic phase was separated and the aqueous solution was extracted with ethyl acetate twice. The combined extracts were dried over Na₂SO₄, filtered and evaporated under reduced pressure. The residue was purified on silica gel column to afford the desired product (300 mg, 68.9%). LC-MS calculated for C₂₇H₂₄N₇O₂: 478.2 (M+H)⁺; found: 478.3 (M+H)⁺.

### Step 5. 3-(4-amino-2-(pyridin-2-ylmethyl)-2H-[1,2,3]triazolo[4,5-c]pyridin-6-yl)benzonitrile

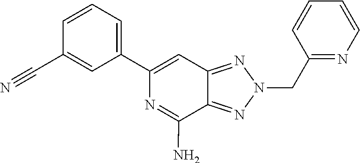 [SMILES: N#Cc1cccc(-c2cc3nn(Cc4ccccn4)nc3c(N)n2)c1]

The solution of 3-(4-((2,4-dimethoxybenzyl)amino)-2-(pyridin-2-ylmethyl)-2H-[1,2,3]triazolo[4,5-c]pyridin-6-yl)benzonitrile (300.3 mg, 0.629 mmol) in TFA (5 mL) was stirred at 100° C. for 30 min. TFA was evaporated under reduced pressure and then 20 mL of saturated NaHCO₃ aqueous solution and 20 mL of ethyl acetate were added. The organic phase was separated and the aqueous solution was extracted with ethyl acetate twice. The combined extracts were dried over Na₂SO₄, filtered and evaporated under reduced pressure. The residue was purified on silica gel column to afford the desired product (175 mg, 85%). LC-MS calculated for C₁₈H₁₄N₇: 328.1 (M+H)⁺; found: 328.2 (M+H)⁺.

### Step 6. 3-(4-amino-7-bromo-2-(pyridin-2-ylmethyl)-2H-[1,2,3]triazolo[4,5-c]pyridin-6-yl)benzonitrile

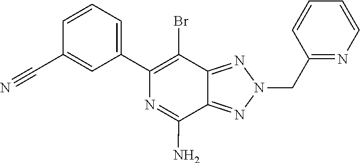 [SMILES: N#Cc1cccc(-c2nc(N)c3nn(Cc4ccccn4)nc3c2Br)c1]

The mixture of 3-(4-amino-2-(pyridin-2-ylmethyl)-2H-[1,2,3]triazolo[4,5-c]pyridin-6-yl)benzonitrile (175 mg, 0.535 mmol) and 1-bromopyrrolidine-2,5-dione (100 mg, 0.561 mmol) in THE (10 mL) was stirred at 0° C. for 30 min and then quenched with saturated NaHCO₃ aqueous solution. The organic phase was separated, dried over Na₂SO₄, filtered and evaporated under reduced pressure. The resulting residue was purified on silica gel column to afforded the desired product (135 mg, 62.2%). LC-MS calculated for C₁₈H₁₃BrN₇: 406.0 (M+H)⁺ and 408.0 (M+H)⁺; found: 406.1 (M+H)⁺ and 408.2 (M+H)⁺.

### Step 7. 3-(4-amino-2-(pyridin-2-ylmethyl)-7-(pyrimidin-4-yl)-2H-[1,2,3]triazolo[4,5-c]pyridin-6-yl)benzonitrile

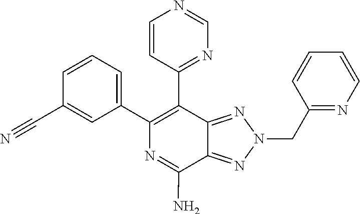 [SMILES: N#Cc1cccc(-c2nc(N)c3nn(Cc4ccccn4)nc3c2-c2ccncn2)c1]

A mixture of 3-(4-amino-7-bromo-2-(pyridin-2-ylmethyl)-2H-[1,2,3]triazolo[4,5-c]pyridin-6-yl)benzonitrile (182 mg, 0.448 mmol), 4-(tributylstannyl)pyrimidine (496 mg, 1.344 mmol), and copper(I) chloride (53.2 mg, 0.538 mmol), lithium chloride (22.79 mg, 0.538 mmol) and tetrakis(triphenylphosphine)palladium(0) (51.8 mg, 0.045 mmol) in THE (1 ml) was first purged with N₂, and then heated and stirred at 90° C. for 2 h. The reaction was diluted with methanol and purified with prep-LCMS (pH=2) to give the desired product. LC-MS calculated for C₂₂H₁₆N₉: 406.2 (M+H)⁺; found: 406.2 (M+H)⁺.

### Example A14: Synthesis of 3-(4-amino-2-((3-fluoropyridin-2-yl)methyl)-7-(pyrimidin-4-yl)-2H-[1,2,3]triazolo[4,5-c]pyridin-6-yl)benzonitrile (Compound 14)

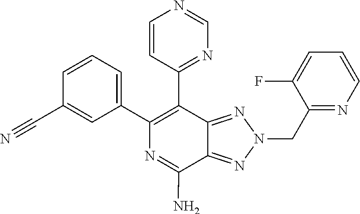 [SMILES: N#Cc1cccc(-c2nc(N)c3nn(Cc4ncccc4F)nc3c2-c2ccncn2)c1]

### Step 1. 6-chloro-N-(2,4-dimethoxybenzyl)-2-((3-fluoropyridin-2-yl)methyl)-2H-[1,2,3]triazolo[4,5-c]pyridin-4-amine

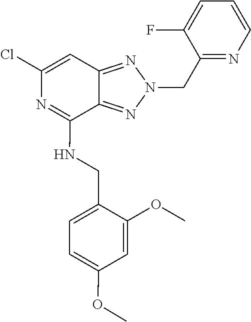 [SMILES: COc1ccc(CNc2nc(Cl)cc3nn(Cc4ncccc4F)nc23)c(OC)c1]

To the mixture of 6-chloro-N-(2,4-dimethoxybenzyl)-3H-[1,2,3]triazolo[4,5-c]pyridin-4-amine (Example A13, Step 2; 1000 mg, 3.13 mmol), (3-fluoropyridin-2-yl)methanol (477 mg, 3.75 mmol) and triphenylphosphine (1641 mg, 6.25 mmol) in DCM (1.7 mL) was added diisopropyl azodicarboxylate (739 μl, 3.75 mmol) at 0° C. The reaction mixture was stirred at 0° C. for 1 h. Direct purification on silica gel column afforded the desired product (433 mg, 32%). LC-MS calculated for C₂₀H₁₉ClFN₆O₂: 429.1 (M+H)⁺; found: 429.3 (M+H)⁺.

### Step 2. 3-(4-((2,4-dimethoxybenzyl)amino)-2-((3-fluoropyridin-2-yl)methyl)-2H-[1,2,3]triazolo[4,5-c]pyridin-6-yl)benzonitrile

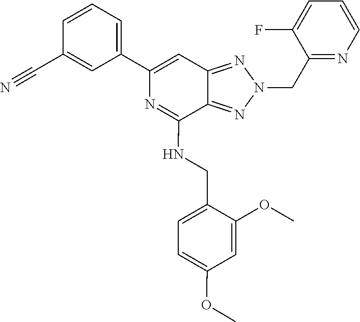 [SMILES: COc1ccc(CNc2nc(-c3cccc(C#N)c3)cc3nn(Cc4ncccc4F)nc23)c(OC)c1]

Cesium carbonate (658 mg, 2.019 mmol) was added to the mixture of 6-chloro-N-(2,4-dimethoxybenzyl)-2-((3-fluoropyridin-2-yl)methyl)-2H-[1,2,3]triazolo[4,5-c]pyridin-4-amine (433 mg, 1.010 mmol) and (3-cyanophenyl)boronic acid (297 mg, 2.019 mmol) in 1,4-dioxane (10.0 mL) and water (1.0 mL). The resulting mixture was sparged with N₂ for 2 min and (SP-4-4)-[2′-Amino[1,1′-biphenyl]-2-yl]chloro[dicyclohexyl[2′,4′,6′-tris(1-methylethyl)[1,1′-biphenyl]-2-yl]phosphine]palladium (79 mg, 0.101 mmol) was added. The reaction mixture was stirred at 120° C. for 1.5 h under microwave irradiation. The reaction was quenched with 20 mL of ethyl acetate and 20 mL of water. The organic phase was separated and the aqueous solution was extracted with ethyl acetate twice. The combined extracts were dried over Na₂SO₄, filtered and evaporated under reduced pressure. The residue was purified on silica gel column to afford the desired product (357 mg, 71%). LC-MS calculated for C₂₇H₂₃FN₇O₂: 496.2 (M+H)⁺; found: 496.3 (M+H)⁺.

### Step 3. 3-(4-amino-2-((3-fluoropyridin-2-yl)methyl)-2H-[1,2,3]triazolo[4,5-c]pyridin-6-yl)benzonitrile

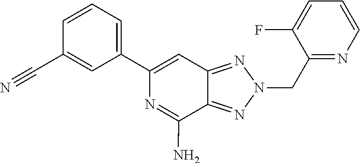 [SMILES: N#Cc1cccc(-c2cc3nn(Cc4ncccc4F)nc3c(N)n2)c1]

The solution of 3-(4-((2,4-dimethoxybenzyl)amino)-2-((3-fluoropyridin-2-yl)methyl)-2H-[1,2,3]triazolo[4,5-c]pyridin-6-yl)benzonitrile (357.3 mg, 0.721 mmol) in TFA (5 mL) was stirred at 100° C. for 1 h. TFA was evaporated under reduced pressure and then 20 mL of saturated NaHCO₃ aqueous solution and 20 mL of ethyl acetate were added. The organic phase was separated and the aqueous solution was extracted with ethyl acetate twice. The combined extracts were dried over Na₂SO₄, filtered and evaporated under reduced pressure. The residue was purified on silica gel column to afford the desired product (213 mg, 61%). LC-MS m/z calculated for C₁₈H₁₃FN₇: 346.1 (M+H)⁺; found: 346.3 (M+H)⁺.

### Step 4. 3-(4-amino-7-bromo-2-((3-fluoropyridin-2-yl)methyl)-2H-[1,2,3]triazolo[4,5-c]pyridin-6-yl)benzonitrile

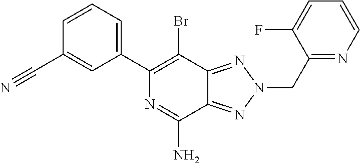 [SMILES: N#Cc1cccc(-c2nc(N)c3nn(Cc4ncccc4F)nc3c2Br)c1]

The mixture of 3-(4-amino-2-((3-fluoropyridin-2-yl)methyl)-2H-[1,2,3]triazolo[4,5-c]pyridin-6-yl)benzonitrile (213 mg, 0.617 mmol) and 1-bromopyrrolidine-2,5-dione (220 mg, 1.234 mmol) in THE (5 mL) was stirred at 0° C. for 1 h. Direct purification on silica gel afforded the desired product (175 mg, 67%). LC-MS calculated for C₁₈H₁₂BrFN₇: 424.0 (M+H)⁺ and 426.0 (M+H)⁺; found: 424.3 (M+H)⁺ and 426.3 (M+H)⁺.

### Step 5. 3-(4-amino-2-((3-fluoropyridin-2-yl)methyl)-7-(pyrimidin-4-yl)-2H-[1,2,3]triazolo[4,5-c]pyridin-6-yl)benzonitrile

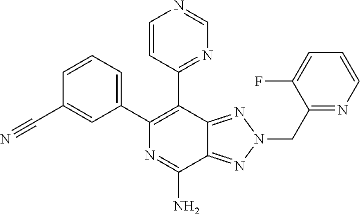 [SMILES: N#Cc1cccc(-c2nc(N)c3nn(Cc4ncccc4F)nc3c2-c2ccncn2)c1]

The mixture of 3-(4-amino-7-bromo-2-((3-fluoropyridin-2-yl)methyl)-2H-[1,2,3]triazolo[4,5-c]pyridin-6-yl)benzonitrile (220 mg, 0.519 mmol), 4-(tributylstannyl)pyrimidine (383 mg, 1.037 mmol), and copper(I) chloride (61.6 mg, 0.622 mmol), lithium chloride (26.4 mg, 0.622 mmol) and tetrakis(triphenylphosphine)palladium(0) (59.9 mg, 0.052 mmol) in THF (1 ml) was first purged with N₂, and then heated and stirred at 90° C. for 2 h. The reaction was diluted with methanol and purified with prep-LCMS (pH=2) to give the desired product. LC-MS calculated for C₂₂H₁₅FN₉: 424.1 (M+H)⁺; found: 424.3 (M+H)⁺. ¹H NMR (500 MHz, DMSO- [symbol] ₆) ppm 8.98 (s, 1H), 8.77 (d, J=5.02 Hz, 1H), 8.38 (dd, J₁=4.60 Hz, J₂=1.32 Hz, 1H), 7.90-8.30 (bs, 2H), 7.76-7.89 (m, 3H), 7.66 (dd, J₁=5.25 Hz, J₂=1.25 Hz, 1H), 7.45-7.58 (m, 3H), 6.25 (s, 2H).

### Example A15: Synthesis of 3-(4-amino-2-((3-fluoropyridin-2-yl)methyl)-7-(pyridin-4-yl)-2H-[1,2,3]triazolo[4,5-c]pyridin-6-yl)benzonitrile (Compound 15)

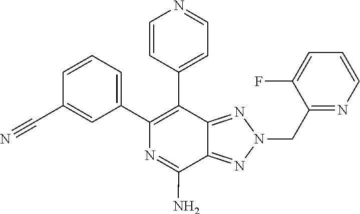 [SMILES: N#Cc1cccc(-c2nc(N)c3nn(Cc4ncccc4F)nc3c2-c2ccncc2)c1]

Cesium carbonate (46.1 mg, 0.141 mmol) was added to a mixture of 3-(4-amino-7-bromo-2-((3-fluoropyridin-2-yl)methyl)-2H-[1,2,3]triazolo[4,5-c]pyridin-6-yl)benzonitrile (30 mg, 0.071 mmol) and pyridin-4-ylboronic acid (17.38 mg, 0.141 mmol) in 1,4-dioxane (2 mL) and water (0.2 mL). The resulting mixture was sparged with N₂ for 2 min and chloro(2-dicyclohexylphosphino-2′,4′,6′-triisopropyl-1,1′-biphenyl)[2-(2′-amino-1,1′-biphenyl)]palladium(II) (5.56 mg, 7.07 μmol) was added. The reaction mixture was stirred at 120° C. for 1.5 h under microwave irradiation. The reaction mixture was diluted with methanol. Direct purification on prep. HPLC afforded the desired product. LC-MS calculated for C₂₃H₁₆FN₈: 423.1 (M+H)⁺; found: 423.3 (M+H)⁺.

### Example A16: Synthesis of 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile (Compound 16)

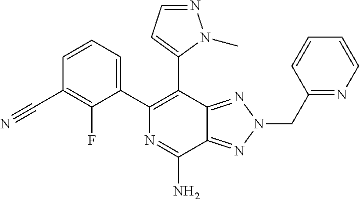 [SMILES: Cn1nccc1-c1c(-c2cccc(C#N)c2F)nc(N)c2nn(Cc3ccccn3)nc12]

### Step 1. 3-(4-amino-7-bromo-2-(pyridin-2-ylmethyl)-2H-[1,2,3]triazolo[4,5-c]pyridin-6-yl)-2-fluorobenzonitrile

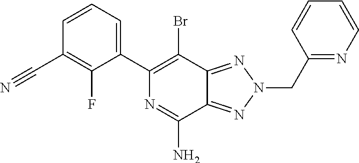 [SMILES: N#Cc1cccc(-c2nc(N)c3nn(Cc4ccccn4)nc3c2Br)c1F]

This compound was prepared by following a similar procedure from Example A13, Step 1 to Step 6, with (3-cyano-2-fluorophenyl)boronic acid replacing (3-cyanophenyl)boronic acid in Step 4. LC-MS calculated for C₁₈H₁₂BrFN₇: 424.0 (M+H)⁺ and 426.0 (M+H)⁺; found: 424.3 (M+H)⁺ and 426.3 (M+H)⁺.

### Step 2. 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile

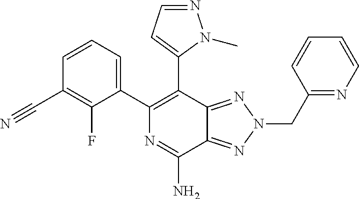 [SMILES: Cn1nccc1-c1c(-c2cccc(C#N)c2F)nc(N)c2nn(Cc3ccccn3)nc12]

This compound was prepared by following a similar procedure in Example A15, with (1-methyl-1H-pyrazol-5-yl)boronic acid replacing pyridin-4-ylboronic acid, and with 3-(4-amino-7-bromo-2-(pyridin-2-ylmethyl)-2H-[1,2,3]triazolo[4,5-c]pyridin-6-yl)-2-fluorobenzonitrile replacing 3-(4-amino-7-bromo-2-((3-fluoropyridin-2-yl)methyl)-2H-[1,2,3]triazolo[4,5-c]pyridin-6-yl)benzonitrile. LC-MS calculated for C₂₂H₁₇FN₉: 426.2 (M+H)⁺; found: 426.3 (M+H)⁺.

### Example A17: Synthesis of 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one (Compound 17)

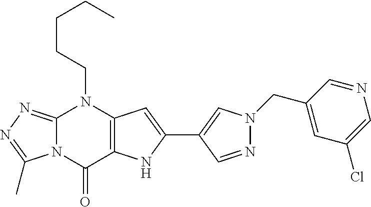 [SMILES: CCCCCn1c2cc(-c3cnn(Cc4cncc(Cl)c4)c3)[nH]c2c(=O)n2c(C)nnc12]

### Step 1: Ethyl 3-(pentylamino)-1H-pyrrole-2-carboxylate

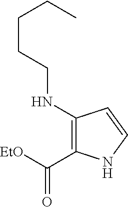 [SMILES: CCCCCNc1cc[nH]c1C(=O)OCC]

Ethyl 3-amino-1H-pyrrole-2-carboxylate (5 g, 32.4 mmol), pentanal (3.79 ml, 35.7 mmol), and sodium cyanoborohydride (2.038 g, 32.4 mmol) were mixed in methanol (64.9 ml) at room temperature overnight. The reaction mixture was concentrated under reduced pressure. The crude residue was purified by flash chromatography (0 to 100% EtOAc in hexanes) to give the desired product (4.4 g, 61%). LCMS calculated for C₁₂H₂₁N₂O₂ (M+H): 225.2. Found: 225.1.

### Step 2: Ethyl 3-(3-(ethoxycarbonyl)-1-pentylthioureido)-1H-pyrrole-2-carboxylate

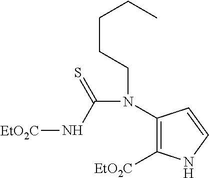 [SMILES: CCCCCN(C(=S)NC(=O)OCC)c1cc[nH]c1C(=O)OCC]

A vial was charged with ethyl 3-(pentylamino)-1H-pyrrole-2-carboxylate (4.4 g, 19.62 mmol), dichloromethane (39.2 ml), and ethoxycarbonyl isothiocyanate (2.78 ml, 23.54 mmol). The reaction mixture was stirred at room temperature overnight. The reaction mixture was quenched with water (40 ml), and the layers were separated. The aqueous layer was extracted with dichloromethane (3×40 mL) and the combined organic fractions were dried over MgSO₄, filtered, and concentrated. The crude material was used in the next step without further purification (7.3 g, quant.). LCMS calculated for C₁₆H₂₆N₃O₄S (M+H): 356.2. Found: 356.1.

### Step 3: 1-Pentyl-2-thioxo-2,3-dihydro-1H-pyrrolo[3,2-d]pyrimidin-4(5H)-one

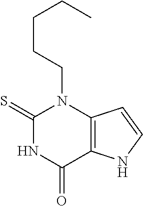 [SMILES: CCCCCn1c(=S)[nH]c(=O)c2[nH]ccc21]

A microwave vial was charged with ethyl 3-(3-(ethoxycarbonyl)-1-pentylthioureido)-1H-pyrrole-2-carboxylate (7.31 g, 20.57 mmol) and sodium ethoxide (21% w/w, 8.45 ml, 22.62 mmol) solution. The vial was capped and heated in a microwave reactor for 10 minutes at 120 degrees Celsius. The reaction mixture was brought to neutral pH on addition of 1M HCl solution and the solid product was filtered and dried (3.1 g, 64%). LCMS calculated for C₁₁H₁₆N₃₀S (M+H): 238.1. Found: 238.1.

### Step 4: 2-Hydrazono-1-pentyl-2,3-dihydro-1H-pyrrolo[3,2-d]pyrimidin-4(5H)-one

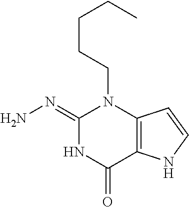 [SMILES: CCCCCn1/c(=N/N)[nH]c(=O)c2[nH]ccc21]

A vial was charged with 1-pentyl-2-thioxo-2,3-dihydro-1H-pyrrolo[3,2-d]pyrimidin-4(5H)-one (3.13 g, 13.19 mmol) and hydrazine hydrate (20 mL). The reaction mixture was stirred at 100 degrees Celsius overnight. The solid formed was filtered and washed with water to give the desired product (2.2 g, 70%). LCMS calculated for C₁₁H₁₈N₅O (M+H): 236.1. Found: 236.1.

### Step 5: 3-Methyl-9-pentyl-6,9-dihydro-5H-pyrrolo[3,2-d][1,2,4]triazolo[4,3-a]pyrimidin-5-one

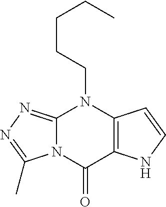 [SMILES: CCCCCn1c2cc[nH]c2c(=O)n2c(C)nnc12]

A vial was charged with (E)-2-hydrazono-1-pentyl-2,3-dihydro-1H-pyrrolo[3,2-d]pyrimidin-4(5H)-one (4.8 g, 20.40 mmol), a drop of trifluoroacetic acid, and triethyl orthoacetate (20 mL). The reaction mixture was heated to 110 degrees Celsius for three hours. The suspension was filtered, washed with hexanes, and dried (4.0 g, 76%). LCMS calculated for C₁₃H₁₈N₅O (M+H): 260.1. Found: 260.2.

### Step 6: 3-Methyl-9-pentyl-6-(phenylsulfonyl)-6,9-dihydro-5H-pyrrolo[3,2-d][1,2,4]triazolo[4,3-a]pyrimidin-5-one

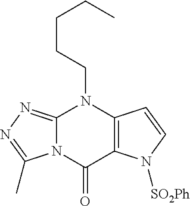

A vial was charged with 3-methyl-9-pentyl-6,9-dihydro-5H-pyrrolo[3,2-d][1,2,4]triazolo[4,3-a]pyrimidin-5-one (from Step 1) (4 g, 15.43 mmol), dichloromethane (40 mL), dimethylaminopyridine (0.188 g, 1.543 mmol), triethylamine (3.23 ml, 23.14 mmol), and benzenesulfonyl chloride (2.187 ml, 16.97 mmol). The reaction mixture was stirred at room temperature for one hour. The reaction mixture was quenched with water, and the layers were separated. The aqueous layer was extracted with dichloromethane (3×40 mL) and the combined organic fractions were dried over MgSO₄, filtered, and concentrated. The crude material was used in the next step without further purification (6.1 g, quant.). LCMS calculated for C₁₉H₂₂N₅O₃S (M+H): 400.1. Found: 400.1.

### Step 7: 7-Bromo-3-methyl-9-pentyl-6-(phenylsulfonyl)-6,9-dihydro-5H-pyrrolo[3,2-d][1,2,4]triazolo[4,3-a]pyrimidin-5-one

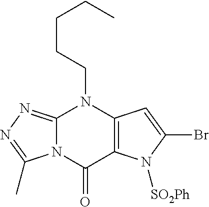

A vial was charged with 3-methyl-9-pentyl-6-(phenylsulfonyl)-6,9-dihydro-5H-pyrrolo[3,2-d][1,2,4]triazolo[4,3-a]pyrimidin-5-one (1 g, 2.503 mmol), dry THF (30 mL) and the mixture was cooled to −78 degrees Celsius. Lithium diisopropylamide solution (1M in hexanes/THF, 3.13 ml, 3.13 mmol) was added dropwise. The reaction mixture was maintained at −78° C. for 1.5 hours. A solution of 1,2-dibromo-1,1,2,2-tetrachloroethane (1.223 g, 3.75 mmol) in dry THF (3 ml) was added dropwise to the reaction mixture and the reaction mixture was maintained at −78° C. for a further 1.5 hours. The reaction mixture was quenched with sat. aq. NH₄Cl solution (30 mL) and diluted with dichloromethane (30 mL). The layers were separated and the aqueous layer was extracted with DCM (3×30 mL). The combined organic fractions were dried over MgSO₄, filtered, and concentrated. The crude residue was purified by automated flash chromatography (0 to 100% EtOAc in DCM) to give the desired product (0.84 g, 70%). LCMS calculated for C₁₉H₂₁BrN₅O₃S (M+H): 478.1. Found: 478.1.

### Step 8: 3-Chloro-5-((4-(4,4,5,5-tetramethyl-1,3,2-dioxaborolan-2-yl)-1H-pyrazol-1-yl)methyl)pyridine

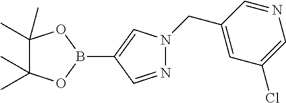 [SMILES: CC1(C)OB(c2cnn(Cc3cncc(Cl)c3)c2)OC1(C)C]

A vial was charged with 4-(4,4,5,5-tetramethyl-1,3,2-dioxaborolan-2-yl)-1H-pyrazole (0.5 g, 2.58 mmol), 3-(bromomethyl)-5-chloropyridine hydrobromide (0.741 g, 2.58 mmol), cesium carbonate (2.52 g, 7.73 mmol), and DMF (6.44 ml). The reaction mixture was stirred at 60 degrees Celsius for one hour. The reaction mixture was quenched with water (10 ml) and diluted with dichloromethane (10 ml). The layers were separated, and the aqueous layer was extracted with dichloromethane (3×10 mL). The combined dichloromethane extracts were dried over MgSO₄, filtered, and concentrated. Purification by automated flash chromatography (0 to 100% EtOAc in DCM) afforded the product (0.548 g, 67%). LCMS calculated for C₁₅H₂₀BClN₃O₂(M+H): 320.1, 322.1. Found: 320.1, 322.1

### Step 9: 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

A vial was charged with 7-bromo-3-methyl-9-pentyl-6-(phenylsulfonyl)-6,9-dihydro-5H-pyrrolo[3,2-d][1,2,4]triazolo[4,3-a]pyrimidin-5-one (0.01 g, 0.021 mmol), 3-chloro-5-((4-(4,4,5,5-tetramethyl-1,3,2-dioxaborolan-2-yl)-1H-pyrazol-1-yl)methyl)pyridine (0.013 g, 0.042 mmol), Chloro(2-dicyclohexylphosphino-2′,4′,6′-triisopropyl-1,1′-biphenyl)[2-(2′-amino-1,1′-biphenyl)]palladium(II) (5.00 mg, 0.006 mmol) and potassium phosphate tribasic (0.016 g, 0.074 mmol). 1,4-dioxane (0.35 ml) and water (0.07 ml) were added and the reaction mixture was sparged with nitrogen gas for 5 minutes then stirred at 90° C. for two hours. The reaction mixture was cooled to room temperature and sodium hydroxide (10 mg) was added. The reaction mixture was stirred at 40 degrees Celsius for 60 minutes. The reaction mixture was cooled to room temperature and diluted with DMF (5 ml). Purification by preparative HPLC (pH 2, acetonitrile/water with TFA) afforded the product as a TFA salt (2 mg, 21%). LCMS calculated for C₂₂H₂₄ClN₈O (M+H): 451.2, 453.2. Found: 451.2, 453.2.

### Example A18: Synthesis of 3-Methyl-7-(1-((5-methylpyridin-3-yl)methyl)-1H-pyrazol-4-yl)-9-pentyl-6,9-dihydro-5H-pyrrolo[3,2-d][1,2,4]triazolo[4,3-a]pyrimidin-5-one (Compound 18)

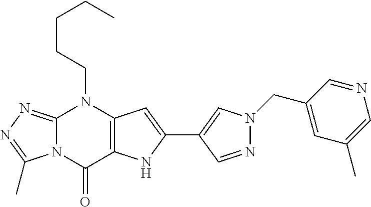 [SMILES: CCCCCn1c2cc(-c3cnn(Cc4cncc(C)c4)c3)[nH]c2c(=O)n2c(C)nnc12]

This compound was prepared using similar procedures as described in Example A17 using 3-(bromomethyl)-5-methylpyridine in place of 3-(bromomethyl)-5-chloropyridine hydrobromide in Step 8. LCMS calculated for C₂₃H₂₇N₈O (M+H): 431.2. Found: 431.3.

### Example A19: Synthesis of 3-Methyl-9-pentyl-7-(1-(thieno[3,2-b]pyridin-6-ylmethyl)-1H-pyrazol-4-yl)-6,9-dihydro-5H-pyrrolo[3,2-d][1,2,4]triazolo[4,3-a]pyrimidin-5-one (Compound 19)

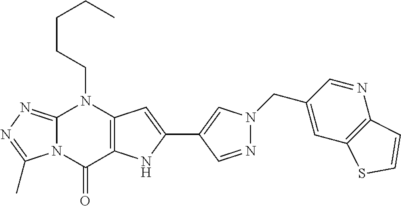 [SMILES: CCCCCn1c2cc(-c3cnn(Cc4cnc5ccsc5c4)c3)[nH]c2c(=O)n2c(C)nnc12]

This compound was prepared using similar procedures as described in Example A17 using 6-(bromomethyl)thieno[3,2-b]pyridine in place of 3-(bromomethyl)-5-chloropyridine hydrobromide in Step 8. LCMS calculated for C₂₄H₂₅N₈OS (M+H): 473.2. Found: 473.3.

### Example A20: 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one (Compound 20)

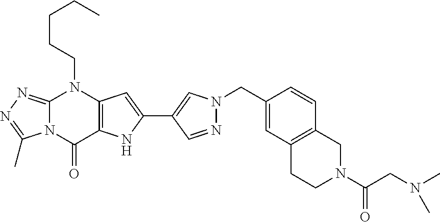 [SMILES: CCCCCn1c2cc(-c3cnn(Cc4ccc5c(c4)CCN(C(=O)CN(C)C)C5)c3)[nH]c2c(=O)n2c(C)nnc12]

### Step 1: tert-Butyl 6-((4-(4,4,5,5-tetramethyl-1,3,2-dioxaborolan-2-yl)-1H-pyrazol-1-yl)methyl)-3,4-dihydroisoquinoline-2(1H)-carboxylate

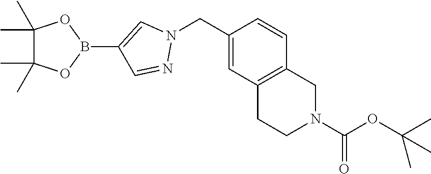 [SMILES: CC(C)(C)OC(=O)N1CCc2cc(Cn3cc(B4OC(C)(C)C(C)(C)O4)cn3)ccc2C1]

A flask was charged with 4-(4,4,5,5-tetramethyl-1,3,2-dioxaborolan-2-yl)-1H-pyrazole (0.5 g, 2.58 mmol), tert-butyl 6-(hydroxymethyl)-3,4-dihydroisoquinoline-2(1H)-carboxylate (0.339 g, 1.288 mmol), triphenylphosphine (0.743 g, 2.83 mmol), and THE (12 ml). The solution was cooled to 0° C. and DIAD (0.601 ml, 3.09 mmol) was added dropwise. The reaction mixture was stirred overnight at room temperature. The mixture was diluted with ethyl acetate and washed with water, dried and concentrated. The product was purified by column chromatography eluting with Hexane/EtOAc (max. EtOAc 60%) to afford the product. LCMS calculated for C₂₄H₃₅BN₃O₄(M+H)⁺: m/z=440.3; found 440.3.

### Step 2: 7-bromo-3-methyl-9-pentyl-6,9-dihydro-5H-pyrrolo[3,2-d][1,2,4]triazolo[4,3-a]pyrimidin-5-one

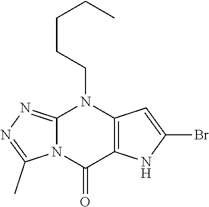 [SMILES: CCCCCn1c2cc(Br)[nH]c2c(=O)n2c(C)nnc12]

TBAF (1.0 M in THF) (2.0 ml, 2.0 mmol) was added to a solution of 7-bromo-3-methyl-9-pentyl-6-(phenylsulfonyl)-6,9-dihydro-5H-pyrrolo[3,2-d][1,2,4]triazolo[4,3-a]pyrimidin-5-one (0.360 g, 0.753 mmol) in THF (4.0 ml), and then the reaction was stirred at 50° C. for 1 h. The solvent was removed and the product was purified by column chromatography eluting with CH₂Cl₂/MeOH (max. MeOH 10%). LCMS calculated for C₁₃H₁₇BrN₅O (M+H)⁺: m/z=338.1; found 338.1.

### Step 3: tert-Butyl 6-((4-(3-methyl-5-oxo-9-pentyl-6,9-dihydro-5H-pyrrolo[3,2-d][1,2,4]triazolo[4,3-a]pyrimidin-7-yl)-1H-pyrazol-1-yl)methyl)-3,4-dihydroisoquinoline-2(1H)-carboxylate

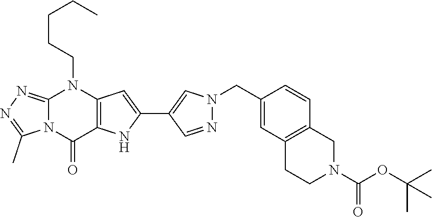 [SMILES: CCCCCn1c2cc(-c3cnn(Cc4ccc5c(c4)CCN(C(=O)OC(C)(C)C)C5)c3)[nH]c2c(=O)n2c(C)nnc12]

A mixture of 7-bromo-3-methyl-9-pentyl-6,9-dihydro-5H-pyrrolo[3,2-d][1,2,4]triazolo[4,3-a]pyrimidin-5-one (from Example A20, Step 2) (0.040 g, 0.118 mmol), tert-butyl 6-((4-(4,4,5,5-tetramethyl-1,3,2-dioxaborolan-2-yl)-1H-pyrazol-1-yl)methyl)-3,4-dihydroisoquinoline-2(1H)-carboxylate (0.062 g, 0.142 mmol), dichloro[1,1′-bis(dicyclohexylphosphino)ferrocene]palladium(II), dichloromethane adduct (Pd-127) (8.94 mg, 0.012 mmol) and cesium fluoride (0.090 g, 0.591 mmol) in t-BuOH (1.5 ml)/Water (0.6 ml) was vacuumed and replaced with N₂ for 3 times. The reaction was then stirred at 105° C. for 2 h, cooled to rt, diluted with ethyl acetate, washed with water, dried and concentrated. The product was purified by column eluting with CH₂Cl₂/MeOH (max. MeOH 10%). LCMS calculated for C₃₁H₃₉N₈O₃ (M+H)⁺: m/z=571.3; found 571.5.

### Step 4: 3-Methyl-9-pentyl-7-(1-((1,2,3,4-tetrahydroisoquinolin-6-yl)methyl)-1H-pyrazol-4-yl)-6,9-dihydro-5H-pyrrolo[3,2-d][1,2,4]triazolo[4,3-a]pyrimidin-5-one

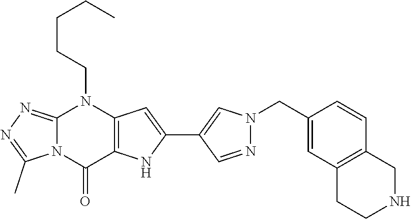 [SMILES: CCCCCn1c2cc(-c3cnn(Cc4ccc5c(c4)CCNC5)c3)[nH]c2c(=O)n2c(C)nnc12]

TFA (0.5 ml, 6.49 mmol) was added to a solution of tert-butyl 6-((4-(3-methyl-5-oxo-9-pentyl-6,9-dihydro-5H-pyrrolo[3,2-d][1,2,4]triazolo[4,3-a]pyrimidin-7-yl)-1H-pyrazol-1-yl)methyl)-3,4-dihydroisoquinoline-2(1H)-carboxylate (50.0 mg, 0.088 mmol) in CH₂Cl₂ (0.5 ml), and then the reaction was stirred at room temperature for 30 min. The solvent was then removed to provide the crude product as TFA salt. LCMS calculated for C₂₆H₃₁N₈O (M+H)⁺: m/z=471.3; found 471.2.

### Step 5: 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

Dimethylglycinoyl chloride (3.10 mg, 0.026 mmol) was added to a solution of 3-methyl-9-pentyl-7-(1-((1,2,3,4-tetrahydroisoquinolin-6-yl)methyl)-1H-pyrazol-4-yl)-6,9-dihydro-5H-pyrrolo[3,2-d][1,2,4]triazolo[4,3-a]pyrimidin-5-one (6.0 mg, 0.013 mmol) and triethylamine (8.89 μl, 0.064 mmol) in CH₂Cl₂ (0.8 ml) at room temperature and stirred for 30 min. The solvent was removed, and the mixture was diluted with acetonitrile/water and purified by prep HPLC (pH 2, acetonitrile/water with TFA) to provide the desired compound as its TFA salt. LC-MS calculated for C₃₀H₃₈N₉O₂ (M+H)⁺: m/z=556.3; found 556.3.

### Example A21. 3-(2-((5-(1H-pyrazol-1-yl)-2H-tetrazol-2-yl)methyl)-5-amino-8-(pyrimidin-4-yl)-[1,2,4]triazolo[1,5-c]pyrimidin-7-yl)benzonitrile (Compound 21A) and 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile (Compound 21B)

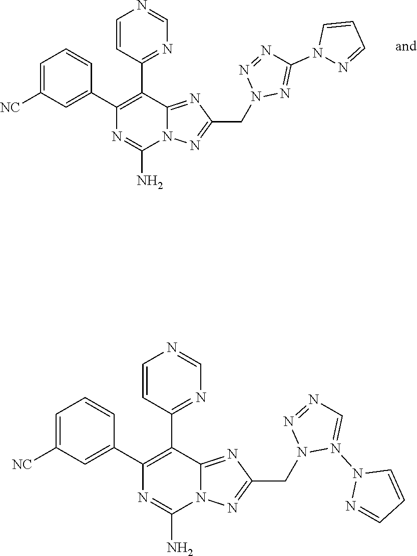

The mixture of title compounds was prepared using similar procedures as described for Example A3, with 5-(1H-pyrazol-1-yl)-1H-tetrazole replacing 2-(1H-tetrazol-5-yl)pyridine. Compound 21A was purified by preparative LC-MS (pH 2, acetonitrile/water with TFA) to afford the product as a TFA salt. LCMS calculated for C₂₁H₁₅N₁₄ (M+H)⁺: 463.2; found 463.2.

### Example 1: Generation of Anti-Human CD73 Monoclonal Antibodies

To generate anti-human CD73 monoclonal antibodies, mice were immunized with recombinant human CD73 (SEQ ID NO:70) protein comprising a C-terminal HIS-tag and B cells were isolated from the mouse spleen and lymph nodes. Antibody sequences of the B cells were determined using 10× Genomics VH/VL paired B cell sequencing. The murine VH/VL pairs were expressed as chimeras with huIgG1 Fc (SEQ ID NOs: 73 and 74) and tested for binding and functionality. An antibody designated CL25 was produced by this process. Table 1, above, shows the amino acid sequences of the CL25 CDRs according to IMGT, Chothia, AbM, Kabat, and Contact numbering and the mature VH, VL, heavy chain, and light chain.

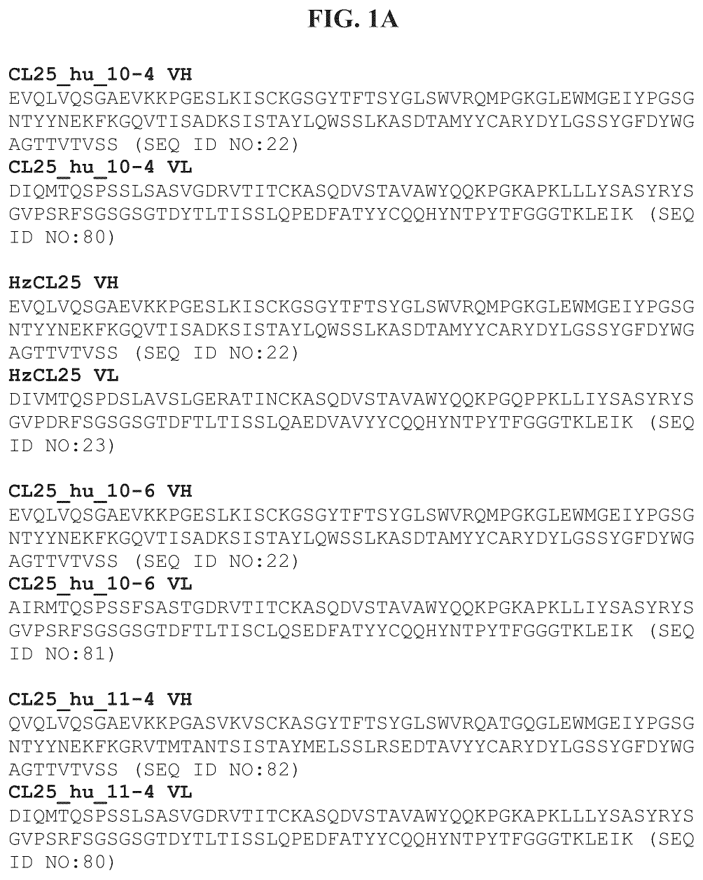

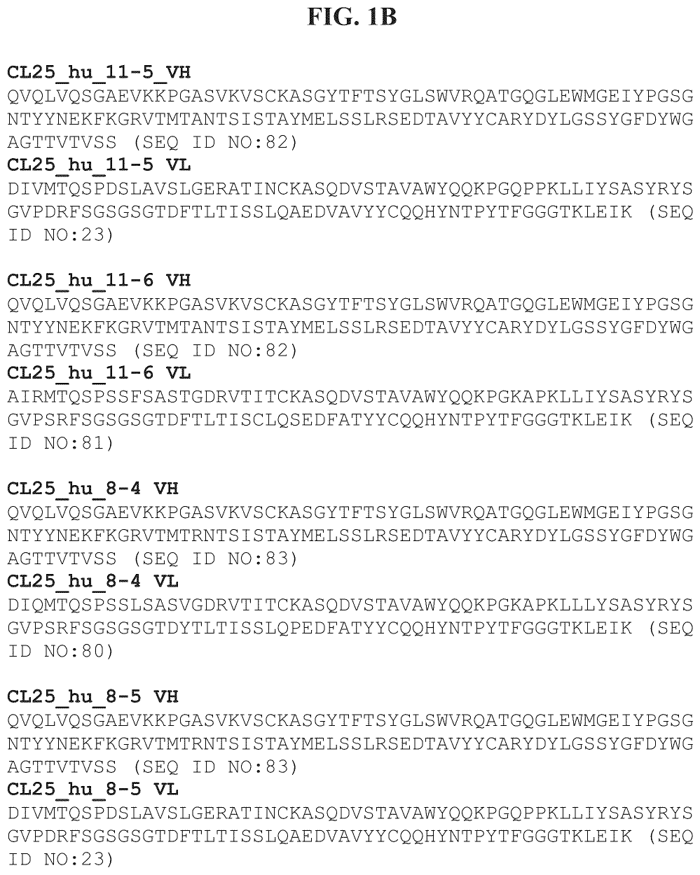

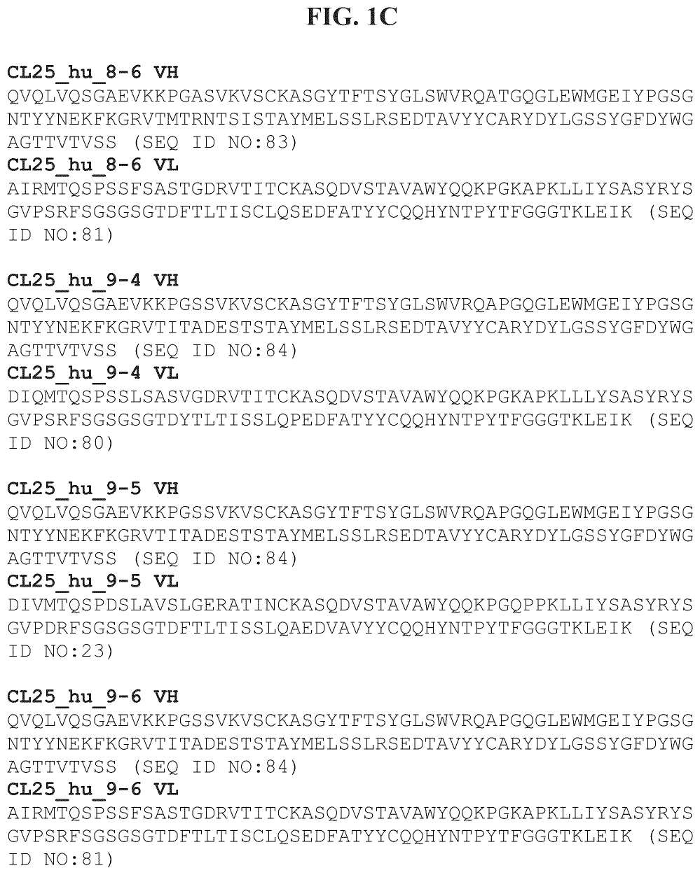

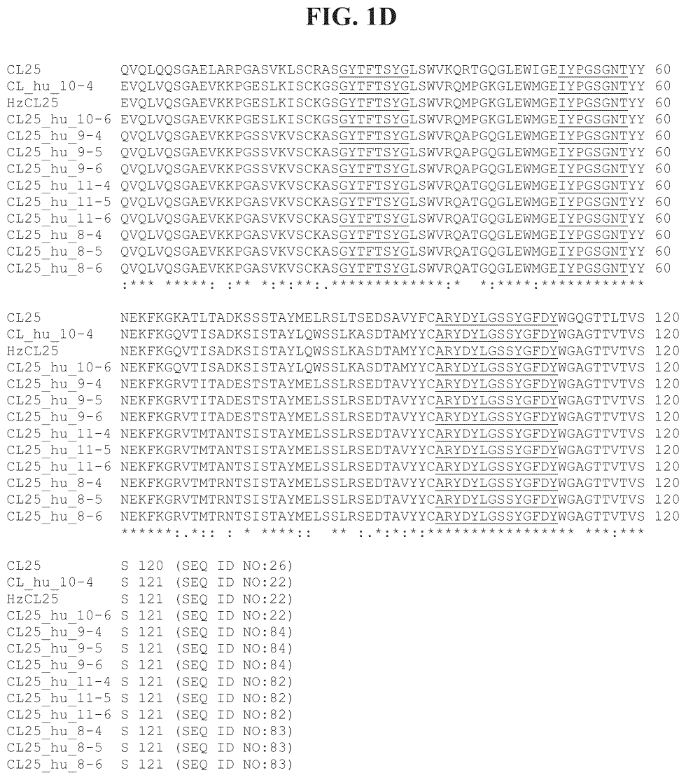

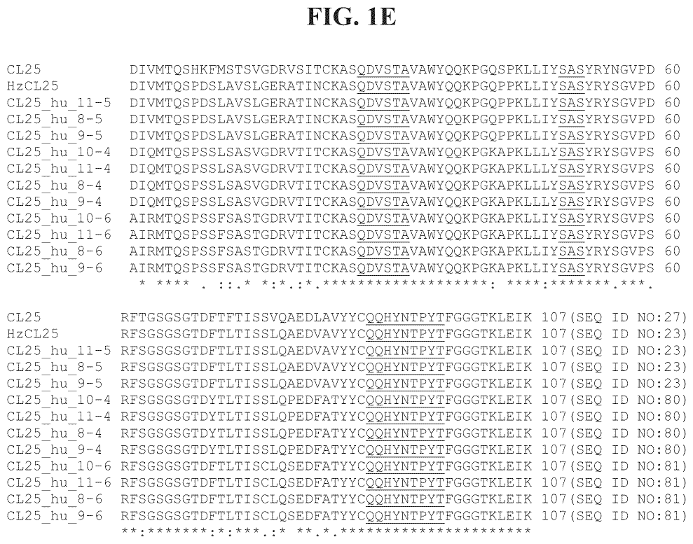

Chimeric antibody CL25 (comprising the murine VH of SEQ ID NO:26 and murine VL of SEQ ID NO:27) was humanized to minimize the immunogenicity of the antibody frameworks while maintaining specific activity. Humanization was conducted by aligning the VH and VL sequences to a database of human VH and VK genes. The CDRs (Table 1) from the murine CL25 antibody were grafted into several top human VH and VK genes. The VH and VL sequences of exemplary humanized CL25 antibodies are depicted in FIG. 1A-FIG. 1C. Alignments of the VH and VL of CL25 and exemplary humanized CL25 antibodies are depicted in FIG. 1D and FIG. 1E, respectively. Several framework mutations present in the murine CL25 were also tested along with the murine CDRs (FIG. 1A-FIG. 1E). The humanized version of CL25 having a VH of SEQ ID NO:22 and a VL of SEQ ID NO:23, referred to herein as “HzCL25”, was selected for further studies. Table 2, above, shows the amino acid sequences of the HzCL25 CDRs according to IMGT, Chothia, AbM, Kabat, and Contact numbering and the mature VH, VL, heavy chain, and light chain.

### Example 2: Binding of Anti-Human CD73 Monoclonal Antibodies to Cell Surface CD73

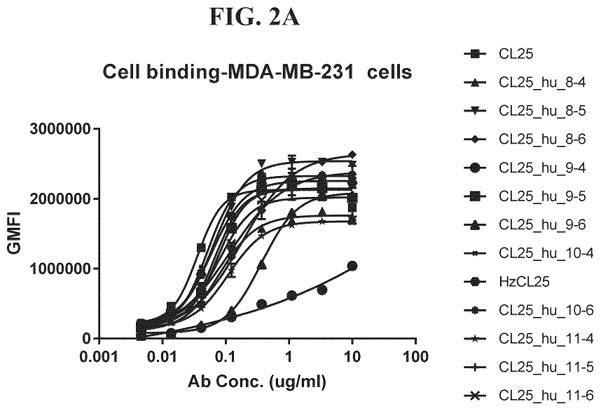

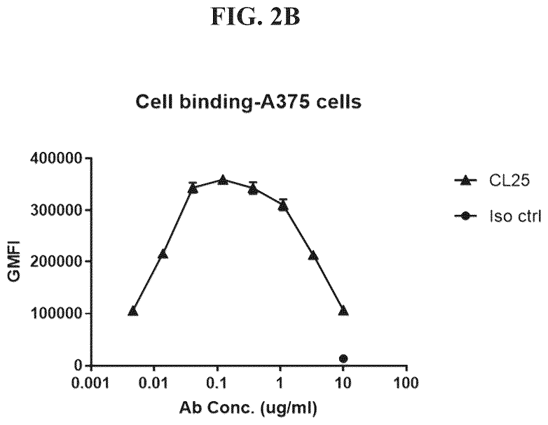

To test the binding of humanized and non-humanized CL25 clones to cell surface CD73, MDA-MB-231 or A375 cells were washed and added to 96-well plates at 5×10⁴ cells/well. The cells were stained with the indicated concentration of antibodies for 30 minutes on ice (FIG. 2A and FIG. 2B). Next, the cells were washed and stained using goat anti-mouse secondary conjugated to phycoerythrin (PE) for 30 minutes on ice. The cells were then washed and analyzed by flow cytometry. Geometric mean fluorescence intensity (GMFI) of CD73 staining was graphed (FIG. 2A and FIG. 2B). Both CL25 and HzCL25 displayed high potency binding to cells with high levels of surface CD73 (MDA-MB-231 cells) and moderate levels of surface CD73 (as tested in A375 cells).

### Example 3: Anti-Human CD73 Monoclonal Antibody-Mediated Cellular CD73 Inhibition

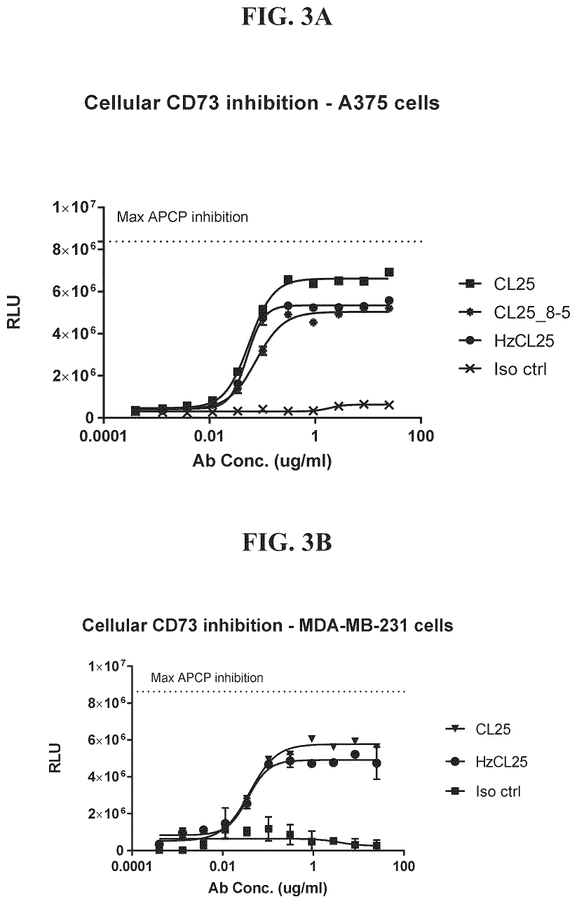

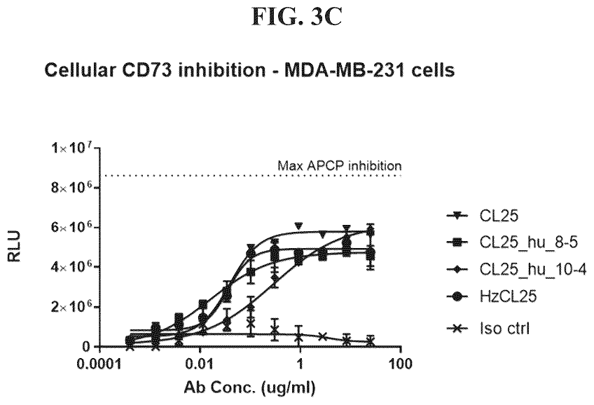

To measure ability of anti-CD73 antibody to inhibit CD73 activity on cells, A375 and MDA-MB-231 cells were washed with serum free RPMI media (ThermoFisher) and plated in 96-well plates at a concentration of 8×10⁴ cells/well for A375 or 1×10⁴ cells/well for MDA-MB-231. The cells were incubated with the indicated concentration of antibodies or APCP at 37° C. 5% CO₂ for 30 minutes (FIG. 3A, FIG. 3B, and FIG. 3C). Next, adenosine monophosphate (AMP) was added to a final concentration of 100 μM and cells were incubated an additional 3 hours at 37° C. 5% CO₂. Plates were centrifuged for 1-2 minutes at 300 g and 25 μL of supernatant was transferred into a new 96-well plates. AMP-Glo Assay was used according to the manufacturer's instructions (Promega). Relative luminescence unit (RLU) is directly correlated with the AMP concentration in this assay. Results are depicted in FIG. 3A, FIG. 3B, and FIG. 3C.

Both CL25 and HzCL25 had good potency in inhibiting cellular CD73 in both tested cell types (FIG. 3A, FIG. 3B, and FIG. 3C). HzCL25 had a similar ability as CL25 to inhibit cellular CD73 (FIG. 3A, FIG. 3B, and FIG. 3C).

### Example 4: Anti-Human CD73 Monoclonal Antibody-Mediated Soluble CD73 Inhibition

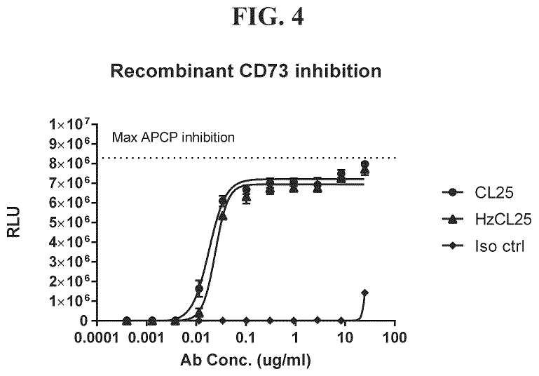

To measure the ability of the CD73 antibodies to inhibit CD73 activity of recombinant protein, recombinant human CD73 (rhuCD73) (SEQ ID NO:70) was added to 96-well plates at a final concentration of 0.008 μg/mL with the indicated concentration of antibodies (FIG. 4) or adenosine 5′-[α,β-methylene]diphosphate (APCP) and incubated at 37° C. 5% CO₂ for 30 minutes. After the 30 minute incubation, AMP was added to a final concentration of 100 μM and the reactions were incubated an additional 3 hours at 37° C. 5% CO₂. 25 μL of supernatant was transferred into new 96-well plates. The AMP-Glo Assay was used according to the manufacturer's instructions. RLU is a directly correlated with the AMP concentration in this assay. Results are depicted in FIG. 4. Both CL25 and HzCL25 showed high potency and no hook-effect (FIG. 4). HzCL25 had a similar ability as CL25 to inhibit cellular CD73 (FIG. 4).

### Example 5: Binding Affinity

CD73 enzymatic activity requires substrate binding in the open conformation. After substrate binding, CD73 has to go through a large conformational change from open to closed conformation to convert AMP to adenosine. Antibody binding that inhibits or modulates this conformational change will potentially decrease the rate of AMP to adenosine conversion.

To assess the binding affinity of HzCL25, surface plasmon resonance (SPR) was performed using a Biacore 8K instrument (GE Healthcare) at 25° C. The SPR running buffer (10 mM HEPES, 150 mM NaCl, 3 mM EDTA and 0.05% v/v Surfactant P20, pH 7.4) was prepared from 10×HBS-EP Buffer (GE Healthcare). Anti-human Fc antibodies (GE Healthcare) were immobilized via amine coupling on all sixteen flow cells of an S series sensor chip CM5 (GE Healthcare). The immobilization levels were ˜9000 RU for all flow cells. The desired capturing level of anti-CD73 antibody was achieved by flowing appropriate concentration of anti-CD73 antibody through the active flow cell of each channel. The non-cleavable ADP analogue APCP (adenosine-5′-(α,β-methylene) diphosphate) with the presence of Zn²⁺ can be used to shift the CD73 conformational equilibrium from open towards closed. Therefore, recombinant CD73 was incubated with SPR running buffer in the presence of 100 μM APCP and 10 μM ZnCl₂ (closed SPR running buffer) to study the binding of anti-CD73 antibody to the CD73 in the closed conformation. To achieve this, the ABA injection feature in Biacore 8K was used. For open conformation, the ABA injection sequence started with 60 seconds injection of running buffer. Then, CD73 3-fold serial dilution concentration series prepared from CD73 stock (BPS Bioscience) and running buffer were injected for 180 seconds immediately followed by running buffer for 240 seconds. For closed conformation, the normal SPR running buffer was replaced by closed SPR running buffer. Surface was regenerated with 30 seconds injection of 3 M MgCl₂. Binding kinetics and affinity parameters were obtained from a global fit of the data to 1 to 1 binding model. Binding affinities and kinetic association and dissociation rate constants to human, cynomolgus, and mouse CD73 in either open or closed conformations are shown in Table 8 below. The results in Table 8 ensure cynomolgus pharmacokinetic data will reflect human pharmacokinetic data.

TABLE 8
Binding affinities and kinetic association and dissociation rate constants to human
(SEQ ID NO: 70), cynomolgus (SEQ ID NO: 72), and mouse (SEQ ID NO: 71)
CD73 in either open or closed conformations for the indicated antibodies (Ab).

| | Open | | | | Closed | | |
|---|---|---|---|---|---|---|---|
| Ab | CD73 | kₐ (1/Ms) | k_{d} (1/s) | K_{D} (M) | kₐ (1/Ms) | k_{d} (1/s) | K_{D} (M) |
| CL25 | human | >1E+06 | 3.94E−04 | <3.94E−10 | 9.03E+05 | 3.44E-04 | 3.81E-10 |
| | Cyno- | >1E+06 | 6.58E−04 | <6.58E−10 | 9.36E+05 | 4.95E-04 | 5.29E-10 |
| | molgus | | | | | | |

| | Murine | No Binding | | | No Binding |
|---|---|---|---|---|---|
| HzCL25 | Human | >1E+06 | 4.49E−04 | <4.49E−10 | Not performed |
| | Cyno- | >1E+06 | 4.68E−04 | <4.68E−10 | Not performed |
| | molgus | | | | |
| | Murine | No binding | | | Not performed |
| CL25_ | Human | 7.70E+05 | 1.76E−03 | 2.29E−09 | Not performed |
| hu_8-4 | | | | | |
| CL25_ | Human | >1E+06 | 9.49E−04 | 7.18E−10 | Not performed |
| hu_8-5 | | | | | |
| CL25_ | Human | 7.15E+05 | 1.11E−03 | 1.55E−09 | Not performed |
| hu_8-6 | | | | | |
| CL25_ | Human | 3.82E+05 | 2.64E−03 | 6.90E−09 | Not performed |
| hu_9-4 | | | | | |
| CL25_ | Human | 5.65E+05 | 1.14E−03 | 2.02E−09 | Not performed |
| hu_9-5 | | | | | |
| CL25_ | Human | 4.24E+05 | 9.53E−04 | 2.25E−09 | Not performed |
| hu_9-6 | | | | | |
| CL25_ | Human | 8.87E+05 | 1.07E−03 | 1.20E−09 | Not performed |
| hu_10-4 | | | | | |
| CL25_ | Human | 5.24E+05 | 9.20E−04 | 1.75E−09 | Not performed |
| hu_10-6 | | | | | |
| CL25_ | Human | 6.24E+05 | 1.55E−03 | 2.48E−09 | Not performed |
| hu_11-4 | | | | | |
| CL25_ | Human | 7.99E+05 | 1.17E−03 | 1.46E−09 | Not performed |
| hu_11-5 | | | | | |
| CL25_ | Human | 5.50E+05 | 1.01E−03 | 1.84E−09 | Not performed |
| hu_11-6 | | | | | |

### Example 6: Epitope Mapping

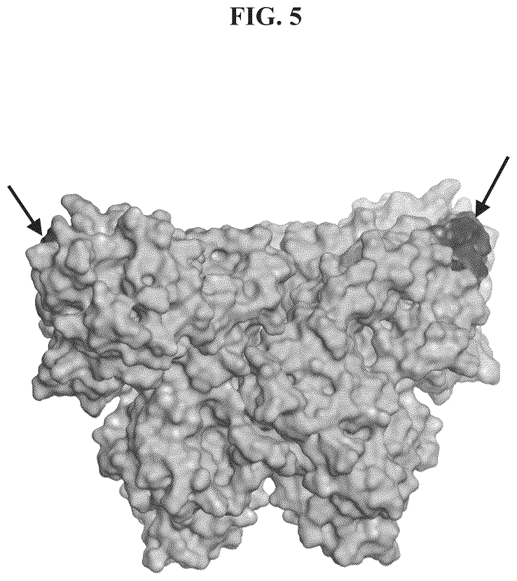

To map the epitope of CL25, Hydrogen-deuterium exchange mass spectrometry (HDX) was performed. CD73 was incubated in deuterium oxide either alone or in complex with CL25 Fab. The deuterium exchange was carried out at 20° C. for 0 seconds, 60 seconds, 600 seconds, or 3600 seconds. The exchange reaction was quenched by low pH and the proteins were digested with pepsin/protease VIII. The deuterium levels at the identified peptides were monitored from the mass shift on LC-MS. The deuterium buildup curves over exchange time for all the peptides were plotted vs time. Peptides with significant reduction in deuterium uptakes upon binding to Fab were assigned as the epitopes for each antibody. The epitope determined by HDX-MS for CL25 is mapped onto the crystal structure of human CD73 (4H₂F.pdb) (FIG. 5) and is TKVQQIRRAEPNVL (SEQ ID NO:76) (i.e., amino acids 40-53 of SEQ ID NO:70).

### Example 7: CD73 Cell Surface Levels

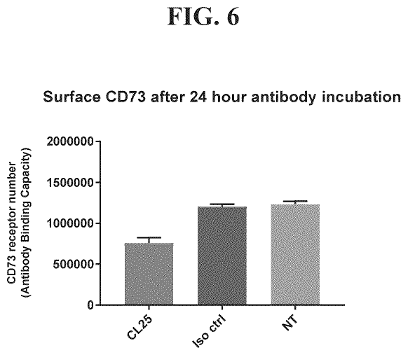

To measure the amount of CD73 on the cell surface after antibody treatment, MDA-MB-231 cells were re-suspended in media (10% FBS RPMI-1640) and plated in 96-well plates at 1×10⁵ cells/well. Indicated antibodies were added at a final concentration of 10 μg/mL and plates were incubated at 37° C. 5% CO₂ for 24 hours. Cells were recovered using Versene and transferred to new 96-well plates. Cells were washed and stained for 30 minutes on ice with 10 μg/mL of non-competing antibody directly conjugated to Dy650. Cells were washed and analyzed by flow cytometry. CD73 cell surface receptor density was determined by Antibody Binding Capacity (ABC) using Quantum Simply Cellular beads. Treatment of cells with CL25 for 24 hours decreased the levels of cell surface CD73 (FIG. 6).

### Example 8: Generation of Anti-Human CD73 Monoclonal Antibody 3-F03

To generate additional anti-human CD73 monoclonal antibodies, multiple selection rounds of single donor library were performed. The library of approximately 1.5E12 phage particles was enriched over three rounds of panning using 200 nM biotinylated human CD73 (SEQ ID NO:70). The scFv cassettes from this pool were then recombined into a yeast display vector and a library of approximately 5.4E7 was created. This library was selected by FACS for three rounds using 100 nM biotinylated murine CD73 (SEQ ID NO:71). Unique sequences were obtained from the final sorting output by Sanger sequencing of yeast colonies. The yeast 3-F03 scFv sequence was identified from this pool and contained a VH of the amino acid sequence set forth in SEQ ID NO:77 and a VL of the amino acid sequence set forth in SEQ ID NO:65.

To construct a full-length human 3-F03 antibody, the yeast 3-F03 scFv sequences were modified prior to cloning into a human IgG1 scaffold comprising the human IgG1 constant region set forth in SEQ ID NO:75 and the human kappa light chain constant region set forth in SEQ ID NO:74. For the VH, the N-terminal glutamate (E) of SEQ ID NO:77 was removed and the threonine (T) at Kabat position H77 of SEQ ID NO:77 (i.e., position 78 of SEQ ID NO:77) was substituted with an alanine (A). For the VL, the N-terminal alanine (A) of SEQ ID NO:65 was removed. The resulting full-length human 3-F03 antibody contains the VH and VL set forth in the amino acid sequences of SEQ ID NOs:60 and 61, respectively. The resulting full-length human 3-F03 antibody contains the heavy chain and light chain set forth in the amino acid sequences of SEQ ID NOs: 66 and 31, respectively.

This antibody is referred to herein as “3-F03”. Table 3, above, shows the amino acid sequences of the 3-F03 CDRs according to IMGT, Chothia, AbM, Kabat, and Contact numbering and of the mature VH, VL, heavy chain, and light chain.

### Example 9: Binding of 3-F03 to Cell Surface CD73

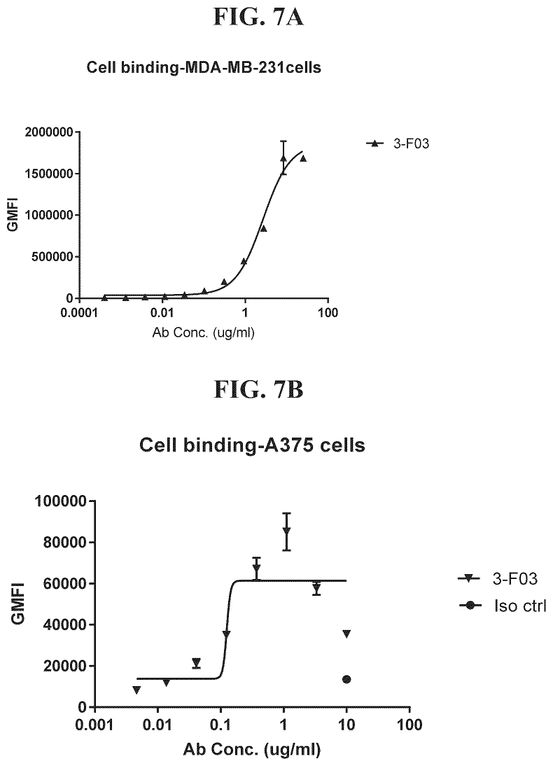

The binding of 3-F03 to cell surface CD73 was performed as described in Example 2, above. 3-F03 displays high potency binding to cells with high levels of surface CD73 (MDA-MB-231), and moderate levels of CD73 (A375 cells) (FIG. 7A and FIG. 7B).

### Example 10: 3-F03-Mediated Cellular CD73 Inhibition

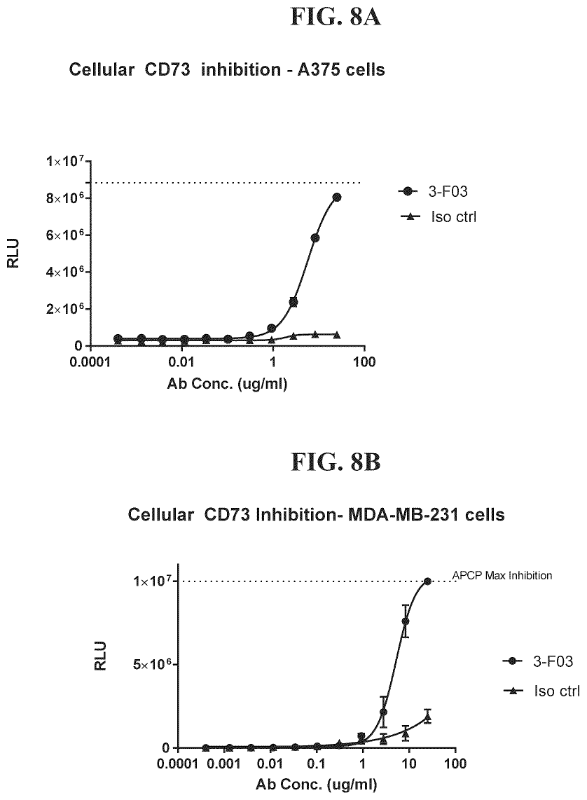

The ability of 3-F03 to inhibit CD73 activity on cells was evaluated as described in Example 3, above. Results are depicted in FIG. 8A and FIG. 8B.

Clone 3-F03 showed maximum inhibition of cellular CD73 in both tested cell types as compared to the small molecule inhibitor of CD73, APCP (FIG. 8A and FIG. 8B).

### Example 11: 3-F03-Mediated Soluble CD73 Inhibition

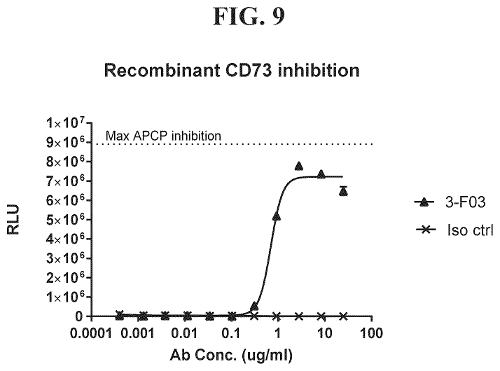

The ability of 3-F03 to inhibit CD73 activity of recombinant protein was evaluated as described in Example 4, above, except that 0.025 ug/mL of rhuCD73 was used. Results are depicted in FIG. 9. Antibody 3-F03 had good potency (FIG. 9). Antibody 3-F03 did not exhibit any hook-effect.

### Example 12: Binding Affinity of Anti-CD73 Antibody

The binding affinity of 3-F03 was evaluated as described in Example 5, above. Binding affinities and kinetic association and dissociation rate constants to human, cynomolgus, and mouse CD73 in either open or closed conformations are shown in Table 9 below.

TABLE 9
Binding affinities and kinetic association and dissociation rate constants to human,
cynomolgus, and mouse CD73 in either open or closed conformations.

| Sample | | Open | | | Closed | | |
|---|---|---|---|---|---|---|---|
| Name | CD73 | kₐ (1/Ms) | k_{d} (1/s) | K_{D} (M) | kₐ (1/Ms) | k_{d} (1/s) | K_{D} (M) |

| | | | | | |
|---|---|---|---|---|---|
| 3-F03 | human | 2.15E+05 | 7.96E−05 | 3.70E−10 | No Binding |
| | Cynomolgus | 3.01E+05 | 2.21E−04 | 7.34E−10 | No Binding |
| | Murine | 2.17E+05 | 3.60E−04 | 1.66E−09 | No Binding |

### Example 13: Epitope Mapping of 3-F03

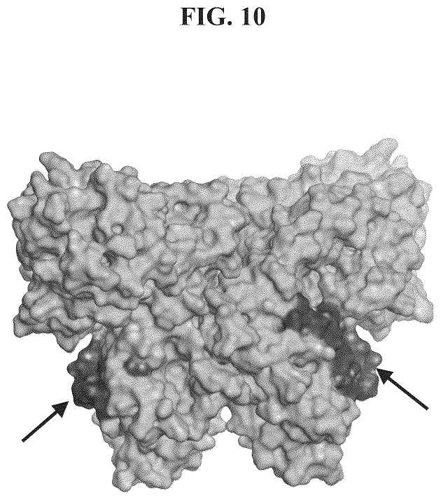

The epitope of 3-F03 was mapped as described in Example 6, above. The epitopes determined by HDX-MS for 3-F03 are mapped onto the crystal structure of human CD73 (4H2F.pdb) (FIG. 10) and are AAVLPFGGTFDLVQ (SEQ ID NO:78) (i.e., amino acids 386-399 of SEQ ID NO:70) and ILPNFLANGGDGFQMIKDEL (SEQ ID NO:79) (i.e., amino acids 470-489 of SEQ ID NO:70).

### Example 14: Effect of 3-F03 on CD73 Cell Surface Levels

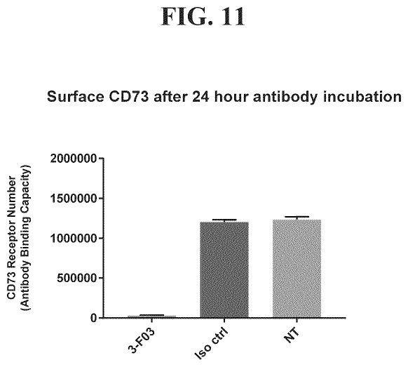

The amount of CD73 on the cell surface after treatment with 3-F03 was evaluated as described in Example 7, above. 3-F03 dramatically decreased the level of detectable CD73 on the cell surface compared to an isotype control antibody or non-treated cells (FIG. 11).

### Example 15: 3-F03 Variants

Sequences of the 3-F03 light chain (LC, SEQ ID NO:66) and heavy chain (HC, SEQ ID NO:31) were used to construct a homology model. FIG. 12A-FIG. 12J provide the amino acid sequences of the VH and VL of exemplary 3-F03 variants. Antibodies comprising these VH and VL sequences contained the heavy chain constant region set forth in SEQ ID NO:73 and light chain constant region set forth in SEQ ID NO:74. Table 10 provides the binding affinity and kinetics of the exemplary 3-F03 variants. None of the mutations tested dramatically impacted binding to CD73 by Biacore. All tested mutations had affinities within tenfold of the 3-F03 antibody, with the majority within two fold of 3-F03.

TABLE 10
Biacore binding affinity and kinetics of 3-F03 variants. — = absent

| | Mutation made (kabat numbering) | | | | | | |
|---|---|---|---|---|---|---|---|
| Sample | VH1 | VH53 | VH77 | VL1 | | | |
| Name | (E or −) | (D, E, or S) | (A or T) | (A, D, or −) | kₐ (1/Ms) | k_{d} (1/s) | K_{D} (M) |
| 3-F03_396 | E | D | T | A | 1.39E+05 | 1.78E−04 | 1.28E−09 |
| 3-F03_408 | E | D | T | — | 1.40E+05 | 1.86E−04 | 1.33E−09 |
| 3-F03_402 | — | D | T | A | 1.32E+05 | 1.84E−04 | 1.39E−09 |
| 3-F03_384 | E | D | T | D | 1.40E+05 | 1.98E−04 | 1.41E−09 |
| 3-F03_399 | E | D | A | A | 1.38E+05 | 1.97E−04 | 1.43E−09 |
| 3-F03_411 | E | D | A | — | 1.39E+05 | 2.04E−04 | 1.47E−09 |
| 3-F03_414 | — | D | T | — | 1.31E+05 | 1.98E−04 | 1.51E−09 |
| 3-F03_390 | — | D | T | D | 1.29E+05 | 2.12E−04 | 1.64E−09 |
| 3-F03_398 | E | E | T | A | 9.26E+04 | 1.59E−04 | 1.71E−09 |
| 3-F03_387 | E | D | A | D | 1.37E+05 | 2.38E−04 | 1.74E−09 |
| 3-F03_386 | E | E | T | D | 9.23E+04 | 1.64E−04 | 1.78E−09 |
| 3-F03_401 | E | E | A | A | 9.15E+04 | 1.67E−04 | 1.82E−09 |
| 3-F03_413 | E | E | A | — | 9.13E+04 | 1.71E−04 | 1.88E−09 |
| 3-F03_405 | — | D | A | A | 1.26E+05 | 2.39E−04 | 1.90E−09 |
| 3-F03_410 | E | E | T | — | 9.01E+04 | 1.76E−04 | 1.95E−09 |
| 3-F03_389 | E | E | A | D | 9.17E+04 | 1.89E−04 | 2.06E−09 |
| 3-F03_393 | — | D | A | D | 1.14E+05 | 2.39E−04 | 2.09E−09 |
| 3-F03_417 | — | D | A | — | 1.34E+05 | 2.84E−04 | 2.12E−09 |
| 3-F03_392 | — | E | T | D | 8.08E+04 | 1.80E−04 | 2.23E−09 |
| 3-F03_404 | — | E | T | A | 8.35E+04 | 1.89E−04 | 2.26E−09 |
| 3-F03_419 | — | E | A | — | 8.28E+04 | 2.00E−04 | 2.41E−09 |
| 3-F03_416 | — | E | T | — | 9.01E+04 | 2.21E−04 | 2.45E−09 |
| 3-F03_407 | — | E | A | A | 8.74E+04 | 2.35E−04 | 2.69E−09 |
| 3-F03_395 | — | E | A | D | 7.12E+04 | 2.10E−04 | 2.94E−09 |
| 3-F03_388 | E | S | A | D | 1.15E+05 | 8.68E−04 | 7.56E−09 |
| 3-F03_397 | E | S | T | A | 6.07E+04 | 4.89E−04 | 8.04E−09 |
| 3-F03_385 | E | S | T | D | 6.33E+04 | 5.38E−04 | 8.50E−09 |
| 3-F03_400 | E | S | A | A | 6.15E+04 | 5.29E−04 | 8.60E−09 |
| 3-F03_409 | E | S | T | — | 6.02E+04 | 5.46E−04 | 9.06E−09 |
| 3-F03_403 | — | S | T | A | 5.79E+04 | 5.41E−04 | 9.34E−09 |
| 3-F03_415 | — | S | T | — | 5.99E+04 | 6.06E−04 | 1.01E−08 |
| 3-F03_391 | — | S | T | D | 5.69E+04 | 5.84E−04 | 1.03E−08 |
| 3-F03_406 | — | S | A | A | 7.41E+04 | 7.65E−04 | 1.03E−08 |
| 3-F03_412 | E | S | A | — | 5.27E+04 | 6.38E−04 | 1.21E−08 |
| 3-F03_394 | — | S | A | D | 4.84E+04 | 6.30E−04 | 1.30E−08 |
| 3-F03_418 | — | S | A | — | 5.55E+04 | 7.99E−04 | 1.44E−08 |

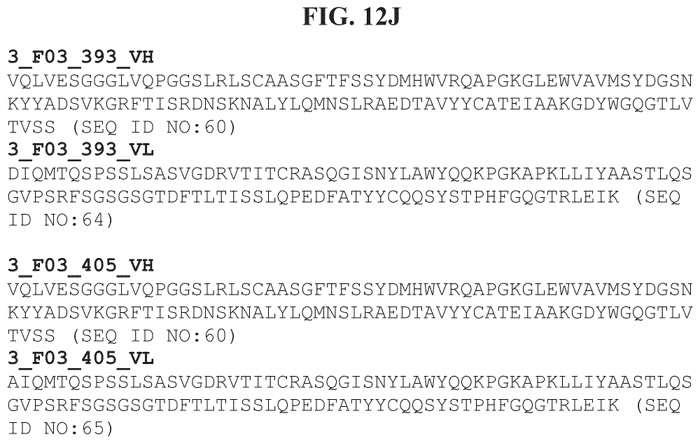

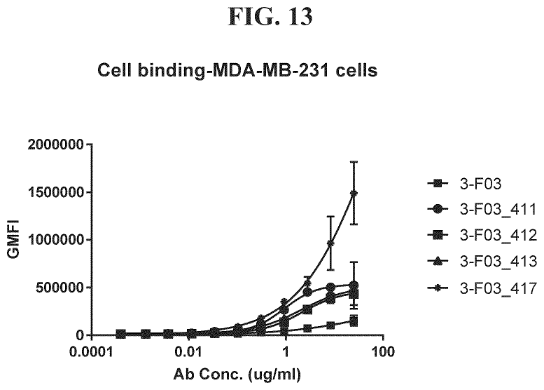

To test the binding of engineered 3-F03 variants to cell surface CD73, MDA-MB-231 cells were washed and added to 96-well plates at 5×10⁴ cells/well. Cells were stained with the indicated concentration of antibodies for 1 hour on ice. Cells were then washed and stained goat anti-mouse secondary conjugated to PE for 30 minute on ice. Cells were then washed and analyzed by flow cytometry. The GMFI of CD73 staining is graphed (FIG. 13). Each of the 3-F03 variants had a similar binding profile, except for 3-F03_417, which showed a slightly higher Ymax (FIG. 13). These data confirm the Biacore studies (Table 10): these mutations did not dramatically alter human CD73 binding for these variant clones.

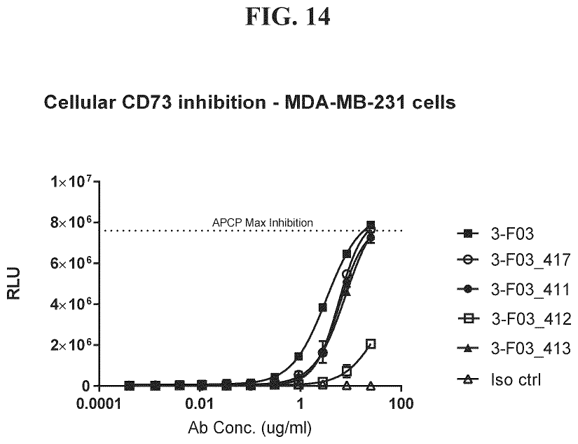

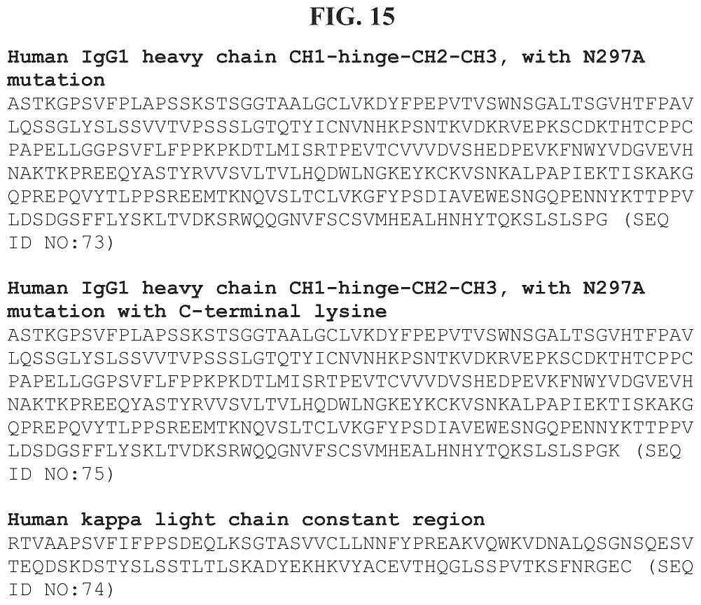

To test the ability of 3-F03 variants to inhibit CD73 activity on cells, MDA-MB-231 cells were washed with serum free RPMI media and plated 1×10⁴ cells/well in 96-well plates. Cells were incubated with the indicated concentration of antibodies or APCP at 37° C. 5% CO₂ for 30 minutes. Next, AMP was added to a final concentration of 100 μM and cells were incubated an additional 3 hours at 37° C. 5% CO₂. Plates were centrifuged for 1-2 minutes at 300 g and 25 μL of supernatant was transferred into a new 96-well plates. AMP-Glo Assay was used according to the manufacturer's instructions. RLU is a directly correlated with the AMP concentration in this assay. 3-F03 showed maximum inhibition among the 3-F03 variants (FIG. 14). Variants 3-F03_417, 3-F03_411 and 3-F03_413 displayed slightly lower potency compared to 3-F03 (FIG. 14). Variant 3-F03_412 did not inhibit membrane bound CD73 on MDA-MB-231 cells (FIG. 14).

### Example 16: Anti-CD73 Antibody Synergizes with A2A Inhibitor

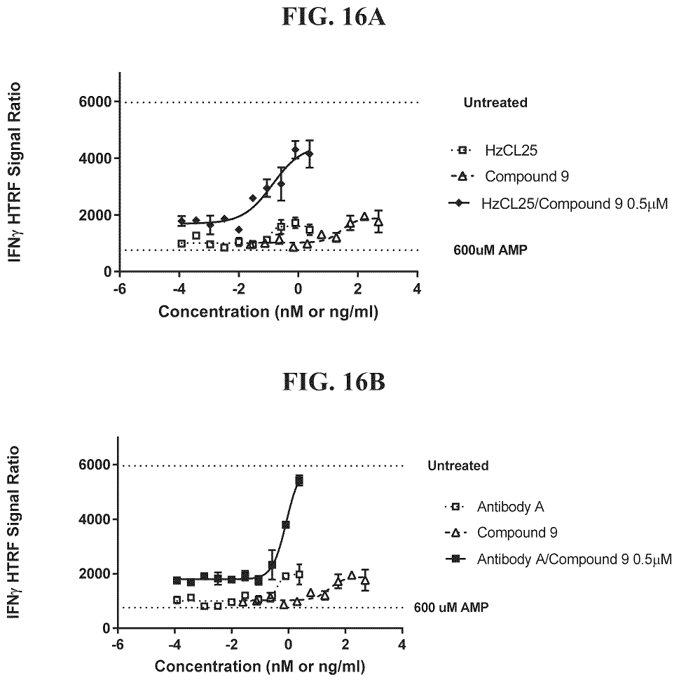

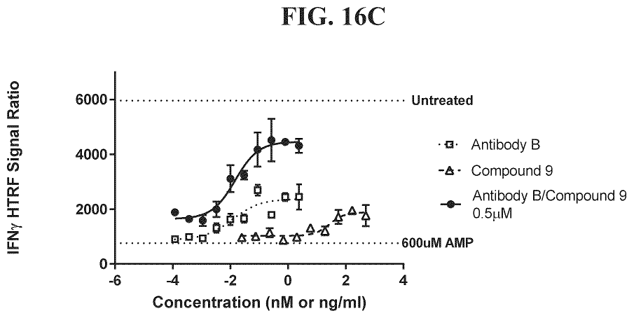

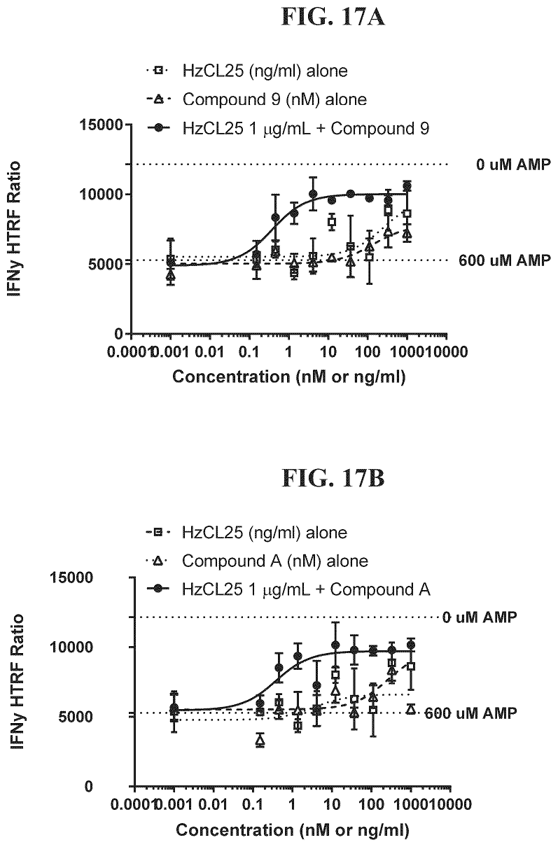

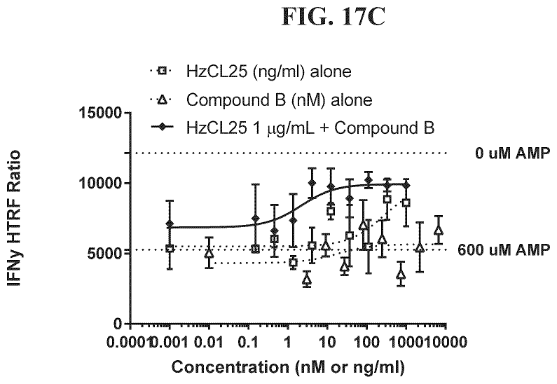

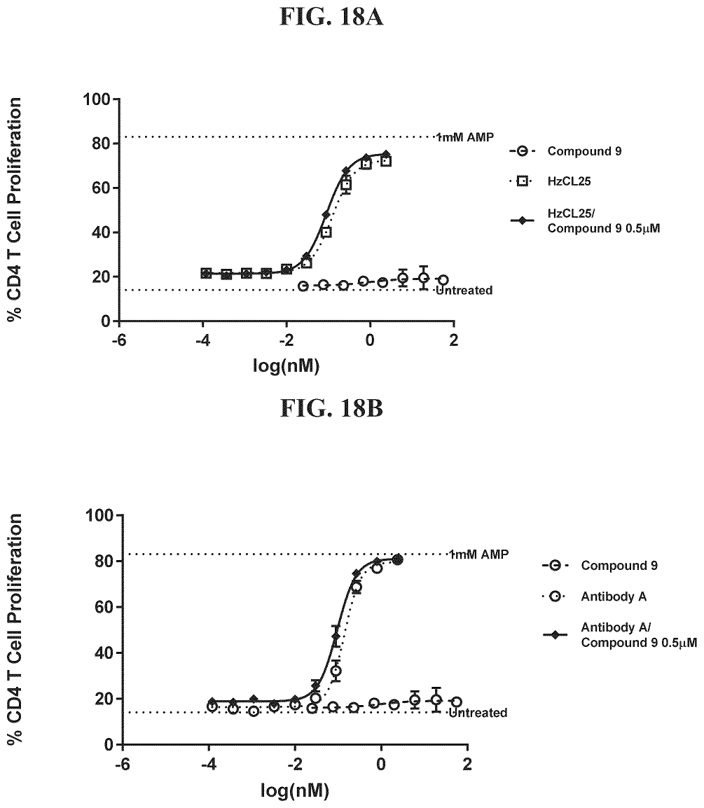

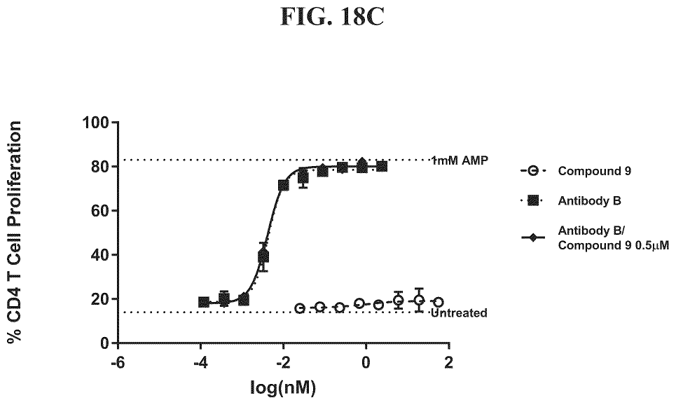

To measure the ability of anti-CD73 antibody and an inhibitor of A2A to reverse AMP-mediated suppression of T cell proliferation, primary human CD4⁺ T cells were purified from peripheral blood mononuclear cells (PBMCs) using a human CD4⁺ T-cell isolation kit (Miltenyi Biotec). Isolated CD4⁺ T cells were labeled with 1 μM of carboxyfluorescein succinimidyl ester (CFSE) (BD Biosciences) according to the manufacturer's protocol. CFSE labeled cells were resuspended in RPMI containing 10% fetal bovine serum. Approximately 50,000 cells/well were added in round bottom 96-well plate. Dynabeads human T activator CD3/CD28 beads were added to cell suspension at bead:cell ratio 1:1 and incubated for 1 hour at 37° C. Serial dilutions of anti-CD73 antibody (HzCL25, Antibody A, or Antibody B) in the presence or absence of 0.5 μM of an A2A inhibitor (Compound 9) were added into the designated wells and incubated for 30 minutes at 37° C.; serial dilutions of the A2A inhibitor were added into the designated wells as controls (FIG. 16A-FIG. 16C and FIG. 18A-FIG. 18C). Alternatively, serial dilutions of A2A inhibitor (Compound 9, Compound A, or Compound B) in the presence or absence of 1 μg/mL anti-CD73 antibody (HzCL25) were added into the designated wells and incubated for 30 minutes at 37° C.; serial dilutions of the CD73 antibody were added to the indicated wells as controls (FIG. 17A-FIG. 17C). Finally AMP was added at a final concentration of 600 μM and the whole culture was incubated for 4 days at 37° C. in the incubator. After 4 days, IF [symbol] production was measured (FIG. 16A-FIG. 16C and FIG. 17A-FIG. 17C) and CD4⁺ T cell proliferation was determined by CFSE based flow cytometry analysis using LSRFORTESSA X-20 analyzer (BD Biosciences) (FIG. 18A-FIG. 18C).

Antibody A Heavy Chain (VH italicized):
(SEQ ID NO: 102)
EIQLQQSGPELVKPGASVKVSCKASGYAFTSYNMYWVKQSHGKSLEWIGY
IDPYNGGTSYNQKFKGKATLTVDKSSSTAYMHLNSLTSEDSAVYYCARGY
GNYKAWFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
Antibody A Light Chain (VL italicized):
(SEQ ID NO: 103)
DAVMTQTPKFLLVSAGDRVTITCKASQSVTNDVAWYQQKPGQSPKLLIYY
ASNRYTGVPDRFTGSGYGTDFTFTISTVQAEDLAVYFCQQDYSSLTFGAG
TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Antibody B Heavy Chain (VH italicized):
(SEQ ID NO: 98)
EVQLLESGGGLVQPGGSLRLSCAASGFTFSSYAYSWVRQAPGKGLEWVSA
ISGSGGRTYYADSVKGRFTISRDNSKNTLYLQMNSLRAEDTAVYYCARLG
YGRVDEWGRGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
Antibody B Light Chain (VL italicized):
(SEQ ID NO: 99)
QSVLTQPPSASGTPGQRVTISCSGSLSNIGRNPVNWYQQLPGTAPKLLIY
LDNLRLSGVPDRFSGSKSGTSASLAISGLQSEDEADYYCATWDDSHPGWT
FGGGTKLTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS

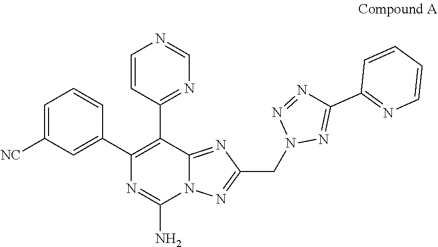 [SMILES: N#Cc1cccc(-c2nc(N)n3nc(Cn4nnc(-c5ccccn5)n4)nc3c2-c2ccncn2)c1]

### (3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile)

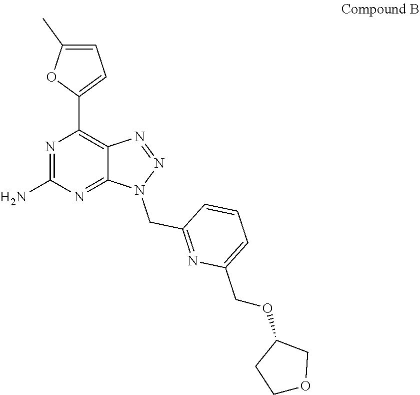 [SMILES: Cc1ccc(-c2nc(N)nc3c2nnn3Cc2cccc(CO[C@H]3CCOC3)n2)o1]

### (7-(5-methylfuran-2-yl)-3-[[6-[[(3S)-oxolan-3-yl]oxymethyl]pyridin-2-yl]methyl]triazolo[4,5-d]pyrimidin-5-amine)

When combined with anti-CD73 antibody, A2A inhibitor increased IF [symbol] production significantly (FIG. 16A-FIG. 16C and FIG. 17A-FIG. 17C).

When combined with anti-CD73 antibody, A2A/A2B inhibitor reversed AMP-mediated suppression of CD4⁺ T cell proliferation in a concentration dependent manner in multiple different human donors (FIG. 18A-FIG. 18C).

### Example 17: In Vivo Combination of Anti-CD73 Antibody and A2A Inhibitor Reduces Tumor Volume

The in vivo efficacy of anti-CD73 antibody in combination with an A2A inhibitor was tested. Anti-CD73 antibody HzCL25 or 3-F03_413 was suspended in 1× phosphate buffered saline (PBS) (Life Technologies) for intraperitoneal dosing of hu-CD34 NSG mice (Jackson laboratories). 1×PBS and Fc disabled human IgG1 suspended in 1×PBS was included in this study as a control. Mice with humanized immune systems were purchased from Jackson Labs (Bar Harbor, Maine). Briefly, 3 week old female NSG/NOD SCID mice received a single dose of irradiation toxic to immune cell precursors and were then “rescued” by injection of human cord blood, CD34⁺ selected cells. The mice comprised recipients of three distinct human immune donors to better represent individual variations in immune response.

The left flank of the mice were shaved the day prior to inoculation with 5×10⁶ cells of the human breast cancer line MDA-MB-231 (ATCC, Manassas Virginia) suspended in matrigel (Corning Life Sciences, Tewksbury, Mass). On day 7, tumor dimensions were measured by Vernier calipers, and volume estimated by the formula Volume=[L (long dimension)×W2 (short dimension)]/2. Mice were randomized into 4 groups of 5 or 6 mice of approximate mean volume (˜200 mm³) and donor representation. Tumors were measured every 5 days for the duration of the study.

Every five days, from day 8, mice were dosed intraperitoneally with (i) 10 mg/kg of human IgG1 and 10 mg/kg of vehicle; (ii) 10 mg/kg of HzCL25; or (iii) 10 mg/kg of 3-F03_413. For treatment groups (ii) and (iii), mice were dosed twice a day (BID) with Compound 9. A total of 8 doses of antibody were given (i.e., antibody was dosed on days 8 13, 18, 23, 29, 34, 39, and 44 and Compound 9 was dosed twice a day, with 8 and 16 hour intervals) starting on day 8 through the last day).

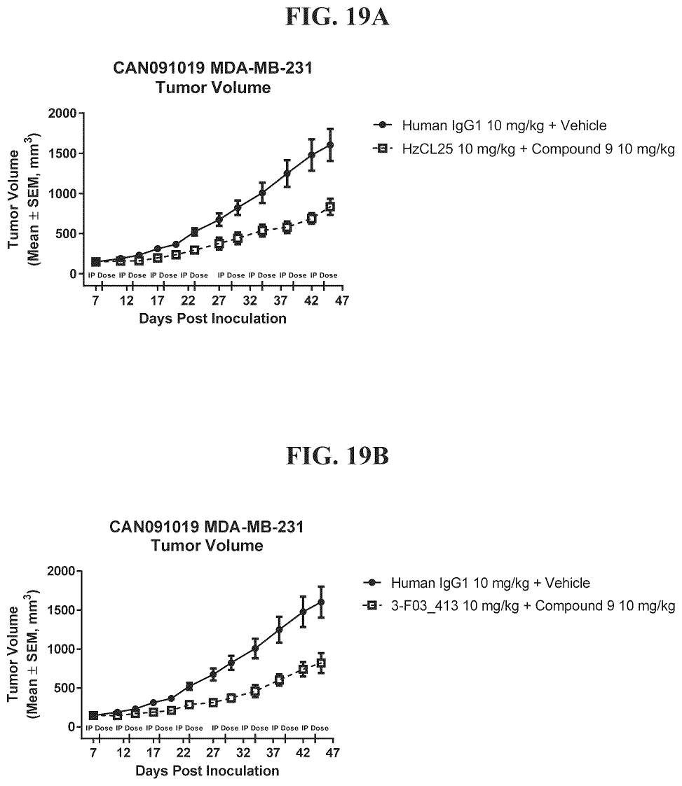

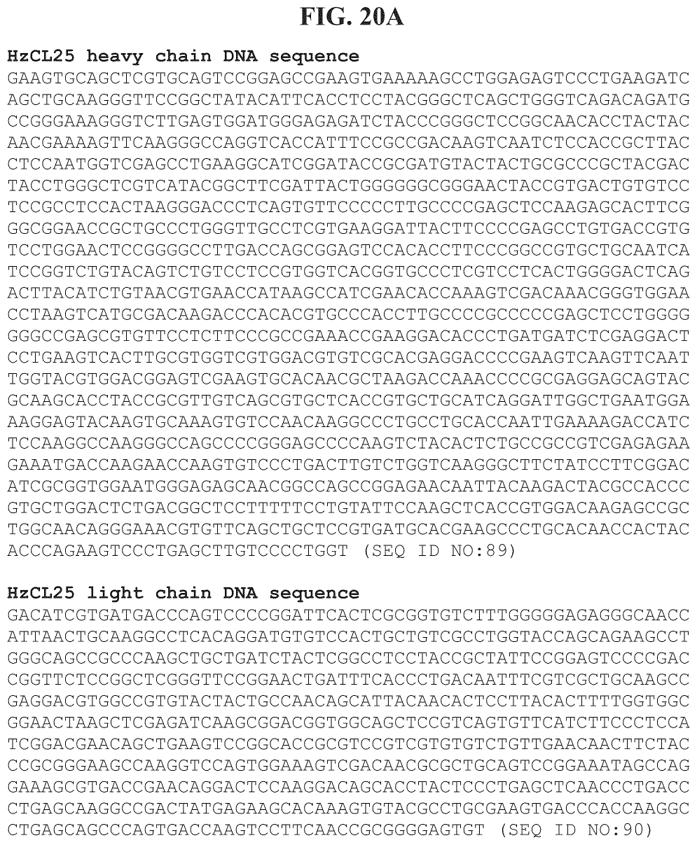

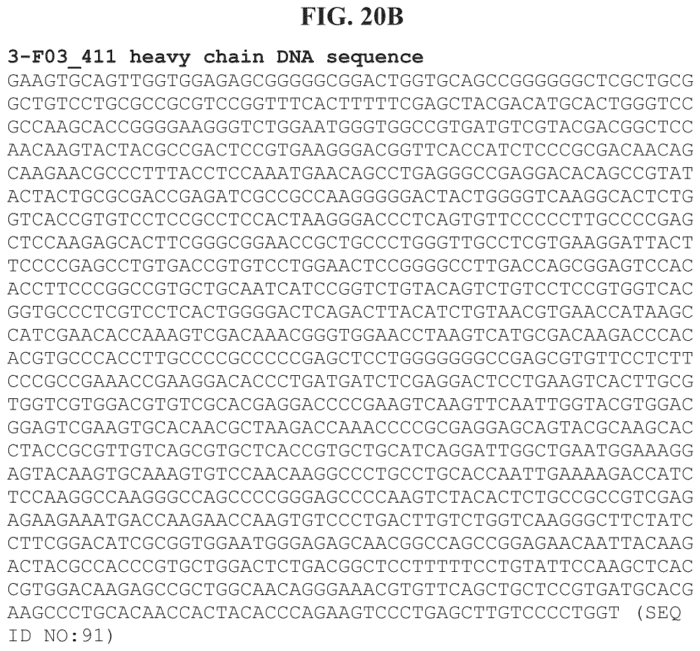

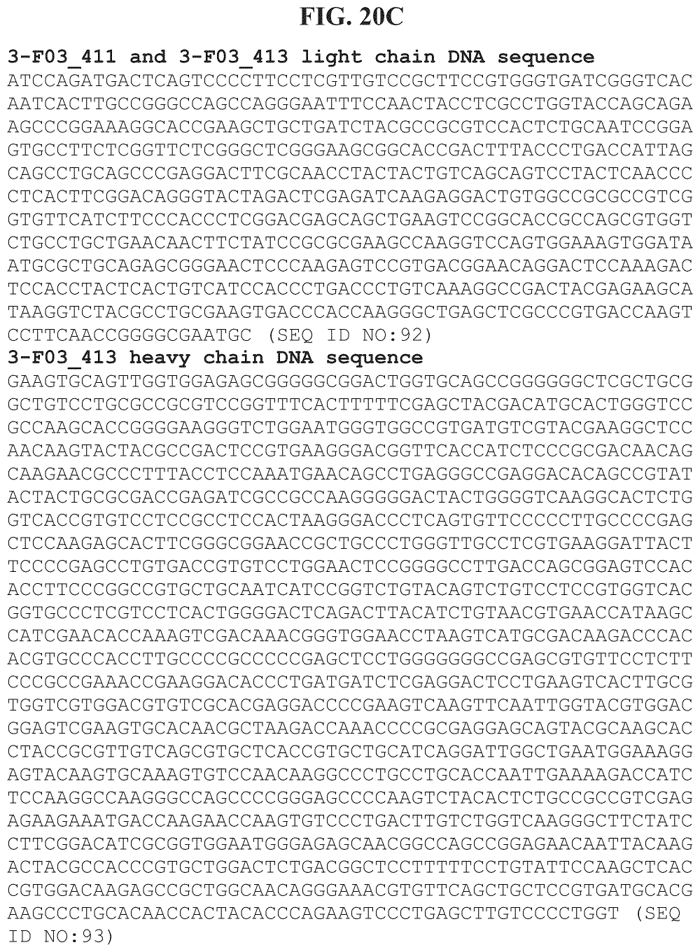

Mice administered anti-CD73 antibody in combination with an A2A inhibitor exhibited statistically significantly slowed tumor growth (FIG. 19A and FIG. 19B).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a cancer in a human subject in need thereof, comprising administering to the human subject an effective amount of an inhibitor of human CD73 and an inhibitor of A2A adenosine receptor and/or A2B adenosine receptor, wherein:
(1) the inhibitor of human CD73 comprises:
(a) an antibody that binds to human CD73 at an epitope within amino acids 40-52 of SEQ ID NO: 70;
(b) an antibody that binds to human CD73 and competes for binding to human CD73 with an antibody that has a heavy chain comprising the amino acid sequence of SEQ ID NO:24 and a light chain comprising the amino acid sequence of SEQ ID NO:25; or
(c) an antibody that binds to human CD73 at an epitope within amino acids 386-399 and 470-489 of SEQ ID NO:70;
(d) an antibody that binds to human CD73 and competes for binding to human CD73 with an antibody that has a heavy chain comprising the amino acid sequence of SEQ ID NO:30 and a light chain comprising the amino acid sequence of SEQ ID NO:31;
(e) an antibody that binds to human CD73 and competes for binding to human CD73 with an antibody that has a heavy chain comprising the amino acid sequence of SEQ ID NO:33 and a light chain comprising the amino acid sequence of SEQ ID NO:31;
(f) an antibody selected from the group consisting of 11E1, Medi9447, CPI-006, and BMS-986179; or
(g) an inhibitor selected from the group consisting of CB-708 and AB680; and
(2) the inhibitor of A2A adenosine receptor and/or A2B adenosine receptor (A2A/A2B) comprises a compound of:
(a) Formula (I): 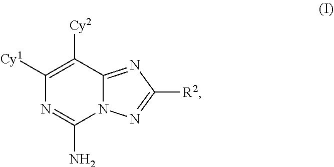
or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl;
(b) Formula (II): 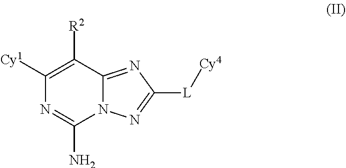
or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl;
(c) Formula (III): 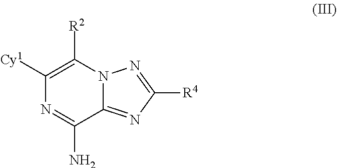
or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl; or
(d) Formula (IV): 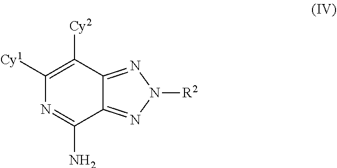
or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl,
or a pharmaceutically acceptable salt thereof.

2. The method of claim 1, wherein the inhibitor of A2A/A2B is a compound of Formula (I): 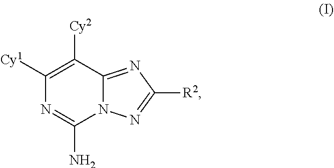
or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl- of R² are each optionally substituted with 1, 2, or 3 independently selected R^{C} substituents;
R^{a2} is (5-7 membered heteroaryl)-C₁₋₃ alkyl- optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl.

3. The method of claim 2, wherein the inhibitor of A2A/A2B is selected from:
3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof;
3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof;
3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof;
3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof; and
3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile, or a pharmaceutically acceptable salt thereof.

4. The method of claim 1, wherein the inhibitor of A2A/A2B is a compound of Formula (II): 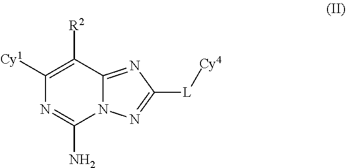
or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl.

5. The method of claim 4, wherein the inhibitor of A2A/A2B is selected from:
3-(5-Amino-2-(hydroxy(phenyl)methyl)-[1,2,4]triazolo[1,5-c]pyrimidin-7-yl)benzonitrile, or a pharmaceutically acceptable salt thereof;
3-(5-Amino-2-((2,6-difluorophenyl)(hydroxy)methyl)-[1,2,4]triazolo[1,5-c]pyrimidin-7-yl)-2-fluorobenzonitrile, or a pharmaceutically acceptable salt thereof;
5-Amino-7-(3-cyano-2-fluorophenyl)-2-((2,6-difluorophenyl)(hydroxy)methyl)-[1,2,4]triazolo[1,5-c]pyrimidine-8-carbonitrile, or a pharmaceutically acceptable salt thereof; and
3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile, or a pharmaceutically acceptable salt thereof.

6. The method of claim 1, wherein the inhibitor of A2A/A2B is a compound of Formula (III): 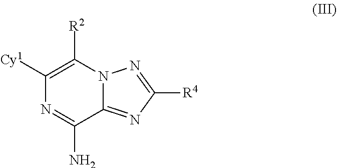
or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl- of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41}, and R^{d41} is independently selected from H and C₁₋₆ alkyl.

7. The method of claim 6, wherein the inhibitor of A2A/A2B is selected from:
3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile;
3-(8-Amino-2-((2,6-difluorophenyl)(hydroxy)methyl)-5-(pyrimidin-4-yl)-[1,2,4]triazolo[1,5-a]pyrazin-6-yl)benzonitrile, or a pharmaceutically acceptable salt thereof;
3-(8-amino-2-(amino(2,6-difluorophenyl)methyl)-5-(4-methyloxazol-5-yl)-[1,2,4]triazolo[1,5-a]pyrazin-6-yl)benzonitrile, or a pharmaceutically acceptable salt thereof; and
3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile, or a pharmaceutically acceptable salt thereof.

8. The method of claim 1, wherein the inhibitor of A2A/A2B is a compound of Formula (IV): 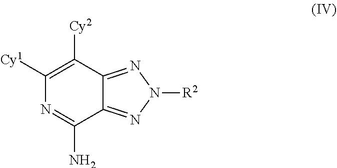
or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl- of R² are each optionally substituted with 1, 2, or 3 independently selected R^{2A} substituents; and
each R^{2A} is independently selected from halo, C₁₋₆ alkyl, and C₁₋₆ haloalkyl,
or a pharmaceutically acceptable salt thereof.

9. The method of claim 8, wherein the inhibitor of A2A/A2B is selected from:
3-(4-amino-2-(pyridin-2-ylmethyl)-7-(pyrimidin-4-yl)-2H-[1,2,3]triazolo[4,5-c]pyridin-6-yl)benzonitrile, or a pharmaceutically acceptable salt thereof;
3-(4-amino-2-((3-fluoropyridin-2-yl)methyl)-7-(pyrimidin-4-yl)-2H-[1,2,3]triazolo[4,5-c]pyridin-6-yl)benzonitrile, or a pharmaceutically acceptable salt thereof;
3-(4-amino-2-((3-fluoropyridin-2-yl)methyl)-7-(pyridin-4-yl)-2H-[1,2,3]triazolo[4,5-c]pyridin-6-yl)benzonitrile, or a pharmaceutically acceptable salt thereof; and
3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile, or a pharmaceutically acceptable salt thereof.

10. The method of claim 1, wherein the inhibitor of A2A/A2B is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof.

11. The method of claim 1, wherein the inhibitor of A2A/A2B is 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof.

12. (canceled)

13. The method of claim 1, wherein the inhibitor of human CD73 comprises an antibody that binds to human CD73 at an epitope within amino acids 40-53 of SEQ ID NO:70.

14. The method of claim 1, wherein the inhibitor of human CD73 comprises an antibody that binds to human CD73 and competes for binding to human CD73 with an antibody that has a heavy chain comprising the amino acid sequence of SEQ ID NO:24 and a light chain comprising the amino acid sequence of SEQ ID NO:25.

15. (canceled)

16. The method of claim 1, wherein the inhibitor of human CD73 comprises an antibody that binds to human CD73 at an epitope within amino acids 386-399 and 470-489 of SEQ ID NO:70.

17. The method of claim 1, wherein the inhibitor of human CD73 comprises an antibody that binds to human CD73 and competes for binding to human CD73 with an antibody that has a heavy chain comprising the amino acid sequence of SEQ ID NO:30 and a light chain comprising the amino acid sequence of SEQ ID NO:31.

18. The method of claim 1, wherein the inhibitor of human CD73 comprises an antibody that binds to human CD73 and competes for binding to human CD73 with an antibody that has a heavy chain comprising the amino acid sequence of SEQ ID NO:33 and a light chain comprising the amino acid sequence of SEQ ID NO:31.

19. The method of claim 1, wherein the inhibitor of human CD73 comprises an antibody selected from the group consisting of 11E1, Medi9447, CPI-006, and BMS-986179.

20. The method of claim 1, wherein the inhibitor of human CD73 is selected from the group consisting of CB-708 and AB680.

21.-26. (canceled)

27. A method for treating a cancer in a human subject in need thereof, comprising administering to the human subject an effective amount of an antibody that binds to human CD73 and an inhibitor of A2A adenosine receptor and/or A2B adenosine receptor, wherein the antibody:
(a) binds to human CD73 at an epitope within amino acids 40-53 of SEQ ID NO:70;
(b) binds to human CD73 and competes for binding to human CD73 with an antibody that has a heavy chain comprising the amino acid sequence of SEQ ID NO:24 and a light chain comprising the amino acid sequence of SEQ ID NO:25;
(c) binds to human CD73 at an epitope within amino acids 386-399 and 470-489 of SEQ ID NO: 70;
(d) binds to human CD73 and competes for binding to human CD73 with an antibody that has a heavy chain comprising the amino acid sequence of SEQ ID NO:30 and a light chain comprising the amino acid sequence of SEQ ID NO:31; or
(e) binds to human CD73 and competes for binding to human CD73 with an antibody that has a heavy chain comprising the amino acid sequence of SEQ ID NO:33 and a light chain comprising the amino acid sequence of SEQ ID NO:31.

28.-57. (canceled)

58. The method of claim 27, wherein the inhibitor is selected from the group consisting of:
7-(5-methylfuran-2-yl)-3-[[6-[[(3S)-oxolan-3-yl]oxymethyl]pyridin-2-yl]methyl]triazolo[4,5-d]pyrimidin-5-amine,
3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile,
3-[2-Amino-6-[1-[[6-(2-hydroxypropan-2-yl)pyridin-2-yl]methyl]triazol-4-yl]pyrimidin-4-yl]-2-methylbenzonitrile,
6-(2-Chloro-6-methylpyridin-4-yl)-5-(4-fluorophenyl)-1,2,4-triazin-3-amine, 5-Bromo-2,6-di(1H-pyrazol-1-yl)pyrimidin-4-amine, and
EOS100850.

59. The method of claim 1, wherein the cancer has a high adenosine signature.

60. The method of claim 1, wherein the cancer is head and neck cancer, colorectal cancer, lung cancer, melanoma, ovarian, bladder, liver cancer, or renal cell carcinoma.

61. The method of claim 27, wherein the cancer has a high adenosine signature.

62. The method of claim 27, wherein the cancer is head and neck cancer, colorectal cancer, lung cancer, melanoma, ovarian, bladder, liver cancer, or renal cell carcinoma.


SEQUENCE LISTING (103 sequences)

SEQ ID NO: 1 - protein, 8 aa, synthetic construct
GYTFTSYG

SEQ ID NO: 2 - protein, 8 aa, synthetic construct
IYPGSGNT

SEQ ID NO: 3 - protein, 14 aa, synthetic construct
ARYDYLGSSYGFDY

SEQ ID NO: 4 - protein, 6 aa, synthetic construct
QDVSTA

SEQ ID NO: 5 - skipped

SEQ ID NO: 6 - protein, 9 aa, synthetic construct
QQHYNTPYT

SEQ ID NO: 7 - protein, 7 aa, synthetic construct
GYTFTSY

SEQ ID NO: 8 - protein, 6 aa, synthetic construct
YPGSGN

SEQ ID NO: 9 - protein, 12 aa, synthetic construct
YDYLGSSYGFDY

SEQ ID NO: 10 - protein, 11 aa, synthetic construct
KASQDVSTAVA

SEQ ID NO: 11 - protein, 7 aa, synthetic construct
SASYRYS

SEQ ID NO: 12 - protein, 10 aa, synthetic construct
GYTFTSYGLS

SEQ ID NO: 13 - protein, 10 aa, synthetic construct
EIYPGSGNTY

SEQ ID NO: 14 - protein, 5 aa, synthetic construct
SYGLS

SEQ ID NO: 15 - protein, 17 aa, synthetic construct
EIYPGSGNTYYNEKFKG

SEQ ID NO: 16 - protein, 6 aa, synthetic construct
TSYGLS

SEQ ID NO: 17 - protein, 13 aa, synthetic construct
WMGEIYPGSGNTY

SEQ ID NO: 18 - protein, 13 aa, synthetic construct
ARYDYLGSSYGFD

SEQ ID NO: 19 - protein, 7 aa, synthetic construct
STAVAWY

SEQ ID NO: 20 - protein, 10 aa, synthetic construct
LLIYSASYRY

SEQ ID NO: 21 - protein, 8 aa, synthetic construct
QQHYNTPY

SEQ ID NO: 22 - protein, 121 aa, synthetic construct
EVQLVQSGAEVKKPGESLKISCKGSGYTFTSYGLSWVRQMPGKGLEWMGEIYPGSGNTYYNEKFKGQVTISADKSISTAYLQWSSLKASDTAMYYCARYDYLGSSYGFDYWGAGTTVTVSS

SEQ ID NO: 23 - protein, 107 aa, synthetic construct
DIVMTQSPDSLAVSLGERATINCKASQDVSTAVAWYQQKPGQPPKLLIYSASYRYSGVPDRFSGSGSGTDFTLTISSLQAEDVAVYYCQQHYNTPYTFGGGTKLEIK

SEQ ID NO: 24 - protein, 450 aa, synthetic construct
EVQLVQSGAEVKKPGESLKISCKGSGYTFTSYGLSWVRQMPGKGLEWMGEIYPGSGNTYYNEKFKGQVTISADKSISTAYLQWSSLKASDTAMYYCARYDYLGSSYGFDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 214 aa, synthetic construct
DIVMTQSPDSLAVSLGERATINCKASQDVSTAVAWYQQKPGQPPKLLIYSASYRYSGVPDRFSGSGSGTDFTLTISSLQAEDVAVYYCQQHYNTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 121 aa, synthetic construct
QVQLQQSGAELARPGASVKLSCRASGYTFTSYGLSWVKQRTGQGLEWIGEIYPGSGNTYYNEKFKGKATLTADKSSSTAYMELRSLTSEDSAVYFCARYDYLGSSYGFDYWGQGTTLTVSS

SEQ ID NO: 27 - protein, 107 aa, synthetic construct
DIVMTQSHKFMSTSVGDRVSITCKASQDVSTAVAWYQQKPGQSPKLLIYSASYRYNGVPDRFTGSGSGTDFTFTISSVQAEDLAVYYCQQHYNTPYTFGGGTKLEIK

SEQ ID NO: 28 - protein, 13 aa, synthetic construct
WIGEIYPGSGNTY

SEQ ID NO: 29 - protein, 7 aa, synthetic construct
SASYRYN

SEQ ID NO: 30 - protein, 446 aa, synthetic construct
EVQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 212 aa, synthetic construct
IQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 116 aa, synthetic construct
VQLVESGGGLVQPGGSLRLSCAASGFTFSSYDMHWVRQAPGKGLEWVAVMSYSGSNKYYADSVKGRFTISRDNSKNTLYLQMNSLRAEDTAVYYCATEIAAKGDYWGQGTLVTVSS

SEQ ID NO: 33 - protein, 446 aa, synthetic construct
EVQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 8 aa, synthetic construct
GFTFSSYD

SEQ ID NO: 35 - protein, 8 aa, synthetic construct
MSYDGSNK

SEQ ID NO: 36 - protein, 10 aa, synthetic construct
ATEIAAKGDY

SEQ ID NO: 37 - protein, 6 aa, synthetic construct
QGISNY

SEQ ID NO: 38 - skipped

SEQ ID NO: 39 - protein, 8 aa, synthetic construct
QQSYSTPH

SEQ ID NO: 40 - protein, 8 aa, synthetic construct
MSYEGSNK

SEQ ID NO: 41 - protein, 7 aa, synthetic construct
GFTFSSY

SEQ ID NO: 42 - protein, 6 aa, synthetic construct
SYDGSN

SEQ ID NO: 43 - protein, 6 aa, synthetic construct
SYEGSN

SEQ ID NO: 44 - protein, 11 aa, synthetic construct
RASQGISNYLA

SEQ ID NO: 45 - protein, 7 aa, synthetic construct
AASTLQS

SEQ ID NO: 46 - protein, 10 aa, synthetic construct
GFTFSSYDMH

SEQ ID NO: 47 - protein, 10 aa, synthetic construct
VMSYDGSNKY

SEQ ID NO: 48 - protein, 10 aa, synthetic construct
VMSYEGSNKY

SEQ ID NO: 49 - protein, 5 aa, synthetic construct
SYDMH

SEQ ID NO: 50 - protein, 17 aa, synthetic construct
VMSYDGSNKYYADSVKG

SEQ ID NO: 51 - protein, 17 aa, synthetic construct
VMSYEGSNKYYADSVKG

SEQ ID NO: 52 - protein, 8 aa, synthetic construct
EIAAKGDY

SEQ ID NO: 53 - protein, 6 aa, synthetic construct
SSYDMH

SEQ ID NO: 54 - protein, 13 aa, synthetic construct
WVAVMSYDGSNKY

SEQ ID NO: 55 - protein, 13 aa, synthetic construct
WVAVMSYEGSNKY

SEQ ID NO: 56 - protein, 9 aa, synthetic construct
ATEIAAKGD

SEQ ID NO: 57 - protein, 7 aa, synthetic construct
SNYLAWY

SEQ ID NO: 58 - protein, 10 aa, synthetic construct
LLIYAASTLQ

SEQ ID NO: 59 - protein, 7 aa, synthetic construct
QQSYSTP

SEQ ID NO: 60 - protein, 116 aa, synthetic construct
VQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSS

SEQ ID NO: 61 - protein, 105 aa, synthetic construct
IQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 62 - protein, 117 aa, synthetic construct
EVQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSS

SEQ ID NO: 63 - protein, 117 aa, synthetic construct
EVQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSS

SEQ ID NO: 64 - protein, 106 aa, synthetic construct
DIQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 65 - protein, 106 aa, synthetic construct
AIQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 66 - protein, 446 aa, synthetic construct
VQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 116 aa, synthetic construct
VQLVESGGGLVQPGGSLRLSCAASGFTFSSYDMHWVRQAPGKGLEWVAVMSYSGSNKYYADSVKGRFTISRDNSKNALYLQMNSLRAEDTAVYYCATEIAAKGDYWGQGTLVTVSS

SEQ ID NO: 68 - protein, 117 aa, synthetic construct
EVQLVESGGGLVQPGGSLRLSCAASGFTFSSYDMHWVRQAPGKGLEWVAVMSYSGSNKYYADSVKGRFTISRDNSKNTLYLQMNSLRAEDTAVYYCATEIAAKGDYWGQGTLVTVSS

SEQ ID NO: 69 - protein, 117 aa, synthetic construct
EVQLVESGGGLVQPGGSLRLSCAASGFTFSSYDMHWVRQAPGKGLEWVAVMSYSGSNKYYADSVKGRFTISRDNSKNALYLQMNSLRAEDTAVYYCATEIAAKGDYWGQGTLVTVSS

SEQ ID NO: 70 - protein, 523 aa, Homo sapiens
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFS

SEQ ID NO: 71 - protein, 523 aa, Mus musculus
WELTILHTNDVHSRLEQTSDDSTKCLNASLCVGGVARLFTKVQQIRKEEPNVLFLDAGDQYQGTIWFTVYKGLEVAHFMNILGYDAMALGNHEFDNGVEGLIDPLLRNVKFPILSANIKARGPLAHQISGLFLPSKVLSVGGEVVGIVGYTSKETPFLSNPGTNLVFEDEISALQPEVDKLKTLNVNKIIALGHSGFEMDKLIAQKVRGVDIVVGGHSNTFLYTGNPPSKEVPAGKYPFIVTADDGRQVPVVQAYAFGKYLGYLKVEFDDKGNVITSYGNPILLNSSIPEDATIKADINQWRIKLDNYSTQELGRTIVYLDGSTQTCRFRECNMGNLICDAMINNNLRHPDEMFWNHVSMCIVNGGGIRSPIDEKNNGTITWENLAAVLPFGGTFDLVQLKGSTLKKAFEHSVHRYGQSTGEFLQVGGIHVVYDINRKPWNRVVQLEVLCTKCRVPIYEPLEMDKVYKVTLPSYLANGGDGFQMIKDELLKHDSGDQDISVVSEYISKMKVVYPAVEGRIKFS

SEQ ID NO: 72 - protein, 523 aa, Macaca fascicularis
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFETDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEIYKVILPNFLANGGDGFQMIKDELLRHDSGDQDINVVSTYISKMKVIYPAVEGRIKFS

SEQ ID NO: 73 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 107 aa, Homo sapiens [sequence omitted]

SEQ ID NO: 75 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 14 aa, Homo sapiens
TKVQQIRRAEPNVL

SEQ ID NO: 77 - protein, 117 aa, synthetic construct
EVQLVESGGGLVQPGGSLRLSCAASGFTFSSYDMHWVRQAPGKGLEWVAVMSYDGSNKYYADSVKGRFTISRDNSKNTLYLQMNSLRAEDTAVYYCATEIAAKGDYWGQGTLVTVSS

SEQ ID NO: 78 - protein, 14 aa, Homo sapiens
AAVLPFGGTFDLVQ

SEQ ID NO: 79 - protein, 20 aa, Homo sapiens
ILPNFLANGGDGFQMIKDEL

SEQ ID NO: 80 - protein, 107 aa, synthetic construct
DIQMTQSPSSLSASVGDRVTITCKASQDVSTAVAWYQQKPGKAPKLLLYSASYRYSGVPSRFSGSGSGTDYTLTISSLQPEDFATYYCQQHYNTPYTFGGGTKLEIK

SEQ ID NO: 81 - protein, 107 aa, synthetic construct
AIRMTQSPSSFSASTGDRVTITCKASQDVSTAVAWYQQKPGKAPKLLIYSASYRYSGVPSRFSGSGSGTDFTLTISCLQSEDFATYYCQQHYNTPYTFGGGTKLEIK

SEQ ID NO: 82 - protein, 121 aa, synthetic construct
QVQLVQSGAEVKKPGASVKVSCKASGYTFTSYGLSWVRQATGQGLEWMGEIYPGSGNTYYNEKFKGRVTMTANTSISTAYMELSSLRSEDTAVYYCARYDYLGSSYGFDYWGAGTTVTVSS

SEQ ID NO: 83 - protein, 121 aa, synthetic construct
QVQLVQSGAEVKKPGASVKVSCKASGYTFTSYGLSWVRQATGQGLEWMGEIYPGSGNTYYNEKFKGRVTMTRNTSISTAYMELSSLRSEDTAVYYCARYDYLGSSYGFDYWGAGTTVTVSS

SEQ ID NO: 84 - protein, 121 aa, synthetic construct
QVQLVQSGAEVKKPGSSVKVSCKASGYTFTSYGLSWVRQAPGQGLEWMGEIYPGSGNTYYNEKFKGRVTITADESTSTAYMELSSLRSEDTAVYYCARYDYLGSSYGFDYWGAGTTVTVSS

SEQ ID NO: 85 - protein, 116 aa, synthetic construct
VQLVESGGGLVQPGGSLRLSCAASGFTFSSYDMHWVRQAPGKGLEWVAVMSYDGSNKYYADSVKGRFTISRDNSKNTLYLQMNSLRAEDTAVYYCATEIAAKGDYWGQGTLVTVSS

SEQ ID NO: 86 - protein, 117 aa, synthetic construct
EVQLVESGGGLVQPGGSLRLSCAASGFTFSSYDMHWVRQAPGKGLEWVAVMSYEGSNKYYADSVKGRFTISRDNSKNTLYLQMNSLRAEDTAVYYCATEIAAKGDYWGQGTLVTVSS

SEQ ID NO: 87 - protein, 116 aa, synthetic construct
VQLVESGGGLVQPGGSLRLSCAASGFTFSSYDMHWVRQAPGKGLEWVAVMSYEGSNKYYADSVKGRFTISRDNSKNTLYLQMNSLRAEDTAVYYCATEIAAKGDYWGQGTLVTVSS

SEQ ID NO: 88 - protein, 116 aa, synthetic construct
VQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSS

SEQ ID NO: 89 - DNA, 1350 bp, synthetic construct
  source 1..1350: mol_type = other DNA
gaagtgcagctcgtgcagtccggagccgaagtgaaaaagcctggagagtccctgaagatcagctgcaagggttccggctatacattcacctcctacgggctcagctgggtcagacagatgccgggaaagggtcttgagtggatgggagagatctacccgggctccggcaacacctactacaacgaaaagttcaagggccaggtcaccatttccgccgacaagtcaatctccaccgcttacctccaatggtcgagcctgaaggcatcggataccgcgatgtactactgcgcccgctacgactacctgggctcgtcatacggcttcgattactggggggcgggaactaccgtgactgtgtcctccgcctccactaagggaccctcagtgttcccccttgccccgagctccaagagcacttcgggcggaaccgctgccctgggttgcctcgtgaaggattacttccccgagcctgtgaccgtgtcctggaactccggggccttgaccagcggagtccacaccttcccggccgtgctgcaatcatccggtctgtacagtctgtcctccgtggtcacggtgccctcgtcctcactggggactcagacttacatctgtaacgtgaaccataagccatcgaacaccaaagtcgacaaacgggtggaacctaagtcatgcgacaagacccacacgtgcccaccttgccccgcccccgagctcctgggggggccgagcgtgttcctcttcccgccgaaaccgaaggacaccctgatgatctcgaggactcctgaagtcacttgcgtggtcgtggacgtgtcgcacgaggaccccgaagtcaagttcaattggtacgtggacggagtcgaagtgcacaacgctaagaccaaaccccgcgaggagcagtacgcaagcacctaccgcgttgtcagcgtgctcaccgtgctgcatcaggattggctgaatggaaaggagtacaagtgcaaagtgtccaacaaggccctgcctgcaccaattgaaaagaccatctccaaggccaagggccagccccgggagccccaagtctacactctgccgccgtcgagagaagaaatgaccaagaaccaagtgtccctgacttgtctggtcaagggcttctatccttcggacatcgcggtggaatgggagagcaacggccagccggagaacaattacaagactacgccacccgtgctggactctgacggctcctttttcctgtattccaagctcaccgtggacaagagccgctggcaacagggaaacgtgttcagctgctccgtgatgcacgaagccctgcacaaccactacacccagaagtccctgagcttgtcccctggt

SEQ ID NO: 90 - DNA, 642 bp, synthetic construct
  source 1..642: mol_type = other DNA
gacatcgtgatgacccagtccccggattcactcgcggtgtctttgggggagagggcaaccattaactgcaaggcctcacaggatgtgtccactgctgtcgcctggtaccagcagaagcctgggcagccgcccaagctgctgatctactcggcctcctaccgctattccggagtccccgaccggttctccggctcgggttccggaactgatttcaccctgacaatttcgtcgctgcaagccgaggacgtggccgtgtactactgccaacagcattacaacactccttacacttttggtggcggaactaagctcgagatcaagcggacggtggcagctccgtcagtgttcatcttccctccatcggacgaacagctgaagtccggcaccgcgtccgtcgtgtgtctgttgaacaacttctacccgcgggaagccaaggtccagtggaaagtcgacaacgcgctgcagtccggaaatagccaggaaagcgtgaccgaacaggactccaaggacagcacctactccctgagctcaaccctgaccctgagcaaggccgactatgagaagcacaaagtgtacgcctgcgaagtgacccaccaaggcctgagcagcccagtgaccaagtccttcaaccgcggggagtgt

SEQ ID NO: 91 - DNA, 1338 bp, synthetic construct
  source 1..1338: mol_type = other DNA
gaagtgcagttggtggagagcgggggcggactggtgcagccggggggctcgctgcggctgtcctgcgccgcgtccggtttcactttttcgagctacgacatgcactgggtccgccaagcaccggggaagggtctggaatgggtggccgtgatgtcgtacgacggctccaacaagtactacgccgactccgtgaagggacggttcaccatctcccgcgacaacagcaagaacgccctttacctccaaatgaacagcctgagggccgaggacacagccgtatactactgcgcgaccgagatcgccgccaagggggactactggggtcaaggcactctggtcaccgtgtcctccgcctccactaagggaccctcagtgttcccccttgccccgagctccaagagcacttcgggcggaaccgctgccctgggttgcctcgtgaaggattacttccccgagcctgtgaccgtgtcctggaactccggggccttgaccagcggagtccacaccttcccggccgtgctgcaatcatccggtctgtacagtctgtcctccgtggtcacggtgccctcgtcctcactggggactcagacttacatctgtaacgtgaaccataagccatcgaacaccaaagtcgacaaacgggtggaacctaagtcatgcgacaagacccacacgtgcccaccttgccccgcccccgagctcctgggggggccgagcgtgttcctcttcccgccgaaaccgaaggacaccctgatgatctcgaggactcctgaagtcacttgcgtggtcgtggacgtgtcgcacgaggaccccgaagtcaagttcaattggtacgtggacggagtcgaagtgcacaacgctaagaccaaaccccgcgaggagcagtacgcaagcacctaccgcgttgtcagcgtgctcaccgtgctgcatcaggattggctgaatggaaaggagtacaagtgcaaagtgtccaacaaggccctgcctgcaccaattgaaaagaccatctccaaggccaagggccagccccgggagccccaagtctacactctgccgccgtcgagagaagaaatgaccaagaaccaagtgtccctgacttgtctggtcaagggcttctatccttcggacatcgcggtggaatgggagagcaacggccagccggagaacaattacaagactacgccacccgtgctggactctgacggctcctttttcctgtattccaagctcaccgtggacaagagccgctggcaacagggaaacgtgttcagctgctccgtgatgcacgaagccctgcacaaccactacacccagaagtccctgagcttgtcccctggt

SEQ ID NO: 92 - DNA, 636 bp, synthetic construct
  source 1..636: mol_type = other DNA
atccagatgactcagtccccttcctcgttgtccgcttccgtgggtgatcgggtcacaatcacttgccgggccagccagggaatttccaactacctcgcctggtaccagcagaagcccggaaaggcaccgaagctgctgatctacgccgcgtccactctgcaatccggagtgccttctcggttctcgggctcgggaagcggcaccgactttaccctgaccattagcagcctgcagcccgaggacttcgcaacctactactgtcagcagtcctactcaacccctcacttcggacagggtactagactcgagatcaagaggactgtggccgcgccgtcggtgttcatcttcccaccctcggacgagcagctgaagtccggcaccgccagcgtggtctgcctgctgaacaacttctatccgcgcgaagccaaggtccagtggaaagtggataatgcgctgcagagcgggaactcccaagagtccgtgacggaacaggactccaaagactccacctactcactgtcatccaccctgaccctgtcaaaggccgactacgagaagcataaggtctacgcctgcgaagtgacccaccaagggctgagctcgcccgtgaccaagtccttcaaccggggcgaatgc

SEQ ID NO: 93 - DNA, 1338 bp, synthetic construct
  source 1..1338: mol_type = other DNA
gaagtgcagttggtggagagcgggggcggactggtgcagccggggggctcgctgcggctgtcctgcgccgcgtccggtttcactttttcgagctacgacatgcactgggtccgccaagcaccggggaagggtctggaatgggtggccgtgatgtcgtacgaaggctccaacaagtactacgccgactccgtgaagggacggttcaccatctcccgcgacaacagcaagaacgccctttacctccaaatgaacagcctgagggccgaggacacagccgtatactactgcgcgaccgagatcgccgccaagggggactactggggtcaaggcactctggtcaccgtgtcctccgcctccactaagggaccctcagtgttcccccttgccccgagctccaagagcacttcgggcggaaccgctgccctgggttgcctcgtgaaggattacttccccgagcctgtgaccgtgtcctggaactccggggccttgaccagcggagtccacaccttcccggccgtgctgcaatcatccggtctgtacagtctgtcctccgtggtcacggtgccctcgtcctcactggggactcagacttacatctgtaacgtgaaccataagccatcgaacaccaaagtcgacaaacgggtggaacctaagtcatgcgacaagacccacacgtgcccaccttgccccgcccccgagctcctgggggggccgagcgtgttcctcttcccgccgaaaccgaaggacaccctgatgatctcgaggactcctgaagtcacttgcgtggtcgtggacgtgtcgcacgaggaccccgaagtcaagttcaattggtacgtggacggagtcgaagtgcacaacgctaagaccaaaccccgcgaggagcagtacgcaagcacctaccgcgttgtcagcgtgctcaccgtgctgcatcaggattggctgaatggaaaggagtacaagtgcaaagtgtccaacaaggccctgcctgcaccaattgaaaagaccatctccaaggccaagggccagccccgggagccccaagtctacactctgccgccgtcgagagaagaaatgaccaagaaccaagtgtccctgacttgtctggtcaagggcttctatccttcggacatcgcggtggaatgggagagcaacggccagccggagaacaattacaagactacgccacccgtgctggactctgacggctcctttttcctgtattccaagctcaccgtggacaagagccgctggcaacagggaaacgtgttcagctgctccgtgatgcacgaagccctgcacaaccactacacccagaagtccctgagcttgtcccctggt

SEQ ID NO: 94 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 95 - protein, 332 aa, Homo sapiens
MLLETQDALYVALELVIAALSVAGNVLVCAAVGTANTLQTPTNYFLVSLAAADVAVGLFAIPFAITISLGFCTDFYGCLFLACFVLVLTQSSIFSLLAVAVDRYLAICVPLRYKSLVTGTRARGVIAVLWVLAFGIGLTPFLGWNSKDSATNNCTEPWDGTTNESCCLVKCLFENVVPMSYMVYFNFFGCVLPPLLIMLVIYIKIFLVACRQLQRTELMDHSRTTLQREIHAAKSLAMIVGIFALCWLPVHAVNCVTLFQPAQGKNKPKWAMNMAILLSHANSVVNPIVYAYRNRDFRYTFHKIISRYLLCQADVKSGNGQAGVQPALGVGL

SEQ ID NO: 96 - protein, 117 aa, synthetic construct
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 97 - protein, 110 aa, synthetic construct
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 98 - protein, 446 aa, synthetic construct
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 216 aa, synthetic construct
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 100 - protein, 120 aa, synthetic construct
EIQLQQSGPELVKPGASVKVSCKASGYAFTSYNMYWVKQSHGKSLEWIGYIDPYNGGTSYNQKFKGKATLTVDKSSSTAYMHLNSLTSEDSAVYYCARGYGNYKAWFAYWGQGTLVTVSA

SEQ ID NO: 101 - protein, 106 aa, synthetic construct
DAVMTQTPKFLLVSAGDRVTITCKASQSVTNDVAWYQQKPGQSPKLLIYYASNRYTGVPDRFTGSGYGTDFTFTISTVQAEDLAVYFCQQDYSSLTFGAGTKLELK

SEQ ID NO: 102 - protein, 449 aa, synthetic construct
EIQLQQSGPELVKPGASVKVSCKASGYAFTSYNMYWVKQSHGKSLEWIGYIDPYNGGTSYNQKFKGKATLTVDKSSSTAYMHLNSLTSEDSAVYYCARGYGNYKAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 213 aa, synthetic construct
DAVMTQTPKFLLVSAGDRVTITCKASQSVTNDVAWYQQKPGQSPKLLIYYASNRYTGVPDRFTGSGYGTDFTFTISTVQAEDLAVYFCQQDYSSLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC